(19) United States
(12) Patent Application Publication
(10) Pub. No.: US 2025/0009813 A1
(43) Pub. Date: Jan. 9, 2025

(54) BROAD HOST RANGE CONJUGATIVE PLASMIDS

(71) Applicants: The Westmead Institute for Medical Research, Westmead, New South Wales (AU); Western Sydney Local Health District, Westmead, New South Wales (AU)

(72) Inventors: Muhammad KAMRUZZAMAN, Westmead, New South Wales (AU); Jonathan IREDELL, Westmead, New South Wales (AU)

(21) Appl. No.: 18/713,524

(22) PCT Filed: Nov. 25, 2022
(86) PCT No.: PCT/AU2022/051416

§ 371 Date: May 24, 2024

(30) Foreign Application Priority Data
Nov. 25, 2021 (AU) 2021903811

(51) Int. Cl. A61K 35/741; A01N 63/20; A01N 63/60; A01P 1/00; A61K 35/00; A61K 38/16; C12N 15/70; C12N 15/74

(52) U.S. Cl. CPC A61K 35/741; A01N 63/20; A01N 63/60; A01P 1/00; A61K 38/164; C12N 15/70; C12N 15/74; A61K 2035/115; C12N 2800/101

(57) ABSTRACT

The present disclosure generally relates to a broad-host-range conjugative system and plasmid comprising same which are capable of efficient transfer in vivo to a wide range of bacteria of the Enterobacteriaceae family. The present disclosure also relates to recombinant plasmid comprising the broad-host-range conjugative system and the use of same for curing or excluding plasmids encoding target antimicrobial resistance (AMR) genes and/or genes conferring other virulence attributes.

### CROSS-REFERENCE TO RELATED APPLICATIONS

The present application claims priority from Australian Provisional Patent Application No 2021903811 filed on 25 Nov. 2021, the content of which is incorporated herein by reference in its entirety.

### TECHNICAL FIELD

The present disclosure generally relates to a broad-host-range conjugative system and plasmid comprising same which are capable of efficient transfer in vivo to a wide range of bacteria of the Enterobacteriaceae family. The present disclosure also relates to recombinant plasmid comprising the broad-host-range conjugative system and the use of same for curing or excluding plasmids encoding target antimicrobial resistance (AMR) genes and/or genes conferring other virulence attributes.

### BACKGROUND

Antimicrobials underpin many facets of modern medicine, including treatment of infections, prevention of infection during surgical procedures and protection of immunocompromised patients from infection. However, over the past several decades, the use of antibiotics and other antimicrobials in human medicine, animal husbandry, veterinary practice and aquaculture has increased dramatically. Despite the growing trend towards improved antimicrobial stewardship in many countries, the historical overuse of antimicrobial agents has been selected for many mutant strains of bacteria which are resistant to the commonly used antimicrobials. As a result, one of the major threats facing society is the rise in the number of antimicrobial-resistant (AMR) bacteria, such as Escherichia coli, Klebsiella pneumoniae and Salmonella. The ability of these bacteria to resist the effect of antibiotics is largely mediated by invasive plasmids carrying antimicrobial resistance genes (ARG) which, once acquired, cannot be removed due to the use of ‘addiction systems’.

One strategy that has been developed for combatting AMR bacteria is plasmid curing. ‘Plasmid curing’ is the process by which plasmids harbouring AMR genes and/or genes conferring virulence traits are removed from bacterial populations e.g., such as in a human gut. This is an attractive strategy to combat AMR since it has the potential to remove AMR and/or virulence genes from a population of bacteria whilst leaving the bacterial community largely intact. Strategies of plasmid curing vary greatly, such as the use of chemicals, drugs, natural products, phage therapies, other plasmids and CRISPR/Cas systems to reduce or inhibit plasmid conjugation, reduce or inhibit plasmid replication, disrupt plasmid segregation or by increasing the fitness cost associated with plasmid carriage. The present inventors have previously demonstrated that it is possible to cure AMR using curing plasmids that target plasmid replication and addiction systems. Notwithstanding its attraction, the success of plasmid curing as a therapeutic approach hinges inter alia on the ability to efficiently transfer a curing plasmid to a broad host range of bacteria (e.g., such as clinically-relevant bacteria), which carry problem AMR plasmids.

It would therefore be desirable to develop conjugative plasmids with high transfer efficiencies across a broad host range of bacteria, particularly those which are most clinically relevant.

### SUMMARY

The present disclosure is based, at least in part, on the recognition by the inventors that known delivery vectors for plasmid curing are limited by their ability to efficiently transfer to a broad host range of clinically relevant bacteria in vivo. In this regard, whilst it was known that most large plasmids are self-transmissible (conjugative) in nature, most have a very narrow-host-range. It was also not previously known which conjugative plasmid(s) are capable of efficient transfer in the complex gut system and therefore suitable to deliver a curing system (e.g., such as a CRISPR-Cas or Rep-TA-based system) in vivo.

The present inventors have, inter alia, identified and demonstrated the use of broad-host-range conjugative plasmid vector to efficiently transfer in vivo to a wide range of bacteria of the Enterobacteriaceae family; including E. coli, K. pneumoniae, Morganella spp. In doing so, the inventors have identified the core genetic elements which confer high-level conjugation efficiency. The region comprising the core genetic elements, referred to herein as the “conjugation system”, may be used to construct a highly-efficient recombinant AMR curing plasmid which is capable of transfer to a broad host range. The inventors have also shown that their recombinant curing plasmid is capable of efficient curing of target AMR plasmids in vitro and in vivo.

In one example, the present disclosure provides a recombinant plasmid comprising a plasmid conjugation system comprising a transfer operon comprising a polynucleotide sequence which is at least 70% identical to the sequence set forth in SEQ ID NO: 4 (tra-mob) and a transfer operon comprising a polynucleotide sequence which is at least 70% identity to the sequence set forth in SEQ ID NO: 5 (trb). For example, the recombinant plasmid may comprise a plasmid conjugation system comprising a transfer operon comprising a polynucleotide sequence which is at least 80% identical to the sequence set forth in SEQ ID NO: 4 (tra-mob) and a transfer operon comprising a polynucleotide sequence which is at least 80% identical to the sequence set forth in SEQ ID NO: 5 (trb). For example, the recombinant plasmid may comprise a plasmid conjugation system comprising a transfer operon comprising a polynucleotide sequence which is at least 90% identical to the sequence set forth in SEQ ID NO: 4 (tra-mob) and a transfer operon comprising a polynucleotide sequence which is at least 90% identity to the sequence set forth in SEQ ID NO: 5 (trb). For example, the recombinant plasmid may comprise a plasmid conjugation system comprising a transfer operon comprising a polynucleotide sequence which is at least 95% identical to the sequence set forth in SEQ ID NO: 4 (tra-mob) and a transfer operon comprising a polynucleotide sequence which is at least 95% identity to the sequence set forth in SEQ ID NO: 5 (trb). In one example, the recombinant plasmid comprises a plasmid conjugation system comprising a transfer operon comprising a polynucleotide sequence set forth in SEQ ID NO: 4 (tra-mob) and a transfer operon comprising a polynucleotide sequence set forth in SEQ ID NO: 5 (trb).

In one example, the recombinant plasmid does not comprise one or more genetic regions from the incompatibility group M (Inc M) plasmid designated pJIBE401, which are selected from the group consisting of: a transfer inhibition (tir) system, toxin-antitoxin system (pemIK), error-prone DNA polymerase (mucAB), an entry exclusion (exc) system, and multi-drug resistance (MDR) region with transposable elements. For example, the recombinant plasmid does not comprise one or more genetic regions selected from the group consisting of: a tir system comprising the sequence set forth in SEQ ID NO: 6, pemIK comprising the sequence set forth in SEQ ID NO: 7, mucAB comprising the sequence set forth in SEQ ID NO: 8, an exc system comprising the sequence set forth in SEQ ID NO: 9, and MDR with transposable elements comprising the sequence set forth in SEQ ID NO: 10. In one example, the recombinant plasmid does not comprise two or more (e.g., 2, or 3, or 4) genetic regions selected from the group consisting of a tir system comprising the sequence set forth in SEQ ID NO: 6, pemIK comprising the sequence set forth in SEQ ID NO: 7, a mucAB comprising the sequence set forth in SEQ ID NO: 8, an exc system comprising the sequence set forth in SEQ ID NO: 9, and a MDR-transposable element comprising the sequence set forth in SEQ ID NO: 10. In one example, the recombinant plasmid does not comprise a tir system comprising the sequence set forth in SEQ ID NO: 6. In one example, the recombinant plasmid does not comprise a pemIK system comprising the sequence set forth in SEQ ID NO: 7. In one example, the recombinant plasmid does not comprise a mucAB comprising the sequence set forth in SEQ ID NO: 8. In one example, the recombinant plasmid does not comprise an exc system comprising the sequence set forth in SEQ ID NO: 9. In one example, the recombinant plasmid does not comprise MDR-transposable elements comprising the sequence set forth in SEQ ID NO: 10.

In one example, the recombinant plasmid does not comprise one or more genetic regions selected from the group consisting of: a tir system, a pemIK, a mucAB, an exc system, and MDR-transposable elements. In one example, the recombinant plasmid does not comprise two or more (e.g., 2, or 3, or 4) genetic regions selected from the group consisting of a tir system, pemIK, a mucAB, an exc system, and MDR-transposable elements. In one example, the recombinant plasmid does not comprise a tir system. In one example, the recombinant plasmid does not comprise a pemIK. In one example, the recombinant plasmid does not comprise a mucAB. In one example, the recombinant plasmid does not comprise an exc system. In one example, the recombinant plasmid does not comprise MDR-transposable elements.

In one example, the recombinant plasmid comprises: (a) one or more plasmid replication genes; and/or (c) a plasmid partitioning system. For example, the recombinant plasmid may comprise a plasmid replication gene; and a plasmid partitioning system. In one example, the plasmid replication gene; and/or the plasmid partitioning system may be derived from a plasmid other than the incompatibility group M (Inc M) plasmid designated pJIBE401.

In one example, the recombinant plasmid further comprises one or more antibiotic resistance genes which is/are not present in the multi-resistance region of the incompatibility group M (Inc M) plasmid designated pJIBE401, wherein the one or more antibiotic resistance genes are selected from tetA conferring tetracycline antibiotic resistance, fosA3 conferring fosfomycin antibiotic resistance, and combinations thereof.

In one example, the recombinant plasmid comprises one or more genes of the parAB operon encoding genes participating in partitioning of the plasmid.

In one example, the recombinant plasmid is a curing plasmid. For example, the recombinant plasmid as described herein may comprise one or more genetic elements which confer a plasmid curing function. In one example, the curing plasmid comprises one or more of the following: an expression cassette encoding a CRISPR-Cas system targeting one or more genes conferring a trait which is undesirable in a bacterial population; a region encoding one or more entry exclusion systems; one or more replication genes, one or more antitoxin genes; and/or one or more antibiotic resistance genes. The trait which is undesirable in a bacterial population may be antimicrobial resistance (AMR), bacterial virulence, or heavy metal resistance. In one example, the trait which is undesirable in a bacterial population is encoded by one or more genes of an AMR plasmid, wherein the one or more genes is selected from an antitoxin gene, a replication gene, a multidrug resistance gene (MDR) and combinations thereof. For example, the curing plasmid may comprise an expression cassette encoding a CRISPR-Cas system targeting one or more genes conferring a trait which is undesirable in a bacterial population. For example, the recombinant plasmid may comprise a region encoding one or more entry exclusion systems. For example, the curing plasmid may comprise one or more replication genes. For example, the recombinant plasmid may comprise one or more antitoxin genes. For example, the recombinant plasmid may comprise one or more antibiotic resistance genes.

In one example, the trait which is undesirable in a bacterial population is antimicrobial resistance (AMR), bacterial virulence, or heavy metal resistance. In one particular example, the trait which is undesirable in a bacterial population is encoded by one or more genes of an AMR plasmid, wherein the one or more genes is selected from a toxin-antitoxin gene (e.g., which is required for retention of the AMR plasmid in the host bacteria), a replication gene, a multidrug resistance gene (MDR) and combinations thereof.

In one example, the recombinant plasmid further comprises a region encoding one or more entry exclusion systems (EES). For example, the recombinant plasmid (probiotic plasmid) may comprise a plurality of entry exclusion systems. The one or more EES may be from a plasmid variant of an incompatibility group (Inc) selected from IncF, IncI, IncA, IncC, IncL, IncM, IncN, IncX and IncH, or plasmids with any other known and unknown Inc groups. For example, one or more of the entry exclusion systems may be from Incompatibility (Inc) Group F (Inc F) plasmids. For example, one or more of the entry exclusion systems may be from incompatibility group L (Inc L) plasmids. For example, one or more of the entry exclusion systems may be from incompatibility group C (Inc C) plasmids. For example, one or more of the entry exclusion systems may be from incompatibility group M (Inc M) plasmids. For example, one or more of the entry exclusion systems may be from incompatibility group A (Inc A) plasmids.

In one example, a curing plasmid comprises an antitoxin gene. For example, the curing plasmid may comprise an antitoxin gene which enables the plasmid to be retained within a host bacteria comprising an AMR plasmid comprising a corresponding toxin gene. That is, the antitoxin gene enables the host bacteria to neutralise an effect of the toxin (encoded by the toxin gene) after AMR plasmid curing. In one example, the antitoxin gene is PemI.

The present disclosure also provides a method of producing a recombinant conjugative plasmid having a broad host range, said method comprising introducing to a plasmid backbone a conjugation system comprising a transfer operon comprising a polynucleotide sequence which is at least 70% identical to the sequence set forth in SEQ ID NO: 4 (tra-mob) and a transfer operon comprising a polynucleotide sequence which is at least 70% identity to the sequence set forth in SEQ ID NO: 5 (trb). For example, the recombinant plasmid may comprise a plasmid conjugation system comprising a transfer operon comprising a polynucleotide sequence which is at least 80% identical to the sequence set forth in SEQ ID 35 NO: 4 (tra-mob) and a transfer operon comprising a polynucleotide sequence which is at least 80% identity to the sequence set forth in SEQ ID NO: 5 (trb). For example, the recombinant plasmid may comprise a plasmid conjugation system comprising a transfer operon comprising a polynucleotide sequence which is at least 90% identical to the sequence set forth in SEQ ID NO: 4 (tra-mob) and a transfer operon comprising a polynucleotide sequence which is at least 90% identity to the sequence set forth in SEQ ID NO: 5 (trb). For example, the recombinant plasmid may comprise a plasmid conjugation system comprising a transfer operon comprising a polynucleotide sequence which is at least 95% identical to the sequence set forth in SEQ ID NO: 4 (tra-mob) and a transfer operon comprising a polynucleotide sequence which is at least 95% identity to the sequence set forth in SEQ ID NO: 5 (trb). In one example, the recombinant plasmid comprises a plasmid conjugation system comprising a transfer operon comprising a polynucleotide sequence set forth in SEQ ID NO: 4 (tra-mob) and a transfer operon comprising a polynucleotide sequence set forth in SEQ ID NO: 5 (trb).

In some examples, the recombinant conjugative plasmid is a recombinant plasmid as described herein.

The present disclosure also provides a method of producing a recombinant plasmid for plasmid curing, comprising: (a) obtaining a recombinant plasmid as described herein; and (b) introducing to the recombinant plasmid one or more of the following: an expression cassette encoding a region encoding a CRISPR-Cas system targeting one or more genes conferring a trait which is undesirable in a bacterial population; a region encoding one or more entry exclusion systems; one or more replication genes; one or more antitoxin genes; and/or one or more AMR genes. For example, the trait which is undesirable in a bacterial population may be AMR, bacterial virulence, or heavy metal resistance. For example, the trait which is undesirable in a bacterial population is AMR.

In one example, the trait which is undesirable in a bacterial population is encoded by one or more genes of an AMR plasmid, wherein the one or more genes is selected from an antitoxin gene, a replication gene, a multidrug resistance gene (MDR) and combinations thereof. Accordingly, the method may comprise introducing to the recombinant plasmid one or more genes selected from an antitoxin gene, a replication gene, a multidrug resistance gene (MDR) and combinations thereof. In one example, the antitoxin gene enables the recombinant plasmid to be retained within a host bacteria comprising an AMR plasmid comprising a corresponding toxin gene. That is, the antitoxin gene enables the host bacteria to neutralise an effect of the toxin (encoded by the toxin gene) after AMR plasmid curing. In one example, the antitoxin gene is PemI.

In one example, the method may comprise introducing to the recombinant plasmid one or more genes selected from one or more region encoding a plurality of entry exclusion systems. For example, the method may comprise introducing to the recombinant plasmid one or more entry exclusion systems are from Incompatibility Group F (Inc F).

Also provided herein is a recombinant plasmid for plasmid curing (i.e., a curing plasmid as described herein) produced by the method of the disclosure.

The present disclosure also provides a bacterial cell or a population of bacterial cells comprising a recombinant plasmid or curing plasmid as described herein. Preferably the bacteria is a probiotic bacteria. For example, the probiotic bacteria may be selected from a group consisting of Escherichia, Klebsiella, any members of the Enterobacteriaceae species, or other suitable probiotic.

The present disclosure also provides a composition comprising one or more viable bacterial cells as described herein.

In one example, the composition comprises a pharmaceutically acceptable carrier or diluent.

In one example, one or more viable bacterial cells are lyophilised. The lyophilised bacterial cells may be microencapsulated and/or provided in a capsule.

In one example, the composition is a food or beverage product. For example, the food or beverage product may be a dairy product (e.g., a yoghurt drink).

The present disclosure also provides a method of plasmid curing in a population of bacteria to reduce the prevalence of a plasmid conferring a trait of interest in the population of bacteria (e.g., a population of gut bacteria), said method comprising introducing to the population a curing plasmid as described herein or a bacterial cell as described herein or a pharmaceutical composition as described herein.

In one example, the trait of interest is AMR, bacterial virulence, or heavy metal resistance. In one example, the method reduces the prevalence of a plasmid conferring two or more of AMR, bacterial virulence, and/or heavy metal resistance in a population of bacteria (e.g., a population of gut bacteria).

In accordance with an example in which the population of bacteria is a population of gut bacteria, the method comprises administering the curing plasmid or the one or more bacterial cells or composition comprising same to the gut of a subject in need thereof.

In one example, the subject is a human.

In one example, the subject is a non-human animal. For example, the non-human animal may be a livestock species or companion animal.

In another example, the bacterial population is a population of bacteria which colonise plants and/or soil and the method comprises contacting the plant or soil with the curing plasmid or the bacterial cell or composition comprising same.

In another example, the population of bacteria is present in an environment selected from a healthcare environment, a soil environment, an environment comprising a water source, an environment comprising waste water, an environment comprising industrial waste, an environment comprising agricultural waste, an environment comprising sewerage and/or an environment comprising bio-solids, and the method comprises introducing curing plasmid or the bacterial cell or the composition to the environment.

In each of the foregoing example, the method of the disclosure may further comprise administering one or more antibiotic agents to the bacterial population. For example, the method may reduce the prevalence of a plasmid conferring AMR in the population of bacteria rendering the bacteria susceptible to one or more antibiotic agents.

The present disclosure also provides for use of a curing plasmid or a bacterial cell or composition comprising same as described herein in the preparation of a medicament for treating or preventing antibiotic resistance in a subject.

In one example, the subject is a human.

In one example, the subject is a non-human animal. For example, the non-human animal may be a livestock species or companion animal.

### BRIEF DESCRIPTION OF THE DRAWINGS

FIG. 1. Conjugation frequency of plasmids to different Enterobacteriaceae species: A) Plasmid transfer rates from donor E. coli to recipient E. coli bacteria; B) Plasmid transfer rates from donor E. coli to recipient K. pneumoniae bacteria; and C) Plasmid transfer rates from donor E. coli to recipient M. morganii bacteria.

FIG. 2. Genetic structure of fully sequenced IncM plasmid pJIBE401.

FIG. 3. Genetic structure of IncM plasmid pJIBE401 with multi-drug resistance region and transposable elements of IncM plasmid pJIBE401 identified by orange boxes. These regions are not essential for conjugative transfer.

FIG. 4. Genetic structure of pJIMK44_ΔMRR_M plasmid.

FIG. 5. Genetic structure of pJIMK44_ΔMRR_M plasmid with additional genes identified as being unnecessary for conjugative transfer identified by purple boxes.

FIG. 6. Genes structure of pJIMKCore_M (core conjugative region of pJIBE401).

FIG. 7. Conjugation frequency of plasmid pJIBE401 and its derivatives.

FIG. 8. Conjugation efficiency of pJIMKCore_M in vivo in mouse gut.

FIG. 9. In vitro curing of plasmids by using pJIMKCore_M-based curing plasmids.

FIG. 10. In vivo curing of IncC and pKPC_UVA01 plasmids using pJIMKCore_M-based curing plasmid in mice.

### KEY TO THE SEQUENCE LISTING

SEQ ID NO: 1 Sequence of incompatibility group M (IncM) plasmid pJIBE401.

SEQ ID NO: 2 Sequence of recombinant conjugative plasmid pJIMKCore_M.

SEQ ID NO: 3 Sequence of core region of the plasmid pJIMKCore_M.

SEQ ID NO: 4 Sequence of tra-mob gene cluster present in the conjugative plasmid pJIMKCore_M.

SEQ ID NO: 5 Sequence of trb operon present in the conjugative plasmid pJIMKCore_M.

SEQ ID NO: 6 Sequence of tir genes present in the conjugative plasmid pJIBE401.

SEQ ID NO 7 Sequence of toxin-antitoxin gene pair pemIK present in the conjugative plasmid pJIBE401.

SEQ ID NO: 8 Sequence of mucAB gene operon present in the conjugative plasmid pJIBE401.

SEQ ID NO: 9 Sequence of exc genes present in the conjugative plasmid pJIBE401.

SEQ ID NO: 10 Sequence of multi-drug resistance and transposons present in the conjugative plasmid pJIBE401.

### DETAILED DESCRIPTION

### General Techniques

Throughout this specification, unless specifically stated otherwise or the context requires otherwise, reference to a single step, feature, composition of matter, group of steps or group of features or compositions of matter shall be taken to encompass one and a plurality (i.e. one or more) of those steps, features, compositions of matter, groups of steps or groups of features or compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant DNA, recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbor Laboratory, (1988), and J. E. Coligan et al. (editors) Current Protocols in Immunology, John Wiley & Sons (including all updates until present).

Throughout this specification, unless the context requires otherwise, the word “comprise”, or variations such as “comprises” or “comprising”, is understood to imply the inclusion of a stated step or element or integer or group of steps or elements or integers but not the exclusion of any other step or element or integer or group of elements or integers.

The term “and/or”, e.g., “X and/or Y” shall be understood to mean either “X and Y” or “X or Y” and shall be taken to provide explicit support for both meanings or for either meaning.

The words “a” and “an” when used in this disclosure, including the claims, denotes “one or more.”

As used herein, the terms “about” and “approximately” are interchangeable, and should generally be understood to refer to a range of numbers around a given number, as well as to all numbers in a recited range of numbers. Moreover, all numerical ranges herein should be understood to include each whole integer within the range.

The terms “e.g.,” and “i.e.” as used herein, is used merely by way of example, without limitation intended, and should not be construed as referring only those items explicitly enumerated in the disclosure.

### Plasmids

Existing delivery vectors for plasmid curing are limited by their ability to efficiently transfer to a broad host range of clinically relevant bacteria in vivo. Whilst it is known that most large plasmids are self-transmissible (conjugative) in nature, most have a very narrow-host-range. It is also not well-characterised, which conjugative plasmid(s) are capable of efficient transfer in the complex gut system and therefore suitable to deliver a curing system (e.g., such as for AMR) in vivo.

The present inventors have identified a minimal plasmid conjugation system, which is capable of efficient transfer in vivo to a wide range of bacteria, including, but not limited to, bacteria of the Enterobacteriaceae family. The inventors have found that this minimal plasmid conjugation system may be used to construct recombinant plasmids e.g., such as for use in ‘plasmid curing’ or delivery of genetic cargoes, which are capable of efficient transfer to a broad host range of clinically relevant bacteria in vivo.

As used herein, a “plasmid” will be understood to mean a circular, double-stranded DNA molecule, which forms an extrachromosomal self-replicating genetic element that can be used as a vehicle for introducing a nucleic acid into bacterial and eukaryotic cells. Bacterial plasmids are usually circularly covalently closed and supercoiled. Plasmids may be transmitted from one bacterium to another (including other species of bacterium) through a process known as “conjugation”. This host-to-host transfer of genetic material is one mechanism of horizontal gene transfer. A “conjugative plasmid” as used herein will therefore be understood to be a plasmid which is capable of host-to-host or horizontal transfer. Similarly, a “conjugation system” as used herein will be understood to mean the genetic region(s) or element(s) which confer the ability of a plasmid to be transmitted from one bacterium to another through the process of “conjugation”.

In general, conjugative transfer of bacterial plasmids is the primary route of broad host range DNA transfer between different genera of bacteria and is the most efficient way of horizontal gene spread. The conjugative transfer is thus considered one of the major reasons for the increase in the number of bacteria exhibiting multiple-antibiotic resistance. Various aspects of the present disclosure are directed to exploiting the natural phenomena of conjugative transfer of plasmids to deliver the desired cargo to microbial cells in vivo, which eliminates and/or reverses, for example, antibiotic resistance in the host bacterial cells.

Plasmid conjugation can be used to introduce a desired plasmid from a “donor” microbial cell to a recipient microbial cell. In some examples, plasmid conjugation can genetically modify a recipient microbial cell by introducing a conjugation plasmid from a donor microbial cell to a recipient microbial cell. Without being limited by any particular theory, conjugative plasmids that comprise the same or functionally same set of replication genes typically cannot coexist in the same microbial cell. As such, in some examples, plasmid conjugation can “reprogram” a recipient microbial cell by introducing a new conjugation plasmid to supplant another conjugation plasmid that was present in the recipient cell. In some examples, plasmid conjugation is used to engineer (or reengineer) a microbial cell with a particular combination of one or more antitoxin genes.

The term “recombinant”, as used in the context of a plasmid of the disclosure, shall be understood to mean a plasmid comprising one or more genetic elements or portions thereof which are not normally found together within a plasmid in nature or which are found in a different orientation within a plasmid in nature. For example, a recombinant plasmid may be made using a plasmid backbone and components from one or more plasmids capable of expressing one or more DNA expression cassettes or operons.

In one example, the present disclosure provides a recombinant plasmid comprising a plasmid conjugation system comprising (i) a transfer operon comprising a polynucleotide sequence which is at least 70% identical to the sequence set forth in SEQ ID NO: 4 (tra-mob) and (ii) a transfer operon comprising a polynucleotide sequence which is at least 70% identity to the sequence set forth in SEQ ID NO: 5 (trb).

Plasmid transfer (tra-mob) genes are necessary for the conjugative transfer of the plasmids. Most tra-mob genes are found in one operon. The ability of the donor bacteria to perform conjugation requires the expression of the transfer genes clustered in the tra-mob region of the plasmid. The transfer genes encode all the protein factors involved in the elaboration of the conjugative pilus and the type 4 secretion system (T4SS) required for the formation of the mating pair, as well as the relaxosome components needed for the processing of the plasmid prior to transfer. Mob genes are required for conjugative DNA processing. Besides, a membrane-associated mating pair formation (MPF) complex, which is a form of a type 4 secretion system (T4SS), provides the mating channel. A plasmid that codes for its own set of MPF genes is called self-transmissible or conjugative. The classification of conjugative systems has been divided into six MOB families: MOB_{F}, MOB_{H}, MOB_{Q}, MOB_{C}, MOB_{P}, and MOB_{V}

In one example, the recombinant plasmid of the disclosure comprises a tra-mob gene cluster comprising a polynucleotide sequence which is at least 70% (e.g., at least 75%, or at least 80%, or at least 85%, or at least 90%, or at least 95%, or at least 96%, or at least 97%, or at least 98%, or at least 99%) identical to the sequence set forth in SEQ ID NO: 4 (tra-mob). For example, the recombinant plasmid of the disclosure may comprise a Tra genes cluster comprising a polynucleotide sequence set forth in SEQ ID NO: 4.

Transfer operon trb genes are also known as plasmid transfer genes and are generally found together in an operon. These genes are involved in conjugative transfer along with tra-mob genes.

In one example, the recombinant plasmid of the disclosure comprises a Trb operon comprising a polynucleotide sequence which is at least 70% (e.g., at least 75%, or at least 80%, or at least 85%, or at least 90%, or at least 95%, or at least 96%, or at least 97%, or at least 98%, or at least 99%) identical to the sequence set forth in SEQ ID NO: 5 (trb). For example, the recombinant plasmid of the disclosure may comprise a Trb operon comprising a polynucleotide sequence set forth in SEQ ID NO: 5.

For example, the recombinant plasmid may comprise a plasmid conjugation system comprising a transfer operon comprising a polynucleotide sequence which is at least 70% identical to the sequence set forth in SEQ ID NO: 4 (tra-mob) and a transfer operon comprising a polynucleotide sequence which is at least 70% identity to the sequence set forth in SEQ ID NO: 5 (trb). For example, the recombinant plasmid may comprise a plasmid conjugation system comprising a transfer operon comprising a polynucleotide sequence which is at least 75% identical to the sequence set forth in SEQ ID NO: 4 (tra-mob) and a transfer operon comprising a polynucleotide sequence which is at least 75% identity to the sequence set forth in SEQ ID NO: 5 (trb). For example, the recombinant plasmid may comprise a plasmid conjugation system comprising a transfer operon comprising a polynucleotide sequence which is at least 80% identical to the sequence set forth in SEQ ID NO: 4 (tra-mob) and a transfer operon comprising a polynucleotide sequence which is at least 80% identity to the sequence set forth in SEQ ID NO: 5 (trb). For example, the recombinant plasmid may comprise a plasmid conjugation system comprising a transfer operon comprising a polynucleotide sequence which is at least 85% identical to the sequence set forth in SEQ ID NO: 4 (tra-mob) and a transfer operon comprising a polynucleotide sequence which is at least 85% identity to the sequence set forth in SEQ ID NO: 5 (trb). For example, the recombinant plasmid may comprise a plasmid conjugation system comprising a transfer operon comprising a polynucleotide sequence which is at least 90% identical to the sequence set forth in SEQ ID NO: 4 (tra-mob) and a transfer operon comprising a polynucleotide sequence which is at least 90% identity to the sequence set forth in SEQ ID NO: 5 (trb). For example, the recombinant plasmid may comprise a plasmid conjugation system comprising a transfer operon comprising a polynucleotide sequence which is at least 95% identical to the sequence set forth in SEQ ID NO: 4 (tra-mob) and a transfer operon comprising a polynucleotide sequence which is at least 95% identity to the sequence set forth in SEQ ID NO: 5 (trb). In one example, the recombinant plasmid comprises a plasmid conjugation system comprising a transfer operon comprising a polynucleotide sequence set forth in SEQ ID NO: 4 (tra-mob) and a transfer operon comprising a polynucleotide sequence set forth in SEQ ID NO: 5 (trb).

Whilst the recombinant plasmid of the disclosure comprises a conjugation system which is genetically identical or similar to that of the IncM plasmid designated pJIBE401 as described herein, it does not comprise one or more other genetic regions of the pJIBE401 plasmid. The one or more genetic regions of the IncM plasmid designated pJIBE401 may be selected from the group consisting of: a transfer inhibition (tir) system, toxin-antitoxin system (pemIK), error-prone DNA polymerase (mucAB), an entry exclusion (exc) system, and MDR with transposable elements. In this regard, the inventors have shown that certain other genetic elements within the IncM plasmid designated pJIBE401 are not essential for efficient conjugation and may be remove or omitted from a recombinant plasmid comprising the conjugation system of the disclosure. This may have the advantage of reducing the size of the plasmid and/or freeing up space in the plasmid for the inclusion of other genetic elements and/or cargos.

For example, the recombinant plasmid may not comprise one or more genetic regions from the incompatibility group M (Inc M) plasmid designated pJIBE401 selected from the group consisting of: a transfer inhibition (tir) system, toxin-antitoxin system (pemIK), error-prone DNA polymerase (mucAB), an entry exclusion (exc) system, and MDR-transposable elements. It is well known in the art that MucAB is error-prone DNA polymerase and works to inhibit the homologous recombination, similar to UmuDC (Claude Venderbure et al., J Bacteriol. 1999 February; 181 (4): 1249-55). Whereas, Exc is a plasmid entry-exclusion gene that inhibits the repetitive transfer of the same plasmid in a recipient bacteria and transfer inhibition (tir) gene inhibits the transfer of the plasmid to recipient bacteria (Potron et al., Antimicrob Agents Chemother. 2014; 58 (1): 467-71).

For example, the recombinant plasmid may not comprise one or more genetic regions selected from the group consisting of: a tir system comprising the sequence set forth in SEQ ID NO: 6, pemIK comprising the sequence set forth in SEQ ID NO: 7, mucAB comprising the sequence set forth in SEQ ID NO: 8, an exc system comprising the sequence set forth in SEQ ID NO: 9, and MDR with transposable element comprising the sequence set forth in SEQ ID NO: 10. In some examples, the recombinant plasmid does not comprise two or more (e.g., 2, or 3, or 4) of the genetic regions selected from the group consisting of a tir system comprising the sequence set forth in SEQ ID NO: 6, pemIK comprising the sequence set forth in SEQ ID NO: 7, mucAB comprising the sequence set forth in SEQ ID NO: 8, an exc system comprising the sequence set forth in SEQ ID NO: 9, and MDR with transposable element comprising the sequence set forth in SEQ ID NO: 10.

In addition to the conjugation system described herein, the recombinant plasmid may comprise (a) one or more plasmid replication genes and/or (b) a plasmid partitioning system.

The recombinant plasmid of the disclosure may comprise a plasmid partitioning system. A plasmid partition system aids to prevent random segregation of expression plasmids, thereby enhancing inheritance and stability. Stable inheritance of a plasmid generally requires that: (1) the plasmid must replicate once each generation, (2) copy number deviations must be rapidly corrected before cell division, and (3) upon cell division, the products of plasmid replication must be distributed to both daughter cells. Exemplary partitioning functions include, without limitation, systems of pSC101, the F factor, the P1 prophage, and incFII drug resistance plasmids, parAB genes in IncM plasmids. For example, the recombinant plasmid may comprise parAB gene from an IncM plasmid. In one example, the plasmid partitioning system comprises a sequence having at least 70% (e.g., at least 70%, or at least 75%, or at least 80%, or at least 85%, or at least 90%, or at least 95%, or at least 96%, or at least 97%, or at least 98%, or at least 99%) identity to the parAB sequence in the pJIMKCore_M core plasmid. For example, the recombinant plasmid may comprise parAB gene from a IncM plasmid.

The recombinant plasmid of the disclosure may comprise an origin of replication (Ori) or replication gene or replicon. The Ori allows many copies of the plasmid to be produced in a bacterial cell without integration of the plasmid into the host cell DNA. The term “origin of replication” therefore refers to a DNA sequence, which promotes the initiation of replication of the plasmid (e.g., genomic DNA, covalently closed circular recombinant DNA). Whilst replication genes are responsible for the initiation and completion of plasmid replication in the cell and determine the plasmid copy number, and they are not essential for conjugation. Thus, a skilled person may select an appropriate replication gene independent of the choice of conjugation system in the recombinant plasmid.

The choice of Ori will depend on a number of factors, including, but not limited to, the host bacteria in which the plasmid will be replicating, whether the plasmid is to replicate in high copy number or low copy number, the conditions under which replication will occur (e.g., growth conditions for the host bacteria), and compatibility/incompatibility between plasmids. A skilled person would be able to select an appropriate ori from those known in the art, depending on the intended use of the recombinant plasmid of the disclosure. The present disclosure contemplates the use of plasmids with broad-host-range origins of replication. Such plasmid origins include, without limitation, those derived from major broad-host-range incompatibility groups IncM (e.g. repA, repC), IncQ (e.g., RSF1010, R300B, R1162), IncW (e.g., pSa, pR388), IncP (e.g., R18, R68, RK2, RP1, RP4), IncN and IncU (e.g., RA3), IncF plasmids or pBBR1 (Lale, R., et al. Strain Engineering, Vol. 765 (ed. Williams, J. A.) 327-343 (Humana Press, 2011)). Other broad-host-range and conjugative plasmids may be used in accordance with the present disclosure.

In some examples, the replication gene is selected on the basis of compatibility/incompatibility between plasmids. In this regard, a skilled person will be aware that two plasmids with the same incompatible replicons cannot exist together in the same cell. This incompatibility property may therefore be exploited to produce a recombinant plasmid of the disclosure, which is useful in plasmid curing (as described herein).

A skilled person will appreciate that any number of plasmid backbones could be used for constructing the recombinant plasmid of the disclosure. However, exemplary conjugative plasmid backbones include pJIBE401 (SEQ ID NO: 1), and pJIMKCore_M (SEQ ID NO: 2) as described herein. Such backbones may be modified to carry, inter alia, genes coding for replication and partitioning systems, as well as one or more antitoxin genes or CRISPR-Cas genes.

As described herein, the recombinant plasmid of the disclosure may be used to produce a plasmid useful in plasmid curing (also referred to herein as a curing plasmid). The term “curing plasmid” as used herein shall be understood to mean a recombinant plasmid of the disclosure capable of removing or lowering the prevalence or spread of one or more plasmids in a bacterial population which confer an undesirable trait of interest. In accordance with examples in which the recombinant plasmid of the disclosure is a curing plasmid, the plasmid may comprise one or more genetic elements which confer a plasmid curing function. In one example, the curing plasmid may comprise one or more of the following: an expression cassette encoding a CRISPR-Cas system targeting one or more genes conferring a trait which is undesirable in a bacterial population; a region encoding one or more entry exclusion systems; one or more replication genes, one or more antitoxin genes; and/or one or more antibiotic resistance genes. The trait which is undesirable in a bacterial population may be antimicrobial resistance (AMR), bacterial virulence, or heavy metal resistance. Accordingly, the trait which is undesirable in a bacterial population may be encoded by one or more genes of an AMR plasmid. For example, the one or more genes may be selected from an antitoxin gene, a replication gene, a multidrug resistance gene (MDR) and combinations thereof. For example, the curing plasmid may comprise an expression cassette encoding a CRISPR-Cas system targeting one or more genes conferring a trait, which is undesirable in a bacterial population. For example, the curing plasmid may comprise a region encoding one or more entry exclusion systems. For example, the curing plasmid may comprise one or more replication genes. For example, the curing plasmid may comprise one or more antitoxin genes. For example, the curing plasmid may comprise one or more antibiotic resistance genes.

In one example, the trait which is undesirable in a bacterial population is antimicrobial resistance (AMR), bacterial virulence, or heavy metal resistance. In one particular example, the trait which is undesirable in a bacterial population is encoded by one or more genes of an AMR plasmid, wherein the one or more genes is selected from an antitoxin gene (e.g., which is required for retention of the AMR plasmid in the host bacteria), a replication gene, a multidrug resistance gene (MDR) and combinations thereof.

The multi-drug resistance may include antibiotic resistant genes selected from one or more of KPC, NDM, OXA, VIM, IMP, CTX-M, IMI, SME, GIM, SPM, NMC or CcrA, SFC, SHV, TEM, BEL, VEB, GES, PER, SFO, BES, TLA, ACC, CMY, MIR, ACT, DHA, MOX, FOX, or CFE or MRSA where these genes are Klebsiella pneumoniae carbapenemase (KPC), New Delhi Metallo-beta-lactamase (NDM) Verona integron-encoded metallo-β-lactamase (VIM), IMP-type carbapenemase (IMP), OXA beta-lactamase (OXA) genes found in carbapenem resistant gram negative bacteria, CTX-M beta-lactamase (CTX-M) gene found in extended spectrum resistant gram-negative bacteria. VanA found in vancomycin resistant Enterococcus, IMI-type carbapenemase (IMI), SME-type carbapenemase (SME), GIM-type carbapenemase (GIM), SPM-type carbapenemase (SPM), NMC-type carbapenemase (NMC), SFC-type carbapenemase (SFC) genes found in carbapenem resistant gram negative bacteria. SHV beta-lactamase (SHV), TEM beta-lactamase (TEM), BEL beta-lactamase (BEL), VEB beta-lactamase (VEB), GES beta-lactamase (GES), PER beta-lactamase (PER), SFO beta-lactamase (SFO), BES beta-lactamase (BES), TLA beta-lactamase (TLA) genes found in extended spectrum resistant gram-negative bacteria, ACC beta-lactamase (ACC), CMY beta-lactamase (CMY), MIR beta-lactamase (MIR), ACT beta-lactamase (ACT), DHA beta-lactamase (DHA), MOX beta-lactamase (MOX), FOX beta-lactamase (FOX), or CFE beta-lactamase (CFE) genes or Methicillin-resistant Staphylococcus aureus (MRSA), which might confer resistance to one or more antibiotics such as but not limited to carbapenem antibiotics, beta-lactamase inhibitors or beta-lactam combination, vancomycin, cephalosporin, cephalothin, cefazolin, cefoxitin, most penicillins.

As described herein, conjugative plasmids of the present disclosure may be used to carry potentially therapeutic payloads such as components of the CRISPR-Cas system into the host bacterial cell or population. In general, bacteria have evolved the CRISPR-Cas system defence mechanism to cope with various environmental stressors, including virus (e.g., bacteriophage) attack. By utilizing components of the CRISPR-Cas system using the specialised curing plasmids of the present disclosure, for example, the AMR resident plasmids could be targeted and eliminated. Gene editing that utilizes RNA-guided DNA that targets the immune principle of CRISPR-Cas (Clustered Regularly Interspersed Short Palindromic Repeats-CRISPR associated proteins) has been widely used. A major advantage provided by the bacterial type II CRISPR-Cas system is that it has minimal requirements for configurable DNA interference (endonucleases, Cas9, customizable duplex RNA structures). Cas9 is a multi-domain that uses an HNH nuclease domain that cleaves the target strand (defined as complementary to the spacer sequence of the crRNA) and a RuvC-like domain that cleaves the non-target strand. It is an enzyme and allows the conversion of dsDNA-cleaved Cas9 to nickase by inactivation of a selective motif. The specificity of DNA cleavage is variable in two parameters: the sequence from the spacer of the crRNA that targets the protospacer (the protospacer is complementary to the spacer of the crRNA, and the short sequence, which is determined by the PAM (the Protospacer Adjunct Motif) located immediately downstream of the protospacer on its non-target DNA strand. Thus, in some examples, the recombinant plasmid could comprise one or more components of the CRISPR-Cas system targeting one or more pathogenic or AMR or virulent plasmids.

In other examples, conjugative plasmids of the present disclosure may comprise one or more antitoxin genes to enable a bacteria comprising the conjugative plasmid to neutralise the effect of toxin expressed from an AMR plasmid after AMR plasmid curing. In this regard, there are strategies adopted by many plasmids that prevent plasmid-free segregants from surviving. Terms such as killer system, killing-anti-killing, post-segregational killing, toxin-antitoxin, poison-antidote, plasmid addiction system or programmed cell death are all used to describe the situation when the host cell is selectively killed if it has not received any copy of the plasmid. The molecular basis of this killing requires the existence of at least two plasmid genes: one specifying a stable toxic agent, and another coding for an unstable factor, which prevents lethal action of the gene encoding the toxic agent. While the toxins identified so far are always proteins, the antidote may be either antisense RNA (which inhibits translation of toxin mRNA) or a protein (that prevents the effect of the toxin in one way or another). Hence, a significant problem encountered by researchers when attempting to cure a bacterium of its endogenous plasmids, is that unfortunately, many plasmids encode a so-called toxin-antitoxin system (TAS), which causes loss of viability of the bacterial cells that have lost their endogenous plasmid. This happens because the endogenous plasmid leaves behind either (i) protein, which becomes toxically active after loss of the plasmid; or (ii) mRNA, which is translated to produce a toxin. The action of the toxin, which is lethal to the host, is normally kept in check either: (i) by regulators, which control the expression of the mRNA that is left behind; by (ii) antidote proteins, which counteract the toxic effects of the toxin; or by (iii) antisense RNA, which binds to and neutralises the effects of the toxic mRNA. The regulators, the antidote proteins, and the antisense RNA are all encoded by the endogenous plasmid, and are unstable, and therefore decay once the endogenous plasmid that encodes them is no longer present in the host. Therefore, the result is death of the bacteria, from which the endogenous plasmid has been displaced. So that the curing plasmid of the disclosure is able to transfer to and produce antitoxin that enables the bacteria to neutralise toxin effect after AMR plasmid curing, the curing plasmid of the present disclosure may comprise one or more antitoxin genes.

As used herein, “antitoxin” refers to a polypeptide or RNA molecule that inhibits the toxic activity of a toxin. The activity of an antitoxin may derive from an RNA transcript of a nucleic acid, an amino acid sequence encoded by a nucleic acid, or an RNA molecule. In some examples, the bacterial host can include one or more antitoxin gene encoding an antitoxin cognate to the toxin encoded by the target pathogenic bacterial population. The exogenous antitoxin gene can be operably linked to a native or heterologous promoter, preferably a regulatable promoter, such as, for example, an inducible promoter or a repressible promoter. The heterologous promoter operably linked to the exogenous antitoxin gene can activate transcription during permissive growth conditions, for example, during contained growth and/or production conditions.

In some examples, the antitoxin gene may encode an antitoxin of the CcdA antitoxin family, RelB antitoxin family, PemI/MazF antitoxin family, MazE antitoxin family, ParD antitoxin family, PIN antitoxin family, MNT antitoxin family, Phd antitoxin family, VapB antitoxin family, zeta antitoxin family, and/or HipB antitoxin family. Additionally or alternatively, the antitoxin gene can be selected from the group consisting of cyanobacterial homologs of axe, phd, mazE, hicB, vapB, pemI, relB, parD, kiS, ccdA, yafN, stbD, yoeM, dinJ, PIN, and combinations thereof. The exogenous antitoxin gene can be homologous or heterologous with respect to the host microorganism.

In one example, the recombinant plasmid could comprise one or more aforementioned antitoxin genes from multiple incompatibility groups of bacteria, including their homologs, orthologs and variants thereof.

The recombinant plasmid of the disclosure may also comprise one or more entry exclusion systems (other than those entry exclusion systems which are explicitly excluded as described herein). As used herein, an “entry exclusion system” or “EES” will be understood to mean a genetic element or system which permits entry exclusion. “Entry exclusion” denotes a property of plasmids by which the cells that contain them become poor recipients to similar plasmids during additional conjugation rounds. A plasmid's entry exclusion system operates by inhibiting the physical entry of an incoming plasmid into a cell where that incoming plasmid exhibits the cognate exclusion phenotype. The inclusion of an entry exclusion system frees a plasmid from competition with related plasmids at segregation during bacterial division. Thus, entry exclusion (i) prevents incompatible incoming plasmids from eliminating a pre-existing (e.g. less undesirable but closely related) plasmid within the host cell, (ii) avoids uneconomical excess of DNA transfer and (iii) averts death of the recipient cell by lethal zygosis.

Entry exclusion proteins from diverse sources are not equal in effectiveness, but exhibit variable exclusion indices that can be increased by over-expressing the exclusion protein. As used herein, the term “exclusion index” refers to the transfer frequency of a given plasmid to a plasmid-free recipient divided by the frequency of transfer to a recipient carrying the same plasmid. For instance, plasmid F showed an EI of 100-300 in mating between Escherichia coli since it transferred 100-300 times better to a plasmid-free recipient than to an F+ recipient (Achtman et al., 1977, Proc Natl Acad Sci USA, 74 (11): 5104-8, and Skurray et al., 1976, Mol Gen Genet, 146 (2): 161-5 In a preferred example, the exclusion index or protection index is the ratio of conjugation frequency to the empty recipient (without exclusion system) and to the recipient bacteria with a probiotic plasmid (with cloned exclusion gene/s). The exclusion index indicates the fold of plasmid transfer inhibition. Thus, the higher exclusion index value indicates stronger protection from the acquisition of plasmid tested.

The recombinant plasmid as described herein may comprise a plurality of EES from a plurality of plasmid variants. In one example, the recombinant conjugative plasmid comprises EES from two plasmid variants comprising a gene conferring the trait of interest. In one example, the recombinant conjugative plasmid comprises EES from at least three (e.g., 3, or 4, or 5, or 6, or 7, or 8, or 9, or 10 or more) plasmid variants comprising a gene conferring the trait of interest. In some examples, the two or more of the EES within the recombinant conjugative plasmid are from the same incompatibility group. In some examples, all of the EES within the recombinant conjugative plasmid are from the same incompatibility group. In other examples, the EES within the recombinant conjugative plasmid are from different incompatibility groups (e.g., the EES may be derived from 2, 3, 4 or more different incompatibility groups).

Incompatibility grouping represents the inability of two plasmids to coexist stably over a number of generations in the same bacterial cell line. Plasmid which are incompatible with one another are assigned to the same “incompatibility group” or “Inc”. Conversely, plasmids which are categorised in different incompatibility groups may be able to co-exist in the same bacterial cell. “Plasmid incompatibility” therefore refers to the inability of plasmids to co-exist, stably, within the same cell when they have similar or identical systems for plasmid replication and/or plasmid partition, i.e. the segregation of each plasmid into daughter cells during cell division along with entry exclusion genes. Two incompatible plasmids, which occupy the same cell would, in the absence of a selective pressure for both plasmids, tend to segregate or partition to different cells during cell division. The stable intracellular co-existence of one plasmid with another requires that each plasmid is able to control, independently of the other, its own replication/partition such that it can establish and maintain a stable copy number. However, the inability of a given plasmid to maintain a stable copy number in the presence of another plasmid is the characteristic feature of incompatibility. Competition for cell resources can result when two plasmids of the same incompatibility group are found in the same cell. Whichever plasmid is able to replicate faster, or provides some other advantage, will typically be represented to a disproportionate degree among the copies allowed by the incompatibility system. Surprisingly, plasmids can also be incompatible when they both possess the same functions for partitioning themselves into daughter cells.

Plasmids typically fall into only one of the many existing incompatibility groups. There are more than 30 known incompatibility groups. Examples include, but are not limited to; IncN, IncW, IncL, IncM, IncT, IncU, IncW, IncY, IncB/O, IncI1, IncK, IncCom9, IncFI, IncFII, IncFIII, IncHI1, IncHI2, IncX, IncA, IncC, IncD, IncFIV, IncFV/FO, IncFVI, IncHI3, IncI2, IncI, IncJ, IncV, IncP, IncQ, and the like, including variants thereof. Accordingly, in some examples, one or more of the EES in the recombinant conjugative plasmid is derived from a plasmid variant of an incompatibility group (Inc) selected from those described hereinabove. In some examples, one or more (or all) of the EES in the recombinant conjugative plasmid are from a plasmid variant of an incompatibility group (Inc) selected from but not limited to IncF, IncI, IncA, IncC, IncL, IncM, IncN, IncX, IncP, IncB/O, and IncH. In one particular example, one or more of the EES are from a plasmid variant of IncF. In another example, all of the EES are from a plasmid variant of IncF. For example, the recombinant conjugative plasmid may comprise EES from at least 2 (e.g., at least 3, or 4, or 5, or 6, or 7, or 8, or 9, or 10 or more) plasmid variants of IncF.

As described herein, in some examples the entry exclusion systems may be derived from plasmid variants of incompatibility group F, which is the largest group in the AMR plasmids. Due to their considerable variations in their replication systems/genes, it has been a challenge up until this point to cure, and/or protect against, all plasmids within IncF using existing plasmid-based approaches (See e.g., Kamruzzaman M et al., PLoS One 12, e0172913, 2017 and Bikard et al., Nat Biotechnol 32, 1146-50, 2014). The F plasmid exerts exclusion using two different EES genes, traT and traS. The majority of F plasmid exclusion activity is thought to be attributable to TraS (EI of around 200, versus 20 for TraT). The EI of F plasmid EES is also thought to be gene dosage dependent since. In this regard, Achtman et al., (1977) and Skurray et al., (1976) showed that when traS and traT were cloned in a multicopy plasmid, the EI increased to 10,000. In one example, one or more of the EES in the recombinant conjugative plasmid are traS gene variants.

Alternatively, or in addition, the EES may be derived from plasmid variants of incompatibility group L (IncL), incompatibility group C (IncC), incompatibility group M (IncM) and/or incompatibility group A (IncA). This may an alternative or in addition to the EES from a plasmid variant of incompatibility group F as described in the foregoing examples.

Any one or more or all of the genetic elements comprised within the recombinant plasmid of the disclosure may be operably-linked to one or more regulatory elements e.g., such as a promoter. The term “promoter” as used herein shall be understood to define a regulatory DNA sequence, generally located upstream of a gene or sequence to be expressed, that mediates the initiation of transcription by directing RNA polymerase to bind to DNA and initiating RNA synthesis. A promoter for inclusion in a recombinant conjugative plasmid of the disclosure can be an endogenous promoter, a heterologous promoter or a combination thereof.

In some examples, the promoter is a constitutive promoter (e.g., a T7, SP6, T3, integron (Pc) or other suitable constitutive promoter). Other constitutive promoters are known in the art and contemplated herein.

In other examples, one or more or all of the genetic elements comprised within the recombinant plasmid of the disclosure may be operably-linked to an inducible promoter. An inducible promoter may be a nucleic acid sequence or an operon system that directs the conditional expression of the gene or system in the presence of a certain compound, nutrient, amino acid, sugar, peptide, protein or condition (e.g., light, oxygen, heat, cold). Alternatively, the inducible promoter may comprise one or more repressor elements such that the absence of a certain compound, nutrient, amino acid, sugar, peptide, protein or condition is required to induce transcription of the gene or system. Any suitable inducible promoter, system or operon known in the art may be used. Non-limiting examples of inducible promoters which are contemplated include lactose regulated systems (e.g., lactose operon systems), sugar regulated systems, metal regulated systems, steroid regulated systems, alcohol regulated systems, IPTG inducible systems, arabinose regulated systems (e.g., arabinose operon systems, e.g., an ARA operon promoter, pBAD, PARA, PARAE, ARAE, ARAR-ParaE, portions thereof, combinations thereof and the like), synthetic amino acid regulated systems (e.g., see Rovner A J, et al., (2015) Nature 518 (7537): 89-93), fructose repressors, a tac promoter/operator (pTac), tryptophan promoters, PhoA promoters, recA promoters, proU promoters, cst-1 promoters, tetA promoters, cadA promoters, nar promoters, PL promoters, cspA promoters, the like or combinations thereof. In one particular example, the inducible promoter is a L-arabinose inducible promoter.

The recombinant plasmid of the disclosure may comprise one or more selectable marker genes. As used herein, the term “antibiotic marker genes” or “antibiotic resistance marker genes” denotes genes that are a detectable genetic trait or segment of DNA that can be identified and tracked. A selectable marker can be a gene, the product of which provides for biocide or viral resistance, resistance to heavy metals, prototrophy to auxotrophs, and the like. Specifically, antibiotic selectable markers confer antibiotic resistance to antibiotics such as but not limited to ampicillin, kanamycin, chloramphenicol, tetracycline, rifampicin, neomycin, hygromycin or methotrexate, gentamycin. In a preferred example, tetracyclin (tetA) and fosfomycin (fosA3) antibiotic resistance markers are used as selection markers for selecting the recombinant plasmid of the current disclosure.

### Bacterial Cells and Compositions

The recombinant plasmids of the disclosure may be loaded into donor bacteria (e.g., probiotic bacteria and/or commensal bacteria) for delivery to microbial cell populations, e.g., endogenous microbial cells. Accordingly, present disclosure provides bacterial cells (or host bacteria) comprising the recombinant conjugative plasmids as described herein, including curing plasmids described herein. That is, bacterial cells into which the recombinant conjugative plasmids of the disclosure have been introduced or progeny of such bacterial cells. Such bacterial cells may be used in plasmid curing and/or as probiotics to prevent or reduce the acquisition and/or spread of plasmids (such as pathogenic plasmids). Accordingly, the bacteria as described herein may be a probiotic bacteria.

The terms “probiotic”, “probiotics” or similar, as used in connection with bacteria of the disclosure, shall be understood to mean bacteria that enhance the growth and/or health of beneficial bacterium in a particular environment (e.g., the gastrointestinal tract of a subject, soil or water system). Alternatively, or in addition, “probiotic”, “probiotics” or similar, may assist in diminishing the growth and/or prevalence of pathogenic bacterium in a particular environment (e.g., the gastrointestinal tract, soil or healthcare environment). For example, a probiotic bacterium which is administered to a subject in an adequate amount should confer a health benefit to the subject (e.g., a human or animal host), such as by improving gastrointestinal microbial balance. In another example, a probiotic bacterium which is applied or otherwise introduced to soil, wastewater, biosolid or other environment in an adequate amount may improve the microbial balance in that environment and result in one or more beneficial outcomes.

Commensal bacteria (also referred to as commensal microflora), as another example, include bacteria present on body surfaces covered by epithelial cells that are exposed to the external environment (e.g., gastrointestinal and respiratory tract, vaginal, skin). Commensal bacteria are found, for example, in normal microflora and indigenous microbiota. As discussed above, bacterial plasmids are transferred from bacteria to bacteria through conjugation. Thus, aspects of the disclosure contemplate the use of donor bacteria (e.g., probiotic bacteria and/or commensal bacteria) loaded with plasmids containing the desired cargo to microbial cells (e.g., pathogenic microbial cells) in vivo.

Exemplary bacteria include Escherichia coli, members of Enterobacteriaceae species and other Bacillus species include, but are not limited to: Bacillus coagulans; Bacillus coagulans Hammer; and Bacillus brevis subspecies coagulans, Bacillus laevolacticus, Bacillus subtilis, Bacillus uniflagellatus, Bacillus lateropsorus, Bacillus laterosporus BOD, Bacillus megaterium, Bacillus polymyxa, Bacillus licheniformis, Bacillus pumilus, and Bacillus stearothermophilus. Exemplary probiotic Lactobacillus species include, but are not limited to: Lactobacillus acidophilus, Lactobacillus casei, Lactobacillus paracasei, Lactobacillus DDS-1, Lactobacillus GG, Lactobacillus rhamnosus, Lactobacillus plantarum, Lactobacillus reuteri, Lactobacillus gasserii, Lactobacillus jensenii, Lactobacillus delbruekii, Lactobacillus, bulgaricus, Lactobacillus salivarius, Lactobacillus plantarum, Lactobacillus fermentum, Lactocobacillus helveticus, and Lactobacillus sporogenes. Exemplary probiotic Sporolactobacillus species include all Sporolactobacillus species, for example, Sporolactobacillus P44. Exemplary probiotic Bifidobacterium species include, but are not limited to: Bifidobacterium adolescentis, Bifidobacterium animalis, Bifidobacterium adolescentis Bifidobacterium bifidum, Bifidobacterium bifidus, Bifidobacterium breve, Bifidobacterium infantis, Bifidobacterium infantus, Bifidobacterium longum, and any genetic variants thereof. Other strains that could be employed due to probiotic activity include members of the Lactococcus such as Lactococcus lactis, Lactococcus diacetylactis, Lactococcus cremoris, and Streptococcus (Enterococcus) genus. For example, Enterococcus faecium, is commonly used as a livestock probiotic and, thus, could be utilized as a co-administration agent. Similarly, Ruminococcus sp and Megasphaera could be utilised in applications where the bacteria is to be administered to a livestock species as a probiotic.

For example, the probiotic bacteria may be selected from a group consisting of Escherichia, Lactobacillus, Bifidobacteria, Streptococcus, Lactococcus, Enterococcus, Propionibacterium, Faecalibacterium, Pediococcus, Ruminococcus, Megasphaera and Bacillus.

Non-pathogenic E. coli exemplary bacterium which may be used as a probiotic in the present disclosure since it is capable of colonization in the highly acidic environment of the gastrointestinal tract, particularly the human gastrointestinal tract.

Transformation of a bacterial cell with the recombinant conjugative plasmid of the disclosure may, for instance, be effected by any means known in art, including, but not limited to, protoplast transformation (see, e.g., Chang and Cohen, 1979, Molecular General Genetics 168:111-115), by using competent cells (see, e.g., Young and Spizizin, 1961, Journal of Bacteriology 81:823-829, or Dubnar and Davidoff-Abelson, 1971, Journal of Molecular Biology 56:209-221), by electroporation (see, e.g., Shigekawa and Dower, 1988, Biotechniques 6:742-751), or by conjugation (see, e.g., Kochler and Thorne, 1987, Journal of Bacteriology 169:5771-5278).

The growth of these various probiotic bacterial species to form cell cultures, cell pastes, and spore preparations is generally well-known within the art. A skilled person will be able to determine appropriate growth/culture conditions depending on whether the bacteria is an aerobe, a facultative anaerobe or an obligate anaerobes. Such culture methods are known in the art.

In some examples, the probiotic bacteria are encapsulated e.g., to protect the bacteria and maintain viability. Protection of the bacteria is achieved if either a majority of cells is still viable or is still metabolically active or if more of the encapsulated cells remain viable when compared with unencapsulated cells, which are treated under the same conditions. Methods of encapsulating (e.g., microencapsulation) of bacteria are known in the art.

Alternatively, or in addition, the probiotic bacteria may be freeze-dried. The term “freeze-drying” (also known as lyophilisation, lyophilization, or cryodesiccation) is used in its regular meaning as the cooling of a liquid sample, resulting in the conversion of freeze-able solution into ice, crystallization of crystallisable solutes and the formation of an amorphous matrix comprising non-crystallizing solutes associated with unfrozen mixture, followed by evaporation (sublimation) of water from amorphous matrix. In this process the evaporation (sublimation) of the frozen water in the material is usually carried out under reducing the surrounding pressure to allow the frozen water in the material to sublimate directly from the solid phase to the gas phase. Freeze-drying typically includes the steps of pretreatment, freezing, primary drying and secondary drying. Methods of freeze-drying are known in the art. An exemplary method is described in WO2015000972, the full contents on which is incorporated by reference herein.

In other examples, the probiotic bacteria may be spray-dried or extruded.

### Compositions

The recombinant plasmids and bacterial cells comprising same may be formulated into a composition and/or provided in a kit. The compositions and kits may further comprise additional reagents such as buffers, salts and the like. In some examples, the compositions containing the purified recombinant plasmid or bacteria carrying recombinant plasmid for research use. Recombinant plasmid pJIMK_Core_M or its derivatives may be available for genetic engineering to construct suitable plasmid to deliver genetic material. In some examples, the compositions are pharmaceutical compositions optionally comprising one or more pharmaceutical carriers and/or excipients.

In some examples, the bacteria of the disclosure may be formulated in a composition suitable for administration to a human or animal subject. In one example, the composition is for administration to a human. In another example, the composition is for administration to an animal. Exemplary animals for which the compositions of the disclosure may be particularly useful include livestock species (e.g. cattle, sheep, horses, pigs, donkeys, poultry), companion animals (e.g. dogs, cats), performance animals (e.g. racehorses, camels, greyhounds) and captive wild animals. In one example, the animal is a ruminant. Exemplary ruminants include cattle, sheep, goats, buffalo, deer or camelids. In another example, the animal may be a hind gut fermenter. An exemplary hindgut fermenter is a horse. In another example, the animal may be an avian species, such as poultry.

In other examples, the compositions may be formulated for administration to an environment selected from a healthcare environment, a soil environment, an environment comprising a water source, an environment comprising wastewater, an environment comprising industrial waste, an environment comprising agricultural waste, an environment comprising sewerage and/or an environment comprising bio-solids.

The recombinant plasmid, bacteria and compositions comprising same of the disclosure may be formulated for administration or application by any route determined to be suitable by a person skilled in the art. For example, in accordance with examples in which the plasmid, bacteria and compositions comprising same are for administration to a subject, the composition may be formulated for oral administration (e.g., as ingestible liquid or solid, an oral drench, a feed additive, a food (e.g., a dairy product such as a drinkable yoghurt), or a capsule), topical administration (e.g., as a lotion or cream), intranasal administration or parenteral administration. In one example, the composition of the disclosure is formulated for oral administration e.g., as a food, beverage, bolus, drench or capsule. Accordingly, the composition may further comprise one or more physiologically acceptable excipients, carriers or additives suitable for ingestion by a human or non-human animal. Physiologically acceptable excipients, carriers or additives suitable for ingestion by human or non-human animals are known in the art and described herein. Such carriers can, for example, allow the probiotic bacteria of the disclosure to be formulated as tablets, pills, dragées, capsules, liquids, gels, syrups, slurries, suspensions and the like. The choice of carrier will be dependent on the form of the composition, the intended method of administration, the intended shelf-life and storage considerations. In some examples, the composition may be a food or beverage product (e.g., a dairy product such as a drinkable yoghurt). In some examples, the composition may be a tablet, pill, caplet, or capsule. Suitable excipients include, but are not limited to, fillers such as sugars, including, but not limited to, lactose, sucrose, mannitol, and sorbitol; cellulose preparations such as, but not limited to, maize starch, wheat starch, rice starch, potato starch, gelatin, gum tragacanth, methyl cellulose, hydroxypropylmethyl cellulose, sodium carboxymethyl cellulose, and polyvinylpyrrolidone (PVP). If desired, disintegrating agents can be added, such as, but not limited to, the cross-linked polyvinylpyrrolidone, agar, or alginic acid or a salt thereof such as sodium alginate. Compositions that can be used orally include, but are not limited to, capsules made of gelatin, as well as soft, sealed capsules made of gelatin and a plasticizer, such as glycerol or sorbitol. In some examples, the composition may be formulated in a buffer. It will be understood by a person skilled in the art that by suitable buffer may be used. Examples of suitable buffers include, but are not limited to phosphate, calcium carbonate, bicarbonate, phosphate citrate and histidine. In other examples, the composition may be formulated with a carrier having a low oxygen diffusion rate e.g., such as ingestible oils. The composition may further comprise an antioxidant.

In some examples, the composition may comprise a preservative or a stabilizer. Furthermore, depending on the method of manufacture, the composition may comprise one or more cryoprotectants known in the art.

In accordance with an example in which the composition is a food product (such as a diary product), feed/nutritional supplement or beverage product, the probiotic bacteria or a composition comprising same may be prepared by, or shipped to, a manufacturer. The probiotic bacteria or composition comprising same may then be formulated into the food product, feed/nutritional supplement or beverage product by the addition of further ingredients which are appropriate to the product.

In some examples, the composition of the disclosure is stable when stored at ambient room temperature (e.g., 20° C. and 25° C.) e.g., stable when stored at ambient room temperature for at least one month or more. In other examples, such as those in which the composition is formulated as a dairy food or beverage product, the composition may be required to be refrigerated in order to maintain viability of the bacteria comprised therein.

In some examples, a composition of the disclosure is packaged in a container. The container may contain a single dose or multiple doses of the composition as described herein.

Preferably the compositions comprise probiotic bacteria of the disclosure in an amount sufficient to at least partially provide a benefit to the health of a subject or the environment to which they are administered.

In accordance with applications in which the composition is formulated for use in a human or non-human animal, the compositions will comprise bacteria of the disclosure in an amount sufficient to at least partially provide a health benefit to the human or non-human animal. An amount adequate to accomplish this is defined as a “therapeutically effective amount”. Effective amounts for this purpose may vary depending on a number of factors known to those skilled in the art, including, but not limited to, the species of subject, anatomy of the digestive system (e.g., four chamber or single chamber stomach), the size/weight of the subject, the composition of the subject's diet (existing and future), whether the subject is lactating, whether the subject is pregnant and the outcome to be achieved. The appropriate dosage of the probiotic bacteria (e.g., CFUs per strain) to be formulated in a composition of the disclosure may therefore be determined by a person skilled in the art taking into account one or more of the above factors.

In one example, a unit or dosage of a composition of the disclosure may comprise between about 10² CFU to about 10¹⁴ CFU, or about 10³ CFU to about 10¹³ CFU, or about 10⁴ CFU to about 10¹³ CFU, or about 10⁵ CFU to about 10¹³ CFU, or about 10⁶ CFU to about 10¹³ CFU, or about 10⁶ CFU to about 10¹² CFU, or about 10⁷ CFU to about 10¹¹ CFU, or about 10⁸ CFU to about 10¹⁰ CFU, or about 10⁹ CFU to about 10¹⁰ CFU of the bacteria of the disclosure. For example, each unit or dosage of a composition of the disclosure may comprise about 5×10⁷ CFU or about 6×10⁸ CFU, or about 10⁹ CFU, or about 10¹⁰ CFU of the bacteria.

### Methods of Use

Also provided herein are methods of introducing a recombinant plasmid to a bacterial population via conjugative transfer, comprising contacting a recombinant plasmid of the disclosure, or a bacteria or composition comprising same, to the population of bacteria. In this regard, the recombinant plasmid, bacteria or compositions of the disclosure may be administered to an individual, animal or other organism or body or surface, to permit displacement or disruption of specific plasmids associated pathogenic traits (e.g., AMR, virulence etc) in target bacteria, thereby allowing said bacteria to be killed or weakened or made more vulnerable to other therapeutic agents. The data in the Examples herein demonstrates the use of the recombinant plasmids of the disclosure to efficiently transfer to and displace incumbent pathogenic plasmids in live bacteria. Thus, the recombinant plasmids, bacteria and compositions of the disclosure may be used to treat or prevent or reduce the spread and/or acquisition of plasmids that confer pathogenic traits, such as multi-drug resistance (also referred to as AMR), virulence and/or metal resistance in pathogenic bacterial populations and assists with the colonization (i.e. re-colonization) of the gastrointestinal tract or other environments (e.g., such as in the case of bioremediation).

In one example, the disclosure provides a method of plasmid curing in a population of bacteria to reduce the prevalence and/or spread of a plasmid, conferring a trait of interest in the population of bacteria (e.g., a population of gut bacteria), said method comprising introducing to the population a curing plasmid as described herein or a bacterial cell as described herein or a pharmaceutical composition as described herein.

In one example, the trait of interest is AMR, bacterial virulence, or heavy metal resistance. In one example, the method reduces the prevalence of a plasmid conferring two or more of AMR, bacterial virulence, and/or heavy metal resistance in a population of bacteria (e.g., a population of gut bacteria).

In accordance with an example in which the population of bacteria is a population of gut bacteria, the method comprises administering the curing plasmid or the one or more bacterial cells or composition comprising same to the gut of a subject in need thereof.

In some examples, bacterial cells to which the curing plasmid of the disclosure, or the one or more bacterial cells or composition comprising same, are introduced may be anaerobic bacterial cells (e.g., cells that do not require oxygen for growth). Anaerobic bacterial cells include facultative anaerobic cells such as, for example, Escherichia coli, Shewanella oneidensis and Listeria monocytogenes. Anaerobic bacterial cells also include obligate anaerobic cells such as, for example, Bacteroides and Clostridium species. In humans, for example, anaerobic bacterial cells are most commonly found in the gastrointestinal tract.

The curing plasmid of the disclosure, or the one or more bacterial cells or composition comprising same, may be delivered alone or together with an antibiotic or other antimicrobial agent. Examples of antibiotics and antimicrobial agents that may be used in accordance with the present disclosure include, without limitation, aminoglycosides, ansamycins, carbapenems, cephalosporins (1st-5th generation), glycopeptides, lincosamides, lipopeptides, macrolides, monobactams, nitrofurans, oxazolidinones, penicillins (and combinations with penicillins), polypeptides, quinolones, sulfonamides, tetracyclines, drugs against mycobacteria, and others.

Examples of aminoglycosides include, without limitation, Amikacin, Gentamicin, Kanamycin, Neomycin, Netilmicin, Tobramycin, Paromomycin and Spectinomycin.

Examples of ansamycins include, without limitation, Geldanamycin, Herbimycin and Rifaximin (streptomycin).

Examples of carbapenems include, without limitation, Ertapenem, Doripenem, Imipenem/Cilastatina and Meropenem.

Examples of cephalosporins include, without limitation, Cefadroxil, Cefazolin, Cefalotin or Cefalothin, Cefalexin, Cefaclor, Cefamandole, Cefoxitin, Cefprozil, Cefuroxime, Cefixime, Cefdinir, Cefditoren, Cefoperazone, Cefotaxime, Cefpodoxime, Ceftazidime, Ceftibuten, Ceftizoxime, Ceftriaxone, Cefepime, Ceftaroline fosamil and Ceftobiprole. Examples of glycopeptides include, without limitation, Teicoplanin, Vancomycin and Telavancin.

Examples of lincosamides include, without limitation, Clindamycin and Lincomycin. An example of a lipopeptide includes, without limitation, Daptomycin. Examples of macrolides include, without limitation, Azithromycin, Clarithromycin, Dirithromycin, Erythromycin, Roxithromycin, Troleandomycin, Telithromycin and Spiramycin.

An example of a monobactams includes, without limitation, Aztreonam.

Examples of nitrofurans include, without limitation, Furazolidone and Nitrofurantoin.

Examples of oxazolidinones include, without limitation, Linezolid, Posizolid, Radezolid and Torezolid.

Examples of penicillins include, without limitation, Amoxicillin, Ampicillin, Azlocillin, Carbenicillin, Cloxacillin, Dicloxacillin, Flucloxacilline, Mezlocillin, Methicillin, Nafcillin, Oxacillin, Penicillin G, Penicillin V, Piperacillin, Penicillin G, Temocillin, Ticarcillin and penicillin combinations (e.g., amoxicillin/clavulanate, ampicillin/sulbactam, piperacillin/tazobactam, ticarcillin/clavulanate).

Examples of polypeptides include, without limitation, bacitracin, colistin and polymyxin B.

Examples of quinolones include, without limitation, Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin and Temafloxacin.

Examples of sulfonamides include, without limitation, Mafenide, Sulfacetamide, Sulfadiazine, Silver sulfadiazine, Sulfadimethoxine, Sulfamethizole, Sulfamethoxazole, Sulfanilamide (archaic), Sulfasalazine, Sulfisoxazole, Trimethoprim-Sulfamethoxazole (Co-trimoxazole) (TMP-SMX) and Sulfonamido chrysoidine (archaic).

Examples of tetracyclines include, without limitation, Demeclocycline, Doxycycline, Minocycline, Oxytetracycline and Tetracycline.

Examples of drugs against mycobacteria include, without limitation, Clofazimine, Dapsone, Capreomycin, Cycloserine, Ethambutol, Ethionamide, Isoniazid, Pyrazinamide, Rifampicin (Rifampin in US), Rifabutin, Rifapentine and Streptomycin.

Examples of other antibiotic and antimicrobial agents include, without limitation, Arsphenamine, Chloramphenicol, Fosfomycin, Fusidic acid, Metronidazole, Mupirocin, Platensimycin, Quinupristin/Dalfopristin, Thiamphenicol, Tigecycline, Tinidazole and Trimethoprim.

In some examples, the curing plasmids of the disclosure, or the one or more bacterial cells or composition comprising same, are administered to a subject are part of treatment for infection or colonisation by a multidrug-resistant organism (MRO) or bacteria expressing antimicrobial resistance (AMR). Accordingly, the method may comprise treating or preventing infection by one or more multidrug-resistant organisms (MRO) in a subject in need thereof. As used herein, the terms “treating”, “treat” or “treatment” and variations thereof, refer to clinical intervention using the plasmids, bacteria and compositions of the disclosure which is designed to alter the natural course of the individual or cell being treated during the course of clinical pathology and can also refer to environmental remediation efforts. Desirable effects of treatment when used in connection with the biological organism or infection, includes the amelioration, elimination, reduction, prevention, or other relief or management from the detrimental effects of a biological organism. via curing the pathogenic population off multi-drug resistance or virulence (for e.g., Enterobacteriaceae) either in the gut of a subject or other environments by ameliorating and/or controlling the colonization of pathogenic bacteria carrying such traits within the gastrointestinal tract of both humans or animals or other environments.

In one example, the multi-drug resistant organisms or pathogens includes, but are not limited to, organisms belonging to the genus Acinetobacter, Citrobacter, Enterobacter, Enteroccus, Escherichia, Kiebsiella, Serratia or Staphylococcus. Exemplary multi-drug resistant organisms include Acinetobacter baumannii such as ATCC isolate #2894233-696-101-1, ATCC isolate #2894257-696-101-1 ATCC isolate #2894255-696-101-1, ATCC isolate #2894253-696-101-1, or ATCC #2894254-696-101-1; Citrobacter freundii such as ATCC isolate #33128, ATCC isolate #2894218-696-101-1, ATCC isolate #2894219-696-101-1, ATCC isolate #2894224-696-101-1, ATCC isolate #2894218-632-101-1, or ATCC isolate #2894218-659-101-1; Enterobacter cloacae such as ATCC isolate #22894251-659-101-1, ATCC isolate #22894264-659-101-1, ATCC isolate #22894246-659-101-1, ATCC isolate #22894243-659-101-1, or ATCC isolate #22894245-659-101-1; Enteroccus faecalis such as ATCC isolate #22894228-659-101-1 ATCC isolate #22894222-659-101-1, ATCC isolate #22894221-659-101-1, ATCC isolate #22894225-659- 101-1, or ATCC isolate #22894245-659-101-1; Enteroccus faecium such as ATCC isolate #51858, ATCC isolate #35667, ATCC isolate #2954833_2694008 ATCC isolate #2954833_2692765, or ATCC isolate #2954836_2694361; Escherichia coli such as ATCC isolate CGUC 11332, CGUC 11350, CGUC 11371, CGUC 11378, or CGUC 11393; Kiebsiella pneumonia such as ATTC isolate #27736, ATTC isolate #29011, ATTC isolate #20013, ATTC isolate #33495, or ATTC isolate #35657; Serratia marcescens such as ATCC isolate #43862, ATCC isolate #2338870, ATCC isolate #2426026, ATCC isolate #SIID 2895511, or ATCC isolate #SIID 2895538; or Staphylococcus aureus such as ATCC isolate #JHH 02, ATCC isolate #JHH 02, ATCC isolate #JHH 03, ATCC isolate #JHH 04, ATCC isolate #JHH 05, or ATCC isolate #JHH 06.

In one example, the bacterial infection is brought about by virulence plasmids which carry a variety of virulence genes that encode virulence factors/virulence determinants. For example, pathogenic E. coli generally rely on plasmid-borne virulence factors. A variety of toxins are found in different pathogenic E. coli strains, including heat-labile enterotoxin, heat-stable enterotoxin, hemolysin (lyses red blood cells), and Shiga-like toxin (similar to the toxin of dysentery-causing Shigella). There is a similar variety of adhesins or “colonization factors,” proteins that enable bacteria to stick to the surface of animal cells. Other enteric bacteria, such as Salmonella typhi (typhoid) and Y. pestis (bubonic plague), cause severe infections. They also carry virulence plasmids.

In one example, the method may be used to reduce the prevalence, or reduce the spread and/or acquisition, of plasmids conferring pathogenic traits to bacteria of the family of Enterobacteriaceae. The family “Enterobacteriaceae” includes 14 main genera and 6 further genera, is known to have different properties. Typical examples are Escherichia, Salmonella and Klebsiella. Virulence genes are usually found in enterobacteria on large plasmids (approx. 60 kb or larger). Enterobacteria are known in the art and are generally pathogens that can infect the gastrointestinal tract of avians and/or mammals. The present disclosure can be utilised to target any population of enterobacteria in the order Enterobacteriales and optionally in the family Enterobacteriaceae, including but not limited to bacteria classified in the following genera: Alishewanella, Alterococcus, Aquamonas, Aranicola, Arsenophonus, Azotivirga, Blochmannia, Brenneria, Buchnera, Budvicia, Buttiauxella, Candidatus, Cedecea, Citrobacter, Dickeya, Edwardsiella, Enterobacter, Erwinia (e.g., E. amylovora), Escherichia, Ewingella, Grimontella, Hafnia, Klebsiella (e.g., K. pneumoniae), Kluyvera, Leclercia, Leminorella, Morganella, Moellerella, Obesumbacterium, Pantoea, Pectobacterium, Candidatus Phlomobacter, Photorhabdus, Plesiomonas (e.g., P. shigelloides), Pragia, Proteus (e.g., P. vulgaris), Providencia, Rahnella, Raoultella, Salmonella, Samsonia, Serratia (e.g., S. marcescens), Shigella, Sodalis, Tatumella, Travulsiella, Wigglesworthia, Xenorhabdus, Yersinia (e.g., Y. pestis), and Yokenella.

In a preferred embodiment, the enterobacterium is an Escherichia spp., Salmonella spp. or a Shigella spp.

A reduction in prevalence, or spread and/or acquisition, of a pathogenic plasmid in a bacterial population may be any reduction, such as a reduction by at least 10% (such as by at least 15%, or at least 20%, or at least 25%, or at least 30%, or at least 35%, or at least 40% or more), relative to the prevalence of the pathogenic plasmid in bacterial population into which the plasmid, bacteria or composition the disclosure has not been introduced. A reduction in spread or acquisition of a pathogenic plasmid in a bacterial population may result in one or more treatment outcomes. As used herein, the terms “treating”, “treat” or “treatment” and variations thereof, refer to clinical intervention using non-pathogenic bacteria designed to alter the natural course of the individual or cell being treated during the course of clinical pathology and can also refer to environmental remediation efforts. Desirable effects of treatment when used in connection with the biological organism or infection, includes the amelioration, elimination, reduction, prevention, or other relief or management from the detrimental effects of a biological organism. via curing the pathogenic population off multi-drug resistance or metal resistance or virulence (for e.g., Enterobacteriaceae) either in the gut of a subject or other environments by ameliorating and/or controlling the colonization of pathogenic bacteria carrying such traits within the gastrointestinal tract of both humans or animals or other environments.

In one example, the recombinant plasmid, bacteria or composition comprising same of the present disclosure is administered once or more daily, weekly, fortnightly, monthly, or bi-monthly, wherein a daily, weekly, fortnightly, monthly, or bi-monthly dosage comprises an amount of the plasmid or bacteria as described above.

In some examples, the recombinant plasmid, bacteria or composition comprising same of the present disclosure is administered to a human or animal subject. In one example, the recombinant plasmid, bacteria or composition comprising same of the present disclosure is administered to a human. In another example, the recombinant plasmid, bacteria or composition comprising same of the present disclosure is administered to an animal. Exemplary animals for which the recombinant plasmid, bacteria or composition comprising same of the present disclosure may be particularly useful are described herein.

As described herein, the recombinant plasmid, bacteria or composition comprising same of the present disclosure may be administered by any route determined to be suitable by a person skilled in the art. For example, in accordance with examples in which the probiotic bacteria and compositions are administered to a human or animal subject, the composition may be formulated for administration orally (e.g., as ingestible liquid or solid, an oral drench, a feed additive, a food (e.g., a dairy product), or a capsule), topically (e.g., as a lotion, cream or gel), intranasally or parenterally. In one example, the composition of the disclosure is administered orally e.g., as a food, beverage, bolus, drench or capsule.

In one example, the recombinant plasmid, bacteria or composition comprising same of the present disclosure may be administered as topical cream, lotion, or gel. For example, creams, lotions or gels comprising the probiotic bacteria may be effective in reducing prevalence, or reducing or preventing the acquisition or spread, of pathogenic plasmids within bacterial populations on the skin, thereby controlling antibiotic resistant pathogens on the skin. In this regard, pathogenic Pseudomonas, Staphylococcus, and/or Enterococci are frequently associated with infections of severe burns.

Preferably the recombinant plasmid, bacteria or composition comprising same of the present disclosure is administered to the subject in a therapeutically effective amount. As described herein, this may vary depending on a number of factors known to those skilled in the art, including, but not limited to, the species of subject, anatomy of the digestive system (e.g., four chamber or single chamber stomach), the size/weight of the subject, the composition of the subject's diet (existing and future), whether the subject is lactating, whether the subject is pregnant and the outcome to be achieved. The appropriate dosage of the probiotic bacteria (e.g., CFUs per strain) to be formulated in a composition of the disclosure may therefore be determined by a person skilled in the art taking into account one or more of the above factors.

In one example, a unit or dosage of a composition administered to a subject may comprise between about 10² CFU to about 10¹⁴ CFU, or about 10³ CFU to about 10¹³ CFU, or about 10⁴ CFU to about 10¹³ CFU, or about 10⁵ CFU to about 10¹³ CFU, or about 10⁶ CFU to about 10¹³ CFU, or about 10⁶ CFU to about 10¹² CFU, or about 10⁷ CFU to about 10¹¹ CFU, or about 10⁸ CFU to about 10¹⁰ CFU, or about 10⁹ CFU to about 10¹⁰ CFU of the probiotic bacteria of the disclosure. For example, the method may comprise administering a composition comprising about 5×10⁷ CFU or about 6×10⁸ CFU, or about 10⁹ CFU, or about 10¹⁰ CFU of the probiotic bacteria.

In another example, the bacterial population is a population of bacteria which colonise plants and/or soil and the method comprises contacting the plant or soil with the curing plasmid or the bacterial cell or composition comprising same.

In another example, the population of bacteria is present in an environment selected from a healthcare environment, a soil environment, an environment comprising a water source, an environment comprising waste water, an environment comprising industrial waste, an environment comprising agricultural waste, an environment comprising sewerage and/or an environment comprising bio-solids, and the method comprises introducing curing plasmid or the bacterial cell or the composition to the environment.

In each of the foregoing example, the method of the disclosure may further comprise administering one or more antibiotic agents to the bacterial population. For example, the method may reduce the prevalence of a plasmid conferring AMR in the population of bacteria rendering the bacteria susceptible to one or more antibiotic agents.

The present disclosure also provides for use of a curing plasmid or a bacterial cell or composition comprising same as described herein in the preparation of a medicament for treating or preventing antibiotic resistance in a subject.

### EXAMPLES

### Example 1: Evaluation of Conjugation Efficiency of IncM Plasmid pJIBE401

In this example, the inventors compare the conjugative transfer frequency of naturally occurring plasmid pJIBE401 to plasmids from different plasmid incompatibility groups such as IncC plasmid pEc158 (GenBank accession no. KY887596.1), promiscuous plasmid pKPC_UVA01 with unknown incompatibility group (based on PlasmidFinder, GenBank accession no. CP017937.1) and IncA plasmid pRA1 (GenBank accession no. NC_012885.1) both in-vitro and in mouse gut.

### Bacterial Strains Used:

The donor bacteria used was Escherichia coli J53 (resistant to sodium azide), and recipient bacteria were E. coli BW25113Rf (rifampicin-resistant), Klebsiella pneumoniae ATCC13883Rf (rifampicin-resistant), Morganella morganii Mm1585Rf (rifampicin-resistant) strains.

### In-Vitro Conjugation:

In vitro plasmid conjugation was measured using standard filter mating experiments as described in Rodriguez-Grande et al. (2020) Methods Mol Biol, 2075:93-98. Briefly, a donor with respective plasmid and recipient bacteria were grown overnight in LB medium with appropriate antibiotics. One mL cultures were harvested by centrifugation at 10,000 g for 5 min, washed twice with 1 mL saline and suspended in 50 μL saline. Donor and recipient suspensions were mixed. The mixture was placed on a nitrocellulose filter (Amersham Hybond-C extra, 82 mm) on a LB agar plate and then incubated at 37° C. overnight. The mixture of cultures was harvested in 5 mL saline, and serial dilution was performed using 100 μL of mating mixture and spread onto nutrient agar plates containing rifampicin (90 μg/mL) and antibiotic which confers resistance to incoming plasmid. These plates were incubated at 37° C. overnight.

Transconjugant colonies were then counted, conjugation frequency was measured by dividing the number of transconjugants by the number of the donor bacteria. Furthermore, colonies were also patched onto CHROMagar Orientation plates containing appropriate antibiotics to confirm that they were not spontaneous rifampicin-resistant mutants of donor strains. The presence of incoming plasmid genes in transconjugants was confirmed by colony PCR using custom primers.

### In Vivo Conjugation:

Conjugation rates were determined in vivo using a method modified from Faruque et al. (2003) Infect Immun 71:1020-1025 and Faruque et al. (2004) Proc Natl Acad Sci USA, 101:2123-2128. Briefly, bacteria with donor and recipient plasmid were grown in LB broth with appropriate antibiotics at stationary phase at 37° C. till the culture reached an optical density of 1. The cultures were pelleted and washed twice in Phosphate-buffered saline (PBS). Donor and recipient cells (both at OD₆₀₀=1.0, ˜108 cfu) were then mixed in a 1:1 ratio, kept on ice to prevent plasmid transfer before inoculating mice. The suspension (50 μL) was used to intragastrically inoculate 5-day-old BALB/c mice (5 mice/group).

In vivo mating was allowed to proceed for ˜20 h (similar to overnight in vitro mating). Mice were sacrificed, and bacteria recovered from the intestines by homogenisation in PBS. Serial dilutions of the homogenates were done and plated onto nutrient agar plates with appropriate antibiotic to count transconjugants. Conjugation rates was calculated as the ratio of the total number of transconjugants to the number of donors.

### Comparison of Conjugation Frequency:

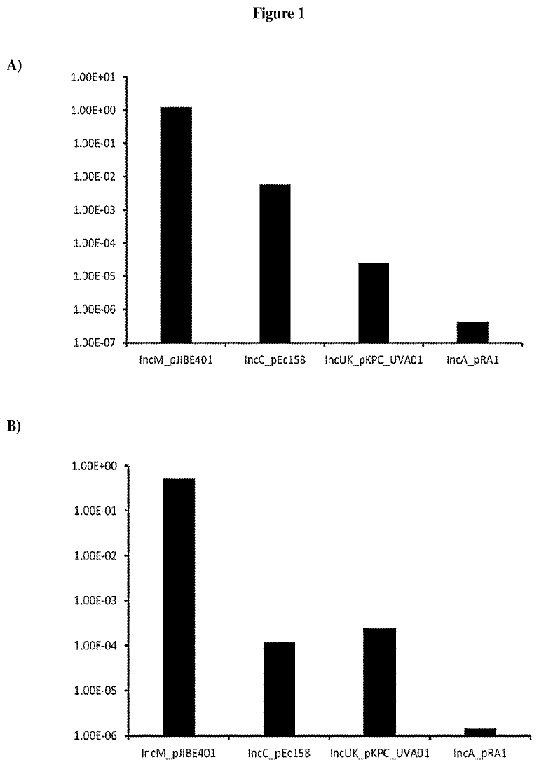

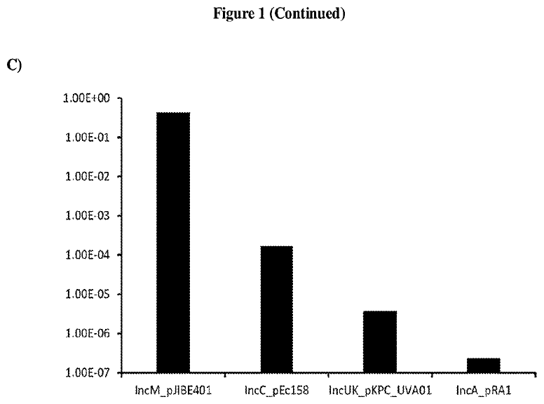

A very high conjugation frequency (>1) was detected for IncM plasmid pJIBE401 into different Enterobacteriaceae species (Table 1, FIG. 1). A significant difference in the conjugation frequency was observed when compared with plasmids of different incompatibility types. These data demonstrated that the IncM plasmid, pJIBE401, is much better in its conjugative transfer efficiency than its counterparts (Table 1, FIG. 1).

TABLE 1
Conjugation frequency of plasmids to different Enterobacteriaceae species in vitro

| Donor^{a} | Recipient^{a} | Conjugation frequency^{b} |
|---|---|---|
| Ec J53 (pJIBE401_IncM plasmid) | Ec BW25113Rf | 1.2 ± 0.6 |
| Ec J53 (pEc158_IncC plasmid) | Ec BW25113Rf | (5.8 ± 1.4) × 10⁻³ |
| Ec J53 (pKPC_UVA01_unkonwn type) | Ec BW25113Rf | (2.5 ± 1.2) × 10⁻⁵ |
| Ec J53 (pRA1_IncA plasmid) | Ec BW25113Rf | (4.2 ± 2.6) × 10⁻⁷ |
| Ec J53 (pJIBE401_IncM plasmid) | Kp ATCC13883Rf | (5.02 ± 0.8) × 10⁻¹ |
| Ec J53 (pEc158_IncC plasmid) | Kp ATCC13883Rf | (1.2 ± 0.4) × 10⁻⁴ |
| Ec J53 (pKPC_UVA01_unkonwn type) | Kp ATCC13883Rf | (2.4 ± 1.2) × 10⁻⁴ |
| Ec J53 (pRA1_IncA plasmid) | Kp ATCC13883Rf | (1.4 ± 1.5) × 10⁻⁶ |
| Ec J53 (pJIBE401_IncM plasmid) | Mm1585Rf | (4.2 ± 2.1) × 10⁻¹ |
| Ec J53 (pEc158_IncC plasmid) | Mm1585Rf | 1.7 ± 0.6) × 10⁻⁴ |
| Ec J53 (pKPC_UVA01_unkonwn type) | Mm1585Rf | (3.6 ± 1.1) × 10⁻⁶ |
| Ec J53 (pRA1_IncA plasmid) | Mm1585Rf | (2.3 ± 1.2) × 10⁻⁷ |

^{a}Ec, Escherichia coli; Kp, Klebsiella pneumoniae; Mm, Morganella morganii
^{b}Conjugation frequency are the mean of 5 individual experiments

### Example 2: Determination of the Core Genetic Region of pJIBE401, Responsible for High-Level Conjugative Transfer

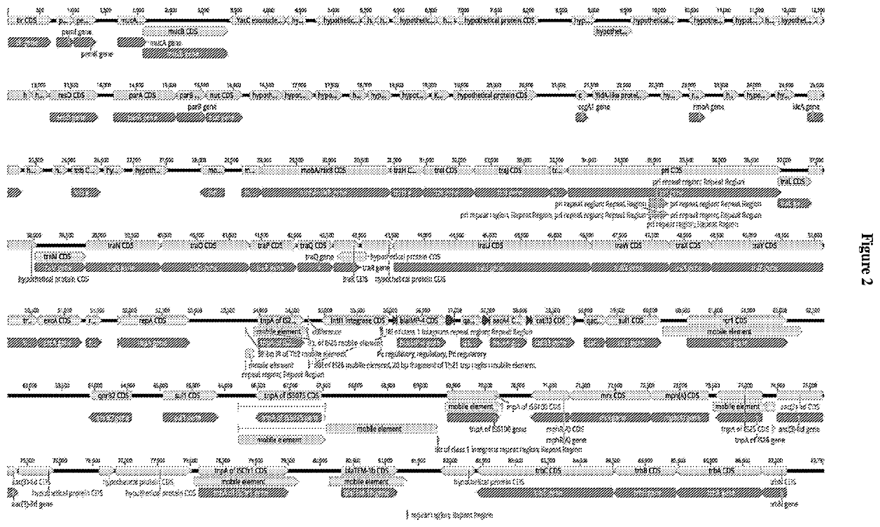

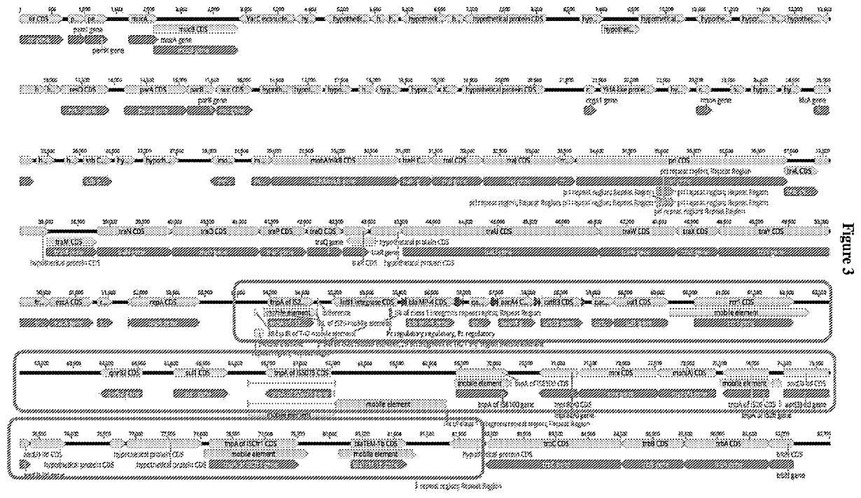

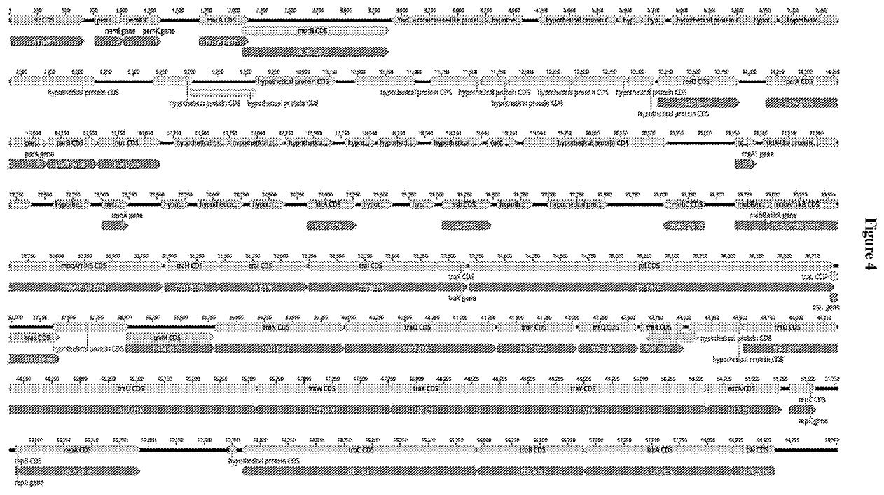

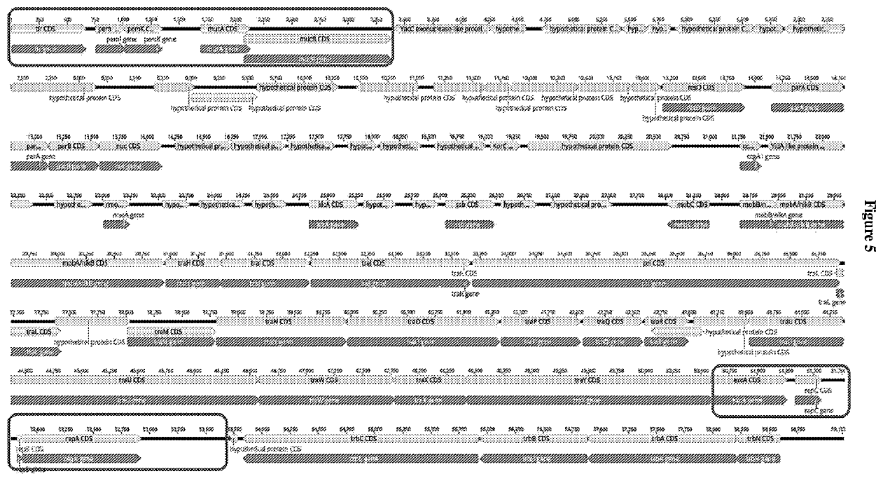

The transfer efficient conjugative IncM plasmid pJIBE401 was fully sequenced. It is 87,731 bp in size and its genetic structure (FIG. 2) shows that this plasmid carries a multi-drug resistance region (MRR) and transposable elements with the size of ˜28 kb (FIG. 3). Assuming that the MRR region, which confers resistance to multiple different antibiotics, is not necessary for conjugative transfer, this ˜28 kb region was deleted from the pJIBE401 to produce a plasmid designated pJIMK44_ΔMRR_M (FIG. 4). Further genetic analysis identified several other genes (tir, pemIK, mucAB, exc, repA), which may not participate or be necessary for conjugative transfer of this plasmid (FIG. 5). The tir is known to encode a transfer inhibition protein, pemIK is a toxin-antitoxin system also known as plasmid addiction system, mucAB encodes error-prone DNA polymerase, exc encodes entry exclusion protein and plays a role in the inhibition of repetitive transfer of the plasmid and repA is a plasmid replication gene. These genetic elements (i.e., the tir, pemIK, mucAB, and exc genes) were also deleted from pJIMK44_ΔMRR_M to produce a smaller vector designated pJIMKCore_M plasmid (FIG. 6).

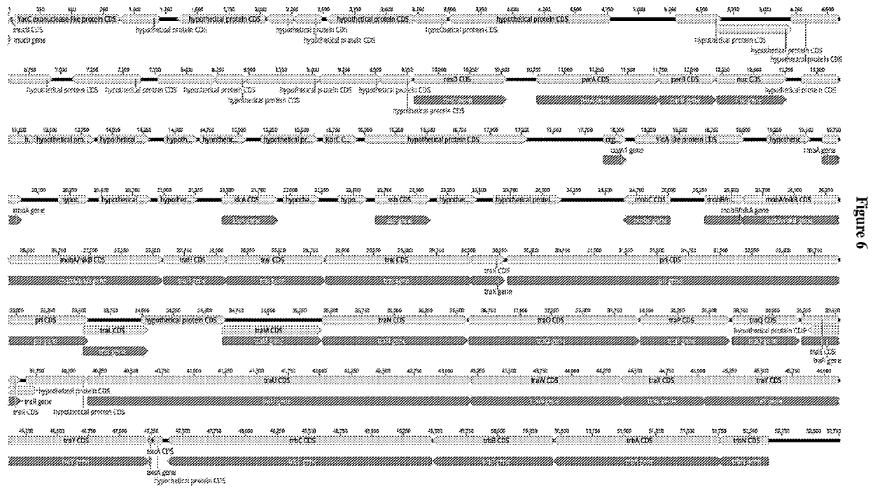

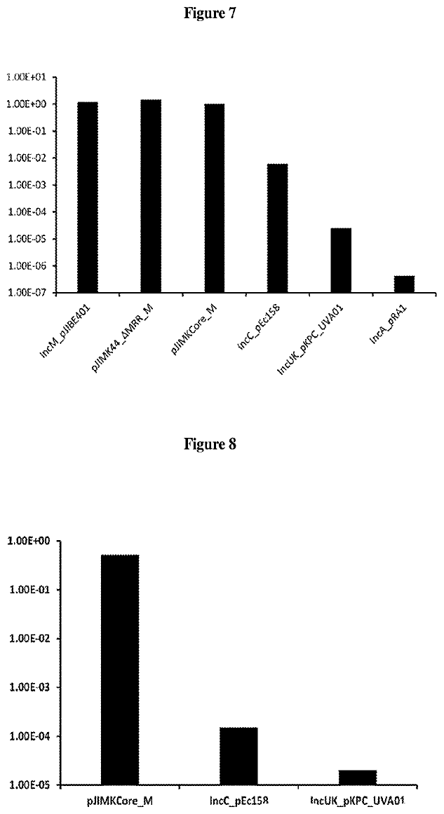

The conjugation frequency of the naturally occurring plasmid pJIBE401 and its derivatives, pJIMK44_ΔMRR_M and pJIMKCore_M, was then assessed. As is evident from the data in Table 2, there was no significant difference in the conjugation frequencies for pJIBE401 and its derivatives, pJIMK44_ΔMRR_M and pJIMKCore_M. This suggests that the core genetic information retained in pJIMKCore_M is sufficient to confer the high-level of conjugative transfer of the native plasmid (FIG. 7).

TABLE 2
Conjugation frequency of IncM plasmid pJIBE401 and
its derivatives vs plasmids of other Inc groups

| Donor^{a} | Recipient^{a} | Conjugation frequency^{b} |
|---|---|---|
| Ec J53 (pJIBE401_IncM plasmid) | Ec BW25113Rf | 1.2 ± 0.6 |
| Ec J53 (pJIMK44_ΔMRR_M) | Ec BW25113Rf | 1.4 ± 1.2 |
| Ec J53 (pJIMKCore_M) | Ec BW25113Rf | 1.0 ± 0.7 |
| Ec J53 (pEc158_IncC plasmid) | Ec BW25113Rf | (5.8 ± 1.4) × 10⁻³ |
| Ec J53 (pKPC_UVA01_unkonwn type) | Ec BW25113Rf | (2.5 ± 1.2) × 10⁻⁵ |
| Ec J53 (pRA1_IncA plasmid) | Ec BW25113Rf | (4.2 ± 2.6) × 10⁻⁷ |

^{a}Ec, Escherichia coli
^{b}Conjugation frequencies are the mean of 3 individual experiments

Efficiency of Plasmid pJIMKCore_M in In Vivo Conjugative Transfer:

The conjugative transfer efficiency of pJIMKCore_M into recipient bacteria in the mouse gut environment was then examined. As shown in Table 3 and FIG. 8, in vivo transfer of pJIMKCore_M was as efficient as its conjugative transfer in vitro.

TABLE 3
Plasmid conjugation frequency in vivo in the mouse gut

| Donor^{a} | Recipient^{a} | Conjugation frequency^{b} |
|---|---|---|
| Ec J53 (pJIMKCore_M) | Ec BW25113Rf | (5.1 ± 1.3) × 10⁻¹ |
| Ec J53 (pEc158_IncC plasmid) | Ec BW25113Rf | (1.5 ± 0.8) × 10⁻⁴ |
| Ec J53 (pKPC_UVA01_unkonwn type) | Ec BW25113Rf | (2.0 ± 1.5) × 10⁻⁵ |

### Example 3: Curing Plasmids Comprising the pJIMKCore_M Conjugation System are Efficient in Curing Target AMR Plasmids In Vitro and In Vivo

Two AMR curing plasmids were constructed using the pJIMKCore_M plasmid conjugation system. These AMR curing plasmids were then used to cure two target AMR plasmids (i.e., IncC and pKPC_UVA01) in vitro and in vivo.

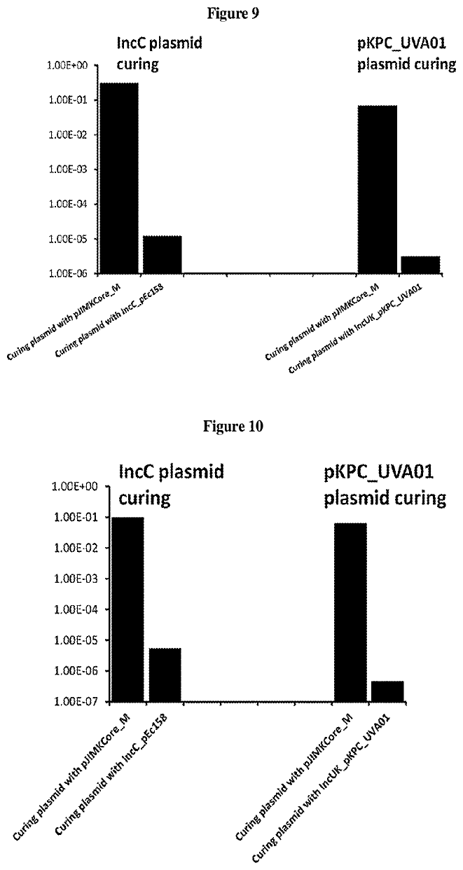

As shown in Tables 4 and 5 and FIGS. 9 and 10, the AMR curing plasmids comprising the pJIMKCore_M conjugation system were very efficient in curing AMR in vitro and in vivo.

TABLE 4
In vitro curing of target IncC and pKPC_UVA01 plasmids,
using curing plasmids based on different conjugative systems

| Donor^{a} | Recipient^{a} | Curing rates (O/N) |
|---|---|---|
| Ec J53 (pJIMKCore_M-based curing | Ec BW25113Rf(IncC plasmid) | (3.1 ± 1.0) × 10⁻¹ |
| plasmid for IncC plasmid curing) | | |
| Ec J53 (pEc158_IncC-based curing | Ec BW25113Rf(IncC plasmid) | (1.2 ± 0.6) × 10⁻⁵ |
| plasmid for IncC plasmid curing) | | |
| Ec J53 (pJIMKCore_M-based curing | Ec BW25113Rf(pKPC_UVA01) | (6.7 ± 1.4) × 10⁻² |
| plasmid for pKPC_UVA01 plasmid | | |
| curing) | | |
| Ec J53 (pKPC_UVA01_unkonwn | Ec BW25113Rf(pKPC_UVA01) | (3.1 ± 1.3) × 10⁻⁶ |
| type-based curing plasmid for | | |
| pKPC_UVA01 plasmid curing) | | |

TABLE 5
In vivo curing of target IncC and pKPC_UVA01 plasmids,
using curing plasmids based on different conjugative systems

| Donor^{a} | Recipient^{a} | Curing rates (O/N) |
|---|---|---|
| Ec J53 (pJIMKCore_M-based curing | Ec BW25113Rf(IncC plasmid) | (9.8 ± 1.5) × 10⁻² |
| plasmid for IncC plasmid curing) | | |
| Ec J53 (pEc158_IncC-based curing | Ec BW25113Rf(IncC plasmid) | (5.5 ± 0.8) × 10⁻⁶ |
| plasmid for IncC plasmid curing) | | |
| Ec J53 (pJIMKCore_M-based curing | Ec BW25113Rf(pKPC_UVA01) | (6.2 ± 0.93) × 10⁻² |
| plasmid for pKPC_UVA01 plasmid | | |
| curing) | | |
| Ec J53 (pKPC_UVA01_unkonwn | Ec BW25113Rf(pKPC_UVA01) | (4.5 ± 1.8) × 10⁻⁷ |
| type-based curing plasmid for | | |
| pPC_UVA01 plasmid curing) | | |

All publications discussed and/or referenced herein are incorporated herein in their entirety.

1. A recombinant plasmid comprising a plasmid conjugation system comprising a transfer operon comprising a polynucleotide sequence which is at least 80% identical to the sequence set forth in SEQ ID NO: 4 (tra-mob) and a transfer operon comprising a polynucleotide sequence which is at least 80% identity to the sequence set forth in SEQ ID NO: 5 (trb).

2. The recombinant plasmid of claim 1, wherein the plasmid does not comprise one or more genetic regions selected from the group consisting of: a transfer inhibition (tir) system comprising the sequence set forth in SEQ ID NO: 6, pemIK comprising the sequence set forth in SEQ ID NO: 7, a mucAB comprising the sequence set forth in SEQ ID NO: 8, an exc system comprising the sequence set forth in SEQ ID NO: 9, and a MDR-transposable element comprising the sequence set forth in SEQ ID NO: 10.

3. The recombinant plasmid of claim 1 or 2, comprising:
(i) one or more plasmid replication genes; and
(ii) a plasmid partitioning system.

4. The recombinant plasmid of any one of claims 1 to 3, wherein the plasmid conjugation system comprises a transfer operon comprising a polynucleotide sequence which is at least 90% identical to the sequence set forth in SEQ ID NO: 4 (tra-mob) and a transfer operon comprising a polynucleotide sequence which is at least 90% identity to the sequence set forth in SEQ ID NO: 5 (trb).

5. The recombinant plasmid of any one of claims 1 to 3, wherein the plasmid conjugation system comprises a transfer operon comprising a polynucleotide sequence which is at least 95% identical to the sequence set forth in SEQ ID NO: 4 (tra-mob) and a transfer operon comprising a polynucleotide sequence which is at least 95% identical to the sequence set forth in SEQ ID NO: 5 (trb).

6. The recombinant plasmid of any one of claims 1 to 3, wherein the plasmid conjugation system comprises a transfer operon comprising a polynucleotide sequence set forth in SEQ ID NO: 4 (tra-mob) and a transfer operon comprising a polynucleotide sequence set forth in SEQ ID NO: 5 (trb).

7. The recombinant plasmid of any one of claims 1 to 6, wherein the plasmid further comprises one or more antibiotic resistance genes which is/are not present in the multi-resistance region of the incompatibility group M (Inc M) plasmid designated pJIBE401.

8. The recombinant plasmid of claim 7, wherein the one or more antibiotic resistance genes are selected from tetA conferring tetracycline antibiotic resistance, fosA3 conferring fosfomycin antibiotic resistance, and combinations thereof.

9. The recombinant plasmid of any one of claims 1 to 8, wherein the plasmid comprises a parAB operon encoding genes participating in partitioning of the plasmid.

10. A curing plasmid which is the recombinant plasmid of any one of claims 1 to 9 further comprising one or more of the following: an expression cassette encoding a CRISPR-Cas system targeting one or more genes conferring a trait which is undesirable in a bacterial population; a region encoding one or more entry exclusion systems; one or more replication genes, one or more antitoxin genes; and/or one or more antibiotic resistance genes.

11. The curing plasmid of claim 10, wherein the trait which is undesirable in a bacterial population is antimicrobial resistance (AMR), bacterial virulence, or heavy metal resistance.

12. The curing plasmid of claim 11, wherein the trait which is undesirable in a bacterial population is AMR.

13. The curing plasmid of any one of claims 10 to 12, wherein the trait which is undesirable in a bacterial population is encoded by one or more genes of an AMR plasmid, wherein the one or more genes is selected from an antitoxin gene, a replication gene, a multidrug resistance gene (MDR) and combinations thereof.

14. The curing plasmid of any one of claims 10 to 13, wherein the plasmid comprises a region encoding one or more entry exclusion systems (EES).

15. The curing plasmid of claim 14, wherein the plasmid comprises one or more regions encoding a plurality of entry exclusion systems.

16. The curing plasmid of claim 14 or 15, wherein one or more entry exclusion systems are from Incompatibility Group F (Inc F).

17. The curing plasmid of any one of claims 10 to 16, wherein the antitoxin gene is PemI.

18. A method of producing a curing plasmid, comprising:
(a) obtaining a recombinant plasmid of any one of claims 1 to 9; and
(b) introducing to the plasmid one or more of the following: an expression cassette encoding a region encoding a CRISPR-Cas system targeting one or more genes conferring a trait which is undesirable in a bacterial population; a region encoding one or more entry exclusion systems; one or more replication genes; one or more antitoxin genes; and/or one or more antibiotic resistance genes.

19. The method of claim 18, wherein the trait which is undesirable in a bacterial population is antimicrobial resistance (AMR), bacterial virulence, or heavy metal resistance.

20. The method of claim 19, wherein the trait which is undesirable in a bacterial population is AMR.

21. The method of any one of claims 18 to 20, wherein the trait which is undesirable in a bacterial population is encoded by one or more genes of an AMR plasmid, wherein the one or more genes is selected from an antitoxin gene, a replication gene, a multidrug resistance gene (MDR) and combinations thereof.

22. The method of claim 21, wherein the antitoxin gene is PemI.

23. The method of any one of claims 18 to 22, comprising introducing one or more region encoding a plurality of entry exclusion systems.

24. The method of claim 23, wherein the one or more entry exclusion systems are from Incompatibility Group F (Inc F).

25. The method of any one of claims 18 to 24, wherein the curing plasmid is a plasmid as defined in any one of claims 10 to 17.

26. A curing plasmid produced by the method of any one of claims 18 to 25.

27. A bacterial cell comprising a plasmid of any one of claims 1 to 9.

28. A bacterial cell comprising a curing plasmid of any one of claim 10 to 17 or 26.

29. A pharmaceutical composition comprising (i) a recombinant plasmid of any one of claims 1 to 9 or a curing plasmid of any one of claims 10 to 17 or 26 or a host bacterial cell of claim 27 or 28 and (ii) a pharmaceutically acceptable carrier or diluent.

30. A method of plasmid curing in a population of bacteria to reduce the prevalence of a plasmid conferring a trait of interest in the population of bacteria, said method comprising introducing to the population a curing plasmid of any one of claim 10 to 17 or 26 or a host bacteria of claim 27 or 28 or a pharmaceutical composition of claim 29.

31. The method of claim 30, wherein the trait of interest is antimicrobial resistance (AMR), bacterial virulence, or heavy metal resistance.

32. The method of claim 30 or 31, wherein the population of bacteria is a population of gut bacteria and the method comprises administering the curing plasmid or the one or more bacterial cells or the composition to a subject in need thereof.

33. The method according to claim 32, wherein the subject is a human.

34. The method of claim 32, wherein the subject is a non-human animal.

35. The method of claim 34, wherein the non-human animal is a livestock species or companion animal.

36. The method of claim 30 or 31, wherein the population of bacteria is a population of bacteria which colonise plants and the method comprises contacting the plant or soil in which the plant is growing with the curing plasmid or the bacterial cell or the composition.

37. The method of claim 30 or 31, wherein the population of bacteria is present in an environment selected from a healthcare environment, a soil environment, an environment comprising a water source, an environment comprising waste water, an environment comprising industrial waste, an environment comprising agricultural waste, an environment comprising sewerage and/or an environment comprising biosolids, and the method comprises introducing curing plasmid or the bacterial cell or the composition to the environment.

38. The method of any one of claims 30 to 37, further comprising administering one or more antibiotic agents to the bacterial population.

39. Use of a curing plasmid of any one of claim 10 to 17 or 26 or a bacterial cell of claim 28 in the preparation of a medicament for treating or preventing antibiotic resistance in a subject.


SEQUENCE LISTING (10 sequences)

SEQ ID NO: 1 - DNA, 87731 bp, synthetic construct
  misc_feature 1..87731: note = Plasmid pJIBE401 complete sequence
  source 1..87731: mol_type = other DNA
aggggataaaatgattcagtcacgtaataaatatctgcaatttatgctggttctgctggcagcctgggccatagcctggggggccctgtttgtcatggggcagaccgttctgctctatgggctggggaaaaatacgctgttcttcattcatggtgctgttctggtgtacctgctgtgtgtgttcctggtttaccgccgctgggtcgcaccgctaccggttgttggccagctgcgtaacgttggcgcaccgtggctggtcggtgcgatggccgtggtgtatgtcggagaatttctgctgggtaaagtgctggacctgcccgctgagccgtttatgacgaaactttttgccgataaatccatacccgacgtgatcctgacgttgctggccatctttatccttgcaccgctgaatgaggaaacgctgttccgggggatcatgctgaacgtcttccgttcacggtaccgctggacgatgtggctgggggcgctgataacgtcattgctgttcgtcgccgcgcacagccagtatcagaacctgctgaccctggcagaactgttcctggtggggttgattacatcagctgcccggatcagaagcggcggcctgctgctgccggtattgctgcatatggaagcaaccgcgctgggtttactgtttggctgaaagttatatttttattaaacattgcgcgttaaagcctggtatgtttttttagtggatgttatatttaaatataacttttatggaggtgaacaatgcataccacccgactgaagaaggttggcggctcagttatgctgaccgttccaccggcactgctgaatgcgctgtcgctgggtacagataatgaagttggcatggtcattgataatggccggctgattgtggagccgcacagacgcccgcagtattcactggctgagctactagcgcagtgcgatccgaatgctgaaatctcggctgaagaacgtgaatggctggatgcgccggtggctggtcaggaggaaatctgacatggaaagaggggaaatctggcttgtctcgcttgaccctaccgcaggtcatgagcagcagggaacgcggccggtgctgattgtcacgccggctgcttttaaccgcgtgacccgtctgcctgttgttgtgcccgtgaccagcggaggcaattttgcccgcactgctggctttgcggtgtcgctggacggcgccggcatacgtaccaccggcgttgtgcgttgcgatcaaccccggacgatcgatatgaaagcccgcggcggcaaacgactcgaacgggtgccagaggctatcatggatgacgttcttggtcgtctggccacgattctgacttgacccattgtttcgacagagaaccatcatgatgaaatcaggaaatttgataaggtatttgtggttttttatctgcgttgtcacatgcctggtgatggcgtatttatgcagcatggttaatccatatgatgacagaactttgtcctccgcctttaccagctttctgaggccaatcggggccgggtttggtacggcgattgtccttatcctgatcactagcattgcgcgagagctgcggtgtttctgcaataaataaaagcgtgccggcccgatatttatgccggcattgcctgtttcctgaagtctgcaatatactgtatataaaaacagttattggaggctagattatggcgctggatctcgtttatccccggccttcagtggaaggcgccgcacttcctttttttgccgaacccgttgcggccggctttcccagtccggccgcaggctatgaggcaaatgaacttaacctgcatgattactgcgtgcggcgtccggccgcaacgtacttcttgcgtgtgtccggagattctatgcgtgatgcccgcattcatgatggcgatgtgctggtcgtcgatcgggcagaagagccccggcacggcagcatcgtgattgccagcatcgataatgaatttacggtcaagcagctgcagctccgcccttacccctgcctgatgccgctgaatccggcttttccgccaatcaggtttgatccggaggcgctgcagatatggggtgtggttacgttcactgttatgagtcaccgcccgtgtacgccttaatcgacgtcaattcgatgtactgcagctgcgaagaggcgttccgccctgatttgcgggggcgccctgtggtcgtcctcagcaacaacgacggggcgcttgttgcggtcaatcgcgcggcgaaagcgctgggagtgcgccgcggggaaccctatttccgctgccgtcgcctgctggaacagcataacgtcgccgtattcagttccaactacaccctttacgcctcattctccgccagatttgcaaccgtagtggaaagtctggcgccgcggacgtttgtttactcaattgatgagctctttgcggatgcgtctcacatggagggcgtgatgtcgccgcaggcgtttggccagcttttgcgggctgaagtgctgcgccagacaacgctcacctgtggagtgggcgtcgcgccaacgaagacgctggccaagctgtgcaaccacgccgcaaagacgtggcctgccaccgggggggttgtggcgctcagtgatcctgtgagactgaaaaggctgatgagcctggtgtcggccggcgatgtgtggggggttggccatcgtaccgaaaaagggctggcgacaatggggatcaaaacggcgctggcgctcgcagagatggatatccgacttgcacgccggctgtacggtgtgacgctggaacggaccatccgtgagctgcgtggcgaggcctgctttgccctggaagaaggtaccggcgcgaaacagcagcttgttgtatcccgctcgtttggatcgcgggtcacgcaactggcgcagatgcagcaggcggtgactaaatatgccacacgggcaggggaaaagttgcgccaggaaaaccgtctggcgaaggtggtaacggtctttatccgcagcagtgcttatgacgccggcggtgtgccctacagtaaccaggctacggaagtgctttccgtgccaagccaggatacccgcgacatcatcgccgcggcgcagctggggctgtcgcgtatctggcgtgagggtgtgcggtatgcaaaagccggcgtgatgctcagcgatctgcagggacatgaaacccagctggatctgtttgcgccggctgcagtgcggccgggcagcgaaaaactgatggccaccatcgacaaaataaacagggaaggaaggtcaagggtcttcttcgccggggaaggtattgatccggaatatgccatgcgccgtgaaatgttgtctccggcctacaccacgcagtggaaggatattcccgcggcgtttatgcgataacagcgatgtgcaggccagtctgtcagccctgcagccgttcctgctcctgcagcaatgccagatggtatgcggcaatggcgttcaccaccccggccgtcatccgtgcatcggcgattgccgagtgtgcctccccttcccaggccaatgctgcctgactggccgcgctggccagcgaaatggtgccatagcgattggtggccccaaaataccctgctgccagctccatcgcgcaatatactgtcagttctcccagccagtcggccggatcgccatgtgcggcggccgtctgtttcagaatgcgggtatcaaagggggcgttaaagataatcactggccggtccccgatggcgtgtcgcagctgcagcgccacatctggccaggatggtgcgccgctgagtgccggctcatcgataccatgcacggcggcggcctgcgcctcgatggcgaccgtcggcttcaggcgggtttcaaatatcacccggccggccgcatcggtcaggccgatttccagcgcttctgcggtattacccagccccgtcgtttcggtatccagaaagagcggcgccagactcagccagtcgtgtgcccgccgtgccatccggccactggtgctgttaagccgggacaggacggaaagacgcaggccggcctgctgctgcttctcggtcggcggccgtttttcccgcatcggaacacagtctgcaatgcggtatacggcgtacttgccgccataggcgcttttgtaccatttcaccggctcagcaccgggagccggcttcattctgaattcatcgcgcaggcgccctttcgtgaagcaccgatcgccggctatcgcctctgcgcgtcgtgtggccagctcatcattcttcatggttattccatctccgtcaggcgtttgtgataaacctctctggcgatctccaggcctgcctctgtcagcccaaacgcgagcgtcagtgactgattatcccggtatttgctgagcaggccgtgacggttcagcgtatggcaggagcgacgaaaattggtatcggcaacgggctggaggcgtcccttattgaggatgctgaaaagctgcatcgccgggaacgggcccgccagctgacgttctgccagaccatacagaacaaacaatacgtctttttgcacaccggagagccgcatcgagggggctttactttgcatcttattcatgtgtcacgtcattaaggaattaatgacaaagtaacctaattgatgtgagacatcaataatattgtgttcataacgggaaatcaggagtccgaaaaagaaaatgccgctcttgacggcggcttgttttccctggtgtcgggagaattatggtttctggtctgggtcaatgcgcagggcgctgtctgtgagcaaatccagtgctgccgtcagatgctgactccgcccgccgtgataatccggccacggaagaaagggttttgacgtatcagacatgaacatgataccggtcgggcagcgccactccgtgggggttcccttgttgtaatgaaccagcagtactgaacggtcacactgcacgtcattactgtcatcgttgttcagtacaaaagtggccacgatgccggcatcgggcctggacacaaaaagcaggctgttggcaaaaataacggcaatcaccactgaggcaatggccgcaccggtataccagaaacggccgtttttccaggcccgatagccatacagaatgcacagcagaatggcggcagcgatacctgcccactgcgcaccgactgacaggcgggagagtgcatccatcgataacggccggctcagcgcataaaatgtgacaaggaaaggtgaagccagcgccagcgtcagcagaacgaaaacgaggggtaagaagcggttgccggtatagctcccattgccaagcgccgtcatcgtgcggatcatctcactgaacgccatgatcatcataaagcagcataaagccatgtataacattaaaacatccccaaactgtccgcgtgatatggccggattttcggcctgttccacatagtcccgaccccttatcggggtgcatagccctcttcagccagtgctttcatgatctggttgattccatccatcatcagatatgcctcaatatctgacgtcgccaggccgcggcagtcactgaatcccagccgcttaaccagttgcgccagagcctgcgcttctctgtgtgtcagctgtatattcacttcaacgggtttttcgtggcgaatttcatctttcatggtcagagtccttttttgatgtttcgtgaaatgtaccaggcttcttttgcctgagctacagcccagatccagacgagagttgacacaatgcacagccccgtcatgttccagaaatgacctgtcgtatattcatggccatgaagcaggaggccggcgccggccgccaggctcatgaccaggaaaaggacggctgcgccggcgtgataaatgatgctgaacatgtaaaacccccttctggcgacggtagcgccagtctctgtctgtattaaatggtcgccgcctgccgggcgtctgcctgcatcatggcgtcaatcagggcgccggcgcaggcaatattcaggcacgtcatgttcgatttcagggtcgccatcatgtccgcgcggcttcttttcagaaacggctgcagcggaccacgatagccctgtgaggtcacgataatcagctgagtggccgcatcatgcagtgccgacacgccggccagaatatcctgctcagccgcgctgatatacacgggtgaaccggcgatcacaaagcgatcgtccgggcagcgttgcatcagctcagccagggaggcgcagcggcccgccagcgggggccgtgccgtcagtgagccccagacggtcgccggcgcgaagggcatggcgtgaaacgttgcctcatacgcgaccgccggatcggtatggtggcgcagccccagaccggccgacatggcccacagctcgatgcccggccaccgcgccgcgataccgcgggcacgcagccagtgtgcgccccggtaaagctctcccgtttccagcaccctgccaggacggcgggcagcctgggcgatcagcctgtaccagccgtcaaaggcgtcatcaagggaagcgtcgcccggaaaaacggtatcctccacgctgcccgccttgcgccgggtacaggtggtgatgaggtgaagacgtttgttcattcagtgcctcccggctctggtgcgtctgtgcctgccgcgcgcggttcgccgggccgccgggcacgaatatcgggcagaccccggcaggcatcgttccagtgcgtgccccgatcggggttattggccagcttactgatccaggtttctgacacattccagcggcgggccagctccttctgcttccagcctttcgccctgagcagcgcccgccactcctgcggggaaagcggcgccgccgtgatgccggccgcatgacaggtcgttttcatcgcgtatgctcccggccctaatcctgccccggccgtgccggctggatttgacgcagtattgttgttgcctgccatcccgtaaacccgaaggtatcggccagttcgccgggcagtgacgcggccaggcgcggcagttcgtccggggattcattgtgaaagacaaaactgaccccctcatccctgatgcgcgacatcaacattgcaataagccggtgcggtccctcatggctggcgtattccgtgaccttttcagtaaaccgccggggattgctgcagctgataccccggtcggccaggaacgccagaaagcgctgcaggaaaccggggagatccctggcggtcagggcgggtttcagcaccgcttctgcctgggcctgtgtgaagccaaacgccgccgccagctcaggggaaaagtccggcgtgtcagcgctggcctcaaaaatgacgccatcgtttatggcctgctgcatgtccgggggcatattcccgatcatattttcacgcacgccccatgcacggccggcgctttcgttcagtctctgcggatcgctgcagctgacgccccgctccgccaggaacgtcaggaaacgctgcaggacggcggcgccgatctcgccggagacgggcgcttctccggagggatagaagaccctgacgttggtgttatccagtaaggcctgttcagcgccggcgtcggacccggtgctttttccgctccgccagaccgtattatttcgactgacgatggccggatcgacatgttcgctgtccacgatgctgttaatgaacgtcactgactgttcttcgctgaaatcaaagtcgtgctgtaacagggacagcagcctgtcatgccagtcatctttcacgcgattggcggcattaaccatgaacgtgacgttcagtacaccggccagtaaacaggcagcctggtcggccacatacaggctgaagtattccagcaccgcctgttcaaatgcagcgggatcgcagcagaccacttctgcagatcccagcgactgcagaaaccgccgggtgttttcggtcacagcgtcggcgggattgagggcgatggcgcggccgcgctgtgactggatgtcaaatgtgacgatacgctccgtgacgggcatccggccgtgatcggcgatgaagcggtaaagctgctccactgcttccgtggcggtcatgcccattgcctccagacgcagttcggcgcgtttcttcagctgctcatcgagacgcacggtcagctgggcgtttttggcccgctttttacgttcaaaaaaaccggtcatgctccttcctttttgtacagtcatttggctttacataatgtacagccatttgtaccgcacttttgcgcgtgggaccacattttttcaccgttgcggtttagcggtattctccgcagtgccgccacttatccccagtttctgtggataacgctatatgtagtggctccgtcagctggaaacggcacaagatatggtgtccgcgccggcagccgtgcggccgttcccctgccttcccttccctgttgatttcgctttttccggtatttacgttttttccgcaatcacggtgccggcgaaaaggtattttccggtggttgcgggcgcaaaagtcacagggtgaaaagataagggcacgaagcattccgcaatataaactgcgtgacagtcacgccgggtgatgactacccgcgggcgattctgccaaattagctattagtgctaaatctaaatatttataatttacgcatgaaacctaatttattagagtgtggttattcagtaaacaacagcaaacggagtgaacgggatggaatatgaagcgtacaaggttcgcggttacggcttcggactgaaaaaagagatcaccggcgtgctggggcgttaccccaccctggaagaggcgcagaaggtcgcggatgactacacgcaggcctgcctgccggataacctgccggacagctacgatatgggcatggtccggccggtcgctgccggcgaacctgccgccctcgatccggacgtgtttcgcggtccgggcgcgccgttcctgatggatgccggccagataaccacgggtcacacggtatgcctgggcaaagcagcggatggaaaacggttttattttggcggggagcattccgatggccaccaaacatataaatgatgagctctggaatcgcattgaggcgctgaccgtcaaagctaacgccaggcatgggttgttgcggccgattaaagaggctgaagtactgcatcttgtcctgcagcgtggcctggaactgcttacggacgacgatctgctgcagctgggcaagtaccggcgtccgataggatttgtgctgcgtcgtccgggcgtggaaatggtgaagctggatatgctgaatatggcagatgcggcaaccgttcttctgcgcagtggacccgcaacgctgtgcatctggagccgcgacgatatcctgcgcgaggccagtgaagcggtgatccgcgaacggctgccggatgcagccctgctgtcggagggggatgaccgggcccggttccagacgctgctgcccggattctggaacgccgcgcaccggggcgaaacggccgtgatttccctgcgtgctgacaatgcggatctcgctattgcacgcatcactgacctgatgtgcgaatccctgctgggttataaggggcagcgggcataccgggcgggtgaaaacgaacagggagaagagtcatgaacatgcgaggcattctttttgtcctgccgctgtgcgccgggctcctggcgggctgtgataacaaacaggaagcgctgcagtttgaagccgcccgcacggcggcgctgaaggccgacgatccgtttctggcgctggactcgttcgaacagcagtccggcgactacggccgggactgcgactcgctgggggcatttctgtttggtaattttgcttccacggataaagcatgtcgggcgcgggatgaggccacgcgggatatggtcctgaaagccgtagaaaagggttccgtgccggcactggtgttcctgtttgacccaggcagcaagacgccggatgtgtatccgcgcgatattgacccacagaaggccgggcaggcggctgagcggcttgttgcactggcagagtcggcgccggtggacagcaaaaatcaggcgctgctgatgcgtgccggcgatgtactgcaggcgggccagtacgttctgcaggattcccggagagcggcggccctgtacgtcaaagcctggcaggcaggggatcagcgtgccgcggacgagctggtcaatgtctatcactttctcaaggacagccggcgtacctacttctgggcggtccggagcaaagacgtgcagcagctggacaatcgggctgagctgaccggagatgagatcattgctattcagaaactggctgcggacagttcacggaccgacatttgatgaaatgaataaggatatacaaaaggcgtccaactggtgaggaagggcgccttttaattactagacatttttactacttaactttctgtcggagtaaacggaaaattaatctgttaatcactgcgagtataaatccccgctaagcgtggtgcaactcagtatcagaaatgaccgccggtggttgaatccagtgccctgaacaggctttcctgggctgcttccagcagacgacctggcagacggagttcatttacgcggtaactctgcccggtctgtgtggtcacggtgagataatactgatcgtcaaactgctgaccagcaccgatatgcacgatggtggccgtgtcgatccagcagaggtttgtgccttccgcgtcctcaagtggtttgattcggcctttcagccacttctgcgggcgacccggccggcatttgatcgtgatgccgtgcttatagccgcgggttctggccacaaaggccgctattttatctttatccagttcctgcgccggggtgagcttactgaaactggacggagagaagttatactgcagttcttcaaacaggttcaggtacgtggcttcatcatccaggttgtccccgcgttctttcagcatggagccccagatcctctccatttcgcccctcagcgccagtttaaccgcctcaaattcttcctgatgttcatcgtagtagttgatatccatcgcgtctcctgttgtgtttatggcgaatttgcggaattaatatacataaaaaatgttcaaatatgaacattttcaggctataaatacaggcagattataacaggccagtccaccaggattcggccaggagctgagtacgcatgataaagaaaccacttattgccggcatcatgctggctgtggcgcacaccgccggcgctgcgcagggtgatgataccgatctgcagcagatttataccgaggcgggcattcaggggatatgcagcgccgggatgctggctgtgccggaaggggccagggatgaagctgtcccgcagaacgtcagacacctcctggttcagtgtgcctatttcgacgagcagcagttgcgccggcgtgccggcctgccggctcagacatttagccactggcgtgaccgcaatgcggtccttaatctgtcgtctgaccaggcatctgatgcgctctggctgtcagcgtggcgtgatttccgggcacaactggcctcggcctacccggccccggcggaaaaaaaggcacaggggaaagaggccgtacccgccggcacgaagcccggcactgagcatgggagtgcggcggattgaccggttttgacttgatcctgtggcgacggggcctgaactggacccaggagcgcgccgcggctgagctcggtatcagccgtaccagcctggtaaaatatgaggacggtgagacagtaccccggaccatacaacttgccaccgtcgcgctgacgctgaaagcggaatggcccaccctgaaaaccatgagcaaagacaggctgctgcggcagctgaagtatgaagtgctgaggatcgataatgaatgagtttatccatgttgtcggctcatttatccttgcgggtatcctgctgtatggcctctggcagggtacacggcgacgccggcgccggcatgaacggaaacaggccagtgcggtgcgtgtgatcgataaaattaatacgttcccgcatttcgggcagaaaattgcctatctgcgcaagattgacccgtttgtgttcgaagagctgctgcttgaaggctttgaacgccggggctttgaagtgatccgtaaccgccggtataccggtgacggcgggattgatggccgcgtgaagattgatggccagacctggcttatccaggccaaacgctataccagctatattgccgttggccacgtccgtgacttcagcgacctgctgaacgccaccggtgcccgcggttttttctgccataccggcaaaacgcgcgaaggcaccaaatcgctcattcgcggagacagccgcatgatcctggtcagtggccagaagctgctagaccttattgccaccgatgcgccgtttatcccgtatccgggataccggccaccggccgaaatggttcatcctgaacaggagacaacatgaaactgaaaggatacgtcgccgccctgccggcactgctgctgacagggtgcgccatgctccccggacagcccaccgattatgaccgcttctgtaacgtctcaggcatcgccagccacggcgaaacctaccgtgtcagtgacagtcaggatttctggttaaccccgaacggccgctatctcagccaggctgagtacagcagtccggcagatacgcttcagaaactgacgggcgtggtcagtggtgaagatcctgaccaggtgcggaagaacgccgtcagggtaagggtgttccgtgttgaaagtgagagcgccagtaaaggggcgtgcctgcccgtgcgttatgacgataaaaacgcgcggcggaaaatggattcgctgactaacggccgccgtatgatggtgtacagtgaagatgaaggccagtctggccagcagatttacaataacagccgcggtgccggcttcagttacaggttgctatgagtggattgaaaggccgccgtatcagtgcggatatgtttgccataatgctgaagatatctgccgggcagctggtttttgcagcacggcatgacaagccgttacgcggactgaccctacccccttacagaatcgaaggcggaaaaagctggagctcgaccacctccacccgcagtatgacgtttgatatgcaggatgcggtcgatttcagcgagaaatttcataaaattcagtcgcctgacggctgattagcggattttaaaaaattgaacactttccccctgcaaatttctcagaattcacgtagcgacgccatcgggacatatgcctacttcgattatgccgatgcgctggcgctgcgggagatggccagccaggctgatttacccaaatacctgctggccccggaggtgtcggtgtttctgcggtatgtgccagacctgcgccagcaggtccttttcgataccctctggaacacgggcgcgcgtattaatgaggcgctggcgctgaccggcgccagctttcagcttgacggttcccggccgtttgtccggctgaagacgcttaaacagcgtcagcgtggccgcggcaggccgggcaaggatgaagaggtgttccgtcttgttcctctgaccgatcctcaatatgtcaggaaggtgcgtgaatttctgaccaccctgcgtattggtaaacagcagctgctctggccggtgcagagtgataacacgccgcgtaactggatcaggaaggcactggacctcgcaaaacgtgactctgtcacttttagtatccctgttacctgccacacgttccggcacagcttctgtatgcacctgatccagcatggggttccgctgaaagttgtacaggcttatgccgggcattcccggctggaaaccacagaaacctacacgcgcgtgtttgcgctcgatgttggtcgtcagtatggcgtccggttcagcctgccagacagtcatttaacgctcccggaataatctgtatccctccctgatatttccatataaatctaaataaatccattttctttctaggtcagaccgttttcagggcggcctggccgtcttttgttggtttttattctatatttttctgtatttttctatattggtgcgtctttttgtctttacacgtcagcagggatgcgtaataatccatttgaaagctgtatttttcctgatttctacggtttttttctaaataatgtgaggttcaaaatgcgtatttttgttgacgacggttccaccaacatcaaactggcatggatggaagacggtgcggtcaaaacgttgatcagtccgaacagcttcaagcccgaatggtccatgacgttgctgacagattcatcagtgccggcttccgccaactatgaaattgacggggaaaagtacagctttgaccagctgagcccggatgccgtgcgcaccacggaaacccgctatcagtacagtgatgtgaatgtcgtcgctatccaccatgccctgatgcagtccggcattgaaccgcagccggttgatgttgtggtgacgctgccgctgggcgagtacctggatgaaaacgatcagcctaatatggccaacatcgagcggaaaaaagcgaacgtgaagcgcgcggtggccgttcaggggcgtgagagctttaccgtgcgtaaagtcagcgtactgccagagtcggtccctgcaggcttcagtgtgctgaaggatctggacgatctggactccctgctgattgtggatatcggtggaaccacgctggatatcgcgcacgttcgcagcaaaatgtccggcttcaccaagacacattgcgatcctaaaaccggcgtatccatcatcaccgaagccgtaaaacatgcactggataccagcgtgagcacccgtaccagttcgtattttgccgaccgtctgatccaggcgcgaaatgacgaaacgtttctttcccgttacctccagaacgctgaccagcgggcccaggtgatgaaggtactgcatgagcgtgaaaaagccctgacgcaccgtgtcaccgattcagttggccgctttgccggcttcacgcacgtcatggtggtcggtggcggtgccagcctggtcgccggcgcagtgaagcaggcaacgggggtgagtgacgatcgtttcttcgtcagtgataacccgcaattcgatctggttctgggtatggtggcaatgaagggctgagtatgactgataaccgtaagtgctcgttttatatctacccggagcgcaatgcggcggaccgggtagccgacggctttcttgagaaactgcctcagaaagagcgtggccgcgcgatgcgcgccatgatgctctgtggtgccgcgttgatgaagcaggacgaacgcctgccgttcctgatcgctgaattcctgacggagagcacctcgatgcaggatattcagcggatcatcagcagtacgctgccgcagcaggatacaggagaaatggcgcgtctggttgaggcgtttctgcaggccaccggcggtggcagcaatacggtgtcgccggcaggcggcgctgcgccggaaaacagtcccgccgtgactcccgaaccggccgcaccggaggataagaatctggaagaaacccgtaagaacgtccagcatctgttcccggatgatgattaagggggcagcgtgaaaaaatatgtgctgttaaccctgaccctcatcgccggcgccgtggcagcaaacccggtaacagcggccggcaatgttcaggcgcaaactgcgactgaagtgaaaaccgggttttcacccggcgggaccgcattaaatctggttctggattcaatcgaccacgcgcagcatgagattctggttgccgcgtacagcctgaccagtcgtcctgttgccggcgcgttgcgtgccgcaaaaaaacggggcgtatcggtggtggtggtggccgatcgcaaagcgaacgacaaccggtacaccgaagtgaactttctggccaataaccgcgtggcggtgcggatcaacgataactacgaggccatgcatgataaattcatggtctttgaccgccgcgccgtcttgaccggatcgtttaacttcagcgccagtgcagacagtcgtaatgcggaaaatgtggtgctgatcagcggcgcgcctgcagtaactgaagcctacgtgaacgagttttcccgtttatggggcgaaggaaaggacgtggccccacggtactgacctacagcagcggcagaaactctgccgcctgatttttaacttgatttatatggctcattcgtcgtattatcaaggctcatttttcggtagcgccccgactggcgaaggagtaaaaccatgcaatcattccccctgttcgtttcccgtgtcgtacctgtgatcaccctgagccattgccttcggcagcggcgctggtccctgcacaacacgtcatgccagattgacctgagatcaggttgtcacctgcgctcacagcctttcggcacgctatccagacgggaaataagcgaccgaccaccggctatgccccgcggttactggttcaggcaatgcagccgggatgtcatgtttacggggcgcatcgatcaggcagatcctatgaaacaggtctattacaacgaaggctggagcggtccgaacaagtatacgtttgaagtttatcagctggagaacgggagttaccgggcgctggcccgcaaatggaatggcaaaatcaacaaggtgcagcaggaaacgcagtacctgtctgacacgcgggaggggctgaagcaccaggactatccccgcacgcgccaggtgaaaatttttctgaattcagacttctgggagaaaggtaatgattaagatggttagcgtcgttccgcagccggaaacagtgaagacgctccgggaaaaaatgggcatgactgaaaccgcattaggggctgtcatggggtacgaactgcgcgcctggcagcgtaaggaagctatcagcgacgatctcagccagtacaacaagaccagtcttcgcccaggcgaatataacatgctgatgctgatcgccggcgtgcatcctgattaccgcctgaacagggcattcagtccggatgatatggtcaaagatcccgccacggccgaagacgttcgccgcctgcgtctggcgctggggctgaagcacgctgaaattgcagcgctgtttggctataaaccggcgtcatggcagaccaaagaaaaggctgctcagcgcggcgtaaagctcaaaaccggggaattcaatttcctgttacttctggccggtgagcatccttcactgcagctggttgagaaggtgaaataaggcccgcggcgaccagagaggaccctttatgatcaccacacgccgaatgttaatgctgtatgcctgccttgtgcccgtagccctgacagggtgtgatctggctgattatttcccgaatgtgccacagcccgtgcccgtgatttcgcccggctatcaggttgatatcgatggccacccggtccgaatatcaggatttgatgactgcccgcgggataaagatgaagcccggcacgcagctgcacaagatgagaaaggctgcattgtgctgtctgcagagcgttccgaggtgaaggtcagactgtatcagccagccggcaccgtcaccgaaacctggcggatcatccgggaaaaagacgctcgccagaacgatattattcgcatgcagcgcccgaacggtgcgcttgtgatgcaaaatttacaggggaaagagtctccttaaaccaggaaaataacaaaattaacttgatttatatggctcattagacgtataataattacatcgaaaggcaatccggtctttcgacggtagcgccccgaccggcgcaggagtcaccttatgactgctctcaattctcagcaacaggctgatgttgcccgctatatccgggaacatggcaataaaaccgttatttcccctgaatggtcacttatttatttctcgctgtgtcagtgtgaattacatcaatcgcatgaagccattatttttcataatggccgacctgcagggctatttcttctgaatgaaacaggcgagctcaccgaattatatctgctggggtcgctttctgacattctcaccgccgctgcggtcaacaaatagctttcagggagtaaatacactatgaccatgcaatcagaacttgtttttactgatcctatgctcaacgtcgttattgctgaagttaagcgttttaattgcccgcttctttttgtcaaagatcatggcgtgtatgtgatggcggcgaaaggtgagaaaaacagtaatgggatgcacaatgtctgttacgccaatggtttcaatccggatacaacggattttgacgagctctgggaccggatgcgtgatgcctgtggcggggatgatttttgcgaatccctggacctcgatccgaggtcgattgagcttctcagccgtaccaaaccctgcctgaaaattatgctttcagagacggaactggaagttattgcagggggacagaaataaaccataaaagcccgttaaatcgggcttttttatatttaactgtatttacatataggacaaattgtcctaatatatatttgtggccgggcatggtgctcaggtcccctaacgagggaaatatatgaaattttcagcagaagaactcaacattatccgccaggcggacgccattattgccggtaaaattaccacaacggattttatcaccggcgccgatgccgcgcgttccttactgcggtttcgtcttgttgcagaaccacgcgaatcattctgcgcgttctttcttaactgccacaacggcgtgattgcgtttgaagaactgtttaagggcactatcagcagcgtcaccgttcatccgcgtatcgtcctgcaacgcgccctgcatcataatgccgccgccgtggtactggcgcataaccatccttccgctaacactgcacccagccaggctgacaagctgatcaccgagcgtctggcgcaggtgctggggatggttgatgtcaaagtcctcgatcacctgattgtgggcggcagcgatatttactcttttgctgaacatggtctgatttgacaactgcggccgcacagtggtgcggccggctctggaggcttgctatgggcaataaatataccgtgccgcttaacgtgggcgcgatccgcgaaagttgtttccgtcccgctgataaatggaccccgccgacgggggatgaactccgctatctgctggaaaatgtgctcaacctcagtcaggaggggctggcgcgacatgttggcgtgaacggcagaacggtgcgccgctgggtgaacggagaaagtgacatcgcgtattccgtctggtgtgtcctttgcatcgatgccggcttaccgccgatctggaaatagccgcgcaaaacaccgacgcgcaaacgtgcttcctgaacgtatcggatatacgaaggaaatattatgaatactgataactattttttcagagtcccggccacacgcggaatacagggcggcatagaacaatacatgctgacggttcctatggtcgtgttacgtcgtattcttgcaatggacaatgatggcgatgttatggatcgcagccagcgagaagcgaacaaaacgcgggccaaaaaaatacggaattatgttgccggcgcaacatcaaaacgagctccctatattttgccgtcaattaccggaaatattgatagtcatgttgaattccttcccagcgaactctcgccggcagtcggtattcttaaaattccgatggacgctgatttaaaattattcgatggccagcaccgtgcgctgggtatttttgaatttgtacgcgattattccaacacggaagatacgatcagcttattgcttaccgttggcctgccacttgagttacgccagcagtttttcgctgatattaataacaacgcgagcaaaccggcagcggcgatcagtatggcatataacaacaatgatccggtcaaccagctggcaatgcatctggcccgaaccgtcaccgggctggcgggcacagtggattttgaacacaacgtggtcccggcaaaaagctcacggcttatcagctttaaagcgctgaacgatgcgacgaaaaaaatgctcaacctgcgggccaacagcattccctcgccgcaacagcgtgacatggcagaaagactctggactgcctgggcgcaggcgatgcgctggaatgatattgcccaggatgatatcgccgcggagtaccgtcaggaggcgctgggactgcatggcatcatgattaatgcgatcggtatggcaacggccaggatgttacggcacagaacccctgaaagcatcgagaacctactggcctgtgctgaaaacggtgacaacggttttcattaccgggagtcgtttgtacctgagtgctgggaagggaagtgtgttgatccggaaacaggcaccatcaaaaccgacaggcggtcgctcgaagcaacggctgaagcgttgcaaaagctgatagatccgttcgcagatgcgctatggttgcgtgattacctgccagctgaggaggcgactgatatggcattactgaaatatgcagccgacatcgagaattataagcagcgtactgccgcgccgatgatcaatatcgtggagaagctgaaggcgctgggtgacggcgaaccgcagttcagagcgtcggtgctggcttccagtgaaggactgagccactatctggccggggctgaaggctgaccaggtgtccgccgcctgcagggcaatacgggcatcgaacccgtattgccctgaaacgcgctgagcagctctctgagcgcttgtttcaaaaacctgtaccccactcgcctctattgatttgacgtgtccagccggccattctggccttatctgatgttattccgacatggcatctgccagttaacctacttctgacatccgtaccgtgtgtcttccctgttcatttctgctcagttgccggcgttttctggcaggcctgtcgtactaaaacacctctgggccccttcggctcctgcccgtcgggcagtcgtcagccacactccgctagccaggcaaagcctggcagctccgccgaaccccctgcggggtttcgggctatcgctagtgtctgatgcctgaacccctgcaccatgccggtaaagtgctgccagctggctgatggggatctgaaagcaaaatttacagggaaaggagtccgggaaaaaacaaaggaaattaatcagataacgcttgattgttatggctcattagacgtataatgatcacatcgaaaggcaatgaggcttttcgcggtagcgccccgactggcgcaggagttacaaattatgagtaaagcactgaccccttcccagcttaaagctgaactggacacgcagaaagatgcgctgctgaaggcgtgcctgtatgccctcaacgaaatccctaaccgccgtttaagtggtccgtattcgtccacgtatgcgctggccgcgcagattagtaaactgcttcagagctgcaaataatcattgatgggctggcattgccagccctgttcaacgctaacggtagcgccccgcatggcgcaggagtctgaaatgtcatctaaaaatactctcccgtcggtcgatctgatgtcctgccagcatggcggtgacgtgatcccgtccattgctattgagcgcattatctcgttgcgggatgaaggtatccgcgtctatctcgacggcatagacaagctgcgaaaagcgcgcgatctgatgcagcaggccggaggctgtcagtacctgtatgactacgacaaagcagtagagagcgccatcaaatggagcgacaccacgccagaccaggccgaaacggctatttcccgcgttatcgacggaaaaatctgggatcggctgatgacggaaaccggcatgtatacgctgatgagcaacaagcaacgtcaggaatgggatcggcaggtggcaggcaaagaaatgccgccgataacgctggataacgttatgagcacgttccggcacctgaacgcaagcaaggcggatactttcacgcaagggctgatagatatctttaaatccctgtcgtgggattataaaaccaataacccctgcatgtttggcaagcgtatcatcatcgcgccgttgctcgatgtctggcgcagtggctgggtgcgcttcagctcagacgggcacaccaaaattgacgatctggcgcgtccgttctacgtgctggacgggcgtaacgtgcctgattaccgtgtttctgacggcgccaaactggatgcttttttcagtgaaaaccagttcaacggtaaggtgttcgagtgtgattacttcagcgtgcgatattacaaaaagggcagcgcgcacattacgtttaaacgcccggatctggttgagaagatcaataatctggtcgccagtcactatcctgggatgttaccccctcgcgtgtaatccagacggcaggccgcatttgcggcctgaaatataaacagggaaaggaagtattccccggcctgagaaaatcatgccgggcatgtatttattgcttgattagtgtggctcattagacgtataatgatcacatcgaaaggcaatgatgcttttcgcggtagcgccccgactggcgcaggagtaataaacatggctattcaatcccttcagttccgtaacggctccgtttctcttggtgatgtttttgtttccagctggggttatgagcagacaaatacctgtttctatcaggttatcgccctgcgcggtaagaaaaccgccgtactgcgccgcatcgctgcgcagcaggtgaaagcagacagtgcgatgtcaggcgagttaaagccggttttgaacgagtttaaaggcgaacccgtgacccgtcgcattcgtgaaaaccatgagcatccctctgtcagcattgacgagtacgaacaggcttataagaccgatcctaacgaaagccatttttacagcacctggggctgaccgcctgctacgccccgcaaggggcgtatagtcaaagacttattcaaccaaaacgtgaatactatgcgaaataacaaggcgctcaggacgcagatacatggcggaaacagtttatcaggcgcggaggcgtttttaccttctccgttttcggcgcagccgtaacccggacacactggaaaaaatgtacgaatccatgcgggatcgtggacaggtgccgccggaggactccgaggcttttgaggctgccgccgaccatcgccgcgcagagctggcatcgggacgcatctgggataagatcccgccgcatgtctggcagtatgtaaaataatcacagcgcccccacgaggggcgtttttttgcttgtatgtatggctcattagacgtataatgtttttactgattcaaatcagtaccttcctaaacagccttacaggtgcattatgacctcttctgaaatcctccataacctccctcagattatcacggccgtggccgccctcattacagccattaccgggctgattaaagtcatgaaggataagggtaaggactgaaccgccgcagggggcgacagccccctgcatcgctaaccggcagcgccccgattggcgcaggagtaaaaaaagtgacaacgctgcatattcgagaaattcccactgatggcgcaccgcctatctgtgcaggccgctcaatctcagatgatgacctttgcgcctcatgccagcatctcgactatcgccctgggcacctcagcgcctgcctgctgtcaggttgcgggcgtgacgactggccttcagaactggatgaaaacggctacagcgttgcctgtagccgctatgagcgtgaaaaattgcttgattaatatggctcattagacgtataataatctcatcgaaaggcatccggtctttcgcggcagcgccccgactggcgcaggagttttacaatgcacggtaacgtaaatgaaatttgcgcaaggcttctcgatagttttgaaccacagcagcggatcagccttctgatctggactgctgaagatgtccatgactgcaccagtgatatgaacctcactgacgatgaggccgaagccgttctggcagaaatcgcggaatgcagttcacacagccgttatggcgtcggaaaagataccgtctggagcctggcaaagcaggtgcgcgaagatgccgcccgcgatcgaaaaatcgaggtcaatgccgaggcgctgcaaaaagtcgtggcgctggcggcgcagtttatccggctggaagagatccagtctggtgaaggagctgcacgccgactttatccgcaggagtctgaggcgctggagtgcatcacgaaggtgattaacggctaattcacgcagtaacgcagcgaactttcacggcagcgccccgactggcgcaggaggaaaaaatatgtcccagaatgaagcctattacgacacgctggccctgattgaaaaggggcaatggtgtttcggcagtatggacgaagaaaacgaagagcgggcaatcaaggccgctaaggcgctggcgctgtttgcgcgcctgaatgagcaggactgcgacggacacccggtttccgaaatcatcgttgattttatcgctaacctgatgcacctgggggaagcgataaattttcgtgtgctcgatgaggaaagcgcggtgatcccgttggtcaggatggcagccgtacactttaacgctgaaacaaagtcatagtaatcatgtgaatcagaggggggcaggcgtgatcctgcccccaaaaatggacaggggaaagagttgtcccgcctgaaaatattcaggcaggcggggatattttgcttgattgatatggctcattagacgtataataaacacatcgaaaggcaatccggtctttcgtcggtagcgccccgactggcgcaggagtacagaaaatgtctactgaactgaatcaacccgccatcactgcacgcattatccccaataaccgccgcatggcctttctgccacgcctgttcggcgcctggtatctcacaggtgaggccggtgtctacaaccacgcacgctctctctgcgccgattatcaaggcggttcctgggagttcgtcgaactgtcttgcggtagcggcttcatgtacccactgagcgcagagcgcttcacggtatcggttagcggtaacttgttcgaaggggaactgtccgcagaagctaccggtatcgtgctgaccctgttcaccctgaaccacatgatctggcacgcacatgaagaaggctatcagcacatctgcgacatgctgatcacccagcaggaaaagctcaaattatatgccgaccagcacgccgaagccggtttgatttaccgcgcgattgactgaaacggcggcccccgcgagggggccaatactgacagggggaaaaatgttaacactaactgatattcgcgccagcaatacggtgctggttacggaatttggcggcgtgcgtgctgtgcatttctgtctgcatgaaaaattaagcggtagcgacaacgatctgtggtttccgctggccaatggggccgacctgttcgaggcgctggaaagcattatgtgcatcaattttgccgctgctaacgttgtctcgctggagttcctgcggcagaacgggaagtgcaaagactaccggatcacctacaacaaggccaaattcaagccattatgttagatttctggtagtattaaatgttcatatttgaacaaatttaattaaaaaaatacgtctgatgagttgataaaatggctcattagacgtataattattacatcgaaaggcaatgaggcttttcgcggtagcgccccgactggcgaaggagttgaaatgaaagaagcagaccaaaaaaaagtgaatgccgtaatggcaattgctgattatctcggtgtaaaaaatcaaatcgaggtgatcgaatactcagccgaatcagtgcaggttgagtggcgaaatcctaaaacaaaacagcttattcatcgtgattacacttttgcaatttcctttgtaaaagactttgaaaaagccctgaaatcgaacgtgaagttttactgataaagagggggagaaatcccccttaatcctcccattttttactgataaacatcaaatatatgaggtgtattatgtggaaacgtggcgaaaacaaagtgattctgatggggcgtgctggcaaagatgcagaagtgcgttacaccccaaacggaacggccattgcgagcctgacgctggctactgaaatcagctacagcgacaacgaagggaaggagcaaaaagaaacggaatggcatgatattgtcatttttggaaagaaagctgaagcagctgggaagtacttcaaaaagggtatgatgctgtattttgtcgggcgcattcgtaataacaaatggcaagggacagacggcaaaatgcgcagcaataaggaaattgtgatcgataataacggcgaaatgcagatgctgccgggagctgggccacgtaacaccagtgctgaaggtctgggaggtactgaaaatcatgatgaaccgccgttcccggatatgaatgattacccgcagtgagtatttgagtgagaacggcccgcaggacgtgggccgccaacccagaaggggggaagtatgaatattagtgaacaatatcgggtggctgtcgtcagtaccggccatgtaaaaaaagaagacgcggagctactggataaggtctgtaacgatgacttgtccggtgaaggtttattctggatcgagaaaaatcgctatgggtggatattgcgtatttctgctactgaacaatggcgtgtggaattaaccagggcggggatcagtgcaagtgctttggataatatagccgcgttaaatggtgccgggttcgactggatttggtttgaccgggatgcagaacaggtgccagaactgaccgtttgggagtggtaaaccggttaaaaatatgcctcgaaaaggggcatttttttcttgcctaaaatggctcattagacgtataataagcatgaagtttaaaagtggatattgattaaaacgctctgtaaataacgtggagagaatattatggcgaccttatacccttatgaggtttatacccttattgtgtcagaattaatcgtaagccattttaatgatgtcgctatggtatatccgggaagcgaagaaattaaaaagaactacgacagtgaattaagtgctgtacaggtagttaatggcatcagagaaaaactgattgaagatggtgttatacagtgcgaacagtgcccactgctatccgagtgggatgagaacaaggttatcaaccagctaattaaagattaccgcctgattctcaatcgtgactatgaactaaaccgtgttcgtcagcagctggccccggagctgatcgcacagatagaaaactggaaagagcagaagtattccgattctgcgaaggaatacgaggtgctctatgaaaccatcaacatcaacggaaaggaggttacaggaaaactaaacgtagttgcagtaagtcgctttgatgctgttttcatcgtaggacagcgtcacgctaacgataactttttggttctggatgtaaaggaggtgatgaatcattgaagtccagaaaaggtgccggattgcgccaacaatccggctgggtaggcaatttaaattgtgaggttcaaaatgccgttgtggattatacactatgctgaagatgtcgcaaacggagtgcaaacaaagtcgcatccgatgaacgcagcgcaatgggatcacttccgtttctggttggcagtgttccggcggattaaagaagccgcgatgtaatctgtaactgtctcaggggcgctgtatgcgcccctttttaatatccgtgcccggactcctttccctgtaaatttatcgggatcaagggcggatcgtggccctgaacgtgcgcaccattgagcgctgcatcgcagcggaaccggaaaattttttccggccaatgagtgcccttgtcaccggaacggtgacaagtatgcacgcactattacacgaacaggcagctcccatgcccggcctgacaggccgggagctatatcataatgtgggatgctatatgcgaaaggtgaaggttaatactgcggacctgaagcatcaggattctgacctgtagagccattttgcgtgcgctgctcttcgtcaactccagtggttttttttcggctgttgctggttcgttttttaccaactcgtccggcagtaatgtccttgatatcacgtaatgtaacatctgaaaaacccgcatcgttgaggaatttcagtatctctttatcttcataccctttcgaatctttcagcttcttaatgtcagacttaagttcatcgagtacgtgctttttctcaagcctttctgacgtcacatcaggcaagtcgtcgagaatgcttttgaattttccgatgtcctcactgctatagaatgctttcttttttgcaggcatatgccctcccaataatgtcaaaatcgaccatgcctaaccggagaaaatcggacaaggctattcaggtgattttacatgatgcccggcatgattcaattgctctgcctgccaggcccttcgctgctggcattcaaacagcgccgatggtagaattcaatgaggtatttaagtacgggacaagatgtgtttttggagtaccgccgacacgcggcggccgtccaaaaacgtcttggtcagggcaagccccgacaccccctaacgaggttagctatctgatgagccgtagcgagagcagaaaaacagacgatcgaatccaggtgagatgttccacagaagtgaaagctaaactcaccgaaaaggcccatgaagcagggcttagtcttagtcaatatcttatcaaatctgggcttggaaagcgcattcaatcgaaaggtaattacaatgcacttgccgctcttgtaaagataacagcccttcaaaaacatctgtttaacgaaggtgccggagttcatagcaaggagtattcagagattctgatcgaggtcaaaaaggccgcacagaaactgcaacaggagatggatggtgatacctaaaatcattgagggccgaagggacaaaaagtccagctttggtcagctgattaagtacatggcagacaagccgtctcaggagttgactgatacagttcagcctactcctgagacggctttagctgttaaatcggatatgttcgaagggttaaacaattacctgacccgtaagcagaaagtgatcagcacaccggtggatgttgagccaggtgtacagcgtgttgtggttggtgatgttacctgccagtacaataccttctcgctcgatggtgcagcacaggaaatgaattcagtttctcagcaaagcacacgctgtaaagatccagttatgcactatgttctttcatggcctgactatgaaaagccgaatgatgatcaggttttcgattcggtgaagtttacactggcatcgatgggcatgtccgatcaccagtatgttgctgccatccaccgggatacagataacctgcatgtgcatgttgctgttaaccgcatcaatccccagacatataaagcagcctcatcgagctttaccaaagacactcttcatcaggcgtgccgcttactggaattgaagaatggttggtcccattcaaacggtgcatatgtggttaatgaccgtcaacagattgtccggaaccctcacagcaagaaagagcggggcaactggcgctcactcgatcgcatcaacaagatggagaataaagagggggttgaaacgctctatcgctatatcgttggtgatgaacaggtaggcggtagtcgtcaaaacttgatccatgtttcagctgggcttcgtgaagccaaaagctgggatgatgtgcataagacatttgcagacatcggtcttcgtgtcgaaaaagcacaggggaaaaagggctacgtcattacccacgaacatcaaaaccagaagacggctgttaaggcgagtctggtatttaacaaggcgcaatataccctgaagtcgatggaagagcgctttggtgaataccagccttcacatattgaaccggccaaagtatcggtatttaaaaccgcatatacccctggtgcttatcgtcgagatgccaataaacgtctgcagcgtaaaattgagcgcgcagaagaaagaatgctgctcaaggggcgctatcgcgcctaccggaacaatttaccggtatattctccggataaggatcgcatcgccgatgaataccggaaaattgcccaacatacccggttggtgaaaaataacgtccggcattctgtttcagatcctcataccagaaagctgatgtacaacctggctgagttcaaacgcctgcaggcggttgccaatctgcggcttagcttgcgtgaagaacgcaacggtttcagagcggctaacccacggctttcttaccgggagtgggtcgaacaggaagcactgaagggtgataaggcagcgctcagccaaatgaggggctttgcctattcctccaggaagaaggagaagtataaacagcagctggttgagcagatcgggttcaaccgtacctttaacgccatcaccagtcatgatcgtgatgatgtggccgtaatggcctcagcacgtcatggtgtcaagccacggcttttgaaagacggtactgttatctttgagcgcgacggtaaaccggtagcggctgatcgtggtcatattgtgctgacagaaagtaatggaatcgacaaagagaaaacggctgatcttgcaattgcattaacgattgctggtaaagccaaatctgtacgtgttgatggtgacggcgagtttaaggaactgtgctgtaatcgtatcgtcgatgcagctgtgaaccacaatcaccctgtcgcgcagggcattaccttcacagatgccgctcagcaggcgtatgctcaaaacgagaaacatcgtttgattcgtgagcaaaataatagtaaaaatgaaatgcaattcagaagtgaaagagacgacaaattcaatccgaagtaaaatggagtaagaaatgaaaatcaaaatgcaaacggtcctcgcggccgtttctgtgttagctcttgccgggtgtgcctctcaaagcgaaaaggaaaaacaaatctctgcacagcagctggtagatcaggaattgcgtgagcaagccattaagataaaactcgcacaggatgagctttaccaggctggagccattaacaggacccgttacaaattccctaccattatcaatgcaaacagtcagtatgttcgtgtgagctggcagggtgatgcttacgaattactggagcaactggctaaacagcgtggccttcaattcaccagtcaggggattaagttaccgcttccactcaatatcgatgttggtggttcgaaggttacgtttgaacagttgctgaccctgatccgacaacaaacgcagtaccgtgccaccatcaaccagaaacccggtgaactgcagctgctttacctcacaccatcgaagaaaggttcattcctcagagggacccgcccatgaaacttaaactgatcgtgcttctgacgttaccgctgcttgctgcgaacgttgcacgcgcggacgactccgatgttaaaggcctggattactacatggacccgccaagccaaagtgacgatgaggccgatgttactggcagtatgctaaacgacgcagcgcacaccattggatttcgcggcggtaaggcagaacgcgctaaagaaattcgcgccgcgctggaaaatcagcgcagcaaccttgattacatgtattcgttccagccgctgatctcttctgaaggctacctgcctccagtcattactgaagctaaagacgttgcacacattaccaatgagcaaatccgaacggccaaccgtgcctatgacattgtggtgccggcgcgctttgtcagtaacccaccgacatggaaaagctatcttctgaccgggctgatggcgcagcggatcgaattgcctgaaccggcggcaatgccgaaggacgggaaacaacgtgatatctggaaaaaagctgtcgcgctgggctggtctgacggtcgccaaaaagctgatgagatattcactgctaatttcaatcgcctcacacgggattacaccggcatgttgcgctacagcacgctactgcagcagggcatgattaaggctccagtcatcacacagcagcagcaaacagttacaggggataaaaaccgcctcatgctgggcgataaaacgaaacgtatgaagcagcaagctgagtttgacatcaacaaacgcagctggaaaccgaccatacgttaaggggcagtagatggaagcattcaattttggtccaacgctgacgccccagacgctggaacgttttctcgtacactgctcaaacaatgaagtgtccgatattctgttgcagggcggagataaaatctgggtagaacgtcatgggcgccagttgcccatgacatcttacccgctcgacaacctggagctggaacgtaccgttgataaggtgttcggtcaggagatcaaagggacgctgaaaagtgctgcggtcgttaataccgctatccagctaagaggtgatgaagcgggcacgattggactcggccgcggcgaaacccgccggttccgcgcgaactttactcaggccgatatttataaaacagaagcggcaatgtctctgacactccgtgttctgaatgaggttattcctcagcttaaacaaatgggtattgagccggacctgtttaattctctgttgccggctgccggcctcggactgatatgcggcgagacgggatcgggcaagtcaaccttgatggccagcatattccagtattgcggtgaaacgtaccaggaccggaaaatcatcacggttgaagatcccatcgagtatcgcctgggaagtcgagactggattgcgccggcgccggcacagtcacagattgggcgcgatattgattccttccacaattttatcactctctccgggctccgtcgttctccgaagattatgggggttggtgagctccttaaccgcctgtcatttgaagctgctgtactggcaggtaaatccggccacttttgtcttggcacactgcacgtcaaaacggtcggagaagccatttcccgatcattgcagaactatccgccggaaatgcgtgaagctgccgcctttgacctgctgagtatcctttcttacatcatcgtgcaacgcttgcttaaaacaaaagacggccggcgcaaggctgtccgtgaatatctcgtcattgataacagcctgcgccgcaaactctatgacgttgattacagcaagtggggccgctacatcgacgacctgttgatcgctgaaaaccgcagtctgattcagaatgcctgggcaatgcaccaggaaggtttgatcgacgaagcggaagtcatcgaggtcgcaggatatatggactatctggcgttaaaagagggcaagtatggacattaacaaatggcgttatgccggccggccactgacggtttttggtgtgcctgttatctcgtttcttgtgtacttcatctggtttccgttcccgtcagtaaaaacctttgttatctgcacctgtgtggttctcttttactttctgctggccatgatgggctacacgttaccggtgctgtaccaggtcattctccgggttatccgcgggaaaaaacttaccgggcggccgtggtggtaccggcgctctcagcgataacacataactacaggggtagacagtatggcgaaacctgaaattgcaactgaaagaacccgtctgagtattccctttgacgatcgccaacgtgcaatacgtgcggccggcaaactggctgatggcagtaatgctctggactacgtgaaggaagaaaaggtgtggtatgcacagccgggggccaacctgagccggctgaaagagtggatttttgaccccaataaggtgatcgaagaaccgcccctggatattcaagccattgaagatgaatttacggcgtggcttgaagagcgcggtgccattatcaaagaccccatagagttcgatggccagaagcattatgtcgatacagtggacggtaaagctggttcaagaaagggggtgtatgccgcctttctggatggccgacctgcagggtggtacagggactataagaacggtggtgaaattcagaaatgggtatctaccggcgcagcccctaaccctgaacaaatggccatgctccgtgctgatgctgcggcaaggcgtgaacaaagagctgcagcgcaagcggataagtttgatcagaccgctaaccggttgatggaagaatataaccggcttccggatgccacgggagatctggcatacctgaaaaacaaacaggtcattgccgctaacggccttaaagctgatgagcggggcaacgttgtgatccctctgtttaatgctgatggcgagttcaggacccttgagcgcatctggtccgacggtagtaaacaccttgaaaaagacggccaggcatgggggagttttttcgttgtgggtggccaactgaaggatggtgacaacctgctttatgccgagggatatgcgacagcagccagtatcagcgaagccatcaatcagcctgtgatcatgacggttaatgccggcaacatggtggaagttgctggccgcctgaaagacacctttccaaacagcactcattacttccttgctgacaatgacatttataaagacgagaacgttgggctagaaaaagcgacagaagctgcagagctgactgccggccatgttctggtccctgcctttagcaacccgaaagaagggcttactgactacaacgatttgcatgtctctgaagggctggagcaagtcagattgcaggtagaaggagccattaatcaaatgaacagggtagacactatgccgactgataacccgaacattactgacgttaatcacagctcgactgacagcgctgccgttgcagctcctgaaaaagccgcgccggtggccagcacccctgcagctgctgaaccggtggaagccgcgccggtggccagcacccctgcagctgctgaagcggtggaagccgcgccggtggccagcgcccctgcagctgctgaagcggtggaagccgcgccggtggccagcgcccctgcagctgctgaaccggtggaaaccgcgccggtggcaagcacccctgaagctgctgaaccggtggaagccgcgccggtggccagcgcccctgcagctgccgaaccggaatacgtcgcaccaacagatcgagacggcgatgatgaagagagtaatagcgccagtgatcttgaggcgtatgccgctatgatggcctttggcggtgaaacaactgacactgctcagcaaaagcctgaagggatagcgcccagcgaacctgtagctaataccgccccggctgaaaatgaaaccgaggagaagaaaaaaacaaacgaagaactggaaaacggcatacgctgggcaaccaacgataatgcagaaacgccaccaaaagaacgtatagacctggaagggttaattgcccggtttgggtacagagcagagaaagactatacggcgtatacgcttgatgggcaggatgcgttttacgattacggttcacatctgcgtatggctacccctgaagccagccagagtgacgagatgatcctggcggccgttcttaccgcggataagcaacatcatcgtggtattgaaatcaccggcagtgacgaatttaaagagcgagtgtttaaccttatcagtgaatacaacattgaggtgaaactgactaacccagaacaaagggttaaattgctcgaactcaaaaaagcaaatgaaacagcaaaagaaacggcaaaagaaaatgttgctgattcaaatgcgaataagcaggaaggggcaaacgaaaaggcaaattcatcagttcagcagaatggaatgcgctgggaagaatcggcctctgtacagccgaagaagcagaatgcatctgatactcaaaaggaagataaagctgcgggtgaatcctgggagaaaggcgttatcgtcgcgcatggacgtgctaagtatgagtggaaagatgatgaatccatgaactactttgtcaccctcaaaaatgaacatggagagaagacactctgggggaaagaccttgagagagcggttaaagatgctggtgtagacaccgaacgtcttgttacagtcagaaagctgggatttgtggatgttgaggtgaatgcacctgtccgtgatgaaagcaacaagatcgttcgttatgaaactatccagaccaaacgtaatgagtgggaagtaaaacctgtatacccacagccaacagcagggaatgaacaggcctataagccttctgaactggttccgtatgatgctgcaaccttccagaagctccgcacaacaattcagcagattaccggtgttgatctcagccgtgaggccgtgcctgaaaaagaactgtattggttcctgccgaatggtaagccagctcctgaaagcatgacaaaacccacaggctataaactccctcaagagtcaaaaaccgcgggtacacctttgctggtcgcaccgcataaaaaaggcgaacgcccggattattaccttgttgagtctcgtaaaggattcatgcaggggatcgcaaaggataaggaatctggcgagtaccatagcgtaattggtaaagtgaacaagcgtattgataaagatggaaactggaaaacgtatatgacactttcagcaatcaataaaaatgcagaaagtggaattgttttgcacggatacggccatgttgatcagggaggtaaaaacctgctttacaaaaacacaattgcaaatgaaaaacagcctcagaatttacaaccggcctcagatgaggtaaaaaacaaaacagtaatgaagcagctttttcatccttctaatgctgcaaaaagaaaagatgatgaaaggagagaacagactcatgcaccagtcgcaaagtcagcgcctcggccataaaacaaaaaccctactcgtagcagtagggttttcctgcatttctgtgagctcatttgcagccacacagacctgtgcagcatacgaactggcaaaagctggtgctgatgctgcctataaacaggaggttgaacggattaacaacgccgctaaaagtgaatccagtacatctgattctctcggacagtgcctggggtcgctatcggtatcactaaaaatccctcaatttccgtctatcagcgacatcattgataaggtgaaggatgaggtatgcaatacggttaaatcgaagataaatgagaagtgggggagcctgactaatacaacgctggacccgtggtcaacgatttcatcccgactgccagatacgtcaggtatcctgcccaacgcatcttctaagaatgtgcaattaaacaatccggcagctgcgagtggaagtggtgaggatgcaaacagtgatgcattcccgttccatttgtaactaccggaagagtcctttcgggattttatcgaatagcttgccggttcgtatttcaacgcggcggtgttccgccgcggttagtaaacgcataaattctgtggcaggtagaacatcacgttttgagtctacatacgtatcaagcgaatcttcattcatgaaacgtctgacagtatgcagccagtcttcagctgatttcatattttttgagctcatcgtcgctcatctcggcaaacgctgcaggtcggcgaccttttcctgtccaggttatggactttccatcaacttcgatccggtacttcacttttccaaaagtggatttacttttcgtggacggaccaaagagattttcgaagatctggagagcttcatcagccgtaatattgtgttcttccatgaaccgctttgtggctgcggctttctcttcagtggccttctgttcttccttaaatttctcgatcattccctgtgcaatgtcggaccacatattaatgagttgttcaaggttttctggcgggattttgcgcaaaacggcggtggcttttcgtcgttcgcacatgataactgcacaggattcgtaatcattatgttcgctcatgtatacctcattgttcattaacgatctgcgtgattttctaacacaaacttttgaaaagagaaactctatgaaaacacaaaatggtgaaaatattatggatcaaactgactcggatcaaaactcagtcgttgatgatgacagcaacgcccttgctccagctctggctgaagagagtaaccgaacggtacaagaaatcgtatcgaaacatggtttaaaagtaattttgattcaaacgctaatcatattagtcgcaatcattatcattggtcttcagggttactggcttcaccagaaaaaaatcaaatatattgcaacgaatgggggcatgatcacagaagttcatccaaccgatgaacccgcttacacggcggaagaggtgatggactttgccaaccgtacaatgatcaaatcgctttcactgaactttgtgaattacgaaaaccaattaaccagcgtccgcggcgattttagcgctgaagggtacgttagtttccgtaaagcgctgtccgatagtgggctgctgaaagatatcagtgaaaaacgtctgaacgtaaaactttccacaactcctggaaccctgatcacggaaggtcttatccgtggcaccaaaacctatgcctggcaataccgcattccggtgactgttcagcttgttgggcaggctcaggactacagaccgcaaccttacgatttgatggttcaggtacgccgcgtcaaagaagacgaagatccacggggtattcaagtagcacaggccattctcaaaccccggaactactgaggtcaatcatgaagattaaactgttaactgtacttatggccgcactgttttcggcaaattctgtatctgcagctgatgcgccgactggtaatggagcgccgcagcaaacggcgccagctgtgcctggtggtgatattgctcaatggcagcaggccagttccgggttgcccgtaaatcagcagtcacaggtgcaacagcctccacagcaaacagcacaggaacaaggtcagcagcaggcgcaaggccaacaaccacaggcacaggcccaacagcggccaacgactgcactgcctcctgcacccatgccgcagattcctccaccacagcagcaggttactcatgttaatccgcagacactggaagagttacctgcacctgttctgcagcagatccagcctattacccctggtcaggtgcggtcggcccgcagcgcaatggaagatatgagcgcggcggccaaaacctcaccattaacgccgatcccacgaatttcaagccagacggtatcactgtcagctggcgcatccattccgcaggtacgggtatttccaaaccaggcaacgacagtgacgttttcagatgcaaccggacacccctgggtattaggtgcccctccatataattcgagtccctgtgcttcaggtggtgaactgtgtgttggctatattccaggatcggcggtattcacaatccagccgaccaatgcctatgccagcgggaacatcacagtgcttctgaaggggctggccacgccggtcattattaacgtgaagggggcagaaccctccgttaaatcaaaaaccgttgatgttgattatcgtctggatctccgtatcccgaagcgcagtcctgacacaccggtttataccgctacgcctgagaaaaaaatctcactgtatgacaagcagctgcagagcgtgctcgatggcatcccgcctgaagatgctcagcggttgaaacttaaaaacgcgccagggagtaccaaagtctggcagatcggtgatgagctctacgtcagaagtaacatgcagctgcaggatgagtttgaaaaaacgctttccgctatcgatggcacacatgtttacgtgctgccggcaacaccagtgctgactttttctgttaatggtcaggcgcgtgacgttgaagttgagctgaattaaggggaacgacgatggctacagataaaggtaaagaaaccagaaaaatgatgctctacgtcggtggtgctgctgcagtggtggcgcttctgggagggtactggttgtttggctcatcagatgagccggcaacgggcgggtcgcaggtgaaagcccaggggctggaagggaaaactggcagggaaaaagctaatgatccccaatacaacaagctgctgaatgagtggaatgttgataattcagataaagcggctctcacaggggatagcttcatctccacgttgtcagccagtaatccggtggccgttgattacggcaccaaagcaccgccaccaaaatattcgtgggaatcgtcagcaaagccgtcgggcgataacagtaaggcgaatgatgctgagcagaaagagcgtgagcgtcaggctaaagcggtggctacgttactgcagaaaatagaccaggccagaacaccagttgtcacgggtgttgctcttgcaacccctctgggagcagaaggaaatgacaagaacggcggtaccttctcagggtggaccgattcggtctatccgcagaacaaaaagccggatgctgaaaaacaacagggggcgaagaataaggccaaagataaaggagcccggcttgtatcggcttacacgcttgtgccggcagtaatggatacggcgatggacagtgacgatcagaacagtattgctattgcccatgttccaactggagcgcaggccggcgccaaattctattctggccaaaaccgtcttgccggcgatggtattcgaattcacttcaccggcatggagctgaacggagtgcagtgtaaggtcgatgcgtatggcgtggcttccgactcgttgcgcgcggccgtttcctcaaatgtaaataaccgctggttcagccgtattattctgccggctgtcgctaacggattaggccgaactggtcagctctatgcggacagcaattcgcagatgattatcactgacggcgggaacgcatatcgttcaactgatacacctgatggcaaagcggtagccggtaccattattggcgggatgggtgagcaagcagggcgtgtcctggccgacgatgcagcccgcctgccgattaagcaggttacggttgaccgtaaccaactcataggcatccagtttgtcgcgccagtgtatgaaagcgactgtggcgagaatttggaaaatgcttctgcagagcaggcgcagcagcaagcaactccgacactatctagtgtgcaacctcagcagatgcctcagcctccggctcctaattaccctcagcaaaactttccaagctaccccggctacgggtatggcaccaataacccgtattaccgttaaggagattgataatgaatctgtttgaagatgacaataaaaaagaagcagctcctgcccaggaaccggtgtctgctaagactgctgaaaagccaaaaccgacagtgacgccagcagcaaaagcaaacaaaaagccgcttaagattagtgactatgcatggattggtggtatcgctgtcatagctatcctgcttttagtctggttgtttgggggctccgatagtggcactgcgcctgcggccgggaatggcacgatccagtcttcagggcaacgtcagaacgtacaggctgcccctgccgggcaggattctggtgacttccgcgagcagatttccgggattttgctgcagcaaaaggaacggctggacgcgatagaatcaacttctaaaaatggaatgatgatcctttccagtcagttacaaagcgctaatgagcaaatcaaaaatctgaacaatcaggttcaggaactgaaaatgaaagccgctggttatgggccttcatttagtaatcagagtggaacaatttcgggggcaacgtcgcaggcattacctttgttgagaggtttttcaattaatgatttatctggcgatcttgcttgggtaaaatacaaaaaccagacgtatgctgtgaaggtcggtagtcaacttggcggagtcaccattacgggtatcgatactgaaaatagaatcgttactacaagtaaaggccttatccgctaaacaggacctaaaaaggcgaaactatggatttagaatcggtattaattcaggttgctaacggtggtcaggaaccgctgacaaggctaatcataggtgtcgctgcagtcgttgggatcattggtgttattggatatctgatgagcctgcggaagcaggccaggcattcaacaaaaggtgtggaagttggcagaatttttgccggtatcatcttctgtgtatgcctcatcacgttgaaggcacagatgaactcaggcggtgccactttaggatttggcgatgttagcttcggacccgtcagttatgcgagcgaatctacgtttggcatgggagccgcagcagtcaacgcagtgctggggattgttagaattctcggcgcatactttttttacaggggtataaaggggttgaaagactcttaccttgagggccatacggagctgtcagcttcgggtacgagaggggcatccattgtgaaaattgtatgtggactacttctgatgttcgatacacaaacgctcgatgcgttgcagagcacattaaacattcactggtaatcaaaaaggatactttatgaaaattgttaagcttatcaaagacaaggcattatctgcatccatcgcagcgtatcagctgcgcggaacggtggcaaaatcgttagtggtattgccgctggcattactgagcaaagaggctgcggctgaaacgactgtctgggagaaaatcaataccttcacggaaggtcttgccagtacccagcctggtctgattaccggcggtaaagtcattggtgttgtgttctttattgtcgggcttgtttctcttttccttaaaaacaaacgcgggaaagatatcagtggcagcttcatcttctggtcaatggttgttggtgcttgcctggtcggcctgaccgcatggatctcttctgttggttcaactgttggcgttgacgcaaccctgtaatgtcccttcggccccgccttgcggggccaatatcggagttccctcatgaaagattttaagctcaccaggaatgaacaacctgccagatgccatgtgtgcgggataaagagcaaagatgttttctccctcgcagaagatggacagaaagagagaatgctttgctctgtatgcctgtcgctcgaacatccggagaaaatgaaactagagactcaaaccggacttctgctgaagagaaatacaaaccgaaaactctacagcctgctgtttcgttcaattgcaaaggcggaaaagtcaacggacgattcttaccgtgaaaaggcgcagaaacttaaacagatattgctcagcacaactgacgtagttctaaacgaaaccggcagtgatgatggatacgatttactgcacctgataaaaaaacaccatcagttacttcccatgaattttgacgatgagtttgtcgcttctgttgcagagtcagacgaattgccggatgtaagtgactggaatgaaatttacaagggtaagagaggttttaaatgatttctttctttgaggatgtgatcgaaggtatctcacgcaatctgacatcaaatgactcgttaaaatactgtgatttgacaacggttgtagggcttacgtcgcaggatcgcgttgagcatcctgaaatcagggctccgtacattctgaccactaagaacaacgattttcttaccgttattgaggttcagggcgctaaatcatcgtttgacgagaagtcgtttaatgacttcatcatgcacctatcaaactccttgtcgaattcctttattaacagcggccataagttgtctgtggtgttcgaacgcgactttaccaagaaccaggaagagctgaataatgtttataaaccgcaggtagcagcaatagagcgtctgcagatagacattaaagacctgatcgccgatgacgcaaaaaaaatcgccgctcattacagccgtgaacgcgcttacatcgtgctgtatacctcgaaggggatgattgcgaaggatgaacttaaaaacgaacaggcaaaagctgccggcgtactgaaggatatcccgcagacgcgcttcagtcagaacccaatcgaatttcagcttgaaggtttgaagatcacgcatgacgcgatgctgtggcgcctgattggtatgcttcagggggatgatgaatcaggaatggggcttctggttgatgtcctcgatgtcaaacacgctggcgcaatgatgcgtcagatgctgttccggaattcaacatcagaaaactggtaccctcgcacgccgtttgaccaaaatctgcgaatttatggcgccccacggaaagacaatatcgactccgtgctgccgccgcggctgaatatccagatcatggaaggggaactggaagaagatggtggcctgatttactgtgatggtaagtattacggctctgttgcccttgagttaccaccacttcaccctgtcaaatttaagcagttattcaacgcgattaaccggaaaatcccgtaccggataaaatatgactttataggcgggggcaaaaaagcactgttctggccatcagtaatcatatctttccttcgtttcattccctcactcggtaatatcgcgcgtgatatggattatgtccgggcccagagcgaaaccgatccaacggctatcatggccatcaactttgccacctggggcgacactaaggatgaagccaaacgcaacctggcggctttaactaaggcgattgaaggctggggtgtatcaggtgtgtccaagacatatggcaatccaggtagcgcactgattgcatccgttccggggctgacgacggccacacctggctcattgcattatccgccgttgagtgaaggcttgcggatgttaccttttgaacgtccggccagtccctggcatggtaaaggcaatatcaacttcatcacgctcgatggcaagctgttcccctatcaaatcgccagtcccctgcaggagaaatttaccgatgtcatcaccggtgttcctggttccggtaaaagcgtgctggccaaccgccttaacctgagttcgatttaccgggctgataaaaaactgccttatctgaccatcattgataagggctacagtgcaaaaggtatcgccgatctgattagcgctgagctgccgtcagagaaacgtcaccagattgccagtatcaccctgaacaatgacgaatcgcactgtattaacatctgcgatacgcagttaggttcaaggtatctgacgcagtttgaggaagtattcttaaagcagatgcttaacgcattctgtattgatccggcaattggccagccgccagatgcaatgagcgttggccaaatcgtcagtaaggttgtatcaaacgtatacaaagcaaaagcgcattcaagtgctaacttgtttaagtataacgacccggtgattaatgaagctctgaaagaatcaggtctggataaagagcttggtgaagactggtttgaaagagctacatggtgggaagtcgttgataagctgtttgccaaaaaatatcttcatgctgcgacggttgctcagcgttatgctgtgccaaccattcacgattttattgctgaactacaaactgaatcatttaaaaaccaatacgctgacgtaaaagttaatgggagtgagcctgtagtaggcttcgttgcacgttgcttccaggctgctgcagctgagtatgccattttctcagggataacggtttatgatttcagcccggagactcgcattgcgatcctggacatgcaaaacgtcctcggtgacagaacgacacctgcaggtaagctcaagtccggcattatgtatctttttgcgcgccagatggctgtgcgtaactactacctgcctcagtccgcagaaaccttcattcctgctttaccggagcagtacagggcatatcaccaggcgcgcatcagggagctttcagaagaggttaaacataccttttatgatgagtgtcataactttgccggcatcgactttattcaaaatgcgctgaacaccgctgaccttgaagatcgtaaattcaacgtccgtactgccttctcgtctcagtatctgagccatatgccttcgagtgtgcttaaaaccatgaacagcctgtttatgatgcgcctgacggaaggggatgaagagtatctgaagcaattagagatcaacatcccggccgatatcctgcgtcgtttcagacagcttccgcaaggggtttatccggacggaagtggtactgcattcctcggtatttttaaaacaaaacgtggtcttatttgccacattctgaaaaacacattaggaccaaaactgctttgggctttgaactcatctgcaaaagatagagcgctcagagatgtgctttatgaagagctgggaacgaaaaaagcaagggaagaattagcaaatagattccctatgggaagtgcatcaagtatcattgatgaaatggtggtcaatcagggtggtgagcgtgattctgaagaagaatcacaaactatggcgatgaagctcgctaaagaaatcatttctgatgtgagaaggggattcagatgataaaaatcggaaaagcaaaacggtcagtgatggccgtttcaattctggccttcagcgtacagtcagcatttgcctatacggtcaacgttaacagcagcattcccatctcaacgcaggttatgccggcgttgagcacagctaatggcctgttaggtaatatccaggccacgctgattaacattggtacggcaatcagccagcagggcgatcgccaggcagcattaatgcagcaggtggctgaaactaaccgacagttcgaagtcgagcaaaaacgtaatgaccggctgctgcaggcacaggataagtaccgtgttccggatgatatctgtgcgcagtctattggcggtggcgccgcgggggtatcctctgcggccgctggcggtcagcgaacgctgggaagcgccagttctgcgaaagatgttaaggctatttttgaagtgccggcacgcgcaaccgacgtggatgcatcttcaacggccgaagttcacagccagtactgcgacagttcggattatgctgcatttggtgggacttcattctgcccaagtgtatctgacatgccgcgggcagatacgtcgctgagctcgttgctttatggcgccggcaaagaaggcaaggcacctgatctgacgtttacagatgagcagacagatgcggcgctgcgctatatgaaaaataccgcgttgcgcagtgctggtcgtcagctgagtcgtggggaagttaaaggcgccagcgggcgaatttacctgggcatgattcagcaataccaggcgctcaccgacgcggccgcacaacctcagatggaaatgatcgccaactccaaaccgaacccggccactgatgccgcgcttgcagatgcgcgcaaagtgccggaagtcgaatcctatttccaggcgacggccagtaacagggcgaaacagcttaaccgtatgtctgccagagagtttgaggaattcgaagtaggtcgccgttacagcaaccctgcttaccaggcgacgctgaccaacaagaacaccgaagatttgatgcgtgagcaaatcaatatcgccaatctgaacaactggttagtgctgaaaaccaaacagcagctggagaaacaaaatgtgctgcttggccagatcctggcgaacgacgcatatcaaacatacaaaccgatgctggcagcacagctggagagtgtgaacgcgggagtatccagaaaatgaccgacgaaaataaaacaggggataaagacacagccaaatcagggaaattaaaaaaaggccttgatgttgtaacaggtgtcaatgacctcccggaagggaaggctaaacgaacgatatattatattactggcatcagcgatatatattttattatcgcgtcagtaaagcaaacattcagcctgctctttcagcgggcttcattcgttaagaagcaaataaaaaaccttgatggtccacctgttgattctgatgcaaatcagccgttcgccgaagttatgaaacggagcaatagacctgtatcagaattgttggataaggccagtttatacaagaaatactggctttgttgtttttttgcattagtcctcattctgctttttttaacgtctggttatgccagattgcttctaaatggctcaccgaacatgagcttgttaagagcaactctcacctgtggcgtcctgtttgctgctgggatctttactttcattaaggctctcacatgtgagttcatggggtggcagttacgaaaccaggctcactctgatgcagagcaaggtacgttacgttatttccttaatgatgggggagtgcgtaatacatttaatttttcgcaggctggtcaagagcgggggccacatgagtaaattaaaaaggttgcttctatctcttttcgttgtggcgtcgccttcatattcagcggatttggggattgatggcataactgaagcggctaaacgttcaggtgacctatcgcgtcaaatgttacataccgtttttggtgaggtagttaataatccgtttagcccttccggcgatggcttacttaataacgttttcttcactgtcaacgaagttatcgcgattcttgcccttgtctatatggggataatcagcattaagaaacttcaccaggcaggtcagttgggaagtttcattgagggtgatggcaataacgcattcaaaattgttaaaacgacttttgggtggttgttgcttgtacctaccgttgtcggttggtctgtggcgcagctgctttttctatggtgcggttcaatcattggcgtaggatctgccaatgttattgcagataaaacggcttcggaattagcgagtggtaaagctgtctatatagcccctgttatgccagaaatggcgtctgttgcaaaagggatgtttgaaacaaacctttgtgctttgggtgtaaatcaggggattgctcaaatggaggcttctggccaacattatgagaataacgccagaatgcaataccaaacaggtgacagcacatattctatatctgtaaacaacggctcagctgtatgtgggaccgtttcgctgccacaacgtccagcaggatggacttctatcttcgctagcggttatgcggattccgtctacagcgttcagcaacaagcaacggacacgttgtggggaaaaatgaagactgctgcagagcagtttaatgcagcatatatggcaaaaatgcgttccggggacggtgagctgccagatgttgaaagcgcgatccagactgctgcgcgtgattaccagcttcaggttcaacatgctgctaattcagcagccggtgacgacgaaattgttcaaaaaatggagtcggatatcaaagagaaagggtggctatatcttggtgtgtactatcacacattagctactgcgaataccgagatgaaagatgttgctaacctgaagccggtagtttcaggaatgagtaacgatggcgatatcggctctatcgattactttaaaggcttgttccaggcttaccattctcaactgaaaaatagtaattatacgccgcctcttggcaccgaatctaacgcacttgcaaggaacattgatcaaaaaagccttgaaaatgccgccacaactggagacggaacgtcccttattacaaaaattttcgacttcaacgttacaaactggcttgcaacgagtaactacggcactggtgatggttacagtgacgtgaccaaccctttactcaaaatgaaagctatcggcgattacacgttaggtgtagctgaggttggactcgccagttggaccgctatcaatgttgcagtgaaggtttcagaagggaagagtctacccggttttgctgcgggaatgatgaacaaacttacagggattcgagatgctattgctggcgtattagaatcggtttcgttcttggtttacccgctgttttatagcctctttggtatcggtcttgcactatctatctggctgccgtttataccgatgatttattggtttgttgctatggcagattggctggttacgcttatgacaggtgtattggcgtcttccctgtgggcagctacccatatcaacattggccagagtaatgatgagcggagtacatatggttatgtgttccttatcgatgtaatgattcggccgatgctcatggtaatggggtttatttttgcttctctggccatcgtagctttgggaacagcgttaaacatgatgtttaaattcgctatggaacaggtccagtctgactctttcactggcctgctgtcatcgatcggatttttgatgttatatgcaaggctttgcacaggtatggtggctcgcgtatttgcacttcctgcgaggatgccgaactatgttataaactggattggacagaagatgaacgattcagtgttgggtgatatgcagaaccatgttcatgacatctttgctgcgttcggtcgtggagccaaaaatatgaaccgtaacaaccctaagcaatttaatcctgacgcaggtgtagataagagcaaggacggcatcaaaggagcttaaatatgaatgcgttatcaaacactgactttaagaaaattagtaacaatgcgcgtcctaaaaggcttggatactacgcatcatggctctggatgatgtttgtattcctctttctgttcgttagcgctttcgtgtttctgagcggcttatactggacggttagagacggttcaattcaggagcactggtttagtacagtcggtatctttgtgatcgatgcagtgctgatctggatgttgaaaaaggacttcaggagccgcaagctctaccaagtaactgcttcaatgaagaactctggtttcttcgagcctcataaggactgcgagcactatgacctggcccggagaacttacatcggctttgacttcagtactggaatcataggagtagcatccctgtatgccacatccagtattaaacgtgagcggctattttttgaagccgaaacagtcgagtcatgggagtcagtcggcagggagttaatcataaacctgcgaaatacggggcttacgacgataacgatcacagctccaaatatcaataaagcgtacagggatatggaaatcatctgcagaacgcataaaaatagggatgaaaactatcagcatctgaagagtaaactgacagacgctggatggttcataaacagcaattattgagtttaacccgccgaatatggcgggttttttgtgtatactcaagtggttatagtcgtatgttgtgggaggaagtagtgaacagagttactgagcgccaggaacagaagagtaacccggagttccagagagagtaccggaaccgtatccgggaaacacacgaccagttaaacctttatctccccaaagatgcaaaaagaatgttgatagagatcacaaaagttaaaaaactcactcaggctgaagtcattgaatttcttgtcaaaagttattacgacggattgaaatttgacgattctgaagagtgatcttactatgtaaaagcgggttcgcaggtattgccccgcttagaaacgcatgatatgttaagcggcgtttacataaccttacggggttattacagaatttagcacgggaagagcggcccggagatgtaccgcttggggtgtggcaggagccacaaaaagtaaaaccccctgattcatcctctcactttggccggtcagagatgaaatcagggggcgcaaactgccttgctggcagaaaggattatagcaaatgcgtaccctattgcaatacagcccggccaataagtgcaggggctggtgaatcaaaagaatccatacctacagctttctgacattgagtcagtagaaggtctttcgccggagttcatctcctggcttgaaagccagtcccctaaagaatcgcccctgcagctgcttctcgatgaaggcaccaggcccgtgcgcaaaacccgccgtcgtcgcggtacgcactcaacggcgtgtctctgcccggagcccagctggtaccgaccggacaatttcaaaaagctccctggccagcttggccatgcctacaaccgccttgttcgccgcgatcgcaaaacgggcgcgttaagtctgcgtatgcggatcagccgccacccgtacttcgtgcagctgcgcgagcaggctggccggcagcgcgacttccgcccggagcgggagaaattactcgatgcgattgtgccgctcctggtcagcaccgttgaccgggctacccatattgacactatcaacctcagtaaaatggcgtggcagctcagcgaaaaggacagcgaagggaacgtgatccggaaagtgacggtaccgcgggtttgccggctgctgcagcacatgatggaattcgggttactggcgctgcctgaaggcgtcacatgggacccgtttaaccgcaagtggttccccaagcatgtggtgctgacggaacgcctctggaagatgatcggtgttgatctcgataagctgtatgcggagcaggccgaacaggtggccgctgaagctgcaggctggataacccgtgatgaacatggccaggcggaagaaatctctgttaaagccgcgcgccggcgctggtatgaaaaaatgatgcacgccacgctggttcgccggcgtgaagccgccctgaagggtaagcgcgagaagaagctgaaagcgctggcagaacagccctatgacgaccgggcttacgccatgtccgttcatctgatacggacgctgccgaaagacgaactgcacgcgctgtcgcctgagcagtttacccagcgagtacgcagccacctgtatcagctcgatctggggctggagagggaatccggtccccctgattatcactgattttctccccctgtaattctcttaccgccgcctggcggttttcgtgctggccagccggcgcgacctgctcaaatttcctccagttttaacgtgtggccaacgtgattttttgacgttctggcgccgccggtggataaccggcagcgaaatcaccactatccacagttatccgcaaaggaggaaataaagtcacctgcaatcgtggtcaaaacaagctgcaatacactcttaacttctttaaaaagatctttattcaataaagagatcactgttattcaggctcaattttctgtggattatgggaatatgcctggcctttcagcgccgctacgcggcacgacggccagaatggtcgctatgctcccaggttagcacccggcaaatgccgggcagggtggccagcagaaacctccagtccggattcttctttgccccaccgtgcttttccccgccgagccaacggctcggcagaaaaaagcatgatggatgttgtaactatgaacacccccgtaagacggcaggggtgcgacacgcccccctgacgtcaacgctactacccacgcgcccggtgtgccgctaacgtcacaccgcccgctcgcagtagccagggggtaatgtggccacagagccatctgcagggcagtctggcaggataatcgtctgcactgacgtgcaatgcctttcaggcaatcagcaccggtgccagctgcccgttttaaaattaacagggaaaagaggatcgatttagccgcggttatccacgtcagccgctctcaggcgcgttaaacagggtgaagacaggggaatgttgctctttttgcagtgaaggccacagagagcgttttaggcgcttattggggtctgacgctcagtggaacgaaaactcacgttaagggattttggtcatgagattatcaaaaaggatcttcacctagatccttttaaattaaaaatgaagttttaaatcaatctaaagtatatatgagtaaacggcactgttgcaaatagtcggtggtgataaacttatcatccccttttgctgatggagctgcacatgaacccattcaaaggccggcattttcagcgtgacatcattctgtgggccgtacgctggtactgcaaatacggcatcagttaccgtgagctgcaggagatgctggctgaacgcggagtgaatgtcgatcactccacgatttaccgctgggttcagcgttatgcgcctgaaatggaaaaacggctgcgctggtactggcgtaacccttccgatctttgcccgtggcacatggatgaaacctacgtgaaggtcaatggccgctgggcgtatctgtaccgggccgtcgacagccggggccgcactgtcgatttttatatctcctcccgtcgtaacagcaaagctgcataccggtttctgggtaaaatcctcaacaacgtgaagaagtggcagatcccgcgattcatcaacacggataaagcgcccgcctatggtcgcgcgcttgctctgctcaaacgcgaaggccggtgcccgtctgacgttgaacaccgacagattaagtaccggaacaacgtgattgaatgcgatcatggcaaactgaaacggataatcggcgccacgctgggatttaaatccatgaagacggcttacgccaccatcaaaggtattgaggtgatgcgtgcactacgcaaaggccaggcctcagcattttattatggtgatcccctgggcgaaatgcgcctggtaagcagagtttttgaaatgtaaggcctttgaataagacaaaaggctgcctcatcgctaactttgcaacagtgccccctcggctaccacctccattgtcgttttcagaagacggctgcactgaacgtcagaagccgactgcactatagcagcggaggggttggatccatcaggcaacgacgggctgctgccggccatcagcggacgcagggaggactttccgcaaccggccgttcgatgcggcaccgatggccttcgcgcaggggtagtgaatccgccaggattgacttgcgctgccctacctctcactagtgaggggcggcagcgcatcaagcggtgagcgcactccggcaccgccaactttcagcacatgcgtgtaaatcatcgtcgtagagacgtcggaatggccgagcagatcctgcacggttcgaatgtcgtaaccgctgcggagcaaggccgtcgcgaacgagtggcggagggtgtgcggtgtggcgggcttcgtgatgcctgcttgttctacggcacgtttgaaggcgcgctgaaaggtctggtcatacatgtgatggcgacgcacgacaccgctccgtggatcggtcgaatgcgtgtgctgcgcaaaaacccagaaccacggccaggaatgcccggcgcgcggatacttccgctcaagggcgtcgggaagcgcaacgccgctgcggccctcggcctggtccttcagccaccatgcccgtgcacgcgacagctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccgcatgcccgttccatacagaagctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttggtaagctgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagggcagtcgccctaaaacaaagttagaaaagggaaagtatgagcaagttatctgtattctttatatttttgttttgtagcattgctaccgcagcagagcctttgccagatttaaaaattgaaaaacttgatgaaggcgtttatgttcatacttcgtttgaagaagttaacgggtggggcgttgttcctaaacatggtttggttgttcttgtagatgctgaagcttatctaattgacactccatttacggctaaagatactgaaaagttagtcacttggtttgtggaacgtggctataaaataaaaggcagtatttcctctcattttcatagtgacagcacgggcggaatagagtggcttaattctcaatccatccccacgtatgcgtctgaattaactaatgagctgcttaaaaaagacggtaaggttcaagctaaaaattcatttggcggggttaactattggctagttaaaaataaaattgaagttttttatccaggcccaggacacactccagataacctagtagtttggctgcctgaaaggaaaatattattcggtggttgttttattaaaccgtacggtctaggtaatttgggtgacgcaaatttagaagcttggccaaagtccgctaaattattaatatccaaatatggtaaggcaaaactggttgttccaagtcacagtgaagctggagacgcatcactcttgaaacttacattagagcaggcggttaaagggttaaacgaaagtaaaaaaccatcaaaactaagcaactaaatttctaacaagtcgttgcagcatcgtgcgctgcgcgcactggacagtttttaagtcgcggttttatggttttgcttcgcaaaaatattccataaaaccacaacttaaaaactgccgctgaactcggcgttagatgctttgctgtgcgcacaaatttcggccagcaacaagactgttttttttcttaaatcgaacctaaaatttattcgcggaactccatggataaatattttgaaaaattggttatttctggctacgtccattatttttgaggtcattgcaacctctgcgctcaagtctagtgagggctttactaggttagtaccgtcttttatcgtcgtagcgggatacgctgctgctttttatttcctgtcgctgacactcaaatcgattcctgttggaatcgcctacgcagtttggtcgggcctcgggatcgtcttggtcactgcgattgcatgggttttgcatggtcaaaaactagatatgtggggatttgttggtgtcggcttcattatcagcggcgttgctgtgctcaacttgctatctaaggcaagtgttcactaaaacggtcgcatctaaccattccgtcgagagggaccgcccacaagctgcgcttgcgggttcccttcgcggcttcgccgctacggcggcccctcacgtcaaacgttaggcatcacaaagtacagcatcgtgaccaacagcaacgattccgtcacactgcgcctcatgactgagcatgaccttgcgatgctctatgagtggctaaatcgatctcatatcgtcgagtggtggggcggagaagaagcacgcccgacacttgctgacgtacaggaacagtacttgccaagcgttttagcgcaagagtccgtcactccatacattgcaatgctgaatggagagccgattgggtatgcccagtcgtacgttgctcttggaagcggggacggatggtgggaagaagaaaccgatccaggagtacgcggaatagaccagtcactggcgaatgcatcacaactgggcaaaggcttgggaaccaagctggttcgagctctggttgagttgctgttcaatgatcccgaggtcaccaagatccaaacggacccgtcgccgagcaacttgcgagcgatccgatgctacgagaaagcggggtttgagaggcaaggtaccgtaaccaccccagatggtccagccgtgtacatggttcaaacacgccaggcattcgagcgaacacgcagtgatgcctaacccttccatcgagggggacgtccaagggctggcgcccttggccgcccctcatgtcaaacgttagacggcaaagtcacagaccgcgggatctcttatgaccaactactttgatagccccttcaaaggcaagctgctttctgagcaagtgaagaaccccaatatcaaagttgggcggtacagctattactctggctactatcatgggcactcattcgatgactgcgcacggtatctgtttccggaccgtgatgacgttgataagttgatcatcggtagtttctgctctatcgggagtggggcttcctttatcatggctggcaatcaggggcatcggtacgactgggcatcatctttcccgttcttttatatgcaggaagaacctgcattctcaagcgcactcgatgccttccaaaaagcaggtaatactgtcattggcaatgacgtttggatcggctctgaggcaatggtcatgcccggaatcaagatcgggcacggtgcggtgataggcagccgctcgttggtgacaaaagatgtggagccttacgctatcgttggcggcaatcccgctaagaagattaagaaacgcttcaccgatgaggaaatttcattgcttctggagatggagtggtggaattggtcactggagaagatcaaagcggcaatgcccatgctgtgctcgtctaatattgttggcctgcacaagtattggctcgagtttgccgtctaacaattcaatcaagccgatgccgcttcgcggcacggcttatttcaggcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagcttgccccttccgccgttgtcataatcggttatggcatcgcattttattttctttctctggttctgaaatccatccctgtcggtgttgcttatgcagtctggtcgggactcggcgtcgtcataattacagccattgcctggttgcttcatgggcaaaagcttgatgcgtggggctttgtaggtatggggctcataattgctgcctttttgctcgcccgatccccatcgtggaagtcgctgcggaggccgacgccatggtgacggtgttcggcattctgaatctcaccgaggactccttcttcgatgagagccggcggctagaccccgccggcgctgtcaccgcggcgatcgaaatgctgcgagtcggatcagacgtcgtggatgtcggaccggccgccagccatccggacgcgaggcctgtatcgccggccgatgagatcagacgtattgcgccgctcttagacgccctgtccgatcagatgcaccgtgtttcaatcgacagcttccaaccggaaacccagcgctatgcgctcaagcgcggcgtgggctacctgaacgatatccaaggatttcctgaccctgcgctctatcccgatattgctgaggcggactgcaggctggtggttatgcactcagcgcagcgggatggcatcgccacccgcaccggtcaccttcgacccgaagacgcgctcgacgagattgtgcggttcttcgaggcgcgggtttccgccttgcgacggagcggggtcgctgccgaccggctcatcctcgatccggggatgggatttttcttgagccccgcaccggaaacatcgctgcacgtgctgtcgaaccttcaaaagctgaagtcggcgttggggcttccgctattggtctcggtgtcgcggaaatccttcttgggcgccaccgttggccttcctgtaaaggatctgggtccagcgagccttgcggcggaacttcacgcgatcggcaatggcgctgactacgtccgcacccacgcgcctggagatctgcgaagcgcaatcaccttctcggaaaccctcgcgaaatttcgcagtcgcgacgccagagaccgagggttagatcatgcctagcattcaccttccggccgcccgctaaatatctccttttgggttgttaataaaacatccaataagttgactgtgcgtgaaaaagaaagttttgtgtgatggcgttgaagatcgcaccgttaagctcttatgtgggatggtgcagagctcgacgactaccgataaaacgcaaccgccgcaaacagacaagaaaaagtcccaactgataacagttggggcttcagtattgtgattggtggagcaatagcaccctgaacccaaaaccttctcgctcaaccggtagtggctgataacaactcgtgagggctattgcgggttaagcatttagcgatgtctagggccagactggacgtctgaacgcaagccgctgatactgtacataaccacagtatcagcggaggatacccatgtcgctggcaaggaacgccacggcgagtcaatcgcccactcaaacaaacggttacgaacgccaccaacccgaccagacgctgctctaccagctggttgagcagcactacccagccttcaaagcctcactcgaagcccaaggtcaacacctgcctcgctacatccaacaagaattcaacgacctcctccaatgtggccgtctggagtatggtttcatgcgggttcgctgcgaggattgtcatcacgagcgtctggtcgccttcagctgtaaacgacgcggcttttgccctagctgcggtgcccgccggatggccgagagtgcggcgctgctgatagacgaagtcttccccaaggagcccattcgccagtgggtgctcagctttcctttccagctacgctttttgctggctcgccatccccagctgatgggccaggtcttgagtatcgtctatcgtacactctcaactcatctgatcaaaaaagccggttacaccaaagcctctgcacaaactggctcagtgactcttatccaacgctttggctccgcgctaaatctcaatgtccactaccacatgctgtttctcgatggtgtctatgccgaagatgactatggcaagcaacgcttccatcgtgtcaaggcacccacttacgatgagctgaatacgctcgctcacaccctcagccatcgcatcgctcgctgcatggaaaagcgtgggattttggagcgtgatgccgagaatacgtggttgacactggaagagggcgaagacgatacgctgactcaattacatggtgcttcggttacgtatcgcattgccgtcggcccccagcaagggcgcaaagtcttcaccctgcaaaccttgccagggcgtgaggataaagccgactcaagcagtcgagtagccaaccatgctggtttctcgctacacgccggtgtgatggccgaagcgcatcagcgggataagcttgagcgcttgtgtcgctacattagtcggccagcggtttcagaaaaacgtctggcattaaccgccaatgggcaggtgcgttacgagctcaaaactccgtaccgcaatggcaccacccatgtgatcttcgagccgctggacttcatcgccaaactcgctgcgttggtacctaagccgcgagtcaacctcacacgcttccacggcgtctttgcaccgaacagcaaacaccgagttcaagtaacacccgccaagcggggcaagaagcccgacaaatcggaaggtctcgatactaactggcgtgacaagagtcctgcagagcgccaccgcgccatgacctggatgcaacgcctcaagcgagtcttcaatattgatattgaagtctgcgaacactgcggcggtcacgtcaaagtgattgccagcatcgaagatccgaaggtcattgagcagattctcaagcatctgaaacagaaaacagccaaggcgaatgccgccaagcagcgtgagctgccaccagaacgagcgccgccactgactcccagcctgttcgatccatcacagagtcgtctctttgactgacgaccccaaatccaacactgctcaacactgccaacttttaaacggggcggtggggcagtttgtatctctcgagctatcaggctagagattttaccgccaaatcgaaccttattagagcggtttaggctggaccggcagttaaaattggggcttgagcggtaaacgagtgagggaatttcaggtaagatacttcggatgaggagcaaaaaggtggtttatacttcctataccccataaaggaggcatccggttgcgttaagcggaattcaacggtgttgttgtccagtttgcgcacgctttggacgttatcggcaaactgcaggctgtcgaagtaggggaaactgctgccgttcacattgtgccacggatggtggcgatcaaagatgcgctcgaaggtaaacaccacatcgtctgcattcagtttgcgggtgggggtgaaccaggccgtcttctgaaactgaacgtcacgacgcagatggaagcggtaggttgcgccgttatccagcacctcccagctttcagcaagctcaggcaccagccgataggtgtagggatctacatcaagcagtcggtcatacaattgggccgctaacgtatcgacgatgagaccgctgctcgttttttgtgggttgaacgtattgacttgcccgctgacgcaatagacaaacccactgtcacggatatcagcgtgcggagcttgctcaggcgcagcagcagcctgaccactcagaaatccagccatcacgatcagagatgataaaaccaggcgcataattttaatggattatataaagaggctatcttactaatacttaatgacatttgccattaccgtttgtttttgggagtaacaggctcgataagcgcgagttcaacaccgcgacatggtcaaaattcatacctgctatccactttgaatctactctgttaactatcttagcattttcatggcccatctgacttgctataaacgtgggggcgtacctgtcgttgtttactgattgatatcagtgtgtgctcggaagatggagtatggcatcgataacgaagtgtggagcgagcatttagagtgtcactatcaatgctgaactataatcattcaggaagcagtacacgttatgtgagttgtaatagctaaagtatctataattatttcaatatgttaattccattttttcttcatggcatcttaatcttctcacccatttatggtgatgatttccattggttattaaatgcattttggctttgatcctggttatgaacggttttctgttgccatttaacttctaatggaatgaattgtatgaataaattactgagccttgttgtttcacttgccctattgcctggagccgtttttgcagcgtcgactccagactgtgtgaaggtgaataaagcgcaaatcgaagcgctatttgataagtggaacgagtcattgaaaacaggtaacgcgcagacggtatctgaaaattatcttagcgatgcggtattgttgcctactgtatccaataaagcgcgtttaacggacgctgaacgtgttgattatttcgagcattttcttgcgaaaaagccgaccgggaaaattgacatgcgtaccattcgcctggggtgtaataaggcgatagacaccggtacctacacatttaccttcgcggacaaatcaacggtttcagcccgctacacattcacttatgcatgggacggtaaagagtggaaaatttccacacaccactcttcagcgatgcctgaagggtaaaaagctgtgcagggggcggaatattcaccgctccctgaagactagccaataatcgcgatgccaagacgttccatgagcaacgatgcctggtagttgtccaacttaacgccttgtaaatcaacgcgccgaatatctaagtcacccaactccgaattggtcagatcgcaatgtgtgaagtttgctgctcgccagtcgaaagtcgaaaactcgccgccggagagatctgaaccactgaacgtcgcgcccagtacctgggcccccatccaacggttttcccacagctcacacttttccaacacgactttcgaaaaattggcgtagcttagatttgtgttagtgatatatgcactgcaaaaccaggtgcgagtagtgatcatattcataaaacttgcgccgcgaaaatctgcgccttgcgcacgacagtggcgaatttcaatgccaagcgcactggcattgcgaaaatccgccatggataaatcacagcttttaaaaatggcatctttcagcatcgcacgactaaaattgcaccctttctggctttcacgatcatagaactgacagccgataaattcagtgccgctcaggtcggcacctgaaaaatcacagttaaaaaatgtactattttcaattttctcaccggtgaagcggtttctgttaattttttcgccaacgagtgccagagccatattttgtgcctgtttttttatacagtaatggcgtcatggtaaaccctgatgaggttatgcgtcaaatccgccaatataacatctgcaaatgtgcgttaaatctggtgttttttcagcaaagcgcgaagctgatggtaagtcagacccagtaattcagcggcttttttctggttaaattttgcctgctgtaagctggtttgtagaaagtctttctcttgctgctgctggaattcacgcagatccagcggtaaccctacagacatctgtttagtttccggcgcctgcggctgcgtctggttctgaaatccatccctgtcggtgttgcttatgcagtctggtcgggactcggcgtcgtcataattacagccattgcctggttgcttcatgggcaaaagcttgatgcgtggggctttgtaggtatggggctcataattgctgcctttttgctcgcccgatccccatcgtggaagtcgctgcggaggccgacgccatggtgacggtgttcggcattctgaatctcaccgaggactccttcttcgatgagagccggcggctagaccccgccggcgctgtcaccgcggcgatcgaaatgctgcgagtcggatcagacgtcgtggatgtcggaccggccgccagccatccggacgcgaggcctgtatcgccggccgatgagatcagacgtattgcgccgctcttagacgccctgtccgatcagatgcaccgtgtttcaatcgacagcttccaaccggaaacccagcgctatgcgctcaagcgcggcgtgggctacctgaacgatatccaaggatttcctgaccctgcgctctatcccgatattgctgaggcggactgcaggctggtggttatgcactcagcgcagcgggatggcatcgccacccgcaccggtcaccttcgacccgaagacgcgctcgacgagattgtgcggttcttcgaggcgcgggtttccgccttgcgacggagcggggtcgctgccgaccggctcatcctcgatccggggatgggatttttcttgagccccgcaccggaaacatcgctgcacgtgctgtcgaaccttcaaaagctgaagtcggcgttggggcttccgctattggtctcggtgtcgcggaaatccttcttgggcgccaccgttggccttcctgtaaaggatctgggtccagcgagccttgcggcggaacttcacgcgatcggcaatggcgctgactacgtccgcacccacgcgcctggagatctgcgaagcgcaatcaccttctcggaaaccctcgcgaaatttcgcagtcgcgacgccagagaccgagggttagatcatgcctagcattcaccttccggccgcccgctagcggaccctggtcaggttccgcgaaggtgggcgcagacatgctgggctcgtcaggatcaaactgcactatgaggcggcggttcataccgcgccaggggagcgaatggacagcgaggagcctccgaacgttcgggtcgcctgctcgggtgatatcgacgaggttgtgcggctgatgcacgacgctgcggcgtggatgtccgccaagggaacgcccgcctgggacgtcgcgcggatcgaccggacattcgcggagaccttcgtcctgagatccgagctcctagggatcgcctcagaaaacggaatctatggtcactcccgtttttgcaacaccgattttgacgacaagttggcttgcttgaatctatccggcgtctgaatgggattttattcccgcgcctcgatgagttccgcgcctgatgaacctccagaaaatatacggcttcaatgagcctttccgttttacaggttcctcaacaggccggtgggccgttagtatcatcaatatcagtattcgcaaaaccagatgaatgattgtttaaactggtgtatttctgcctttatgcttcgtaagtttgctgtcgcgccgtcagtgcccaggctattctggccagcttgtttgccagagcacaggtgacgacaaagttgcttttccgacacaacaactccctgacccagtcggccaacttgccagactggtgttccagtttttgtatgaataccctggcacactgaaccaacaaagttcggatctttttgttgccccgcttgctaatccctaacaatgtcgtccgacctcccgtgctgtactgtcggggtaccagccctgttgccgccgcaaagtcacggctgctggcgtactgcttcccgtcgccaatctcagttgaaatagtactggcagtcagcgttccaacgcagggaatactcagcaagcgctgtccaacctcatcttcgtccaactttcgtttcaactgagattccagatctttaatctgctcaacaagatagtgataatgctgttgtaatttcagcagtaactggctgagataaagaggcaaactactgtcctcaagaagggtactcagtcgactaataacggcagcacctcgcggaacgctgataccaaattccagcagaaaagcatgcatctgattagttgttttcaccttatcctgaaccagggattcacggacacgatgcagagctcgcattgcctgctgagattcggttctgggctgcacgaaacgcatagatggacgtgatgctgcttcacagatagcttcagcatcaacgaagtcatttttgttgcttttaacgaatgggcggacaaattgcggtgatatcagctttggaaaatgccctaactctgccagcttgcgtgccataaagtgagaaccgccacaggcttccatcgcgatggttgttgccgggcatgtcgccagaaattcgattagctttggtcgggtgaattttttacggtaaacggccttcccacgatgatcctgacaatgaatatggaaagagttcttacccagatcgataccaataagcgcaatgttttccatgatggttctccgaatgaaagcctgtcctcagcatagtactgggaaggagggagtgaccatctcattaaataaagcacgctaagccgtaagtaagcgtgctcctgtgaaagccacagctaaaactgcgtagtacacatagaggtatttttttctagatttatatcgaatttcgttatcgaatttcgcttattattgatcaactcgttgcagatcatgaaggcgaaagtatgacgaataaccctaccgatgacagcagaccatggtcggtctttcttatttttctgcggcttggattgacatcttttggcggccccattgcgcacttgggctacttccgcgccgaatttgtcacacggcggcgctggctctccgaacggagctatgctgacttggtcgcgctttgtcagttcttgccagggcctgcaagcagccaggtcggcatagcggtaggactgtctcgggctggatacagcggggcgctggctgcttgggctggcttcacgctgccgtctgccatagccttgatcctttttgcgctcggcatctccagctatggcgattacgtctcgcagggcgcgttgcatggcttaaaagtggtggctgtggccgtggtcgctcaagcagtatggggcatggcgcgtaacctatgcacggatgggctgcgagtcaccatcatggcaattgctacctgcgtcgttttacttgtgccgtccgcgtggggacaggttggcgtgattgctatcgcaggcatcgcaggccggttattgttcaagccagcgaaagttgttgagcatgaccccctacctatcacggtcagtcaccgggccggcgtgctttggctctcgctgttctttgtcttgctgattggcctgccggtgttggccgaactgatgccaagtcaaaccatggcaatggtggattccttctatcgtgtcggatcactggtgttcggcggtggtcacgttgtgctgccattactgcaagccgaagtggtgccctccggctgggtcaacaatgaatcctttctcgcggggtacggggcagctcaagcggtgcccggccctttgttcacgttcgccgcgtttcttggtgcctcgatgaacaccgccccgtcgggctggatcggcggcattgtgtgtctgctggctatcttcgcgccctcgttcttgctggtcgtcggatcaatgccattttgggagcgtttgcgccgcaatacaggcatccaagctgcgctggccgggatcaatgccgctgtagtcggcttgctgctggccgcgctgtatcagcctgtatggactagcgccatctttcagccgcaagacttcggcttggcattagttgcccttgtcgcacttatgttctggaagctcccgccgtggctggttgtcctcggtagcggtgcagctgggtggctgttgagcgtcgctctgtgaggcacaaaaaatgactgacaaagacctctacggcggtttgatccgcctgcacatccttcaccatgcagccgaggaacctgtctttgggctggggatcatcgaagagctacgccgacacggctacgagatgagcgctggcaccgtgtacccgatgctgcacggcctggaaaaaaaaggctatctgacctcacgccacgaacgcaccgggcgacgtgaacggcgtgtttatgacatcactgaacaaggcagaactgcacttgctgatgcgaaaacaaaggtcaaggagctgttcggagagttggtagaaggtggttgagtttttgcttctatgtcaggttgagctataccctaactggattgtcattttcagaagacgactgcaccagttgattgggcgtaatggctgttgtgcagccagctcctgacagttcaatatcagaagtgatctgcaccaatctcgactatgctcaatactcgtgtgggctctgttgcaaaaatcgtgaagcttgagcatgcttggcggagattggacggacggaacgatgacggatttcaagtggcgccatttccagggtgatgtgatcctgtgggcggtgcgctggtattgtcgctatccgatcagctatcgcgaccttgaggaaatgctggcggaacgcggcatttcggtcgaccatacgacgatctatcgctgggtccagtgctacgccccggagatggagaagcggctgcgctggttctggcggcgtggctttgatccgagctggcgcctggatgaaacctacgtcaaggtgcggggcaagtggacctacctgtaccgggcagtcgacaagcggggcgacacgatcgatttctacctgtcgccgacccgcagcgccaaggcagcgaagcggttcctgggcaaggccctgcgaggcctgaagcactgggaaaagcctgccacgctcaataccgacaaagcgccgagctatggtgcagcgatcaccgaattgaagcgcgaaggaaagctggaccgggagacggcccaccggcaggtgaagtatctcaataacgtgatcgaggccgatcacggaaagctcaagatactgatcaagccggtgcgcggtttcaaatcgatccccacggcctatgccacgatcaagggattcgaagtcatgcgagccctgcgcaaaggacaggctcgcccctggtgcctgcagcccggcatcaggggcgaggtgcgccttgtggagagagcttttggcattgggccctcggcgctgacggaggccatgggcatgctcaaccaccatttcgcagcagccgcctgatcggcgcagagcgacagcctacctctgactgccgccaatctttgcaacagagcccgccgtgctagtctgctcggtgatggtggagtgaagccaacccgcaatcgggttatgaatctgcatcgcgattcgcaatcagctgtctcttgagcatgtcgaactcctgcgatgttatctcgccgtgctcgtgctgctacaccaacttgtccaactcatccgccacaccgctccccaccacctgcgatggccaggttgtttgctgggcgcattggcggagtatcgtctcgacccgggacacccacaccggcacaaccttacgggagtgattcactgtcaaagaatcggcccggtgctctgacgcaagtatcgggatggtcaccatttgtaagccgtagacctgagtggtgatcaagacttcgataccaccgaccgtaccggtactaatcgacgacggtcgtgttcgtcgcctgccgcagggactctgcacacctccgtttacgcatgtgcctggaggagttggaaatcgtcgtgttcgggaaacattaaacacaggatggcagcgatctgagccagcacatgatcagctagctcaccatccggatcgacggcccactgcatcgtcgcgccagcgatgaccgagtgcaggagcaactcagctgccgcaggagcacctgggggcagtcgcttgcggatcccctccaccaccgcgcggttccgctggatcgcaagcgtgcgtagctccggcacctggagctcgtaccaggagatgagatagttcaccgagaagtcgttgcgagtgttcatgctccgaacgagcacctgcaaaaattcccagagcccttgcggccctgcgcctatcggtatcgcattcaggtaatgccgcacctgctcgacgccgcgctccatcatcctcaccagcagcgtatcgcggttggtgaagcgctggattaacgctgcgcgggagagccccacctcctttgctactccgctgagcgtgaactctatgggaccgcaacgcttcagcactacggtggcggcctcgagtacctcgtcatcggacttgagcttggggcggggcatcagtgttcaccttctgtatgggttggggcggaggctgtggctgccgccgccattgtagcaaattgaagacggagcgagagtagagccacgagccccgcaaacacggccgatacaacgaggccagggagcggaccagcaaggtcgacaaacgcgccggccgcaagcataaccatgggcgaggctgacagcatcaccgccgagaccgtgccgagtacccggccgagaagttctggcggcgtgcggttgtagatggcagcgttgagaatgggagagactgagccggtcagcagtcccacgagcgcgcccaacaacatcagcaccggcacgcctggcaactgtgaaagcagaagcgagcccaccgcagagccacaaaatgccaccgccagccagttctgcgctgatatccgggcgccgaccgacgcatgaatggcaatgccaaggagaccaccagcccccatcattgaggagaacagcccgagctctgctacttggcgtcctgcatctacaaacagcgcaggcatgatgacgctgccgttggcgccaacgatgcccacgaagatcatcactataccaaagagagggcgcagcaggggttcgctccagagaaaagcgacgccggcgcgcatggagagagtcgccgtcgtggtcatcgtccgagcggcacgcgcgggaagcacccacgcgccgagcagacctgcaaggacggagcagaacgccgtcagcccgagcgttggcgcagcgccaagcaggccgattgcggcccccccaagggccgggccacctagaatcgcgacgttcccgatcaccgctttcagtgacgagacgcgctcaacggagagcccggcgacgtggccgagtttgggcagctcactgtcctgcgcggccataccgggtgcgtcgaacgcggcaccgagcaccacgcaagcgatcagcccagtgttcgagagggcgccaacggcatcgagcagtgggatgctcgccatggccacgccgcccaccacacccgagatcaatgcgacgggcgcgcgcccgaaccgatcgacgaggccaccaccaacccacgcgccgatgatggtcgcgatgacgctgctagcggccgtggcgcccgcccaggccgcgctctttgtatgagacaggacgaaccatggaagcgcgagggccgccaccgcgttgccgatccggaagagaaaggtcgccgcgaacagcgtcgcgagcgggctatatcgacgttcgctcattccgctgcggcgagctgcgccttcgccgcagcgaggtactcttcgttacccgagtcgagggcgaagagtgcgtaggtgaccgccccgaacgcaaggcgctccgcgatgtggtgggcgagccgcggccacacccggccaccggccgcttcatacgtgaggaggagcttcgcgagcccctcttcaccaaagaccataaggtgcgcggccatgtcgatggcagggtcatcaacgcgggcctcgctccagtcgatcatcccgctgacgcgctccgtgttgtcgatgagcacatggcccacgtagagatcgccatgcaccaccacggagaaatctggccacgacgaatcgtcgtcgagccagcgctgccaccggtggaggcgcttgtcgttcaccacgaactcgcgtcggacgcggtcaacgtcgtcggccaccttctgacgggcctgcgtcggtgtacggatgagcatccccgcatccacggcggcggaaatggggacggcatgcagggcggcgagcgcggtcgcgaagctctccgcgaagacctccgagtcctgcggcacgacccagtcgggcgtggacgaaccaggctggatgaccatcgcagtcgagtcttcgagcatgggataggcaacgagctcggcgttggccacgcgccagtccggcaccgcgaacggcaggcgattcttgagcattgccagcacccgcgcctctggttcgaccttcgcgcttacctcggctcggcgcgggatgcgcagcacccaccgacgtccatcgtcgacggtggcgatcacgatcctatagtcgagcccaagctcattgacagtcagcgggccatggagcttgagcccatgtcgggctgcaagtgcgtacagttgggaggtatcggcggtcgtgactacggtcatgattcactcctgagggcttgacgggtttagccacctaaatgtaacagtcacgtcggttatattcaatccggcactgttgcaaagttagcgatgaggcagccttttgtcttattcaaaggccttacatttcaaaaactctgcttaccaggcgcatttcgcccaggggatcaccataataaaatgctgaggcctggcctttgcgtagtgcacgcatcacctcaatacctttgatggtggcgtaagccgtcttcatggatttaaatcccagcgtggcgccgattatccgtttcagtttgccatgatcgcattcaatcacgttgttccggtacttaatctgtcggtgttcaacgtcagacgggcaccggccttcgcgtttgagcagagcaagcgcgcgaccataggcgggcgctttatccgtgttgatgaatcgcgggatctgccacttcttcacgttgttgaggattttacccagaaaccggtatgcagctttgctgttacgacgggaggagatataaaaatcgacagtgcggccccggctgtcgacggcccggtacagatacgcccagcggccattgaccttcacgtaggtttcatccatgtgccacgggcaaagatcggaagggttacgccagtaccagcgcagccgtttttccatttcaggcgcataacgctgaacccagcggtaaatcgtggagtgatcgacattcactccgcgttcagccagcatctcctgcagctcacggtaactgatgccgtatttgcagtaccagcgtacggcccacagaatgatgtcacgctgaaaatgccggcctttgaatgggttcatgtgcagctccatcagcaaaaggggatgataagtttatcaccaccgactatttgcaacagtgccgtttactcatatatactttagattgatttaaaacttcatttttaatttaaaaggatctaggtgaagatcctttttgataatctcatgaccaaaatcccttaacgtgagttttcgttccactgagcgtcagaccccgtatagtgttttgcagtttagaggagatatcgcgatgcatacgcggaaggcaataacggaggcgcttcaaaaactcggagtccaaaccggtgacctcttgatggtgcatgcctcacttaaagcgattggtccggtcgaaggaggagcggagacggtcgttgccgcgttacgctccgcggttgggccgactggcactgtgatgggatacgcgtcgtgggaccgatcaccctacgaggagactctgaatggcgctcggctggatgacgaagcccgccgtacctggctgccgttcgatcccgcaacagccgggacttaccgtgggttcggcctgctgaatcaatttctggttcaagcccccggcgcgcggcgcagcgcgcaccccgatgcatcgatggtcgcggttggtccgctggctgaaacgctgacggagcctcacgaactcggtcacgccttgggggaaggatcgcccgtcgagcggttcgttcgccttggcgggaaggccctgctgttgggtgcgccgctaaactccgttaccgcattgcactacgccgaggcggttgccgatatccccaacaaacggtgggtgacgtatgagatgccgatgcttggaagagacggtgaagtcgcctggaaaacggcatcggattacgattcaaacggcattctcgattgctttgctatcgaaggaaagccggatgcggttgaaactatagcaaatgcttacgtgaagctcggtcgccatcgagaaggtgtcgtgggctttgctcagtgctacctgttcgacgcgcaggacatcgtgacgttcggcgtcacctatcttgagaagcatttcggaaccactccgatcgtgcctccgcacgaggccgtcgagcgctcttgcgagccttcaggttagaggccgtcgacaatgataatctggatcaacggacctttcggcgccggaaagacgacgctcgctaagcggctgcgcgatcggcgttccaaatcgctgatctttgaccccgaggaaatcgggttcgtggtgaaagaaacggtccccatgccagcgagcggagactatcaggatctccccttgtggaggggacttacgatcgcggcggtcagggagattcgaaggaattactcgcaggacatcatcatcccaatgacgctcgtgcacccggactatctgactgagatactcgacggggtaaggcggatcgacgatcagctgctgcacatctttctgacgctcaacgaggacctattgcgtcaccggatcgcgaaccagaccatgcatcctgacccgaatcgaaatgcggagattcgagagtggcgattagcgaatgtcgcccgatgcttggccgcaagggaacggcttccatgcacaacccgtgttctcgatagtggtgcacacaccagcgatgaactcgcagcgatggtgctcgacggaatcgatgggcgcacctgatcgccttcgacgcctgcgcaaagcgtagcgcgagggtggcgggctcacgaccaaacgcccagaggtcgatcatcgcagggatgtttggctttgtggtgcggacgacgggactcgaacccgtactctcacagagaagcagattttcgtaccacctcgactttcgccgccgtctgatgacgttcgtggtctggactgtcccttcgccattgcccgaaggctttaggcgccgcccgtccagtctctacaccttcccccgaaggggcttggctcgggattggcttagggtattgcccgttagcgttccccgactttgagcggttctactccgcggatttccccgcgggcactccaattttaaagtctgctgcgtctaccgatttcgccacgtccgccttttttcgccgttcctagcgctcgtgcgatgcacctatgttgcacctagcgccgaatcgttcttcgtcatcctgaaaaaccacgtctcctaaagccttgcatagcttatcttttctccaccacgaacttttttgtgggatggtagaaaaaaagactttttaagtccgctggcttgccaggccttgttagcttgtacggtcatggttatcgggtaaagaatattgacggcatcgctggtgtcggtggctgaaaagccggctcccatcagggcaatagccatttcagatgcaggcgtacagggcaatggtcaacagctacagcctgtctgacgattccggcgtcatggctgcggcggctatcacgcattttttgttcggtcaggcggtgttttcgtacctcaatggttggagcgtgttgatcggacctggtacaggtttggacagcacgggctgcaaatacgcaagggatttaatgggcctggtggcgttcacggcttttatcgtgacgtttctgttcaggggctactcataatctcgtggctcggcggttcccggcacaccatgacagtaaggaaggaccctgtgtctcaactctcccagcttcgaagccccgccgccgtgcaggctgccatcgatgagttcgtgcaactgggccgcacgaaattcctggcgcgccacggctacggcaagtcccgcgacttcctggtacgtgatccgaagaccggcaccgattgcgattccaaggccatcgccggtgtggccttcggcaagcaatttcccgagcagggcccgctcactgctgacagcttctccggtggcgagacgaccgtcgttccggcgctgacgcggctcgggtttcgcatcattcgcatcggcgaagactggtccgaagaagaggtcctggccacggtcgaagactatttcgacatgctgcgtgccgaggcggctggggagccgtaccacaagtccgagcacaaccaggcactgcgccaactgctgaacggtcgcagcaagtcttcagtcgagctcaagcaccagaacattagcgccgtactcgatgccctgggcctgccctatatcaacggctacaagccacgcggcaacagccaactgctgctgcgtaaatccgtacacgcctacgttctggaacatcagcagacggtcggcgctcttgtcgatgccctggaggaggtaaaacttccgggtgacaaaacctaccgagcggctttggtagaaccacccgcccgtgaagtgcttgtgcgtaccccggcatctctacggcaacgcctaccgcgaaagttcgattatgccgctcgcgatgaagccaaccgcaagctgggccgggcaggggagcagtgggtgattggctacgaacagcaacgcctgaccgagctcggccacccagagctttttcagcggctggattgggtgtccgacacccagggagacggtgcggggttcgacatcctgtcgttcgaagaggacgcccatgagcgcttcatcgaggtgaaaaccaccaatggcggggtaggctcgtctttcttggtcagccacaacgaactcgaattctccaaggaggcgggcgatcaattccatctgtatcgcgtgttccagtttcgggacggtccgcgcctgttcacgctacccggcgacctcagccaacatgtgcatctcaagccgacggactaccgggcgagtttccggagtttggtggggtaaaggcagggttctgttgagccgaatggctgtgtgcggccgattctgttgaaaaagtagcggcctccccatgccgttggcaaaattgctttgtcagcgagcgtgggggcgaacagcatgatgggacagttaccgggaggacagcagcgcctgttctactcgttcaatctggaagatcacgtcccggcccaacatctcctgcgcagcatcgaccagtgcttggatctcagtgatctacgtgcctacctggcagatttctatagccccatcgggcgtccctcgattgacccggagttgatggtgcgcatgctggtcgtcggctactgctatggcattcgttccgagcggcgattgtgcgaagaggtgcacctgaacctggcctatcgctggttctgccggttgggtctggaagacgaagtccccaatcactcgaccttctcgaagaatcgccatgggcgttttcgtgacagcgatctattccgctggttattcaatgaggtgctgcggcgctgcatggcagccggcctagtcaagggtgaaggtttcgccgtcgacgccagcatcattaaggcggatgccagccggcaacgtggggtggcgggagatgaggtcgattggaacgatccaaagctcagcagccgcgcagtgcgcgagtacctcgaagcccttgatgaagaggcgctggctgaggctcttcccaagaaaatttcgctcactgatcctcagtcccgttggacagcagcgccaggtggcccggccttttttgcctactccacgaattacctgatcgacactgagcacggtgtgatcatggacgtggaagctaccccggcgcaccgtaccgccgaagtcgattcgactaggacgatggtcgagcgtgtcgaggcgcagttcgatctcacaccggaacgccttatcggcgataccgcttatggcaccgccccgatgctggcctggatggtcgaagaaaaggacatcgaaccgcatgtgccggtgtgggacaagaccgagcgcaaggacgacagcctctccagtaacgactttcactggagtcaggacgccaatgaatatcgctgcccagccggcaaaccgctacgcagtgaatggcgcgccttcacccagcaaaggtcgcgggtaactaaggccaaaaccgtcatttaccgctccagccaaaccgactgcgccacctgcccgttgaaagcgaaatgctgccccaacacgccgaatcggaagatcgtccgcagcatccatgaggctgcccgcgacgtggctcgacgcatcgccaagacaccggagtacctcgtctctcgctgcgaacgaaagaaggtggagatgcttttcgcccacctcaaacggatcatgaaactcgaccgtttacgactgcgtggcctaacgggtgccactgacgaattcaccttggctgcgatggtgcagaacctgcgccgcatggccaagcttttgcctcaagggccaccgctgacgggataggtatgcctgctacgagcagaaaccctcaaattaacccttaaacctgagcaaggacgctcagtgaaacgccggaaggcaacttgaagtggcttgcagccacttcgacagcaggcacacctgatcggcaggctgccgctaaagctactttttcaacagaatcggccgatttgtgccggtcgtcaaggaccgcttcggatcactctcaaccggatcgcgggccagctgatttgctaaaacccgcgccaaactcaacgaaggatttcgcatgatccgttttttttggcgatcacgctcggcctgatggttggctgctcgtcgagttcaagttcgatctgatcagtgagcggtagcgcaagtggatcgcctcagccaagcagcgttgttgccggcggccggaagaaagccttgaactagtagcggaaaggatgaaactttatttttacgattctcagcattttccaaggttgagcgaaaattaggatgatactttaatgattccggcctccatccccacaaaacgcttgcagatggcagaattgaaataagtcattgttttaaatggatttttgcaggaattgctacgttgcagggtcctgtagttaggatgacactttattttcctcccacaggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctggtaaatgcttcaataatattgaaaaaggaagagtatgagtattcaacattttcgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggtgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgctgccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttatttgagtaaatgcgatttctgccagaagggcggcacaaaaaaacccgactggtgagatcgggtttttatgttttgccccggatggaggcgggaacacctaaacaattgacggttaaatattacgccatgagtgcagtaaatgcaaaccacccggaatagggtatactgcgcgggtcaatctgttgacgttcttctccagcccccgcttacgcggcgtgaacggattaaagccctggttgaaaaacaggtggtgctgtttcaatgccctggctggagtgggaacatctaaacaattgacgtgaggtagccgctgtgacaggcaatgtaagggcgatcagcggtgttcctgctctgtatggagcaaacggatgattaacttgttaatcgtcgttctgcgggctgttgttgcagtgtctaacgctgtgatagcggtcctggagctgatccggcagtattttgactgaccggaaacgtaactaaaggcggggggaccactccccgccttttaacacaaaccttcagtttacagtctgtttagcggccgacacccacttatacagacacccaaccgacaggttcagatcagcagcaattgccttaactttttcccccgttacttttctggcctccatccttttcacggcttcagccctgagcgcttctggtaccactttcactactggcgagtgcttccagatctgatgtgtctgcgctacgtttcgcgcagccactttatgcagcctggtgctggttgcacgttccggggcgtgcttccttccttcagccatgagcgcgagctgttcatcgggattaatctcgcctcgccgtatgcggttcaatacattctctctgcactgacgggaatggctgacgctggcaaggggggtgtgtaacggaatagcccagcgttcgcggtattccctggcgttcatctgatgggttttgttcaggtgcggagccaggaacgagaagtattttccacactccaggcattgaattttttccgttaattcttctttatcagccatcgtttggcctcctgattaacaacgccggcaaacgccggcgttcagggttacatctcgatcatcggaaacagatcgtcttcctggtggtaaagcacgtcgtctgtgtccgtcccatcaagccagtcagcgaacagttcaaacaggcacaccgacatttcctccggtgaaattgcgccacactgcagctcgtagaagacctcaattttttccagcaggctgttggccgctttgtcgtagccgaacagcttgatcggcgggaagtgcattgcatccgggttcgtttccgccagttcatcatccttacgttccggatagatgctgtacagcgtcgccggcgatggcggtaatacctcgataaagcggtttatccgcatcggcagttcgctggaaaacttctcattgtccggaatgtaaaacgctgcgctgcgtaccagggcatcctggaaagaaattaccccctgaccttcggtctgcttacgcagctcacgaatgttcacgcggtccttttcacggatttggtacagatccccttcccggaccgttttggaaaacgtgccgtgttcggcttcctgcccggagagctctgaataataatcctggccgacggtttcgcgcagtatttcaaaggttttccggtcttcaatcgactcaaaatatttcacgcgggtgttggcgatcagcgaatccacctcgccgttcgcgccgcgggcgagcgacgggtgatcctgggaggaaaatacgcccattttttccagtgaacgcatctgcgcactcagcgtatccataccgctggtgaaatactggcccagctcgtcataaatcagcccgtatggcgcctgattattcagcgcgtgagtgagcaggacttcctcttttttcccttccagctggtagcccaggtccttactgatggtcattcgctgcagcgtcacatataaacggccaagcgtggccgcttcaccgcgtgacagctccagcgacgggatcatgacgatgagaatgcggtcattgtggaaaatatcgtccatccggatatcgccgctgtcttccggaaaaacatggccataagtttcattgaacagggagagcgtcttcaggaactgacggctcatgtagttatgctgttcgaaggcgcgggcttcccacgtttccgggtattccgcgttggccagcaggaagcctggtgtattttccagataactttccagcgcggcgtaaccctcgctgtgccagccgtttttctttgcctcaacgtacagcgcggccattgccggcagcgacatgtttttctgaatagtgcgctgtgacaaaagcagttcgccgcgggcgcgcttataacacagggcattgatcagcgccgacatcatggcttttgccgtatcctgccactgtgccgaatctccgcccacctgggggagcatggattccatcaactggatgatgaaggtcggcggggcaacgctgaagaggttgactgagttggattgcgcggggatgctttcctgcttcaccaggttcacgaacctgtcaatactgccggtcagcaggttgaggacgtaataatcatcctcacggccgaaacggcgcgccagcgaccaggtggcgaaggccagcttgttatctgctttaccgtctgagaggcagtagccacggcaccacagcaggaagttaacgataaacccatagaaggtttcggttttacccgacccggtggtaccaaaaaataccatgtggcgcagcaggtccgtcatgttcagccacagctcgcggccgaacaggcgtccccgctcatacccgacatataaaattcccttcgctttctttcgctcccggaccttatgcgggatgcgaatgggccccagacggtattccgcctgggtttccgtggtcagggtgtcgtcgaacatttcgcagtctttcggcattcgcagcggtggccggaatttacgatccgtgaatgccaccatcagcggcggaaggatgaggatgctcagcagcatggtaaaggggagaaacagacccagcacggtcagtatggccagcaggccaaactgtacgttcggagtgaggaaccactccatgaacgaaccatctgggcggatgacttttttactgtctacagattcctgttgcatagcggctccgggttattctcctattgcctgctgcaggcgggcatcgtctttgacgacggaaaccgggatcacatcacccgaatcggcaacgatggacggggtgccgataaacccgaatttcgcaaatgccatattgttcgcgtcaaccaggcgggcgccggtggtatccggggctgttttcggatcgtcaattttcgggttcatggtggtgtatcccatgttaatcgtgtcttcccagcttttcagtcgggtatctttcccggtatcgagtacaggagcggcgcggaccatcgagcgcaggtcgccataaacggacacggggaaaatctcgacgttatagcgtttgctcagttcctgcagccggggttccagcgcgcggcagtgcgggcacatcggatcggaaaacacatacagggtgtgtttatgcccggttgaaagcgagatcgtaaagcgtccgctggtgacggcatctttcagtgacagcaagtcgttatcagccagttttttaggggcttcaggtgctgcctgaacctgcagctgctccggcgcagtgacaggctgctgcagacccggcgcgggagcatacgagtttgcctgcagtggctgtgccggctgatgcatattcatcaacgcgccgttgatggagacgataaagcccagcgcggcgactggccagaagataaagttagacagccggcgcacggcattgagtccgcgctgctgtttcagctttttacggatattgttcagcgcctcgatggcctgatattcgttttcaaagcggaacagctgcaccgcttcaccagaaaacgtattgtgcgtcaggatgtggccgctgccggcatcatggccaagtccaaaatgacaggctgacagggggaaggagtgcagggcttgttcctggtagcttccgtcagcggaaagataaagtgtgttgcgatcggtttttacggtgagtttcatacttcctcgcttagagttccaggtgatcgggacgggttgccggctgaggtttagccggcgtcggttcatcgtctgtcagtcgcagactgttgttcggggtgtcgtgcggagctgcaggcggcgtagccggctcgtcttccgcttcagcgtcagattcaaatgcaaatggcggtataacgtcttcctgccagtgagcgaccggaatggcgggatcgcggcggtcaatatggcggtaatggagcagatccagctcaaggcccagcacggccgcccgcacggttggcgggaatttcgccgccttttcttcatcgagcgtcagccggtaactttcgcactgactccaggcaataacgccacctccttcaatgaagaacttcccgcggccggcgcttgaaagtgcataccataacgtgcggtccacgcctttcagccaccggaaatttgcggatggcagctgcaggtcatcatcgaacagcgcgtagagtagcgaacgggagctgcggcgcccctttgcaaattctctgaacgcttcgcactggctggcgacggtccagagcccggcggccagcgaataatcagggaagccgtctttagtccgcaggcaggaacggttgagatcgtcgagaatggatttagccgttggccggtcgcccataaaccgcagatgtacaaacgaggctgcgagtgctttttcccagggtgccagggaaagggccgccccttcgggagccaggtttacctgtgacaggaataccttatgtgcggcttttttacggaggcggcgtttagcggtatcaatcaggttatgttcctgggcaaactcaacgggtgactgcgcgctgcgtgcttcttccggatcttcattcagcagcagctgatcgaggtggccaaaccgggcgaggatatgggctatccccggatagttttttaccatcacccagggcaggttccggatggtcagtttacgggtcagacggtagtaaggattgcgcgagataacccagacgaacaacccgatgattaaaatgaacaggggaaagagcacgctggccgtgtcgttcatcacgctgataaagtcccacagcgaaacctggtgattataccgcgccgccgacagcagcatgtcccggcgctcggtcgcattacccggtgtaatggcgtccggcagtacaccgtagatccagtaaagcaggcgacaggctccgtaaatcacataatcacgaaagccaaaccacatcactgtgatcatgatgagaagggccccgataatcatcaggccatcatcccctccccctttccgttgtggctggttttgcatcagaacctcagagaattttggtaaaccttcccgatataccaggccttacgcgccggcgtattggagtgatagcggccgacaccctgccagaaatcccccccttcgagaatgatgttgtatttgaggatgtaggcactggccattgcagaggcgcaaaagtcgtgctgcagctgctccgctctcacaccaaaatcagccgccagcttattcgcccagatggtgtttatctggaatgcgccgtaatcgtaagtaccgtttttgtttttatttttcattcccggtttcccgccttcagtcagccacaaggcgaggaatccacggagaggaatgtgataaatctgtgctgcttcaaccatgcaggttggcagcgggattgagggcggggcatcaaccataacaatctcctaatgatttctatttcgagtaaaaaatataccatcaaaaggagatttttttaaatcattttggtgcgcataattgagagggtgtcagaggaattttatttgccttcgacagagacaatgttgccctcattcgcattttcccatgtcgggatttcgccactcagcgtgcagcgaagcgtctcttttccgacggtaagccgtgcgccactgctgtagtaatgactttcatacaggcaggcggttttcttttgttccggggtcagtgtaagactggcattctgatcctttgccagcagttgtacaagatgttggtttgtctgtacctgttcacccagaatgctgctgagcaacgaatcggttttcggcgcggatacactctgagcaaaggcactgcaactgattacggatgcggtcagaatgagagcaatcttcgtgttggatttcatttcactttctccattaaaaaaatcggtaatcattttctggatcatttaaattaggtctgaaaaccggcctgagtgtcaagccgattctgtttaaaaattaac

SEQ ID NO: 2 - DNA, 57539 bp, synthetic construct
  misc_feature 1..57539: note = pJIMK_Core_M recombinant plasmid
  source 1..57539: mol_type = other DNA
agggatagagcggggattagtgtggcgaggcgcaggtttccgatggcaggctaaacgcaaaaaatgcgctttttagccggtgatgaggtgaggccgggaggggatgcgtcgccgatcacagtttacaacagggtttgataacgggaggaaaagtcatgctgcagggattgaatcatctgacgctggcggtcagcgatctggcgtcaagcctggcattttatcagcagttacctggaatgcgcctgcacgccagctgggatagcggagcctatctctcctgtggggcgctgtggctgtgcttgtcgctggatgagcagcggcgtaaaacgccccctcaggaaagcgactatacccactacgccttcagcgtggcggaagaagagtttgccggggtggtggctctgctggcgcaggcgggggctgaggtatggaaagataaccgcagtgaaggggcgtcttactattttctcgaccctgacggccataagctggagctgcatgtggggaatctggcgcagcggctggccgcctgtcgcgaacgcccctacaaggggatggtcttttttgattgacgggttagttcagcttactgccggatttcaacgtgcagatccacagcgccaccatcgacaaaataaacagggaaggaaggtcaagggtcttcttcgccggggaaggtattgatccggaatatgccatgcgccgtgaaatgttgtctccggcctacaccacgcagtggaaggatattcccgcggcgtttatgcgataacagcgatgtgcaggccagtctgtcagccctgcagccgttcctgctcctgcagcaatgccagatggtatgcggcaatggcgttcaccaccccggccgtcatccgtgcatcggcgattgccgagtgtgcctccccttcccaggccaatgctgcctgactggccgcgctggccagcgaaatggtgccatagcgattggtggccccaaaataccctgctgccagctccatcgcgcaatatactgtcagttctcccagccagtcggccggatcgccatgtgcggcggccgtctgtttcagaatgcgggtatcaaagggggcgttaaagataatcactggccggtccccgatggcgtgtcgcagctgcagcgccacatctggccaggatggtgcgccgctgagtgccggctcatcgataccatgcacggcggcggcctgcgcctcgatggcgaccgtcggcttcaggcgggtttcaaatatcacccggccggccgcatcggtcaggccgatttccagcgcttctgcggtattacccagccccgtcgtttcggtatccagaaagagcggcgccagactcagccagtcgtgtgcccgccgtgccatccggccactggtgctgttaagccgggacaggacggaaagacgcaggccggcctgctgctgcttctcggtcggcggccgtttttcccgcatcggaacacagtctgcaatgcggtatacggcgtacttgccgccataggcgcttttgtaccatttcaccggctcagcaccgggagccggcttcattctgaattcatcgcgcaggcgccctttcgtgaagcaccgatcgccggctatcgcctctgcgcgtcgtgtggccagctcatcattcttcatggttattccatctccgtcaggcgtttgtgataaacctctctggcgatctccaggcctgcctctgtcagcccaaacgcgagcgtcagtgactgattatcccggtatttgctgagcaggccgtgacggttcagcgtatggcaggagcgacgaaaattggtatcggcaacgggctggaggcgtcccttattgaggatgctgaaaagctgcatcgccgggaacgggcccgccagctgacgttctgccagaccatacagaacaaacaatacgtctttttgcacaccggagagccgcatcgagggggctttactttgcatcttattcatgtgtcacgtcattaaggaattaatgacaaagtaacctaattgatgtgagacatcaataatattgtgttcataacgggaaatcaggagtccgaaaaagaaaatgccgctcttgacggcggcttgttttccctggtgtcgggagaattatggtttctggtctgggtcaatgcgcagggcgctgtctgtgagcaaatccagtgctgccgtcagatgctgactccgcccgccgtgataatccggccacggaagaaagggttttgacgtatcagacatgaacatgataccggtcgggcagcgccactccgtgggggttcccttgttgtaatgaaccagcagtactgaacggtcacactgcacgtcattactgtcatcgttgttcagtacaaaagtggccacgatgccggcatcgggcctggacacaaaaagcaggctgttggcaaaaataacggcaatcaccactgaggcaatggccgcaccggtataccagaaacggccgtttttccaggcccgatagccatacagaatgcacagcagaatggcggcagcgatacctgcccactgcgcaccgactgacaggcgggagagtgcatccatcgataacggccggctcagcgcataaaatgtgacaaggaaaggtgaagccagcgccagcgtcagcagaacgaaaacgaggggtaagaagcggttgccggtatagctcccattgccaagcgccgtcatcgtgcggatcatctcactgaacgccatgatcatcataaagcagcataaagccatgtataacattaaaacatccccaaactgtccgcgtgatatggccggattttcggcctgttccacatagtcccgaccccttatcggggtgcatagccctcttcagccagtgctttcatgatctggttgattccatccatcatcagatatgcctcaatatctgacgtcgccaggccgcggcagtcactgaatcccagccgcttaaccagttgcgccagagcctgcgcttctctgtgtgtcagctgtatattcacttcaacgggtttttcgtggcgaatttcatctttcatggtcagagtccttttttgatgtttcgtgaaatgtaccaggcttcttttgcctgagctacagcccagatccagacgagagttgacacaatgcacagccccgtcatgttccagaaatgacctgtcgtatattcatggccatgaagcaggaggccggcgccggccgccaggctcatgaccaggaaaaggacggctgcgccggcgtgataaatgatgctgaacatgtaaaacccccttctggcgacggtagcgccagtctctgtctgtattaaatggtcgccgcctgccgggcgtctgcctgcatcatggcgtcaatcagggcgccggcgcaggcaatattcaggcacgtcatgttcgatttcagggtcgccatcatgtccgcgcggcttcttttcagaaacggctgcagcggaccacgatagccctgtgaggtcacgataatcagctgagtggccgcatcatgcagtgccgacacgccggccagaatatcctgctcagccgcgctgatatacacgggtgaaccggcgatcacaaagcgatcgtccgggcagcgttgcatcagctcagccagggaggcgcagcggcccgccagcgggggccgtgccgtcagtgagccccagacggtcgccggcgcgaagggcatggcgtgaaacgttgcctcatacgcgaccgccggatcggtatggtggcgcagccccagaccggccgacatggcccacagctcgatgcccggccaccgcgccgcgataccgcgggcacgcagccagtgtgcgccccggtaaagctctcccgtttccagcaccctgccaggacggcgggcagcctgggcgatcagcctgtaccagccgtcaaaggcgtcatcaagggaagcgtcgcccggaaaaacggtatcctccacgctgcccgccttgcgccgggtacaggtggtgatgaggtgaagacgtttgttcattcagtgcctcccggctctggtgcgtctgtgcctgccgcgcgcggttcgccgggccgccgggcacgaatatcgggcagaccccggcaggcatcgttccagtgcgtgccccgatcggggttattggccagcttactgatccaggtttctgacacattccagcggcgggccagctccttctgcttccagcctttcgccctgagcagcgcccgccactcctgcggggaaagcggcgccgccgtgatgccggccgcatgacaggtcgttttcatcgcgtatgctcccggccctaatcctgccccggccgtgccggctggatttgacgcagtattgttgttgcctgccatcccgtaaacccgaaggtatcggccagttcgccgggcagtgacgcggccaggcgcggcagttcgtccggggattcattgtgaaagacaaaactgaccccctcatccctgatgcgcgacatcaacattgcaataagccggtgcggtccctcatggctggcgtattccgtgaccttttcagtaaaccgccggggattgctgcagctgataccccggtcggccaggaacgccagaaagcgctgcaggaaaccggggagatccctggcggtcagggcgggtttcagcaccgcttctgcctgggcctgtgtgaagccaaacgccgccgccagctcaggggaaaagtccggcgtgtcagcgctggcctcaaaaatgacgccatcgtttatggcctgctgcatgtccgggggcatattcccgatcatattttcacgcacgccccatgcacggccggcgctttcgttcagtctctgcggatcgctgcagctgacgccccgctccgccaggaacgtcaggaaacgctgcaggacggcggcgccgatctcgccggagacgggcgcttctccggagggatagaagaccctgacgttggtgttatccagtaaggcctgttcagcgccggcgtcggacccggtgctttttccgctccgccagaccgtattatttcgactgacgatggccggatcgacatgttcgctgtccacgatgctgttaatgaacgtcactgactgttcttcgctgaaatcaaagtcgtgctgtaacagggacagcagcctgtcatgccagtcatctttcacgcgattggcggcattaaccatgaacgtgacgttcagtacaccggccagtaaacaggcagcctggtcggccacatacaggctgaagtattccagcaccgcctgttcaaatgcagcgggatcgcagcagaccacttctgcagatcccagcgactgcagaaaccgccgggtgttttcggtcacagcgtcggcgggattgagggcgatggcgcggccgcgctgtgactggatgtcaaatgtgacgatacgctccgtgacgggcatccggccgtgatcggcgatgaagcggtaaagctgctccactgcttccgtggcggtcatgcccattgcctccagacgcagttcggcgcgtttcttcagctgctcatcgagacgcacggtcagctgggcgtttttggcccgctttttacgttcaaaaaaaccggtcatgctccttcctttttgtacagtcatttggctttacataatgtacagccatttgtaccgcacttttgcgcgtgggaccacattttttcaccgttgcggtttagcggtattctccgcagtgccgccacttatccccagtttctgtggataacgctatatgtagtggctccgtcagctggaaacggcacaagatatggtgtccgcgccggcagccgtgcggccgttcccctgccttcccttccctgttgatttcgctttttccggtatttacgttttttccgcaatcacggtgccggcgaaaaggtattttccggtggttgcgggcgcaaaagtcacagggtgaaaagataagggcacgaagcattccgcaatataaactgcgtgacagtcacgccgggtgatgactacccgcgggcgattctgccaaattagctattagtgctaaatctaaatatttataatttacgcatgaaacctaatttattagagtgtggttattcagtaaacaacagcaaacggagtgaacgggatggaatatgaagcgtacaaggttcgcggttacggcttcggactgaaaaaagagatcaccggcgtgctggggcgttaccccaccctggaagaggcgcagaaggtcgcggatgactacacgcaggcctgcctgccggataacctgccggacagctacgatatgggcatggtccggccggtcgctgccggcgaacctgccgccctcgatccggacgtgtttcgcggtccgggcgcgccgttcctgatggatgccggccagataaccacgggtcacacggtatgcctgggcaaagcagcggatggaaaacggttttattttggcggggagcattccgatggccaccaaacatataaatgatgagctctggaatcgcattgaggcgctgaccgtcaaagctaacgccaggcatgggttgttgcggccgattaaagaggctgaagtactgcatcttgtcctgcagcgtggcctggaactgcttacggacgacgatctgctgcagctgggcaagtaccggcgtccgataggatttgtgctgcgtcgtccgggcgtggaaatggtgaagctggatatgctgaatatggcagatgcggcaaccgttcttctgcgcagtggacccgcaacgctgtgcatctggagccgcgacgatatcctgcgcgaggccagtgaagcggtgatccgcgaacggctgccggatgcagccctgctgtcggagggggatgaccgggcccggttccagacgctgctgcccggattctggaacgccgcgcaccggggcgaaacggccgtgatttccctgcgtgctgacaatgcggatctcgctattgcacgcatcactgacctgatgtgcgaatccctgctgggttataaggggcagcgggcataccgggcgggtgaaaacgaacagggagaagagtcatgaacatgcgaggcattctttttgtcctgccgctgtgcgccgggctcctggcgggctgtgataacaaacaggaagcgctgcagtttgaagccgcccgcacggcggcgctgaaggccgacgatccgtttctggcgctggactcgttcgaacagcagtccggcgactacggccgggactgcgactcgctgggggcatttctgtttggtaattttgcttccacggataaagcatgtcgggcgcgggatgaggccacgcgggatatggtcctgaaagccgtagaaaagggttccgtgccggcactggtgttcctgtttgacccaggcagcaagacgccggatgtgtatccgcgcgatattgacccacagaaggccgggcaggcggctgagcggcttgttgcactggcagagtcggcgccggtggacagcaaaaatcaggcgctgctgatgcgtgccggcgatgtactgcaggcgggccagtacgttctgcaggattcccggagagcggcggccctgtacgtcaaagcctggcaggcaggggatcagcgtgccgcggacgagctggtcaatgtctatcactttctcaaggacagccggcgtacctacttctgggcggtccggagcaaagacgtgcagcagctggacaatcgggctgagctgaccggagatgagatcattgctattcagaaactggctgcggacagttcacggaccgacatttgatgaaatgaataaggatatacaaaaggcgtccaactggtgaggaagggcgccttttaattactagacatttttactacttaactttctgtcggagtaaacggaaaattaatctgttaatcactgcgagtataaatccccgctaagcgtggtgcaactcagtatcagaaatgaccgccggtggttgaatccagtgccctgaacaggctttcctgggctgcttccagcagacgacctggcagacggagttcatttacgcggtaactctgcccggtctgtgtggtcacggtgagataatactgatcgtcaaactgctgaccagcaccgatatgcacgatggtggccgtgtcgatccagcagaggtttgtgccttccgcgtcctcaagtggtttgattcggcctttcagccacttctgcgggcgacccggccggcatttgatcgtgatgccgtgcttatagccgcgggttctggccacaaaggccgctattttatctttatccagttcctgcgccggggtgagcttactgaaactggacggagagaagttatactgcagttcttcaaacaggttcaggtacgtggcttcatcatccaggttgtccccgcgttctttcagcatggagccccagatcctctccatttcgcccctcagcgccagtttaaccgcctcaaattcttcctgatgttcatcgtagtagttgatatccatcgcgtctcctgttgtgtttatggcgaatttgcggaattaatatacataaaaaatgttcaaatatgaacattttcaggctataaatacaggcagattataacaggccagtccaccaggattcggccaggagctgagtacgcatgataaagaaaccacttattgccggcatcatgctggctgtggcgcacaccgccggcgctgcgcagggtgatgataccgatctgcagcagatttataccgaggcgggcattcaggggatatgcagcgccgggatgctggctgtgccggaaggggccagggatgaagctgtcccgcagaacgtcagacacctcctggttcagtgtgcctatttcgacgagcagcagttgcgccggcgtgccggcctgccggctcagacatttagccactggcgtgaccgcaatgcggtccttaatctgtcgtctgaccaggcatctgatgcgctctggctgtcagcgtggcgtgatttccgggcacaactggcctcggcctacccggccccggcggaaaaaaaggcacaggggaaagaggccgtacccgccggcacgaagcccggcactgagcatgggagtgcggcggattgaccggttttgacttgatcctgtggcgacggggcctgaactggacccaggagcgcgccgcggctgagctcggtatcagccgtaccagcctggtaaaatatgaggacggtgagacagtaccccggaccatacaacttgccaccgtcgcgctgacgctgaaagcggaatggcccaccctgaaaaccatgagcaaagacaggctgctgcggcagctgaagtatgaagtgctgaggatcgataatgaatgagtttatccatgttgtcggctcatttatccttgcgggtatcctgctgtatggcctctggcagggtacacggcgacgccggcgccggcatgaacggaaacaggccagtgcggtgcgtgtgatcgataaaattaatacgttcccgcatttcgggcagaaaattgcctatctgcgcaagattgacccgtttgtgttcgaagagctgctgcttgaaggctttgaacgccggggctttgaagtgatccgtaaccgccggtataccggtgacggcgggattgatggccgcgtgaagattgatggccagacctggcttatccaggccaaacgctataccagctatattgccgttggccacgtccgtgacttcagcgacctgctgaacgccaccggtgcccgcggttttttctgccataccggcaaaacgcgcgaaggcaccaaatcgctcattcgcggagacagccgcatgatcctggtcagtggccagaagctgctagaccttattgccaccgatgcgccgtttatcccgtatccgggataccggccaccggccgaaatggttcatcctgaacaggagacaacatgaaactgaaaggatacgtcgccgccctgccggcactgctgctgacagggtgcgccatgctccccggacagcccaccgattatgaccgcttctgtaacgtctcaggcatcgccagccacggcgaaacctaccgtgtcagtgacagtcaggatttctggttaaccccgaacggccgctatctcagccaggctgagtacagcagtccggcagatacgcttcagaaactgacgggcgtggtcagtggtgaagatcctgaccaggtgcggaagaacgccgtcagggtaagggtgttccgtgttgaaagtgagagcgccagtaaaggggcgtgcctgcccgtgcgttatgacgataaaaacgcgcggcggaaaatggattcgctgactaacggccgccgtatgatggtgtacagtgaagatgaaggccagtctggccagcagatttacaataacagccgcggtgccggcttcagttacaggttgctatgagtggattgaaaggccgccgtatcagtgcggatatgtttgccataatgctgaagatatctgccgggcagctggtttttgcagcacggcatgacaagccgttacgcggactgaccctacccccttacagaatcgaaggcggaaaaagctggagctcgaccacctccacccgcagtatgacgtttgatatgcaggatgcggtcgatttcagcgagaaatttcataaaattcagtcgcctgacggctgattagcggattttaaaaaattgaacactttccccctgcaaatttctcagaattcacgtagcgacgccatcgggacatatgcctacttcgattatgccgatgcgctggcgctgcgggagatggccagccaggctgatttacccaaatacctgctggccccggaggtgtcggtgtttctgcggtatgtgccagacctgcgccagcaggtccttttcgataccctctggaacacgggcgcgcgtattaatgaggcgctggcgctgaccggcgccagctttcagcttgacggttcccggccgtttgtccggctgaagacgcttaaacagcgtcagcgtggccgcggcaggccgggcaaggatgaagaggtgttccgtcttgttcctctgaccgatcctcaatatgtcaggaaggtgcgtgaatttctgaccaccctgcgtattggtaaacagcagctgctctggccggtgcagagtgataacacgccgcgtaactggatcaggaaggcactggacctcgcaaaacgtgactctgtcacttttagtatccctgttacctgccacacgttccggcacagcttctgtatgcacctgatccagcatggggttccgctgaaagttgtacaggcttatgccgggcattcccggctggaaaccacagaaacctacacgcgcgtgtttgcgctcgatgttggtcgtcagtatggcgtccggttcagcctgccagacagtcatttaacgctcccggaataatctgtatccctccctgatatttccatataaatctaaataaatccattttctttctaggtcagaccgttttcagggcggcctggccgtcttttgttggtttttattctatatttttctgtatttttctatattggtgcgtctttttgtctttacacgtcagcagggatgcgtaataatccatttgaaagctgtatttttcctgatttctacggtttttttctaaataatgtgaggttcaaaatgcgtatttttgttgacgacggttccaccaacatcaaactggcatggatggaagacggtgcggtcaaaacgttgatcagtccgaacagcttcaagcccgaatggtccatgacgttgctgacagattcatcagtgccggcttccgccaactatgaaattgacggggaaaagtacagctttgaccagctgagcccggatgccgtgcgcaccacggaaacccgctatcagtacagtgatgtgaatgtcgtcgctatccaccatgccctgatgcagtccggcattgaaccgcagccggttgatgttgtggtgacgctgccgctgggcgagtacctggatgaaaacgatcagcctaatatggccaacatcgagcggaaaaaagcgaacgtgaagcgcgcggtggccgttcaggggcgtgagagctttaccgtgcgtaaagtcagcgtactgccagagtcggtccctgcaggcttcagtgtgctgaaggatctggacgatctggactccctgctgattgtggatatcggtggaaccacgctggatatcgcgcacgttcgcagcaaaatgtccggcttcaccaagacacattgcgatcctaaaaccggcgtatccatcatcaccgaagccgtaaaacatgcactggataccagcgtgagcacccgtaccagttcgtattttgccgaccgtctgatccaggcgcgaaatgacgaaacgtttctttcccgttacctccagaacgctgaccagcgggcccaggtgatgaaggtactgcatgagcgtgaaaaagccctgacgcaccgtgtcaccgattcagttggccgctttgccggcttcacgcacgtcatggtggtcggtggcggtgccagcctggtcgccggcgcagtgaagcaggcaacgggggtgagtgacgatcgtttcttcgtcagtgataacccgcaattcgatctggttctgggtatggtggcaatgaagggctgagtatgactgataaccgtaagtgctcgttttatatctacccggagcgcaatgcggcggaccgggtagccgacggctttcttgagaaactgcctcagaaagagcgtggccgcgcgatgcgcgccatgatgctctgtggtgccgcgttgatgaagcaggacgaacgcctgccgttcctgatcgctgaattcctgacggagagcacctcgatgcaggatattcagcggatcatcagcagtacgctgccgcagcaggatacaggagaaatggcgcgtctggttgaggcgtttctgcaggccaccggcggtggcagcaatacggtgtcgccggcaggcggcgctgcgccggaaaacagtcccgccgtgactcccgaaccggccgcaccggaggataagaatctggaagaaacccgtaagaacgtccagcatctgttcccggatgatgattaagggggcagcgtgaaaaaatatgtgctgttaaccctgaccctcatcgccggcgccgtggcagcaaacccggtaacagcggccggcaatgttcaggcgcaaactgcgactgaagtgaaaaccgggttttcacccggcgggaccgcattaaatctggttctggattcaatcgaccacgcgcagcatgagattctggttgccgcgtacagcctgaccagtcgtcctgttgccggcgcgttgcgtgccgcaaaaaaacggggcgtatcggtggtggtggtggccgatcgcaaagcgaacgacaaccggtacaccgaagtgaactttctggccaataaccgcgtggcggtgcggatcaacgataactacgaggccatgcatgataaattcatggtctttgaccgccgcgccgtcttgaccggatcgtttaacttcagcgccagtgcagacagtcgtaatgcggaaaatgtggtgctgatcagcggcgcgcctgcagtaactgaagcctacgtgaacgagttttcccgtttatggggcgaaggaaaggacgtggccccacggtactgacctacagcagcggcagaaactctgccgcctgatttttaacttgatttatatggctcattcgtcgtattatcaaggctcatttttcggtagcgccccgactggcgaaggagtaaaaccatgcaatcattccccctgttcgtttcccgtgtcgtacctgtgatcaccctgagccattgccttcggcagcggcgctggtccctgcacaacacgtcatgccagattgacctgagatcaggttgtcacctgcgctcacagcctttcggcacgctatccagacgggaaataagcgaccgaccaccggctatgccccgcggttactggttcaggcaatgcagccgggatgtcatgtttacggggcgcatcgatcaggcagatcctatgaaacaggtctattacaacgaaggctggagcggtccgaacaagtatacgtttgaagtttatcagctggagaacgggagttaccgggcgctggcccgcaaatggaatggcaaaatcaacaaggtgcagcaggaaacgcagtacctgtctgacacgcgggaggggctgaagcaccaggactatccccgcacgcgccaggtgaaaatttttctgaattcagacttctgggagaaaggtaatgattaagatggttagcgtcgttccgcagccggaaacagtgaagacgctccgggaaaaaatgggcatgactgaaaccgcattaggggctgtcatggggtacgaactgcgcgcctggcagcgtaaggaagctatcagcgacgatctcagccagtacaacaagaccagtcttcgcccaggcgaatataacatgctgatgctgatcgccggcgtgcatcctgattaccgcctgaacagggcattcagtccggatgatatggtcaaagatcccgccacggccgaagacgttcgccgcctgcgtctggcgctggggctgaagcacgctgaaattgcagcgctgtttggctataaaccggcgtcatggcagaccaaagaaaaggctgctcagcgcggcgtaaagctcaaaaccggggaattcaatttcctgttacttctggccggtgagcatccttcactgcagctggttgagaaggtgaaataaggcccgcggcgaccagagaggaccctttatgatcaccacacgccgaatgttaatgctgtatgcctgccttgtgcccgtagccctgacagggtgtgatctggctgattatttcccgaatgtgccacagcccgtgcccgtgatttcgcccggctatcaggttgatatcgatggccacccggtccgaatatcaggatttgatgactgcccgcgggataaagatgaagcccggcacgcagctgcacaagatgagaaaggctgcattgtgctgtctgcagagcgttccgaggtgaaggtcagactgtatcagccagccggcaccgtcaccgaaacctggcggatcatccgggaaaaagacgctcgccagaacgatattattcgcatgcagcgcccgaacggtgcgcttgtgatgcaaaatttacaggggaaagagtctccttaaaccaggaaaataacaaaattaacttgatttatatggctcattagacgtataataattacatcgaaaggcaatccggtctttcgacggtagcgccccgaccggcgcaggagtcaccttatgactgctctcaattctcagcaacaggctgatgttgcccgctatatccgggaacatggcaataaaaccgttatttcccctgaatggtcacttatttatttctcgctgtgtcagtgtgaattacatcaatcgcatgaagccattatttttcataatggccgacctgcagggctatttcttctgaatgaaacaggcgagctcaccgaattatatctgctggggtcgctttctgacattctcaccgccgctgcggtcaacaaatagctttcagggagtaaatacactatgaccatgcaatcagaacttgtttttactgatcctatgctcaacgtcgttattgctgaagttaagcgttttaattgcccgcttctttttgtcaaagatcatggcgtgtatgtgatggcggcgaaaggtgagaaaaacagtaatgggatgcacaatgtctgttacgccaatggtttcaatccggatacaacggattttgacgagctctgggaccggatgcgtgatgcctgtggcggggatgatttttgcgaatccctggacctcgatccgaggtcgattgagcttctcagccgtaccaaaccctgcctgaaaattatgctttcagagacggaactggaagttattgcagggggacagaaataaaccataaaagcccgttaaatcgggcttttttatatttaactgtatttacatataggacaaattgtcctaatatatatttgtggccgggcatggtgctcaggtcccctaacgagggaaatatatgaaattttcagcagaagaactcaacattatccgccaggcggacgccattattgccggtaaaattaccacaacggattttatcaccggcgccgatgccgcgcgttccttactgcggtttcgtcttgttgcagaaccacgcgaatcattctgcgcgttctttcttaactgccacaacggcgtgattgcgtttgaagaactgtttaagggcactatcagcagcgtcaccgttcatccgcgtatcgtcctgcaacgcgccctgcatcataatgccgccgccgtggtactggcgcataaccatccttccgctaacactgcacccagccaggctgacaagctgatcaccgagcgtctggcgcaggtgctggggatggttgatgtcaaagtcctcgatcacctgattgtgggcggcagcgatatttactcttttgctgaacatggtctgatttgacaactgcggccgcacagtggtgcggccggctctggaggcttgctatgggcaataaatataccgtgccgcttaacgtgggcgcgatccgcgaaagttgtttccgtcccgctgataaatggaccccgccgacgggggatgaactccgctatctgctggaaaatgtgctcaacctcagtcaggaggggctggcgcgacatgttggcgtgaacggcagaacggtgcgccgctgggtgaacggagaaagtgacatcgcgtattccgtctggtgtgtcctttgcatcgatgccggcttaccgccgatctggaaatagccgcgcaaaacaccgacgcgcaaacgtgcttcctgaacgtatcggatatacgaaggaaatattatgaatactgataactattttttcagagtcccggccacacgcggaatacagggcggcatagaacaatacatgctgacggttcctatggtcgtgttacgtcgtattcttgcaatggacaatgatggcgatgttatggatcgcagccagcgagaagcgaacaaaacgcgggccaaaaaaatacggaattatgttgccggcgcaacatcaaaacgagctccctatattttgccgtcaattaccggaaatattgatagtcatgttgaattccttcccagcgaactctcgccggcagtcggtattcttaaaattccgatggacgctgatttaaaattattcgatggccagcaccgtgcgctgggtatttttgaatttgtacgcgattattccaacacggaagatacgatcagcttattgcttaccgttggcctgccacttgagttacgccagcagtttttcgctgatattaataacaacgcgagcaaaccggcagcggcgatcagtatggcatataacaacaatgatccggtcaaccagctggcaatgcatctggcccgaaccgtcaccgggctggcgggcacagtggattttgaacacaacgtggtcccggcaaaaagctcacggcttatcagctttaaagcgctgaacgatgcgacgaaaaaaatgctcaacctgcgggccaacagcattccctcgccgcaacagcgtgacatggcagaaagactctggactgcctgggcgcaggcgatgcgctggaatgatattgcccaggatgatatcgccgcggagtaccgtcaggaggcgctgggactgcatggcatcatgattaatgcgatcggtatggcaacggccaggatgttacggcacagaacccctgaaagcatcgagaacctactggcctgtgctgaaaacggtgacaacggttttcattaccgggagtcgtttgtacctgagtgctgggaagggaagtgtgttgatccggaaacaggcaccatcaaaaccgacaggcggtcgctcgaagcaacggctgaagcgttgcaaaagctgatagatccgttcgcagatgcgctatggttgcgtgattacctgccagctgaggaggcgactgatatggcattactgaaatatgcagccgacatcgagaattataagcagcgtactgccgcgccgatgatcaatatcgtggagaagctgaaggcgctgggtgacggcgaaccgcagttcagagcgtcggtgctggcttccagtgaaggactgagccactatctggccggggctgaaggctgaccaggtgtccgccgcctgcagggcaatacgggcatcgaacccgtattgccctgaaacgcgctgagcagctctctgagcgcttgtttcaaaaacctgtaccccactcgcctctattgatttgacgtgtccagccggccattctggccttatctgatgttattccgacatggcatctgccagttaacctacttctgacatccgtaccgtgtgtcttccctgttcatttctgctcagttgccggcgttttctggcaggcctgtcgtactaaaacacctctgggccccttcggctcctgcccgtcgggcagtcgtcagccacactccgctagccaggcaaagcctggcagctccgccgaaccccctgcggggtttcgggctatcgctagtgtctgatgcctgaacccctgcaccatgccggtaaagtgctgccagctggctgatggggatctgaaagcaaaatttacagggaaaggagtccgggaaaaaacaaaggaaattaatcagataacgcttgattgttatggctcattagacgtataatgatcacatcgaaaggcaatgaggcttttcgcggtagcgccccgactggcgcaggagttacaaattatgagtaaagcactgaccccttcccagcttaaagctgaactggacacgcagaaagatgcgctgctgaaggcgtgcctgtatgccctcaacgaaatccctaaccgccgtttaagtggtccgtattcgtccacgtatgcgctggccgcgcagattagtaaactgcttcagagctgcaaataatcattgatgggctggcattgccagccctgttcaacgctaacggtagcgccccgcatggcgcaggagtctgaaatgtcatctaaaaatactctcccgtcggtcgatctgatgtcctgccagcatggcggtgacgtgatcccgtccattgctattgagcgcattatctcgttgcgggatgaaggtatccgcgtctatctcgacggcatagacaagctgcgaaaagcgcgcgatctgatgcagcaggccggaggctgtcagtacctgtatgactacgacaaagcagtagagagcgccatcaaatggagcgacaccacgccagaccaggccgaaacggctatttcccgcgttatcgacggaaaaatctgggatcggctgatgacggaaaccggcatgtatacgctgatgagcaacaagcaacgtcaggaatgggatcggcaggtggcaggcaaagaaatgccgccgataacgctggataacgttatgagcacgttccggcacctgaacgcaagcaaggcggatactttcacgcaagggctgatagatatctttaaatccctgtcgtgggattataaaaccaataacccctgcatgtttggcaagcgtatcatcatcgcgccgttgctcgatgtctggcgcagtggctgggtgcgcttcagctcagacgggcacaccaaaattgacgatctggcgcgtccgttctacgtgctggacgggcgtaacgtgcctgattaccgtgtttctgacggcgccaaactggatgcttttttcagtgaaaaccagttcaacggtaaggtgttcgagtgtgattacttcagcgtgcgatattacaaaaagggcagcgcgcacattacgtttaaacgcccggatctggttgagaagatcaataatctggtcgccagtcactatcctgggatgttaccccctcgcgtgtaatccagacggcaggccgcatttgcggcctgaaatataaacagggaaaggaagtattccccggcctgagaaaatcatgccgggcatgtatttattgcttgattagtgtggctcattagacgtataatgatcacatcgaaaggcaatgatgcttttcgcggtagcgccccgactggcgcaggagtaataaacatggctattcaatcccttcagttccgtaacggctccgtttctcttggtgatgtttttgtttccagctggggttatgagcagacaaatacctgtttctatcaggttatcgccctgcgcggtaagaaaaccgccgtactgcgccgcatcgctgcgcagcaggtgaaagcagacagtgcgatgtcaggcgagttaaagccggttttgaacgagtttaaaggcgaacccgtgacccgtcgcattcgtgaaaaccatgagcatccctctgtcagcattgacgagtacgaacaggcttataagaccgatcctaacgaaagccatttttacagcacctggggctgaccgcctgctacgccccgcaaggggcgtatagtcaaagacttattcaaccaaaacgtgaatactatgcgaaataacaaggcgctcaggacgcagatacatggcggaaacagtttatcaggcgcggaggcgtttttaccttctccgttttcggcgcagccgtaacccggacacactggaaaaaatgtacgaatccatgcgggatcgtggacaggtgccgccggaggactccgaggcttttgaggctgccgccgaccatcgccgcgcagagctggcatcgggacgcatctgggataagatcccgccgcatgtctggcagtatgtaaaataatcacagcgcccccacgaggggcgtttttttgcttgtatgtatggctcattagacgtataatgtttttactgattcaaatcagtaccttcctaaacagccttacaggtgcattatgacctcttctgaaatcctccataacctccctcagattatcacggccgtggccgccctcattacagccattaccgggctgattaaagtcatgaaggataagggtaaggactgaaccgccgcagggggcgacagccccctgcatcgctaaccggcagcgccccgattggcgcaggagtaaaaaaagtgacaacgctgcatattcgagaaattcccactgatggcgcaccgcctatctgtgcaggccgctcaatctcagatgatgacctttgcgcctcatgccagcatctcgactatcgccctgggcacctcagcgcctgcctgctgtcaggttgcgggcgtgacgactggccttcagaactggatgaaaacggctacagcgttgcctgtagccgctatgagcgtgaaaaattgcttgattaatatggctcattagacgtataataatctcatcgaaaggcatccggtctttcgcggcagcgccccgactggcgcaggagttttacaatgcacggtaacgtaaatgaaatttgcgcaaggcttctcgatagttttgaaccacagcagcggatcagccttctgatctggactgctgaagatgtccatgactgcaccagtgatatgaacctcactgacgatgaggccgaagccgttctggcagaaatcgcggaatgcagttcacacagccgttatggcgtcggaaaagataccgtctggagcctggcaaagcaggtgcgcgaagatgccgcccgcgatcgaaaaatcgaggtcaatgccgaggcgctgcaaaaagtcgtggcgctggcggcgcagtttatccggctggaagagatccagtctggtgaaggagctgcacgccgactttatccgcaggagtctgaggcgctggagtgcatcacgaaggtgattaacggctaattcacgcagtaacgcagcgaactttcacggcagcgccccgactggcgcaggaggaaaaaatatgtcccagaatgaagcctattacgacacgctggccctgattgaaaaggggcaatggtgtttcggcagtatggacgaagaaaacgaagagcgggcaatcaaggccgctaaggcgctggcgctgtttgcgcgcctgaatgagcaggactgcgacggacacccggtttccgaaatcatcgttgattttatcgctaacctgatgcacctgggggaagcgataaattttcgtgtgctcgatgaggaaagcgcggtgatcccgttggtcaggatggcagccgtacactttaacgctgaaacaaagtcatagtaatcatgtgaatcagaggggggcaggcgtgatcctgcccccaaaaatggacaggggaaagagttgtcccgcctgaaaatattcaggcaggcggggatattttgcttgattgatatggctcattagacgtataataaacacatcgaaaggcaatccggtctttcgtcggtagcgccccgactggcgcaggagtacagaaaatgtctactgaactgaatcaacccgccatcactgcacgcattatccccaataaccgccgcatggcctttctgccacgcctgttcggcgcctggtatctcacaggtgaggccggtgtctacaaccacgcacgctctctctgcgccgattatcaaggcggttcctgggagttcgtcgaactgtcttgcggtagcggcttcatgtacccactgagcgcagagcgcttcacggtatcggttagcggtaacttgttcgaaggggaactgtccgcagaagctaccggtatcgtgctgaccctgttcaccctgaaccacatgatctggcacgcacatgaagaaggctatcagcacatctgcgacatgctgatcacccagcaggaaaagctcaaattatatgccgaccagcacgccgaagccggtttgatttaccgcgcgattgactgaaacggcggcccccgcgagggggccaatactgacagggggaaaaatgttaacactaactgatattcgcgccagcaatacggtgctggttacggaatttggcggcgtgcgtgctgtgcatttctgtctgcatgaaaaattaagcggtagcgacaacgatctgtggtttccgctggccaatggggccgacctgttcgaggcgctggaaagcattatgtgcatcaattttgccgctgctaacgttgtctcgctggagttcctgcggcagaacgggaagtgcaaagactaccggatcacctacaacaaggccaaattcaagccattatgttagatttctggtagtattaaatgttcatatttgaacaaatttaattaaaaaaatacgtctgatgagttgataaaatggctcattagacgtataattattacatcgaaaggcaatgaggcttttcgcggtagcgccccgactggcgaaggagttgaaatgaaagaagcagaccaaaaaaaagtgaatgccgtaatggcaattgctgattatctcggtgtaaaaaatcaaatcgaggtgatcgaatactcagccgaatcagtgcaggttgagtggcgaaatcctaaaacaaaacagcttattcatcgtgattacacttttgcaatttcctttgtaaaagactttgaaaaagccctgaaatcgaacgtgaagttttactgataaagagggggagaaatcccccttaatcctcccattttttactgataaacatcaaatatatgaggtgtattatgtggaaacgtggcgaaaacaaagtgattctgatggggcgtgctggcaaagatgcagaagtgcgttacaccccaaacggaacggccattgcgagcctgacgctggctactgaaatcagctacagcgacaacgaagggaaggagcaaaaagaaacggaatggcatgatattgtcatttttggaaagaaagctgaagcagctgggaagtacttcaaaaagggtatgatgctgtattttgtcgggcgcattcgtaataacaaatggcaagggacagacggcaaaatgcgcagcaataaggaaattgtgatcgataataacggcgaaatgcagatgctgccgggagctgggccacgtaacaccagtgctgaaggtctgggaggtactgaaaatcatgatgaaccgccgttcccggatatgaatgattacccgcagtgagtatttgagtgagaacggcccgcaggacgtgggccgccaacccagaaggggggaagtatgaatattagtgaacaatatcgggtggctgtcgtcagtaccggccatgtaaaaaaagaagacgcggagctactggataaggtctgtaacgatgacttgtccggtgaaggtttattctggatcgagaaaaatcgctatgggtggatattgcgtatttctgctactgaacaatggcgtgtggaattaaccagggcggggatcagtgcaagtgctttggataatatagccgcgttaaatggtgccgggttcgactggatttggtttgaccgggatgcagaacaggtgccagaactgaccgtttgggagtggtaaaccggttaaaaatatgcctcgaaaaggggcatttttttcttgcctaaaatggctcattagacgtataataagcatgaagtttaaaagtggatattgattaaaacgctctgtaaataacgtggagagaatattatggcgaccttatacccttatgaggtttatacccttattgtgtcagaattaatcgtaagccattttaatgatgtcgctatggtatatccgggaagcgaagaaattaaaaagaactacgacagtgaattaagtgctgtacaggtagttaatggcatcagagaaaaactgattgaagatggtgttatacagtgcgaacagtgcccactgctatccgagtgggatgagaacaaggttatcaaccagctaattaaagattaccgcctgattctcaatcgtgactatgaactaaaccgtgttcgtcagcagctggccccggagctgatcgcacagatagaaaactggaaagagcagaagtattccgattctgcgaaggaatacgaggtgctctatgaaaccatcaacatcaacggaaaggaggttacaggaaaactaaacgtagttgcagtaagtcgctttgatgctgttttcatcgtaggacagcgtcacgctaacgataactttttggttctggatgtaaaggaggtgatgaatcattgaagtccagaaaaggtgccggattgcgccaacaatccggctgggtaggcaatttaaattgtgaggttcaaaatgccgttgtggattatacactatgctgaagatgtcgcaaacggagtgcaaacaaagtcgcatccgatgaacgcagcgcaatgggatcacttccgtttctggttggcagtgttccggcggattaaagaagccgcgatgtaatctgtaactgtctcaggggcgctgtatgcgcccctttttaatatccgtgcccggactcctttccctgtaaatttatcgggatcaagggcggatcgtggccctgaacgtgcgcaccattgagcgctgcatcgcagcggaaccggaaaattttttccggccaatgagtgcccttgtcaccggaacggtgacaagtatgcacgcactattacacgaacaggcagctcccatgcccggcctgacaggccgggagctatatcataatgtgggatgctatatgcgaaaggtgaaggttaatactgcggacctgaagcatcaggattctgacctgtagagccattttgcgtgcgctgctcttcgtcaactccagtggttttttttcggctgttgctggttcgttttttaccaactcgtccggcagtaatgtccttgatatcacgtaatgtaacatctgaaaaacccgcatcgttgaggaatttcagtatctctttatcttcataccctttcgaatctttcagcttcttaatgtcagacttaagttcatcgagtacgtgctttttctcaagcctttctgacgtcacatcaggcaagtcgtcgagaatgcttttgaattttccgatgtcctcactgctatagaatgctttcttttttgcaggcatatgccctcccaataatgtcaaaatcgaccatgcctaaccggagaaaatcggacaaggctattcaggtgattttacatgatgcccggcatgattcaattgctctgcctgccaggcccttcgctgctggcattcaaacagcgccgatggtagaattcaatgaggtatttaagtacgggacaagatgtgtttttggagtaccgccgacacgcggcggccgtccaaaaacgtcttggtcagggcaagccccgacaccccctaacgaggttagctatctgatgagccgtagcgagagcagaaaaacagacgatcgaatccaggtgagatgttccacagaagtgaaagctaaactcaccgaaaaggcccatgaagcagggcttagtcttagtcaatatcttatcaaatctgggcttggaaagcgcattcaatcgaaaggtaattacaatgcacttgccgctcttgtaaagataacagcccttcaaaaacatctgtttaacgaaggtgccggagttcatagcaaggagtattcagagattctgatcgaggtcaaaaaggccgcacagaaactgcaacaggagatggatggtgatacctaaaatcattgagggccgaagggacaaaaagtccagctttggtcagctgattaagtacatggcagacaagccgtctcaggagttgactgatacagttcagcctactcctgagacggctttagctgttaaatcggatatgttcgaagggttaaacaattacctgacccgtaagcagaaagtgatcagcacaccggtggatgttgagccaggtgtacagcgtgttgtggttggtgatgttacctgccagtacaataccttctcgctcgatggtgcagcacaggaaatgaattcagtttctcagcaaagcacacgctgtaaagatccagttatgcactatgttctttcatggcctgactatgaaaagccgaatgatgatcaggttttcgattcggtgaagtttacactggcatcgatgggcatgtccgatcaccagtatgttgctgccatccaccgggatacagataacctgcatgtgcatgttgctgttaaccgcatcaatccccagacatataaagcagcctcatcgagctttaccaaagacactcttcatcaggcgtgccgcttactggaattgaagaatggttggtcccattcaaacggtgcatatgtggttaatgaccgtcaacagattgtccggaaccctcacagcaagaaagagcggggcaactggcgctcactcgatcgcatcaacaagatggagaataaagagggggttgaaacgctctatcgctatatcgttggtgatgaacaggtaggcggtagtcgtcaaaacttgatccatgtttcagctgggcttcgtgaagccaaaagctgggatgatgtgcataagacatttgcagacatcggtcttcgtgtcgaaaaagcacaggggaaaaagggctacgtcattacccacgaacatcaaaaccagaagacggctgttaaggcgagtctggtatttaacaaggcgcaatataccctgaagtcgatggaagagcgctttggtgaataccagccttcacatattgaaccggccaaagtatcggtatttaaaaccgcatatacccctggtgcttatcgtcgagatgccaataaacgtctgcagcgtaaaattgagcgcgcagaagaaagaatgctgctcaaggggcgctatcgcgcctaccggaacaatttaccggtatattctccggataaggatcgcatcgccgatgaataccggaaaattgcccaacatacccggttggtgaaaaataacgtccggcattctgtttcagatcctcataccagaaagctgatgtacaacctggctgagttcaaacgcctgcaggcggttgccaatctgcggcttagcttgcgtgaagaacgcaacggtttcagagcggctaacccacggctttcttaccgggagtgggtcgaacaggaagcactgaagggtgataaggcagcgctcagccaaatgaggggctttgcctattcctccaggaagaaggagaagtataaacagcagctggttgagcagatcgggttcaaccgtacctttaacgccatcaccagtcatgatcgtgatgatgtggccgtaatggcctcagcacgtcatggtgtcaagccacggcttttgaaagacggtactgttatctttgagcgcgacggtaaaccggtagcggctgatcgtggtcatattgtgctgacagaaagtaatggaatcgacaaagagaaaacggctgatcttgcaattgcattaacgattgctggtaaagccaaatctgtacgtgttgatggtgacggcgagtttaaggaactgtgctgtaatcgtatcgtcgatgcagctgtgaaccacaatcaccctgtcgcgcagggcattaccttcacagatgccgctcagcaggcgtatgctcaaaacgagaaacatcgtttgattcgtgagcaaaataatagtaaaaatgaaatgcaattcagaagtgaaagagacgacaaattcaatccgaagtaaaatggagtaagaaatgaaaatcaaaatgcaaacggtcctcgcggccgtttctgtgttagctcttgccgggtgtgcctctcaaagcgaaaaggaaaaacaaatctctgcacagcagctggtagatcaggaattgcgtgagcaagccattaagataaaactcgcacaggatgagctttaccaggctggagccattaacaggacccgttacaaattccctaccattatcaatgcaaacagtcagtatgttcgtgtgagctggcagggtgatgcttacgaattactggagcaactggctaaacagcgtggccttcaattcaccagtcaggggattaagttaccgcttccactcaatatcgatgttggtggttcgaaggttacgtttgaacagttgctgaccctgatccgacaacaaacgcagtaccgtgccaccatcaaccagaaacccggtgaactgcagctgctttacctcacaccatcgaagaaaggttcattcctcagagggacccgcccatgaaacttaaactgatcgtgcttctgacgttaccgctgcttgctgcgaacgttgcacgcgcggacgactccgatgttaaaggcctggattactacatggacccgccaagccaaagtgacgatgaggccgatgttactggcagtatgctaaacgacgcagcgcacaccattggatttcgcggcggtaaggcagaacgcgctaaagaaattcgcgccgcgctggaaaatcagcgcagcaaccttgattacatgtattcgttccagccgctgatctcttctgaaggctacctgcctccagtcattactgaagctaaagacgttgcacacattaccaatgagcaaatccgaacggccaaccgtgcctatgacattgtggtgccggcgcgctttgtcagtaacccaccgacatggaaaagctatcttctgaccgggctgatggcgcagcggatcgaattgcctgaaccggcggcaatgccgaaggacgggaaacaacgtgatatctggaaaaaagctgtcgcgctgggctggtctgacggtcgccaaaaagctgatgagatattcactgctaatttcaatcgcctcacacgggattacaccggcatgttgcgctacagcacgctactgcagcagggcatgattaaggctccagtcatcacacagcagcagcaaacagttacaggggataaaaaccgcctcatgctgggcgataaaacgaaacgtatgaagcagcaagctgagtttgacatcaacaaacgcagctggaaaccgaccatacgttaaggggcagtagatggaagcattcaattttggtccaacgctgacgccccagacgctggaacgttttctcgtacactgctcaaacaatgaagtgtccgatattctgttgcagggcggagataaaatctgggtagaacgtcatgggcgccagttgcccatgacatcttacccgctcgacaacctggagctggaacgtaccgttgataaggtgttcggtcaggagatcaaagggacgctgaaaagtgctgcggtcgttaataccgctatccagctaagaggtgatgaagcgggcacgattggactcggccgcggcgaaacccgccggttccgcgcgaactttactcaggccgatatttataaaacagaagcggcaatgtctctgacactccgtgttctgaatgaggttattcctcagcttaaacaaatgggtattgagccggacctgtttaattctctgttgccggctgccggcctcggactgatatgcggcgagacgggatcgggcaagtcaaccttgatggccagcatattccagtattgcggtgaaacgtaccaggaccggaaaatcatcacggttgaagatcccatcgagtatcgcctgggaagtcgagactggattgcgccggcgccggcacagtcacagattgggcgcgatattgattccttccacaattttatcactctctccgggctccgtcgttctccgaagattatgggggttggtgagctccttaaccgcctgtcatttgaagctgctgtactggcaggtaaatccggccacttttgtcttggcacactgcacgtcaaaacggtcggagaagccatttcccgatcattgcagaactatccgccggaaatgcgtgaagctgccgcctttgacctgctgagtatcctttcttacatcatcgtgcaacgcttgcttaaaacaaaagacggccggcgcaaggctgtccgtgaatatctcgtcattgataacagcctgcgccgcaaactctatgacgttgattacagcaagtggggccgctacatcgacgacctgttgatcgctgaaaaccgcagtctgattcagaatgcctgggcaatgcaccaggaaggtttgatcgacgaagcggaagtcatcgaggtcgcaggatatatggactatctggcgttaaaagagggcaagtatggacattaacaaatggcgttatgccggccggccactgacggtttttggtgtgcctgttatctcgtttcttgtgtacttcatctggtttccgttcccgtcagtaaaaacctttgttatctgcacctgtgtggttctcttttactttctgctggccatgatgggctacacgttaccggtgctgtaccaggtcattctccgggttatccgcgggaaaaaacttaccgggcggccgtggtggtaccggcgctctcagcgataacacataactacaggggtagacagtatggcgaaacctgaaattgcaactgaaagaacccgtctgagtattccctttgacgatcgccaacgtgcaatacgtgcggccggcaaactggctgatggcagtaatgctctggactacgtgaaggaagaaaaggtgtggtatgcacagccgggggccaacctgagccggctgaaagagtggatttttgaccccaataaggtgatcgaagaaccgcccctggatattcaagccattgaagatgaatttacggcgtggcttgaagagcgcggtgccattatcaaagaccccatagagttcgatggccagaagcattatgtcgatacagtggacggtaaagctggttcaagaaagggggtgtatgccgcctttctggatggccgacctgcagggtggtacagggactataagaacggtggtgaaattcagaaatgggtatctaccggcgcagcccctaaccctgaacaaatggccatgctccgtgctgatgctgcggcaaggcgtgaacaaagagctgcagcgcaagcggataagtttgatcagaccgctaaccggttgatggaagaatataaccggcttccggatgccacgggagatctggcatacctgaaaaacaaacaggtcattgccgctaacggccttaaagctgatgagcggggcaacgttgtgatccctctgtttaatgctgatggcgagttcaggacccttgagcgcatctggtccgacggtagtaaacaccttgaaaaagacggccaggcatgggggagttttttcgttgtgggtggccaactgaaggatggtgacaacctgctttatgccgagggatatgcgacagcagccagtatcagcgaagccatcaatcagcctgtgatcatgacggttaatgccggcaacatggtggaagttgctggccgcctgaaagacacctttccaaacagcactcattacttccttgctgacaatgacatttataaagacgagaacgttgggctagaaaaagcgacagaagctgcagagctgactgccggccatgttctggtccctgcctttagcaacccgaaagaagggcttactgactacaacgatttgcatgtctctgaagggctggagcaagtcagattgcaggtagaaggagccattaatcaaatgaacagggtagacactatgccgactgataacccgaacattactgacgttaatcacagctcgactgacagcgctgccgttgcagctcctgaaaaagccgcgccggtggccagcacccctgcagctgctgaaccggtggaagccgcgccggtggccagcacccctgcagctgctgaagcggtggaagccgcgccggtggccagcgcccctgcagctgctgaagcggtggaagccgcgccggtggccagcgcccctgcagctgctgaaccggtggaaaccgcgccggtggcaagcacccctgaagctgctgaaccggtggaagccgcgccggtggccagcgcccctgcagctgccgaaccggaatacgtcgcaccaacagatcgagacggcgatgatgaagagagtaatagcgccagtgatcttgaggcgtatgccgctatgatggcctttggcggtgaaacaactgacactgctcagcaaaagcctgaagggatagcgcccagcgaacctgtagctaataccgccccggctgaaaatgaaaccgaggagaagaaaaaaacaaacgaagaactggaaaacggcatacgctgggcaaccaacgataatgcagaaacgccaccaaaagaacgtatagacctggaagggttaattgcccggtttgggtacagagcagagaaagactatacggcgtatacgcttgatgggcaggatgcgttttacgattacggttcacatctgcgtatggctacccctgaagccagccagagtgacgagatgatcctggcggccgttcttaccgcggataagcaacatcatcgtggtattgaaatcaccggcagtgacgaatttaaagagcgagtgtttaaccttatcagtgaatacaacattgaggtgaaactgactaacccagaacaaagggttaaattgctcgaactcaaaaaagcaaatgaaacagcaaaagaaacggcaaaagaaaatgttgctgattcaaatgcgaataagcaggaaggggcaaacgaaaaggcaaattcatcagttcagcagaatggaatgcgctgggaagaatcggcctctgtacagccgaagaagcagaatgcatctgatactcaaaaggaagataaagctgcgggtgaatcctgggagaaaggcgttatcgtcgcgcatggacgtgctaagtatgagtggaaagatgatgaatccatgaactactttgtcaccctcaaaaatgaacatggagagaagacactctgggggaaagaccttgagagagcggttaaagatgctggtgtagacaccgaacgtcttgttacagtcagaaagctgggatttgtggatgttgaggtgaatgcacctgtccgtgatgaaagcaacaagatcgttcgttatgaaactatccagaccaaacgtaatgagtgggaagtaaaacctgtatacccacagccaacagcagggaatgaacaggcctataagccttctgaactggttccgtatgatgctgcaaccttccagaagctccgcacaacaattcagcagattaccggtgttgatctcagccgtgaggccgtgcctgaaaaagaactgtattggttcctgccgaatggtaagccagctcctgaaagcatgacaaaacccacaggctataaactccctcaagagtcaaaaaccgcgggtacacctttgctggtcgcaccgcataaaaaaggcgaacgcccggattattaccttgttgagtctcgtaaaggattcatgcaggggatcgcaaaggataaggaatctggcgagtaccatagcgtaattggtaaagtgaacaagcgtattgataaagatggaaactggaaaacgtatatgacactttcagcaatcaataaaaatgcagaaagtggaattgttttgcacggatacggccatgttgatcagggaggtaaaaacctgctttacaaaaacacaattgcaaatgaaaaacagcctcagaatttacaaccggcctcagatgaggtaaaaaacaaaacagtaatgaagcagctttttcatccttctaatgctgcaaaaagaaaagatgatgaaaggagagaacagactcatgcaccagtcgcaaagtcagcgcctcggccataaaacaaaaaccctactcgtagcagtagggttttcctgcatttctgtgagctcatttgcagccacacagacctgtgcagcatacgaactggcaaaagctggtgctgatgctgcctataaacaggaggttgaacggattaacaacgccgctaaaagtgaatccagtacatctgattctctcggacagtgcctggggtcgctatcggtatcactaaaaatccctcaatttccgtctatcagcgacatcattgataaggtgaaggatgaggtatgcaatacggttaaatcgaagataaatgagaagtgggggagcctgactaatacaacgctggacccgtggtcaacgatttcatcccgactgccagatacgtcaggtatcctgcccaacgcatcttctaagaatgtgcaattaaacaatccggcagctgcgagtggaagtggtgaggatgcaaacagtgatgcattcccgttccatttgtaactaccggaagagtcctttcgggattttatcgaatagcttgccggttcgtatttcaacgcggcggtgttccgccgcggttagtaaacgcataaattctgtggcaggtagaacatcacgttttgagtctacatacgtatcaagcgaatcttcattcatgaaacgtctgacagtatgcagccagtcttcagctgatttcatattttttgagctcatcgtcgctcatctcggcaaacgctgcaggtcggcgaccttttcctgtccaggttatggactttccatcaacttcgatccggtacttcacttttccaaaagtggatttacttttcgtggacggaccaaagagattttcgaagatctggagagcttcatcagccgtaatattgtgttcttccatgaaccgctttgtggctgcggctttctcttcagtggccttctgttcttccttaaatttctcgatcattccctgtgcaatgtcggaccacatattaatgagttgttcaaggttttctggcgggattttgcgcaaaacggcggtggcttttcgtcgttcgcacatgataactgcacaggattcgtaatcattatgttcgctcatgtatacctcattgttcattaacgatctgcgtgattttctaacacaaacttttgaaaagagaaactctatgaaaacacaaaatggtgaaaatattatggatcaaactgactcggatcaaaactcagtcgttgatgatgacagcaacgcccttgctccagctctggctgaagagagtaaccgaacggtacaagaaatcgtatcgaaacatggtttaaaagtaattttgattcaaacgctaatcatattagtcgcaatcattatcattggtcttcagggttactggcttcaccagaaaaaaatcaaatatattgcaacgaatgggggcatgatcacagaagttcatccaaccgatgaacccgcttacacggcggaagaggtgatggactttgccaaccgtacaatgatcaaatcgctttcactgaactttgtgaattacgaaaaccaattaaccagcgtccgcggcgattttagcgctgaagggtacgttagtttccgtaaagcgctgtccgatagtgggctgctgaaagatatcagtgaaaaacgtctgaacgtaaaactttccacaactcctggaaccctgatcacggaaggtcttatccgtggcaccaaaacctatgcctggcaataccgcattccggtgactgttcagcttgttgggcaggctcaggactacagaccgcaaccttacgatttgatggttcaggtacgccgcgtcaaagaagacgaagatccacggggtattcaagtagcacaggccattctcaaaccccggaactactgaggtcaatcatgaagattaaactgttaactgtacttatggccgcactgttttcggcaaattctgtatctgcagctgatgcgccgactggtaatggagcgccgcagcaaacggcgccagctgtgcctggtggtgatattgctcaatggcagcaggccagttccgggttgcccgtaaatcagcagtcacaggtgcaacagcctccacagcaaacagcacaggaacaaggtcagcagcaggcgcaaggccaacaaccacaggcacaggcccaacagcggccaacgactgcactgcctcctgcacccatgccgcagattcctccaccacagcagcaggttactcatgttaatccgcagacactggaagagttacctgcacctgttctgcagcagatccagcctattacccctggtcaggtgcggtcggcccgcagcgcaatggaagatatgagcgcggcggccaaaacctcaccattaacgccgatcccacgaatttcaagccagacggtatcactgtcagctggcgcatccattccgcaggtacgggtatttccaaaccaggcaacgacagtgacgttttcagatgcaaccggacacccctgggtattaggtgcccctccatataattcgagtccctgtgcttcaggtggtgaactgtgtgttggctatattccaggatcggcggtattcacaatccagccgaccaatgcctatgccagcgggaacatcacagtgcttctgaaggggctggccacgccggtcattattaacgtgaagggggcagaaccctccgttaaatcaaaaaccgttgatgttgattatcgtctggatctccgtatcccgaagcgcagtcctgacacaccggtttataccgctacgcctgagaaaaaaatctcactgtatgacaagcagctgcagagcgtgctcgatggcatcccgcctgaagatgctcagcggttgaaacttaaaaacgcgccagggagtaccaaagtctggcagatcggtgatgagctctacgtcagaagtaacatgcagctgcaggatgagtttgaaaaaacgctttccgctatcgatggcacacatgtttacgtgctgccggcaacaccagtgctgactttttctgttaatggtcaggcgcgtgacgttgaagttgagctgaattaaggggaacgacgatggctacagataaaggtaaagaaaccagaaaaatgatgctctacgtcggtggtgctgctgcagtggtggcgcttctgggagggtactggttgtttggctcatcagatgagccggcaacgggcgggtcgcaggtgaaagcccaggggctggaagggaaaactggcagggaaaaagctaatgatccccaatacaacaagctgctgaatgagtggaatgttgataattcagataaagcggctctcacaggggatagcttcatctccacgttgtcagccagtaatccggtggccgttgattacggcaccaaagcaccgccaccaaaatattcgtgggaatcgtcagcaaagccgtcgggcgataacagtaaggcgaatgatgctgagcagaaagagcgtgagcgtcaggctaaagcggtggctacgttactgcagaaaatagaccaggccagaacaccagttgtcacgggtgttgctcttgcaacccctctgggagcagaaggaaatgacaagaacggcggtaccttctcagggtggaccgattcggtctatccgcagaacaaaaagccggatgctgaaaaacaacagggggcgaagaataaggccaaagataaaggagcccggcttgtatcggcttacacgcttgtgccggcagtaatggatacggcgatggacagtgacgatcagaacagtattgctattgcccatgttccaactggagcgcaggccggcgccaaattctattctggccaaaaccgtcttgccggcgatggtattcgaattcacttcaccggcatggagctgaacggagtgcagtgtaaggtcgatgcgtatggcgtggcttccgactcgttgcgcgcggccgtttcctcaaatgtaaataaccgctggttcagccgtattattctgccggctgtcgctaacggattaggccgaactggtcagctctatgcggacagcaattcgcagatgattatcactgacggcgggaacgcatatcgttcaactgatacacctgatggcaaagcggtagccggtaccattattggcgggatgggtgagcaagcagggcgtgtcctggccgacgatgcagcccgcctgccgattaagcaggttacggttgaccgtaaccaactcataggcatccagtttgtcgcgccagtgtatgaaagcgactgtggcgagaatttggaaaatgcttctgcagagcaggcgcagcagcaagcaactccgacactatctagtgtgcaacctcagcagatgcctcagcctccggctcctaattaccctcagcaaaactttccaagctaccccggctacgggtatggcaccaataacccgtattaccgttaaggagattgataatgaatctgtttgaagatgacaataaaaaagaagcagctcctgcccaggaaccggtgtctgctaagactgctgaaaagccaaaaccgacagtgacgccagcagcaaaagcaaacaaaaagccgcttaagattagtgactatgcatggattggtggtatcgctgtcatagctatcctgcttttagtctggttgtttgggggctccgatagtggcactgcgcctgcggccgggaatggcacgatccagtcttcagggcaacgtcagaacgtacaggctgcccctgccgggcaggattctggtgacttccgcgagcagatttccgggattttgctgcagcaaaaggaacggctggacgcgatagaatcaacttctaaaaatggaatgatgatcctttccagtcagttacaaagcgctaatgagcaaatcaaaaatctgaacaatcaggttcaggaactgaaaatgaaagccgctggttatgggccttcatttagtaatcagagtggaacaatttcgggggcaacgtcgcaggcattacctttgttgagaggtttttcaattaatgatttatctggcgatcttgcttgggtaaaatacaaaaaccagacgtatgctgtgaaggtcggtagtcaacttggcggagtcaccattacgggtatcgatactgaaaatagaatcgttactacaagtaaaggccttatccgctaaacaggacctaaaaaggcgaaactatggatttagaatcggtattaattcaggttgctaacggtggtcaggaaccgctgacaaggctaatcataggtgtcgctgcagtcgttgggatcattggtgttattggatatctgatgagcctgcggaagcaggccaggcattcaacaaaaggtgtggaagttggcagaatttttgccggtatcatcttctgtgtatgcctcatcacgttgaaggcacagatgaactcaggcggtgccactttaggatttggcgatgttagcttcggacccgtcagttatgcgagcgaatctacgtttggcatgggagccgcagcagtcaacgcagtgctggggattgttagaattctcggcgcatactttttttacaggggtataaaggggttgaaagactcttaccttgagggccatacggagctgtcagcttcgggtacgagaggggcatccattgtgaaaattgtatgtggactacttctgatgttcgatacacaaacgctcgatgcgttgcagagcacattaaacattcactggtaatcaaaaaggatactttatgaaaattgttaagcttatcaaagacaaggcattatctgcatccatcgcagcgtatcagctgcgcggaacggtggcaaaatcgttagtggtattgccgctggcattactgagcaaagaggctgcggctgaaacgactgtctgggagaaaatcaataccttcacggaaggtcttgccagtacccagcctggtctgattaccggcggtaaagtcattggtgttgtgttctttattgtcgggcttgtttctcttttccttaaaaacaaacgcgggaaagatatcagtggcagcttcatcttctggtcaatggttgttggtgcttgcctggtcggcctgaccgcatggatctcttctgttggttcaactgttggcgttgacgcaaccctgtaatgtcccttcggccccgccttgcggggccaatatcggagttccctcatgaaagattttaagctcaccaggaatgaacaacctgccagatgccatgtgtgcgggataaagagcaaagatgttttctccctcgcagaagatggacagaaagagagaatgctttgctctgtatgcctgtcgctcgaacatccggagaaaatgaaactagagactcaaaccggacttctgctgaagagaaatacaaaccgaaaactctacagcctgctgtttcgttcaattgcaaaggcggaaaagtcaacggacgattcttaccgtgaaaaggcgcagaaacttaaacagatattgctcagcacaactgacgtagttctaaacgaaaccggcagtgatgatggatacgatttactgcacctgataaaaaaacaccatcagttacttcccatgaattttgacgatgagtttgtcgcttctgttgcagagtcagacgaattgccggatgtaagtgactggaatgaaatttacaagggtaagagaggttttaaatgatttctttctttgaggatgtgatcgaaggtatctcacgcaatctgacatcaaatgactcgttaaaatactgtgatttgacaacggttgtagggcttacgtcgcaggatcgcgttgagcatcctgaaatcagggctccgtacattctgaccactaagaacaacgattttcttaccgttattgaggttcagggcgctaaatcatcgtttgacgagaagtcgtttaatgacttcatcatgcacctatcaaactccttgtcgaattcctttattaacagcggccataagttgtctgtggtgttcgaacgcgactttaccaagaaccaggaagagctgaataatgtttataaaccgcaggtagcagcaatagagcgtctgcagatagacattaaagacctgatcgccgatgacgcaaaaaaaatcgccgctcattacagccgtgaacgcgcttacatcgtgctgtatacctcgaaggggatgattgcgaaggatgaacttaaaaacgaacaggcaaaagctgccggcgtactgaaggatatcccgcagacgcgcttcagtcagaacccaatcgaatttcagcttgaaggtttgaagatcacgcatgacgcgatgctgtggcgcctgattggtatgcttcagggggatgatgaatcaggaatggggcttctggttgatgtcctcgatgtcaaacacgctggcgcaatgatgcgtcagatgctgttccggaattcaacatcagaaaactggtaccctcgcacgccgtttgaccaaaatctgcgaatttatggcgccccacggaaagacaatatcgactccgtgctgccgccgcggctgaatatccagatcatggaaggggaactggaagaagatggtggcctgatttactgtgatggtaagtattacggctctgttgcccttgagttaccaccacttcaccctgtcaaatttaagcagttattcaacgcgattaaccggaaaatcccgtaccggataaaatatgactttataggcgggggcaaaaaagcactgttctggccatcagtaatcatatctttccttcgtttcattccctcactcggtaatatcgcgcgtgatatggattatgtccgggcccagagcgaaaccgatccaacggctatcatggccatcaactttgccacctggggcgacactaaggatgaagccaaacgcaacctggcggctttaactaaggcgattgaaggctggggtgtatcaggtgtgtccaagacatatggcaatccaggtagcgcactgattgcatccgttccggggctgacgacggccacacctggctcattgcattatccgccgttgagtgaaggcttgcggatgttaccttttgaacgtccggccagtccctggcatggtaaaggcaatatcaacttcatcacgctcgatggcaagctgttcccctatcaaatcgccagtcccctgcaggagaaatttaccgatgtcatcaccggtgttcctggttccggtaaaagcgtgctggccaaccgccttaacctgagttcgatttaccgggctgataaaaaactgccttatctgaccatcattgataagggctacagtgcaaaaggtatcgccgatctgattagcgctgagctgccgtcagagaaacgtcaccagattgccagtatcaccctgaacaatgacgaatcgcactgtattaacatctgcgatacgcagttaggttcaaggtatctgacgcagtttgaggaagtattcttaaagcagatgcttaacgcattctgtattgatccggcaattggccagccgccagatgcaatgagcgttggccaaatcgtcagtaaggttgtatcaaacgtatacaaagcaaaagcgcattcaagtgctaacttgtttaagtataacgacccggtgattaatgaagctctgaaagaatcaggtctggataaagagcttggtgaagactggtttgaaagagctacatggtgggaagtcgttgataagctgtttgccaaaaaatatcttcatgctgcgacggttgctcagcgttatgctgtgccaaccattcacgattttattgctgaactacaaactgaatcatttaaaaaccaatacgctgacgtaaaagttaatgggagtgagcctgtagtaggcttcgttgcacgttgcttccaggctgctgcagctgagtatgccattttctcagggataacggtttatgatttcagcccggagactcgcattgcgatcctggacatgcaaaacgtcctcggtgacagaacgacacctgcaggtaagctcaagtccggcattatgtatctttttgcgcgccagatggctgtgcgtaactactacctgcctcagtccgcagaaaccttcattcctgctttaccggagcagtacagggcatatcaccaggcgcgcatcagggagctttcagaagaggttaaacataccttttatgatgagtgtcataactttgccggcatcgactttattcaaaatgcgctgaacaccgctgaccttgaagatcgtaaattcaacgtccgtactgccttctcgtctcagtatctgagccatatgccttcgagtgtgcttaaaaccatgaacagcctgtttatgatgcgcctgacggaaggggatgaagagtatctgaagcaattagagatcaacatcccggccgatatcctgcgtcgtttcagacagcttccgcaaggggtttatccggacggaagtggtactgcattcctcggtatttttaaaacaaaacgtggtcttatttgccacattctgaaaaacacattaggaccaaaactgctttgggctttgaactcatctgcaaaagatagagcgctcagagatgtgctttatgaagagctgggaacgaaaaaagcaagggaagaattagcaaatagattccctatgggaagtgcatcaagtatcattgatgaaatggtggtcaatcagggtggtgagcgtgattctgaagaagaatcacaaactatggcgatgaagctcgctaaagaaatcatttctgatgtgagaaggggattcagatgataaaaatcggaaaagcaaaacggtcagtgatggccgtttcaattctggccttcagcgtacagtcagcatttgcctatacggtcaacgttaacagcagcattcccatctcaacgcaggttatgccggcgttgagcacagctaatggcctgttaggtaatatccaggccacgctgattaacattggtacggcaatcagccagcagggcgatcgccaggcagcattaatgcagcaggtggctgaaactaaccgacagttcgaagtcgagcaaaaacgtaatgaccggctgctgcaggcacaggataagtaccgtgttccggatgatatctgtgcgcagtctattggcggtggcgccgcgggggtatcctctgcggccgctggcggtcagcgaacgctgggaagcgccagttctgcgaaagatgttaaggctatttttgaagtgccggcacgcgcaaccgacgtggatgcatcttcaacggccgaagttcacagccagtactgcgacagttcggattatgctgcatttggtgggacttcattctgcccaagtgtatctgacatgccgcgggcagatacgtcgctgagctcgttgctttatggcgccggcaaagaaggcaaggcacctgatctgacgtttacagatgagcagacagatgcggcgctgcgctatatgaaaaataccgcgttgcgcagtgctggtcgtcagctgagtcgtggggaagttaaaggcgccagcgggcgaatttacctgggcatgattcagcaataccaggcgctcaccgacgcggccgcacaacctcagatggaaatgatcgccaactccaaaccgaacccggccactgatgccgcgcttgcagatgcgcgcaaagtgccggaagtcgaatcctatttccaggcgacggccagtaacagggcgaaacagcttaaccgtatgtctgccagagagtttgaggaattcgaagtaggtcgccgttacagcaaccctgcttaccaggcgacgctgaccaacaagaacaccgaagatttgatgcgtgagcaaatcaatatcgccaatctgaacaactggttagtgctgaaaaccaaacagcagctggagaaacaaaatgtgctgcttggccagatcctggcgaacgacgcatatcaaacatacaaaccgatgctggcagcacagctggagagtgtgaacgcgggagtatccagaaaatgaccgacgaaaataaaacaggggataaagacacagccaaatcagggaaattaaaaaaaggccttgatgttgtaacaggtgtcaatgacctcccggaagggaaggctaaacgaacgatatattatattactggcatcagcgatatatattttattatcgcgtcagtaaagcaaacattcagcctgctctttcagcgggcttcattcgttaagaagcaaataaaaaaccttgatggtccacctgttgattctgatgcaaatcagccgttcgccgaagttatgaaacggagcaatagacctgtatcagaattgttggataaggccagtttatacaagaaatactggctttgttgtttttttgcattagtcctcattctgctttttttaacgtctggttatgccagattgcttctaaatggctcaccgaacatgagcttgttaagagcaactctcacctgtggcgtcctgtttgctgctgggatctttactttcattaaggctctcacatgtgagttcatggggtggcagttacgaaaccaggctcactctgatgcagagcaaggtacgttacgttatttccttaatgatgggggagtgcgtaatacatttaatttttcgcaggctggtcaagagcgggggccacatgagtaaattaaaaaggttgcttctatctcttttcgttgtggcgtcgccttcatattcagcggatttggggattgatggcataactgaagcggctaaacgttcaggtgacctatcgcgtcaaatgttacataccgtttttggtgaggtagttaataatccgtttagcccttccggcgatggcttacttaataacgttttcttcactgtcaacgaagttatcgcgattcttgcccttgtctatatggggataatcagcattaagaaacttcaccaggcaggtcagttgggaagtttcattgagggtgatggcaataacgcattcaaaattgttaaaacgacttttgggtggttgttgcttgtacctaccgttgtcggttggtctgtggcgcagctgctttttctatggtgcggttcaatcattggcgtaggatctgccaatgttattgcagataaaacggcttcggaattagcgagtggtaaagctgtctatatagcccctgttatgccagaaatggcgtctgttgcaaaagggatgtttgaaacaaacctttgtgctttgggtgtaaatcaggggattgctcaaatggaggcttctggccaacattatgagaataacgccagaatgcaataccaaacaggtgacagcacatattctatatctgtaaacaacggctcagctgtatgtgggaccgtttcgctgccacaacgtccagcaggatggacttctatcttcgctagcggttatgcggattccgtctacagcgttcagcaacaagcaacggacacgttgtggggaaaaatgaagactgctgcagagcagtttaatgcagcatatatggcaaaaatgcgttccggggacggtgagctgccagatgttgaaagcgcgatccagactgctgcgcgtgattaccagcttcaggttcaacatgctgctaattcagcagccggtgacgacgaaattgttcaaaaaatggagtcggatatcaaagagaaagggtggctatatcttggtgtgtactatcacacattagctactgcgaataccgagatgaaagatgttgctaacctgaagccggtagtttcaggaatgagtaacgatggcgatatcggctctatcgattactttaaaggcttgttccaggcttaccattctcaactgaaaaatagtaattatacgccgcctcttggcaccgaatctaacgcacttgcaaggaacattgatcaaaaaagccttgaaaatgccgccacaactggagacggaacgtcccttattacaaaaattttcgacttcaacgttacaaactggcttgcaacgagtaactacggcactggtgatggttacagtgacgtgaccaaccctttactcaaaatgaaagctatcggcgattacacgttaggtgtagctgaggttggactcgccagttggaccgctatcaatgttgcagtgaaggtttcagaagggaagagtctacccggttttgctgcgggaatgatgaacaaacttacagggattcgagatgctattgctggcgtattagaatcggtttcgttcttggtttacccgctgttttatagcctctttggtatcggtcttgcactatctatctggctgccgtttataccgatgatttattggtttgttgctatggcagattggctggttacgcttatgacaggtgtattggcgtcttccctgtgggcagctacccatatcaacattggccagagtaatgatgagcggagtacatatggttatgtgttccttatcgatgtaatgattcggccgatgctcatggtaatggggtttatttttgcttctctggccatcgtagctttgggaacagcgttaaacatgatgtttaaattcgctatggaacaggtccagtctgactctttcactggcctgctgtcatcgatcggatttttgatgttatatgcaaggctttgcacaggtatggtggctcgcgtatttgcacttcctgcgaggatgccgaactatgttataaactggattggacagaagatgaacgattcagtgttgggtgatatgcagaaccatgttcatgacatctttgctgcgttcggtcgtggagccaaaaatatgaaccgtaacaaccctaagcaatttaatcctgacgcaggtgtagataagagcaaggacggcatcaaaggagcttaaatatgaatgcgttatcaaacactgactttaagaaaattagtaacaatgcgcgtccgctccaaatatcaataaagcgtacagggatatggaaatcatctgcagaacgcataaaaatagggatgaaaactatcagcatctgaagagtaaactgacagacgctggatggttcataaacagcaattattgagtttaacccgccgaatatggcgggttttttgtgtatactcaagtggttatagtcgtatgttgtgggaggaagtagtgaacagagttactgagcgccaggaacagaagagtaacccggagttccagagagagtaccggaaccgtatccgggaaacacacgaccagttaaacctttatctccccaaagatgcaaaaagaatgttgatagagatcacaaaagttaaaaaactcactcaggctgaagtcattgaatttcttgtcaaaagttattacgacggattgaaatttgacgattctgaagagtgatcttactatgtaaaagcgggttcgcaggtattgccccgcttagaaacgcatgatatgttaagcggcgtttacataaccttacggggttattacagaatttagcacgggaagagcggcccggagatgtaccgcttggggtgtggcaggagccacaaaaagtaaaaccccctgattcatcctctcactttggccggtcagagatgaaatcagggggcgcaaactgccttgctggcagaaaggattatagcaaatgcgtaccctattgcaatacagcccggccaataagtgcaggggctggtgaatcaaaagaatccatacctacagctttctgacattgagtcagtagaaggtctttcgccggagttcatctcctggcttgaaagccagtcccctaaagaatcgcccctgcagctgcttctcgatgaaggcaccaggcccgtgcgcaaaacccgccgtcgtcgcggtacgcactcaacggcgtgtctctgcccggagcccagctggtaccgaccggacaatttcaaaaagctccctggccagcttggccatgcctacaaccgccttgttcgccgcgatcgcaaaacgggcgcgttaagtctgcgtatgcggatcagccgccacccgtacttcgtgcagctgcgcgagcaggctggccggcagcgcgacttccgcccggagcgggagaaattactcgatgcgattgtgccgctcctggtcagcaccgttgaccgggctacccatattgacactatcaacctcagtaaaatggcgtggcagctcagcgaaaaggacagcgaagggaacgtgatccggaaagtgacggtaccgcgggtttgccggctgctgcagcacatgatggaattcgggttactggcgctgcctgaaggcgtcacatgggacccgtttaaccgcaagtggttccccaagcatgtggtgctgacggaacgcctctggaagatgatcggtgttgatctcgataagctgtatgcggagcaggccgaacaggtggccgctgaagctgcaggctggataacccgtgatgaacatggccaggcggaagaaatctctgttaaagccgcgcgccggcgctggtatgaaaaaatgatgcacgccacgctggttcgccggcgtgaagccgccctgaagggtaagcgcgagaagaagctgaaagcgctggcagaacagccctatgacgaccgggcttacgccatgtccgttcatctgatacggacgctgccgaaagacgaactgcacgcgctgtcgcctgagcagtttacccagcgagtacgcagccacctgtatcagctcgatctggggctggagagggaatccggtccccctgattatcactgattttctccccctgtaattctcttaccgccgcctggcggttttcgtgctggccagccggcgcgacctgctcaaatttcctccagttttaacgtgtggccaacgtgattttttgacgttctggcgccgccggtggataaccggcagcgaaatcaccactatccacagttatccgcaaaggaggaaataaagtcacctgcaatcgtggtcaaaacaagctgcaatacactcttaacttctttaaaaagatctttattcaataaagagatcactgttattcaggctcaattttctgtggattatgggaatatgcctggcctttcagcgccgctacgcggcacgacggccagaatggtcgctatgctcccaggttagcacccggcaaatgccgggcagggtggccagcagaaacctccagtccggattcttctttgccccaccgtgcttttccccgccgagccaacggctcggcagaaaaaagcatgatggatgttgtaactatgaacacccccgtaagacggcaggggtgcgacacgcccccctgacgtcaacgctactacccacgcgcccggtgtgccgctaacgtcacaccgcccgctcgcagtagccagggggtaatgtggccacagagccatctgcagggcagtctggcaggataatcgtctgcactgacgtgcaatgcctttcaggcaatcagcaccggtgccagctgcccgttttaaaattaacagggaaaagaggatcgatttagccgcggttatccacgtcagccgctctcaggcgcgttaaacaggggagcctctacgccgacctcattcaacaggtccagggcgacacggataactgtattaggctgcaactttatcatgcttgacactttatcactgataaacataatatgtccaccaacttatcagtgataaaggatccgtgagttctatcggaccagcggaggctggtccggaggccagcggaggctggtccggaggccagacgtgaaacccaacagacccctgatcgtaatactgtgcactgtcgcgctcgacgctgtcggcatcggcctgattatgccggtgctgccgggcctcctgcgagatctggttcactcgaacgacgtcaccgcccactatggcattttgctggcgctgtatgcgttgatgcaatttgcctgcgcgcctgtgctgggtgcgctatcggatcgtttcggccggcggccggtcttgctcgtctcgctgtccggcgccgctatcgactacgccatcatggcgacggcgcctttcctttgggttctctatatcgggcgcatcgtggccggcatcaccggggcgactggtgcggtagccggcgcctatattgccgatatcacagatggggatgagcgcgcgcggtacttcggcttcatgagcgcctgtttcgggttcgggatggtcgcgggacctgtgctcggtgggctgatgagcagtttctccccccatgctccgttcttcgccgcagcagccttgaatggcctcaatttcctgatgggcattttccttttgccggagtcgcacaaaggcgaacgtcgaccattacgccgggaggctctcaacccgctcgcttcgttccggtgggtccggggcatgaccgtcatcgccgccctgatggctgtcttcttcatcatgcaactcgtcggacaggtgccggccacgctttgggtcatcttcggcgaggatcgctttcattgggacacgagcttgatcggcatttcgcttgccgcatttggtattctacattcactcgcccaggcaatgatcaccggccctgtaaccaccaggctcggcgaaaggcgggcactcatgctcggaatgattgccgacggcgcaggctacatcctgcttgccttggcgacaaggggatggatggcgttcccgattatggtcctgcttgcttcgggtggcatcggaatgccggcgctgcaagcagtgttgtccaggcaggtagatgaggaacgtcaggggcagcttcaaggatctcttgcggcgctcaccagcctgacctcgatcgtcgggcccctcctcttcacggcgatctatgcggcctctataacaacgtggaacgggtgggcatggattgcaggtgccgccctctacttgctctgcctgccggcgctgcgtcgcgggttttggagcggcgtagggcaacgagccgatcgctgatcgtgggaacgataggcctatgccatgcgggtcaaggcgacttccggctactttgttcaggtgcggagccaggaacgagaagtattttccacactccaggcattgaattttttccgttaattcttctttatcagccatcgtttggcctcctgattaacaacgccggcaaacgccggcgttcagggttacatctcgatcatcggaaacagatcgtcttcctggtggtaaagcacgtcgtctgtgtccgtcccatcaagccagtcagcgaacagttcaaacaggcacaccgacatttcctccggtgaaattgcgccacactgcagctcgtagaagacctcaattttttccagcaggctgttggccgctttgtcgtagccgaacagcttgatcggcgggaagtgcattgcatccgggttcgtttccgccagttcatcatccttacgttccggatagatgctgtacagcgtcgccggcgatggcggtaatacctcgataaagcggtttatccgcatcggcagttcgctggaaaacttctcattgtccggaatgtaaaacgctgcgctgcgtaccagggcatcctggaaagaaattaccccctgaccttcggtctgcttacgcagctcacgaatgttcacgcggtccttttcacggatttggtacagatccccttcccggaccgttttggaaaacgtgccgtgttcggcttcctgcccggagagctctgaataataatcctggccgacggtttcgcgcagtatttcaaaggttttccggtcttcaatcgactcaaaatatttcacgcgggtgttggcgatcagcgaatccacctcgccgttcgcgccgcgggcgagcgacgggtgatcctgggaggaaaatacgcccattttttccagtgaacgcatctgcgcactcagcgtatccataccgctggtgaaatactggcccagctcgtcataaatcagcccgtatggcgcctgattattcagcgcgtgagtgagcaggacttcctcttttttcccttccagctggtagcccaggtccttactgatggtcattcgctgcagcgtcacatataaacggccaagcgtggccgcttcaccgcgtgacagctccagcgacgggatcatgacgatgagaatgcggtcattgtggaaaatatcgtccatccggatatcgccgctgtcttccggaaaaacatggccataagtttcattgaacagggagagcgtcttcaggaactgacggctcatgtagttatgctgttcgaaggcgcgggcttcccacgtttccgggtattccgcgttggccagcaggaagcctggtgtattttccagataactttccagcgcggcgtaaccctcgctgtgccagccgtttttctttgcctcaacgtacagcgcggccattgccggcagcgacatgtttttctgaatagtgcgctgtgacaaaagcagttcgccgcgggcgcgcttataacacagggcattgatcagcgccgacatcatggcttttgccgtatcctgccactgtgccgaatctccgcccacctgggggagcatggattccatcaactggatgatgaaggtcggcggggcaacgctgaagaggttgactgagttggattgcgcggggatgctttcctgcttcaccaggttcacgaacctgtcaatactgccggtcagcaggttgaggacgtaataatcatcctcacggccgaaacggcgcgccagcgaccaggtggcgaaggccagcttgttatctgctttaccgtctgagaggcagtagccacggcaccacagcaggaagttaacgataaacccatagaaggtttcggttttacccgacccggtggtaccaaaaaataccatgtggcgcagcaggtccgtcatgttcagccacagctcgcggccgaacaggcgtccccgctcatacccgacatataaaattcccttcgctttctttcgctcccggaccttatgcgggatgcgaatgggccccagacggtattccgcctgggtttccgtggtcagggtgtcgtcgaacatttcgcagtctttcggcattcgcagcggtggccggaatttacgatccgtgaatgccaccatcagcggcggaaggatgaggatgctcagcagcatggtaaaggggagaaacagacccagcacggtcagtatggccagcaggccaaactgtacgttcggagtgaggaaccactccatgaacgaaccatctgggcggatgacttttttactgtctacagattcctgttgcatagcggctccgggttattctcctattgcctgctgcaggcgggcatcgtctttgacgacggaaaccgggatcacatcacccgaatcggcaacgatggacggggtgccgataaacccgaatttcgcaaatgccatattgttcgcgtcaaccaggcgggcgccggtggtatccggggctgttttcggatcgtcaattttcgggttcatggtggtgtatcccatgttaatcgtgtcttcccagcttttcagtcgggtatctttcccggtatcgagtacaggagcggcgcggaccatcgagcgcaggtcgccataaacggacacggggaaaatctcgacgttatagcgtttgctcagttcctgcagccggggttccagcgcgcggcagtgcgggcacatcggatcggaaaacacatacagggtgtgtttatgcccggttgaaagcgagatcgtaaagcgtccgctggtgacggcatctttcagtgacagcaagtcgttatcagccagttttttaggggcttcaggtgctgcctgaacctgcagctgctccggcgcagtgacaggctgctgcagacccggcgcgggagcatacgagtttgcctgcagtggctgtgccggctgatgcatattcatcaacgcgccgttgatggagacgataaagcccagcgcggcgactggccagaagataaagttagacagccggcgcacggcattgagtccgcgctgctgtttcagctttttacggatattgttcagcgcctcgatggcctgatattcgttttcaaagcggaacagctgcaccgcttcaccagaaaacgtattgtgcgtcaggatgtggccgctgccggcatcatggccaagtccaaaatgacaggctgacagggggaaggagtgcagggcttgttcctggtagcttccgtcagcggaaagataaagtgtgttgcgatcggtttttacggtgagtttcatacttcctcgcttagagttccaggtgatcgggacgggttgccggctgaggtttagccggcgtcggttcatcgtctgtcagtcgcagactgttgttcggggtgtcgtgcggagctgcaggcggcgtagccggctcgtcttccgcttcagcgtcagattcaaatgcaaatggcggtataacgtcttcctgccagtgagcgaccggaatggcgggatcgcggcggtcaatatggcggtaatggagcagatccagctcaaggcccagcacggccgcccgcacggttggcgggaatttcgccgccttttcttcatcgagcgtcagccggtaactttcgcactgactccaggcaataacgccacctccttcaatgaagaacttcccgcggccggcgcttgaaagtgcataccataacgtgcggtccacgcctttcagccaccggaaatttgcggatggcagctgcaggtcatcatcgaacagcgcgtagagtagcgaacgggagctgcggcgcccctttgcaaattctctgaacgcttcgcactggctggcgacggtccagagcccggcggccagcgaataatcagggaagccgtctttagtccgcaggcaggaacggttgagatcgtcgagaatggatttagccgttggccggtcgcccataaaccgcagatgtacaaacgaggctgcgagtgctttttcccagggtgccagggaaagggccgccccttcgggagccaggtttacctgtgacaggaataccttatgtgcggcttttttacggaggcggcgtttagcggtatcaatcaggttatgttcctgggcaaactcaacgggtgactgcgcgctgcgtgcttcttccggatcttcattcagcagcagctgatcgaggtggccaaaccgggcgaggatatgggctatccccggatagttttttaccatcacccagggcaggttccggatggtcagtttacgggtcagacggtagtaaggattgcgcgagataacccagacgaacaacccgatgattaaaatgaacaggggaaagagcacgctggccgtgtcgttcatcacgctgataaagtcccacagcgaaacctggtgattataccgcgccgccgacagcagcatgtcccggcgctcggtcgcattacccggtgtaatggcgtccggcagtacaccgtagatccagtaaagcaggcgacaggctccgtaaatcacataatcacgaaagccaaaccacatcactgtgatcatgatgagaagggccccgataatcatcaggccatcatcccctccccctttccgttgtggctggttttgcatcagaacctcagagaattttggtaaaccttcccgatataccaggccttacgcgccggcgtattggagtgatagcggccgacaccctgccagaaatcccccccttcgagaatgatgttgtatttgaggatgtaggcactggccattgcagaggcgcaaaagtcgtgctgcagctgctccgctctcacaccaaaatcagccgccagcttattcgcccagatggtgtttatctggaatgcgccgtaatcgtaagtaccgtttttgtttttatttttcattcccggtttcccgccttcagtcagccacaaggcgaggaatccacggagaggaatgtgataaatctgtgctgcttcaaccatgcaggttggcagcgggattgagggcggggcatcaaccataacaatctcctaatgatttctatttcgagtaaaaaatataccatcaaaaggagatttttttaaatcattttggtgcgcataattgagagggtgtcagaggaattttatttgccttcgacagagacaatgttgccctcattcgcattttcccatgtcgggatttcgccactcagcgtgcagcgaagcgtctcttttccgacggtaagccgtgcgccactgctgtagtaatgactttcatacaggcaggcggttttcttttgttccggggtcagtgtaagactggcattctgatcctttgccagcagttgtacaagatgttggtttgtctgtacctgttcacccagaatgctgctgagcaacgaatcggttttcggcgcggatacactctgagcaaaggcactgcaactgattacggatgcggtcagaatgagagcaatcttcgtgttggatttcatttcactttctccattaaaaaaatcggtaatcattttctggatcatttaaattaggtctgaaaaccggcctgagtgtcaagccgattctgtttaaaaattaac

SEQ ID NO: 3 - DNA, 55473 bp, synthetic construct
  misc_feature 1..55473: note = pJIMK_Core_M sequences
  source 1..55473: mol_type = other DNA
aggggccaccatcgacaaaataaacagggaaggaaggtcaagggtcttcttcgccggggaaggtattgatccggaatatgccatgcgccgtgaaatgttgtctccggcctacaccacgcagtggaaggatattcccgcggcgtttatgcgataacagcgatgtgcaggccagtctgtcagccctgcagccgttcctgctcctgcagcaatgccagatggtatgcggcaatggcgttcaccaccccggccgtcatccgtgcatcggcgattgccgagtgtgcctccccttcccaggccaatgctgcctgactggccgcgctggccagcgaaatggtgccatagcgattggtggccccaaaataccctgctgccagctccatcgcgcaatatactgtcagttctcccagccagtcggccggatcgccatgtgcggcggccgtctgtttcagaatgcgggtatcaaagggggcgttaaagataatcactggccggtccccgatggcgtgtcgcagctgcagcgccacatctggccaggatggtgcgccgctgagtgccggctcatcgataccatgcacggcggcggcctgcgcctcgatggcgaccgtcggcttcaggcgggtttcaaatatcacccggccggccgcatcggtcaggccgatttccagcgcttctgcggtattacccagccccgtcgtttcggtatccagaaagagcggcgccagactcagccagtcgtgtgcccgccgtgccatccggccactggtgctgttaagccgggacaggacggaaagacgcaggccggcctgctgctgcttctcggtcggcggccgtttttcccgcatcggaacacagtctgcaatgcggtatacggcgtacttgccgccataggcgcttttgtaccatttcaccggctcagcaccgggagccggcttcattctgaattcatcgcgcaggcgccctttcgtgaagcaccgatcgccggctatcgcctctgcgcgtcgtgtggccagctcatcattcttcatggttattccatctccgtcaggcgtttgtgataaacctctctggcgatctccaggcctgcctctgtcagcccaaacgcgagcgtcagtgactgattatcccggtatttgctgagcaggccgtgacggttcagcgtatggcaggagcgacgaaaattggtatcggcaacgggctggaggcgtcccttattgaggatgctgaaaagctgcatcgccgggaacgggcccgccagctgacgttctgccagaccatacagaacaaacaatacgtctttttgcacaccggagagccgcatcgagggggctttactttgcatcttattcatgtgtcacgtcattaaggaattaatgacaaagtaacctaattgatgtgagacatcaataatattgtgttcataacgggaaatcaggagtccgaaaaagaaaatgccgctcttgacggcggcttgttttccctggtgtcgggagaattatggtttctggtctgggtcaatgcgcagggcgctgtctgtgagcaaatccagtgctgccgtcagatgctgactccgcccgccgtgataatccggccacggaagaaagggttttgacgtatcagacatgaacatgataccggtcgggcagcgccactccgtgggggttcccttgttgtaatgaaccagcagtactgaacggtcacactgcacgtcattactgtcatcgttgttcagtacaaaagtggccacgatgccggcatcgggcctggacacaaaaagcaggctgttggcaaaaataacggcaatcaccactgaggcaatggccgcaccggtataccagaaacggccgtttttccaggcccgatagccatacagaatgcacagcagaatggcggcagcgatacctgcccactgcgcaccgactgacaggcgggagagtgcatccatcgataacggccggctcagcgcataaaatgtgacaaggaaaggtgaagccagcgccagcgtcagcagaacgaaaacgaggggtaagaagcggttgccggtatagctcccattgccaagcgccgtcatcgtgcggatcatctcactgaacgccatgatcatcataaagcagcataaagccatgtataacattaaaacatccccaaactgtccgcgtgatatggccggattttcggcctgttccacatagtcccgaccccttatcggggtgcatagccctcttcagccagtgctttcatgatctggttgattccatccatcatcagatatgcctcaatatctgacgtcgccaggccgcggcagtcactgaatcccagccgcttaaccagttgcgccagagcctgcgcttctctgtgtgtcagctgtatattcacttcaacgggtttttcgtggcgaatttcatctttcatggtcagagtccttttttgatgtttcgtgaaatgtaccaggcttcttttgcctgagctacagcccagatccagacgagagttgacacaatgcacagccccgtcatgttccagaaatgacctgtcgtatattcatggccatgaagcaggaggccggcgccggccgccaggctcatgaccaggaaaaggacggctgcgccggcgtgataaatgatgctgaacatgtaaaacccccttctggcgacggtagcgccagtctctgtctgtattaaatggtcgccgcctgccgggcgtctgcctgcatcatggcgtcaatcagggcgccggcgcaggcaatattcaggcacgtcatgttcgatttcagggtcgccatcatgtccgcgcggcttcttttcagaaacggctgcagcggaccacgatagccctgtgaggtcacgataatcagctgagtggccgcatcatgcagtgccgacacgccggccagaatatcctgctcagccgcgctgatatacacgggtgaaccggcgatcacaaagcgatcgtccgggcagcgttgcatcagctcagccagggaggcgcagcggcccgccagcgggggccgtgccgtcagtgagccccagacggtcgccggcgcgaagggcatggcgtgaaacgttgcctcatacgcgaccgccggatcggtatggtggcgcagccccagaccggccgacatggcccacagctcgatgcccggccaccgcgccgcgataccgcgggcacgcagccagtgtgcgccccggtaaagctctcccgtttccagcaccctgccaggacggcgggcagcctgggcgatcagcctgtaccagccgtcaaaggcgtcatcaagggaagcgtcgcccggaaaaacggtatcctccacgctgcccgccttgcgccgggtacaggtggtgatgaggtgaagacgtttgttcattcagtgcctcccggctctggtgcgtctgtgcctgccgcgcgcggttcgccgggccgccgggcacgaatatcgggcagaccccggcaggcatcgttccagtgcgtgccccgatcggggttattggccagcttactgatccaggtttctgacacattccagcggcgggccagctccttctgcttccagcctttcgccctgagcagcgcccgccactcctgcggggaaagcggcgccgccgtgatgccggccgcatgacaggtcgttttcatcgcgtatgctcccggccctaatcctgccccggccgtgccggctggatttgacgcagtattgttgttgcctgccatcccgtaaacccgaaggtatcggccagttcgccgggcagtgacgcggccaggcgcggcagttcgtccggggattcattgtgaaagacaaaactgaccccctcatccctgatgcgcgacatcaacattgcaataagccggtgcggtccctcatggctggcgtattccgtgaccttttcagtaaaccgccggggattgctgcagctgataccccggtcggccaggaacgccagaaagcgctgcaggaaaccggggagatccctggcggtcagggcgggtttcagcaccgcttctgcctgggcctgtgtgaagccaaacgccgccgccagctcaggggaaaagtccggcgtgtcagcgctggcctcaaaaatgacgccatcgtttatggcctgctgcatgtccgggggcatattcccgatcatattttcacgcacgccccatgcacggccggcgctttcgttcagtctctgcggatcgctgcagctgacgccccgctccgccaggaacgtcaggaaacgctgcaggacggcggcgccgatctcgccggagacgggcgcttctccggagggatagaagaccctgacgttggtgttatccagtaaggcctgttcagcgccggcgtcggacccggtgctttttccgctccgccagaccgtattatttcgactgacgatggccggatcgacatgttcgctgtccacgatgctgttaatgaacgtcactgactgttcttcgctgaaatcaaagtcgtgctgtaacagggacagcagcctgtcatgccagtcatctttcacgcgattggcggcattaaccatgaacgtgacgttcagtacaccggccagtaaacaggcagcctggtcggccacatacaggctgaagtattccagcaccgcctgttcaaatgcagcgggatcgcagcagaccacttctgcagatcccagcgactgcagaaaccgccgggtgttttcggtcacagcgtcggcgggattgagggcgatggcgcggccgcgctgtgactggatgtcaaatgtgacgatacgctccgtgacgggcatccggccgtgatcggcgatgaagcggtaaagctgctccactgcttccgtggcggtcatgcccattgcctccagacgcagttcggcgcgtttcttcagctgctcatcgagacgcacggtcagctgggcgtttttggcccgctttttacgttcaaaaaaaccggtcatgctccttcctttttgtacagtcatttggctttacataatgtacagccatttgtaccgcacttttgcgcgtgggaccacattttttcaccgttgcggtttagcggtattctccgcagtgccgccacttatccccagtttctgtggataacgctatatgtagtggctccgtcagctggaaacggcacaagatatggtgtccgcgccggcagccgtgcggccgttcccctgccttcccttccctgttgatttcgctttttccggtatttacgttttttccgcaatcacggtgccggcgaaaaggtattttccggtggttgcgggcgcaaaagtcacagggtgaaaagataagggcacgaagcattccgcaatataaactgcgtgacagtcacgccgggtgatgactacccgcgggcgattctgccaaattagctattagtgctaaatctaaatatttataatttacgcatgaaacctaatttattagagtgtggttattcagtaaacaacagcaaacggagtgaacgggatggaatatgaagcgtacaaggttcgcggttacggcttcggactgaaaaaagagatcaccggcgtgctggggcgttaccccaccctggaagaggcgcagaaggtcgcggatgactacacgcaggcctgcctgccggataacctgccggacagctacgatatgggcatggtccggccggtcgctgccggcgaacctgccgccctcgatccggacgtgtttcgcggtccgggcgcgccgttcctgatggatgccggccagataaccacgggtcacacggtatgcctgggcaaagcagcggatggaaaacggttttattttggcggggagcattccgatggccaccaaacatataaatgatgagctctggaatcgcattgaggcgctgaccgtcaaagctaacgccaggcatgggttgttgcggccgattaaagaggctgaagtactgcatcttgtcctgcagcgtggcctggaactgcttacggacgacgatctgctgcagctgggcaagtaccggcgtccgataggatttgtgctgcgtcgtccgggcgtggaaatggtgaagctggatatgctgaatatggcagatgcggcaaccgttcttctgcgcagtggacccgcaacgctgtgcatctggagccgcgacgatatcctgcgcgaggccagtgaagcggtgatccgcgaacggctgccggatgcagccctgctgtcggagggggatgaccgggcccggttccagacgctgctgcccggattctggaacgccgcgcaccggggcgaaacggccgtgatttccctgcgtgctgacaatgcggatctcgctattgcacgcatcactgacctgatgtgcgaatccctgctgggttataaggggcagcgggcataccgggcgggtgaaaacgaacagggagaagagtcatgaacatgcgaggcattctttttgtcctgccgctgtgcgccgggctcctggcgggctgtgataacaaacaggaagcgctgcagtttgaagccgcccgcacggcggcgctgaaggccgacgatccgtttctggcgctggactcgttcgaacagcagtccggcgactacggccgggactgcgactcgctgggggcatttctgtttggtaattttgcttccacggataaagcatgtcgggcgcgggatgaggccacgcgggatatggtcctgaaagccgtagaaaagggttccgtgccggcactggtgttcctgtttgacccaggcagcaagacgccggatgtgtatccgcgcgatattgacccacagaaggccgggcaggcggctgagcggcttgttgcactggcagagtcggcgccggtggacagcaaaaatcaggcgctgctgatgcgtgccggcgatgtactgcaggcgggccagtacgttctgcaggattcccggagagcggcggccctgtacgtcaaagcctggcaggcaggggatcagcgtgccgcggacgagctggtcaatgtctatcactttctcaaggacagccggcgtacctacttctgggcggtccggagcaaagacgtgcagcagctggacaatcgggctgagctgaccggagatgagatcattgctattcagaaactggctgcggacagttcacggaccgacatttgatgaaatgaataaggatatacaaaaggcgtccaactggtgaggaagggcgccttttaattactagacatttttactacttaactttctgtcggagtaaacggaaaattaatctgttaatcactgcgagtataaatccccgctaagcgtggtgcaactcagtatcagaaatgaccgccggtggttgaatccagtgccctgaacaggctttcctgggctgcttccagcagacgacctggcagacggagttcatttacgcggtaactctgcccggtctgtgtggtcacggtgagataatactgatcgtcaaactgctgaccagcaccgatatgcacgatggtggccgtgtcgatccagcagaggtttgtgccttccgcgtcctcaagtggtttgattcggcctttcagccacttctgcgggcgacccggccggcatttgatcgtgatgccgtgcttatagccgcgggttctggccacaaaggccgctattttatctttatccagttcctgcgccggggtgagcttactgaaactggacggagagaagttatactgcagttcttcaaacaggttcaggtacgtggcttcatcatccaggttgtccccgcgttctttcagcatggagccccagatcctctccatttcgcccctcagcgccagtttaaccgcctcaaattcttcctgatgttcatcgtagtagttgatatccatcgcgtctcctgttgtgtttatggcgaatttgcggaattaatatacataaaaaatgttcaaatatgaacattttcaggctataaatacaggcagattataacaggccagtccaccaggattcggccaggagctgagtacgcatgataaagaaaccacttattgccggcatcatgctggctgtggcgcacaccgccggcgctgcgcagggtgatgataccgatctgcagcagatttataccgaggcgggcattcaggggatatgcagcgccgggatgctggctgtgccggaaggggccagggatgaagctgtcccgcagaacgtcagacacctcctggttcagtgtgcctatttcgacgagcagcagttgcgccggcgtgccggcctgccggctcagacatttagccactggcgtgaccgcaatgcggtccttaatctgtcgtctgaccaggcatctgatgcgctctggctgtcagcgtggcgtgatttccgggcacaactggcctcggcctacccggccccggcggaaaaaaaggcacaggggaaagaggccgtacccgccggcacgaagcccggcactgagcatgggagtgcggcggattgaccggttttgacttgatcctgtggcgacggggcctgaactggacccaggagcgcgccgcggctgagctcggtatcagccgtaccagcctggtaaaatatgaggacggtgagacagtaccccggaccatacaacttgccaccgtcgcgctgacgctgaaagcggaatggcccaccctgaaaaccatgagcaaagacaggctgctgcggcagctgaagtatgaagtgctgaggatcgataatgaatgagtttatccatgttgtcggctcatttatccttgcgggtatcctgctgtatggcctctggcagggtacacggcgacgccggcgccggcatgaacggaaacaggccagtgcggtgcgtgtgatcgataaaattaatacgttcccgcatttcgggcagaaaattgcctatctgcgcaagattgacccgtttgtgttcgaagagctgctgcttgaaggctttgaacgccggggctttgaagtgatccgtaaccgccggtataccggtgacggcgggattgatggccgcgtgaagattgatggccagacctggcttatccaggccaaacgctataccagctatattgccgttggccacgtccgtgacttcagcgacctgctgaacgccaccggtgcccgcggttttttctgccataccggcaaaacgcgcgaaggcaccaaatcgctcattcgcggagacagccgcatgatcctggtcagtggccagaagctgctagaccttattgccaccgatgcgccgtttatcccgtatccgggataccggccaccggccgaaatggttcatcctgaacaggagacaacatgaaactgaaaggatacgtcgccgccctgccggcactgctgctgacagggtgcgccatgctccccggacagcccaccgattatgaccgcttctgtaacgtctcaggcatcgccagccacggcgaaacctaccgtgtcagtgacagtcaggatttctggttaaccccgaacggccgctatctcagccaggctgagtacagcagtccggcagatacgcttcagaaactgacgggcgtggtcagtggtgaagatcctgaccaggtgcggaagaacgccgtcagggtaagggtgttccgtgttgaaagtgagagcgccagtaaaggggcgtgcctgcccgtgcgttatgacgataaaaacgcgcggcggaaaatggattcgctgactaacggccgccgtatgatggtgtacagtgaagatgaaggccagtctggccagcagatttacaataacagccgcggtgccggcttcagttacaggttgctatgagtggattgaaaggccgccgtatcagtgcggatatgtttgccataatgctgaagatatctgccgggcagctggtttttgcagcacggcatgacaagccgttacgcggactgaccctacccccttacagaatcgaaggcggaaaaagctggagctcgaccacctccacccgcagtatgacgtttgatatgcaggatgcggtcgatttcagcgagaaatttcataaaattcagtcgcctgacggctgattagcggattttaaaaaattgaacactttccccctgcaaatttctcagaattcacgtagcgacgccatcgggacatatgcctacttcgattatgccgatgcgctggcgctgcgggagatggccagccaggctgatttacccaaatacctgctggccccggaggtgtcggtgtttctgcggtatgtgccagacctgcgccagcaggtccttttcgataccctctggaacacgggcgcgcgtattaatgaggcgctggcgctgaccggcgccagctttcagcttgacggttcccggccgtttgtccggctgaagacgcttaaacagcgtcagcgtggccgcggcaggccgggcaaggatgaagaggtgttccgtcttgttcctctgaccgatcctcaatatgtcaggaaggtgcgtgaatttctgaccaccctgcgtattggtaaacagcagctgctctggccggtgcagagtgataacacgccgcgtaactggatcaggaaggcactggacctcgcaaaacgtgactctgtcacttttagtatccctgttacctgccacacgttccggcacagcttctgtatgcacctgatccagcatggggttccgctgaaagttgtacaggcttatgccgggcattcccggctggaaaccacagaaacctacacgcgcgtgtttgcgctcgatgttggtcgtcagtatggcgtccggttcagcctgccagacagtcatttaacgctcccggaataatctgtatccctccctgatatttccatataaatctaaataaatccattttctttctaggtcagaccgttttcagggcggcctggccgtcttttgttggtttttattctatatttttctgtatttttctatattggtgcgtctttttgtctttacacgtcagcagggatgcgtaataatccatttgaaagctgtatttttcctgatttctacggtttttttctaaataatgtgaggttcaaaatgcgtatttttgttgacgacggttccaccaacatcaaactggcatggatggaagacggtgcggtcaaaacgttgatcagtccgaacagcttcaagcccgaatggtccatgacgttgctgacagattcatcagtgccggcttccgccaactatgaaattgacggggaaaagtacagctttgaccagctgagcccggatgccgtgcgcaccacggaaacccgctatcagtacagtgatgtgaatgtcgtcgctatccaccatgccctgatgcagtccggcattgaaccgcagccggttgatgttgtggtgacgctgccgctgggcgagtacctggatgaaaacgatcagcctaatatggccaacatcgagcggaaaaaagcgaacgtgaagcgcgcggtggccgttcaggggcgtgagagctttaccgtgcgtaaagtcagcgtactgccagagtcggtccctgcaggcttcagtgtgctgaaggatctggacgatctggactccctgctgattgtggatatcggtggaaccacgctggatatcgcgcacgttcgcagcaaaatgtccggcttcaccaagacacattgcgatcctaaaaccggcgtatccatcatcaccgaagccgtaaaacatgcactggataccagcgtgagcacccgtaccagttcgtattttgccgaccgtctgatccaggcgcgaaatgacgaaacgtttctttcccgttacctccagaacgctgaccagcgggcccaggtgatgaaggtactgcatgagcgtgaaaaagccctgacgcaccgtgtcaccgattcagttggccgctttgccggcttcacgcacgtcatggtggtcggtggcggtgccagcctggtcgccggcgcagtgaagcaggcaacgggggtgagtgacgatcgtttcttcgtcagtgataacccgcaattcgatctggttctgggtatggtggcaatgaagggctgagtatgactgataaccgtaagtgctcgttttatatctacccggagcgcaatgcggcggaccgggtagccgacggctttcttgagaaactgcctcagaaagagcgtggccgcgcgatgcgcgccatgatgctctgtggtgccgcgttgatgaagcaggacgaacgcctgccgttcctgatcgctgaattcctgacggagagcacctcgatgcaggatattcagcggatcatcagcagtacgctgccgcagcaggatacaggagaaatggcgcgtctggttgaggcgtttctgcaggccaccggcggtggcagcaatacggtgtcgccggcaggcggcgctgcgccggaaaacagtcccgccgtgactcccgaaccggccgcaccggaggataagaatctggaagaaacccgtaagaacgtccagcatctgttcccggatgatgattaagggggcagcgtgaaaaaatatgtgctgttaaccctgaccctcatcgccggcgccgtggcagcaaacccggtaacagcggccggcaatgttcaggcgcaaactgcgactgaagtgaaaaccgggttttcacccggcgggaccgcattaaatctggttctggattcaatcgaccacgcgcagcatgagattctggttgccgcgtacagcctgaccagtcgtcctgttgccggcgcgttgcgtgccgcaaaaaaacggggcgtatcggtggtggtggtggccgatcgcaaagcgaacgacaaccggtacaccgaagtgaactttctggccaataaccgcgtggcggtgcggatcaacgataactacgaggccatgcatgataaattcatggtctttgaccgccgcgccgtcttgaccggatcgtttaacttcagcgccagtgcagacagtcgtaatgcggaaaatgtggtgctgatcagcggcgcgcctgcagtaactgaagcctacgtgaacgagttttcccgtttatggggcgaaggaaaggacgtggccccacggtactgacctacagcagcggcagaaactctgccgcctgatttttaacttgatttatatggctcattcgtcgtattatcaaggctcatttttcggtagcgccccgactggcgaaggagtaaaaccatgcaatcattccccctgttcgtttcccgtgtcgtacctgtgatcaccctgagccattgccttcggcagcggcgctggtccctgcacaacacgtcatgccagattgacctgagatcaggttgtcacctgcgctcacagcctttcggcacgctatccagacgggaaataagcgaccgaccaccggctatgccccgcggttactggttcaggcaatgcagccgggatgtcatgtttacggggcgcatcgatcaggcagatcctatgaaacaggtctattacaacgaaggctggagcggtccgaacaagtatacgtttgaagtttatcagctggagaacgggagttaccgggcgctggcccgcaaatggaatggcaaaatcaacaaggtgcagcaggaaacgcagtacctgtctgacacgcgggaggggctgaagcaccaggactatccccgcacgcgccaggtgaaaatttttctgaattcagacttctgggagaaaggtaatgattaagatggttagcgtcgttccgcagccggaaacagtgaagacgctccgggaaaaaatgggcatgactgaaaccgcattaggggctgtcatggggtacgaactgcgcgcctggcagcgtaaggaagctatcagcgacgatctcagccagtacaacaagaccagtcttcgcccaggcgaatataacatgctgatgctgatcgccggcgtgcatcctgattaccgcctgaacagggcattcagtccggatgatatggtcaaagatcccgccacggccgaagacgttcgccgcctgcgtctggcgctggggctgaagcacgctgaaattgcagcgctgtttggctataaaccggcgtcatggcagaccaaagaaaaggctgctcagcgcggcgtaaagctcaaaaccggggaattcaatttcctgttacttctggccggtgagcatccttcactgcagctggttgagaaggtgaaataaggcccgcggcgaccagagaggaccctttatgatcaccacacgccgaatgttaatgctgtatgcctgccttgtgcccgtagccctgacagggtgtgatctggctgattatttcccgaatgtgccacagcccgtgcccgtgatttcgcccggctatcaggttgatatcgatggccacccggtccgaatatcaggatttgatgactgcccgcgggataaagatgaagcccggcacgcagctgcacaagatgagaaaggctgcattgtgctgtctgcagagcgttccgaggtgaaggtcagactgtatcagccagccggcaccgtcaccgaaacctggcggatcatccgggaaaaagacgctcgccagaacgatattattcgcatgcagcgcccgaacggtgcgcttgtgatgcaaaatttacaggggaaagagtctccttaaaccaggaaaataacaaaattaacttgatttatatggctcattagacgtataataattacatcgaaaggcaatccggtctttcgacggtagcgccccgaccggcgcaggagtcaccttatgactgctctcaattctcagcaacaggctgatgttgcccgctatatccgggaacatggcaataaaaccgttatttcccctgaatggtcacttatttatttctcgctgtgtcagtgtgaattacatcaatcgcatgaagccattatttttcataatggccgacctgcagggctatttcttctgaatgaaacaggcgagctcaccgaattatatctgctggggtcgctttctgacattctcaccgccgctgcggtcaacaaatagctttcagggagtaaatacactatgaccatgcaatcagaacttgtttttactgatcctatgctcaacgtcgttattgctgaagttaagcgttttaattgcccgcttctttttgtcaaagatcatggcgtgtatgtgatggcggcgaaaggtgagaaaaacagtaatgggatgcacaatgtctgttacgccaatggtttcaatccggatacaacggattttgacgagctctgggaccggatgcgtgatgcctgtggcggggatgatttttgcgaatccctggacctcgatccgaggtcgattgagcttctcagccgtaccaaaccctgcctgaaaattatgctttcagagacggaactggaagttattgcagggggacagaaataaaccataaaagcccgttaaatcgggcttttttatatttaactgtatttacatataggacaaattgtcctaatatatatttgtggccgggcatggtgctcaggtcccctaacgagggaaatatatgaaattttcagcagaagaactcaacattatccgccaggcggacgccattattgccggtaaaattaccacaacggattttatcaccggcgccgatgccgcgcgttccttactgcggtttcgtcttgttgcagaaccacgcgaatcattctgcgcgttctttcttaactgccacaacggcgtgattgcgtttgaagaactgtttaagggcactatcagcagcgtcaccgttcatccgcgtatcgtcctgcaacgcgccctgcatcataatgccgccgccgtggtactggcgcataaccatccttccgctaacactgcacccagccaggctgacaagctgatcaccgagcgtctggcgcaggtgctggggatggttgatgtcaaagtcctcgatcacctgattgtgggcggcagcgatatttactcttttgctgaacatggtctgatttgacaactgcggccgcacagtggtgcggccggctctggaggcttgctatgggcaataaatataccgtgccgcttaacgtgggcgcgatccgcgaaagttgtttccgtcccgctgataaatggaccccgccgacgggggatgaactccgctatctgctggaaaatgtgctcaacctcagtcaggaggggctggcgcgacatgttggcgtgaacggcagaacggtgcgccgctgggtgaacggagaaagtgacatcgcgtattccgtctggtgtgtcctttgcatcgatgccggcttaccgccgatctggaaatagccgcgcaaaacaccgacgcgcaaacgtgcttcctgaacgtatcggatatacgaaggaaatattatgaatactgataactattttttcagagtcccggccacacgcggaatacagggcggcatagaacaatacatgctgacggttcctatggtcgtgttacgtcgtattcttgcaatggacaatgatggcgatgttatggatcgcagccagcgagaagcgaacaaaacgcgggccaaaaaaatacggaattatgttgccggcgcaacatcaaaacgagctccctatattttgccgtcaattaccggaaatattgatagtcatgttgaattccttcccagcgaactctcgccggcagtcggtattcttaaaattccgatggacgctgatttaaaattattcgatggccagcaccgtgcgctgggtatttttgaatttgtacgcgattattccaacacggaagatacgatcagcttattgcttaccgttggcctgccacttgagttacgccagcagtttttcgctgatattaataacaacgcgagcaaaccggcagcggcgatcagtatggcatataacaacaatgatccggtcaaccagctggcaatgcatctggcccgaaccgtcaccgggctggcgggcacagtggattttgaacacaacgtggtcccggcaaaaagctcacggcttatcagctttaaagcgctgaacgatgcgacgaaaaaaatgctcaacctgcgggccaacagcattccctcgccgcaacagcgtgacatggcagaaagactctggactgcctgggcgcaggcgatgcgctggaatgatattgcccaggatgatatcgccgcggagtaccgtcaggaggcgctgggactgcatggcatcatgattaatgcgatcggtatggcaacggccaggatgttacggcacagaacccctgaaagcatcgagaacctactggcctgtgctgaaaacggtgacaacggttttcattaccgggagtcgtttgtacctgagtgctgggaagggaagtgtgttgatccggaaacaggcaccatcaaaaccgacaggcggtcgctcgaagcaacggctgaagcgttgcaaaagctgatagatccgttcgcagatgcgctatggttgcgtgattacctgccagctgaggaggcgactgatatggcattactgaaatatgcagccgacatcgagaattataagcagcgtactgccgcgccgatgatcaatatcgtggagaagctgaaggcgctgggtgacggcgaaccgcagttcagagcgtcggtgctggcttccagtgaaggactgagccactatctggccggggctgaaggctgaccaggtgtccgccgcctgcagggcaatacgggcatcgaacccgtattgccctgaaacgcgctgagcagctctctgagcgcttgtttcaaaaacctgtaccccactcgcctctattgatttgacgtgtccagccggccattctggccttatctgatgttattccgacatggcatctgccagttaacctacttctgacatccgtaccgtgtgtcttccctgttcatttctgctcagttgccggcgttttctggcaggcctgtcgtactaaaacacctctgggccccttcggctcctgcccgtcgggcagtcgtcagccacactccgctagccaggcaaagcctggcagctccgccgaaccccctgcggggtttcgggctatcgctagtgtctgatgcctgaacccctgcaccatgccggtaaagtgctgccagctggctgatggggatctgaaagcaaaatttacagggaaaggagtccgggaaaaaacaaaggaaattaatcagataacgcttgattgttatggctcattagacgtataatgatcacatcgaaaggcaatgaggcttttcgcggtagcgccccgactggcgcaggagttacaaattatgagtaaagcactgaccccttcccagcttaaagctgaactggacacgcagaaagatgcgctgctgaaggcgtgcctgtatgccctcaacgaaatccctaaccgccgtttaagtggtccgtattcgtccacgtatgcgctggccgcgcagattagtaaactgcttcagagctgcaaataatcattgatgggctggcattgccagccctgttcaacgctaacggtagcgccccgcatggcgcaggagtctgaaatgtcatctaaaaatactctcccgtcggtcgatctgatgtcctgccagcatggcggtgacgtgatcccgtccattgctattgagcgcattatctcgttgcgggatgaaggtatccgcgtctatctcgacggcatagacaagctgcgaaaagcgcgcgatctgatgcagcaggccggaggctgtcagtacctgtatgactacgacaaagcagtagagagcgccatcaaatggagcgacaccacgccagaccaggccgaaacggctatttcccgcgttatcgacggaaaaatctgggatcggctgatgacggaaaccggcatgtatacgctgatgagcaacaagcaacgtcaggaatgggatcggcaggtggcaggcaaagaaatgccgccgataacgctggataacgttatgagcacgttccggcacctgaacgcaagcaaggcggatactttcacgcaagggctgatagatatctttaaatccctgtcgtgggattataaaaccaataacccctgcatgtttggcaagcgtatcatcatcgcgccgttgctcgatgtctggcgcagtggctgggtgcgcttcagctcagacgggcacaccaaaattgacgatctggcgcgtccgttctacgtgctggacgggcgtaacgtgcctgattaccgtgtttctgacggcgccaaactggatgcttttttcagtgaaaaccagttcaacggtaaggtgttcgagtgtgattacttcagcgtgcgatattacaaaaagggcagcgcgcacattacgtttaaacgcccggatctggttgagaagatcaataatctggtcgccagtcactatcctgggatgttaccccctcgcgtgtaatccagacggcaggccgcatttgcggcctgaaatataaacagggaaaggaagtattccccggcctgagaaaatcatgccgggcatgtatttattgcttgattagtgtggctcattagacgtataatgatcacatcgaaaggcaatgatgcttttcgcggtagcgccccgactggcgcaggagtaataaacatggctattcaatcccttcagttccgtaacggctccgtttctcttggtgatgtttttgtttccagctggggttatgagcagacaaatacctgtttctatcaggttatcgccctgcgcggtaagaaaaccgccgtactgcgccgcatcgctgcgcagcaggtgaaagcagacagtgcgatgtcaggcgagttaaagccggttttgaacgagtttaaaggcgaacccgtgacccgtcgcattcgtgaaaaccatgagcatccctctgtcagcattgacgagtacgaacaggcttataagaccgatcctaacgaaagccatttttacagcacctggggctgaccgcctgctacgccccgcaaggggcgtatagtcaaagacttattcaaccaaaacgtgaatactatgcgaaataacaaggcgctcaggacgcagatacatggcggaaacagtttatcaggcgcggaggcgtttttaccttctccgttttcggcgcagccgtaacccggacacactggaaaaaatgtacgaatccatgcgggatcgtggacaggtgccgccggaggactccgaggcttttgaggctgccgccgaccatcgccgcgcagagctggcatcgggacgcatctgggataagatcccgccgcatgtctggcagtatgtaaaataatcacagcgcccccacgaggggcgtttttttgcttgtatgtatggctcattagacgtataatgtttttactgattcaaatcagtaccttcctaaacagccttacaggtgcattatgacctcttctgaaatcctccataacctccctcagattatcacggccgtggccgccctcattacagccattaccgggctgattaaagtcatgaaggataagggtaaggactgaaccgccgcagggggcgacagccccctgcatcgctaaccggcagcgccccgattggcgcaggagtaaaaaaagtgacaacgctgcatattcgagaaattcccactgatggcgcaccgcctatctgtgcaggccgctcaatctcagatgatgacctttgcgcctcatgccagcatctcgactatcgccctgggcacctcagcgcctgcctgctgtcaggttgcgggcgtgacgactggccttcagaactggatgaaaacggctacagcgttgcctgtagccgctatgagcgtgaaaaattgcttgattaatatggctcattagacgtataataatctcatcgaaaggcatccggtctttcgcggcagcgccccgactggcgcaggagttttacaatgcacggtaacgtaaatgaaatttgcgcaaggcttctcgatagttttgaaccacagcagcggatcagccttctgatctggactgctgaagatgtccatgactgcaccagtgatatgaacctcactgacgatgaggccgaagccgttctggcagaaatcgcggaatgcagttcacacagccgttatggcgtcggaaaagataccgtctggagcctggcaaagcaggtgcgcgaagatgccgcccgcgatcgaaaaatcgaggtcaatgccgaggcgctgcaaaaagtcgtggcgctggcggcgcagtttatccggctggaagagatccagtctggtgaaggagctgcacgccgactttatccgcaggagtctgaggcgctggagtgcatcacgaaggtgattaacggctaattcacgcagtaacgcagcgaactttcacggcagcgccccgactggcgcaggaggaaaaaatatgtcccagaatgaagcctattacgacacgctggccctgattgaaaaggggcaatggtgtttcggcagtatggacgaagaaaacgaagagcgggcaatcaaggccgctaaggcgctggcgctgtttgcgcgcctgaatgagcaggactgcgacggacacccggtttccgaaatcatcgttgattttatcgctaacctgatgcacctgggggaagcgataaattttcgtgtgctcgatgaggaaagcgcggtgatcccgttggtcaggatggcagccgtacactttaacgctgaaacaaagtcatagtaatcatgtgaatcagaggggggcaggcgtgatcctgcccccaaaaatggacaggggaaagagttgtcccgcctgaaaatattcaggcaggcggggatattttgcttgattgatatggctcattagacgtataataaacacatcgaaaggcaatccggtctttcgtcggtagcgccccgactggcgcaggagtacagaaaatgtctactgaactgaatcaacccgccatcactgcacgcattatccccaataaccgccgcatggcctttctgccacgcctgttcggcgcctggtatctcacaggtgaggccggtgtctacaaccacgcacgctctctctgcgccgattatcaaggcggttcctgggagttcgtcgaactgtcttgcggtagcggcttcatgtacccactgagcgcagagcgcttcacggtatcggttagcggtaacttgttcgaaggggaactgtccgcagaagctaccggtatcgtgctgaccctgttcaccctgaaccacatgatctggcacgcacatgaagaaggctatcagcacatctgcgacatgctgatcacccagcaggaaaagctcaaattatatgccgaccagcacgccgaagccggtttgatttaccgcgcgattgactgaaacggcggcccccgcgagggggccaatactgacagggggaaaaatgttaacactaactgatattcgcgccagcaatacggtgctggttacggaatttggcggcgtgcgtgctgtgcatttctgtctgcatgaaaaattaagcggtagcgacaacgatctgtggtttccgctggccaatggggccgacctgttcgaggcgctggaaagcattatgtgcatcaattttgccgctgctaacgttgtctcgctggagttcctgcggcagaacgggaagtgcaaagactaccggatcacctacaacaaggccaaattcaagccattatgttagatttctggtagtattaaatgttcatatttgaacaaatttaattaaaaaaatacgtctgatgagttgataaaatggctcattagacgtataattattacatcgaaaggcaatgaggcttttcgcggtagcgccccgactggcgaaggagttgaaatgaaagaagcagaccaaaaaaaagtgaatgccgtaatggcaattgctgattatctcggtgtaaaaaatcaaatcgaggtgatcgaatactcagccgaatcagtgcaggttgagtggcgaaatcctaaaacaaaacagcttattcatcgtgattacacttttgcaatttcctttgtaaaagactttgaaaaagccctgaaatcgaacgtgaagttttactgataaagagggggagaaatcccccttaatcctcccattttttactgataaacatcaaatatatgaggtgtattatgtggaaacgtggcgaaaacaaagtgattctgatggggcgtgctggcaaagatgcagaagtgcgttacaccccaaacggaacggccattgcgagcctgacgctggctactgaaatcagctacagcgacaacgaagggaaggagcaaaaagaaacggaatggcatgatattgtcatttttggaaagaaagctgaagcagctgggaagtacttcaaaaagggtatgatgctgtattttgtcgggcgcattcgtaataacaaatggcaagggacagacggcaaaatgcgcagcaataaggaaattgtgatcgataataacggcgaaatgcagatgctgccgggagctgggccacgtaacaccagtgctgaaggtctgggaggtactgaaaatcatgatgaaccgccgttcccggatatgaatgattacccgcagtgagtatttgagtgagaacggcccgcaggacgtgggccgccaacccagaaggggggaagtatgaatattagtgaacaatatcgggtggctgtcgtcagtaccggccatgtaaaaaaagaagacgcggagctactggataaggtctgtaacgatgacttgtccggtgaaggtttattctggatcgagaaaaatcgctatgggtggatattgcgtatttctgctactgaacaatggcgtgtggaattaaccagggcggggatcagtgcaagtgctttggataatatagccgcgttaaatggtgccgggttcgactggatttggtttgaccgggatgcagaacaggtgccagaactgaccgtttgggagtggtaaaccggttaaaaatatgcctcgaaaaggggcatttttttcttgcctaaaatggctcattagacgtataataagcatgaagtttaaaagtggatattgattaaaacgctctgtaaataacgtggagagaatattatggcgaccttatacccttatgaggtttatacccttattgtgtcagaattaatcgtaagccattttaatgatgtcgctatggtatatccgggaagcgaagaaattaaaaagaactacgacagtgaattaagtgctgtacaggtagttaatggcatcagagaaaaactgattgaagatggtgttatacagtgcgaacagtgcccactgctatccgagtgggatgagaacaaggttatcaaccagctaattaaagattaccgcctgattctcaatcgtgactatgaactaaaccgtgttcgtcagcagctggccccggagctgatcgcacagatagaaaactggaaagagcagaagtattccgattctgcgaaggaatacgaggtgctctatgaaaccatcaacatcaacggaaaggaggttacaggaaaactaaacgtagttgcagtaagtcgctttgatgctgttttcatcgtaggacagcgtcacgctaacgataactttttggttctggatgtaaaggaggtgatgaatcattgaagtccagaaaaggtgccggattgcgccaacaatccggctgggtaggcaatttaaattgtgaggttcaaaatgccgttgtggattatacactatgctgaagatgtcgcaaacggagtgcaaacaaagtcgcatccgatgaacgcagcgcaatgggatcacttccgtttctggttggcagtgttccggcggattaaagaagccgcgatgtaatctgtaactgtctcaggggcgctgtatgcgcccctttttaatatccgtgcccggactcctttccctgtaaatttatcgggatcaagggcggatcgtggccctgaacgtgcgcaccattgagcgctgcatcgcagcggaaccggaaaattttttccggccaatgagtgcccttgtcaccggaacggtgacaagtatgcacgcactattacacgaacaggcagctcccatgcccggcctgacaggccgggagctatatcataatgtgggatgctatatgcgaaaggtgaaggttaatactgcggacctgaagcatcaggattctgacctgtagagccattttgcgtgcgctgctcttcgtcaactccagtggttttttttcggctgttgctggttcgttttttaccaactcgtccggcagtaatgtccttgatatcacgtaatgtaacatctgaaaaacccgcatcgttgaggaatttcagtatctctttatcttcataccctttcgaatctttcagcttcttaatgtcagacttaagttcatcgagtacgtgctttttctcaagcctttctgacgtcacatcaggcaagtcgtcgagaatgcttttgaattttccgatgtcctcactgctatagaatgctttcttttttgcaggcatatgccctcccaataatgtcaaaatcgaccatgcctaaccggagaaaatcggacaaggctattcaggtgattttacatgatgcccggcatgattcaattgctctgcctgccaggcccttcgctgctggcattcaaacagcgccgatggtagaattcaatgaggtatttaagtacgggacaagatgtgtttttggagtaccgccgacacgcggcggccgtccaaaaacgtcttggtcagggcaagccccgacaccccctaacgaggttagctatctgatgagccgtagcgagagcagaaaaacagacgatcgaatccaggtgagatgttccacagaagtgaaagctaaactcaccgaaaaggcccatgaagcagggcttagtcttagtcaatatcttatcaaatctgggcttggaaagcgcattcaatcgaaaggtaattacaatgcacttgccgctcttgtaaagataacagcccttcaaaaacatctgtttaacgaaggtgccggagttcatagcaaggagtattcagagattctgatcgaggtcaaaaaggccgcacagaaactgcaacaggagatggatggtgatacctaaaatcattgagggccgaagggacaaaaagtccagctttggtcagctgattaagtacatggcagacaagccgtctcaggagttgactgatacagttcagcctactcctgagacggctttagctgttaaatcggatatgttcgaagggttaaacaattacctgacccgtaagcagaaagtgatcagcacaccggtggatgttgagccaggtgtacagcgtgttgtggttggtgatgttacctgccagtacaataccttctcgctcgatggtgcagcacaggaaatgaattcagtttctcagcaaagcacacgctgtaaagatccagttatgcactatgttctttcatggcctgactatgaaaagccgaatgatgatcaggttttcgattcggtgaagtttacactggcatcgatgggcatgtccgatcaccagtatgttgctgccatccaccgggatacagataacctgcatgtgcatgttgctgttaaccgcatcaatccccagacatataaagcagcctcatcgagctttaccaaagacactcttcatcaggcgtgccgcttactggaattgaagaatggttggtcccattcaaacggtgcatatgtggttaatgaccgtcaacagattgtccggaaccctcacagcaagaaagagcggggcaactggcgctcactcgatcgcatcaacaagatggagaataaagagggggttgaaacgctctatcgctatatcgttggtgatgaacaggtaggcggtagtcgtcaaaacttgatccatgtttcagctgggcttcgtgaagccaaaagctgggatgatgtgcataagacatttgcagacatcggtcttcgtgtcgaaaaagcacaggggaaaaagggctacgtcattacccacgaacatcaaaaccagaagacggctgttaaggcgagtctggtatttaacaaggcgcaatataccctgaagtcgatggaagagcgctttggtgaataccagccttcacatattgaaccggccaaagtatcggtatttaaaaccgcatatacccctggtgcttatcgtcgagatgccaataaacgtctgcagcgtaaaattgagcgcgcagaagaaagaatgctgctcaaggggcgctatcgcgcctaccggaacaatttaccggtatattctccggataaggatcgcatcgccgatgaataccggaaaattgcccaacatacccggttggtgaaaaataacgtccggcattctgtttcagatcctcataccagaaagctgatgtacaacctggctgagttcaaacgcctgcaggcggttgccaatctgcggcttagcttgcgtgaagaacgcaacggtttcagagcggctaacccacggctttcttaccgggagtgggtcgaacaggaagcactgaagggtgataaggcagcgctcagccaaatgaggggctttgcctattcctccaggaagaaggagaagtataaacagcagctggttgagcagatcgggttcaaccgtacctttaacgccatcaccagtcatgatcgtgatgatgtggccgtaatggcctcagcacgtcatggtgtcaagccacggcttttgaaagacggtactgttatctttgagcgcgacggtaaaccggtagcggctgatcgtggtcatattgtgctgacagaaagtaatggaatcgacaaagagaaaacggctgatcttgcaattgcattaacgattgctggtaaagccaaatctgtacgtgttgatggtgacggcgagtttaaggaactgtgctgtaatcgtatcgtcgatgcagctgtgaaccacaatcaccctgtcgcgcagggcattaccttcacagatgccgctcagcaggcgtatgctcaaaacgagaaacatcgtttgattcgtgagcaaaataatagtaaaaatgaaatgcaattcagaagtgaaagagacgacaaattcaatccgaagtaaaatggagtaagaaatgaaaatcaaaatgcaaacggtcctcgcggccgtttctgtgttagctcttgccgggtgtgcctctcaaagcgaaaaggaaaaacaaatctctgcacagcagctggtagatcaggaattgcgtgagcaagccattaagataaaactcgcacaggatgagctttaccaggctggagccattaacaggacccgttacaaattccctaccattatcaatgcaaacagtcagtatgttcgtgtgagctggcagggtgatgcttacgaattactggagcaactggctaaacagcgtggccttcaattcaccagtcaggggattaagttaccgcttccactcaatatcgatgttggtggttcgaaggttacgtttgaacagttgctgaccctgatccgacaacaaacgcagtaccgtgccaccatcaaccagaaacccggtgaactgcagctgctttacctcacaccatcgaagaaaggttcattcctcagagggacccgcccatgaaacttaaactgatcgtgcttctgacgttaccgctgcttgctgcgaacgttgcacgcgcggacgactccgatgttaaaggcctggattactacatggacccgccaagccaaagtgacgatgaggccgatgttactggcagtatgctaaacgacgcagcgcacaccattggatttcgcggcggtaaggcagaacgcgctaaagaaattcgcgccgcgctggaaaatcagcgcagcaaccttgattacatgtattcgttccagccgctgatctcttctgaaggctacctgcctccagtcattactgaagctaaagacgttgcacacattaccaatgagcaaatccgaacggccaaccgtgcctatgacattgtggtgccggcgcgctttgtcagtaacccaccgacatggaaaagctatcttctgaccgggctgatggcgcagcggatcgaattgcctgaaccggcggcaatgccgaaggacgggaaacaacgtgatatctggaaaaaagctgtcgcgctgggctggtctgacggtcgccaaaaagctgatgagatattcactgctaatttcaatcgcctcacacgggattacaccggcatgttgcgctacagcacgctactgcagcagggcatgattaaggctccagtcatcacacagcagcagcaaacagttacaggggataaaaaccgcctcatgctgggcgataaaacgaaacgtatgaagcagcaagctgagtttgacatcaacaaacgcagctggaaaccgaccatacgttaaggggcagtagatggaagcattcaattttggtccaacgctgacgccccagacgctggaacgttttctcgtacactgctcaaacaatgaagtgtccgatattctgttgcagggcggagataaaatctgggtagaacgtcatgggcgccagttgcccatgacatcttacccgctcgacaacctggagctggaacgtaccgttgataaggtgttcggtcaggagatcaaagggacgctgaaaagtgctgcggtcgttaataccgctatccagctaagaggtgatgaagcgggcacgattggactcggccgcggcgaaacccgccggttccgcgcgaactttactcaggccgatatttataaaacagaagcggcaatgtctctgacactccgtgttctgaatgaggttattcctcagcttaaacaaatgggtattgagccggacctgtttaattctctgttgccggctgccggcctcggactgatatgcggcgagacgggatcgggcaagtcaaccttgatggccagcatattccagtattgcggtgaaacgtaccaggaccggaaaatcatcacggttgaagatcccatcgagtatcgcctgggaagtcgagactggattgcgccggcgccggcacagtcacagattgggcgcgatattgattccttccacaattttatcactctctccgggctccgtcgttctccgaagattatgggggttggtgagctccttaaccgcctgtcatttgaagctgctgtactggcaggtaaatccggccacttttgtcttggcacactgcacgtcaaaacggtcggagaagccatttcccgatcattgcagaactatccgccggaaatgcgtgaagctgccgcctttgacctgctgagtatcctttcttacatcatcgtgcaacgcttgcttaaaacaaaagacggccggcgcaaggctgtccgtgaatatctcgtcattgataacagcctgcgccgcaaactctatgacgttgattacagcaagtggggccgctacatcgacgacctgttgatcgctgaaaaccgcagtctgattcagaatgcctgggcaatgcaccaggaaggtttgatcgacgaagcggaagtcatcgaggtcgcaggatatatggactatctggcgttaaaagagggcaagtatggacattaacaaatggcgttatgccggccggccactgacggtttttggtgtgcctgttatctcgtttcttgtgtacttcatctggtttccgttcccgtcagtaaaaacctttgttatctgcacctgtgtggttctcttttactttctgctggccatgatgggctacacgttaccggtgctgtaccaggtcattctccgggttatccgcgggaaaaaacttaccgggcggccgtggtggtaccggcgctctcagcgataacacataactacaggggtagacagtatggcgaaacctgaaattgcaactgaaagaacccgtctgagtattccctttgacgatcgccaacgtgcaatacgtgcggccggcaaactggctgatggcagtaatgctctggactacgtgaaggaagaaaaggtgtggtatgcacagccgggggccaacctgagccggctgaaagagtggatttttgaccccaataaggtgatcgaagaaccgcccctggatattcaagccattgaagatgaatttacggcgtggcttgaagagcgcggtgccattatcaaagaccccatagagttcgatggccagaagcattatgtcgatacagtggacggtaaagctggttcaagaaagggggtgtatgccgcctttctggatggccgacctgcagggtggtacagggactataagaacggtggtgaaattcagaaatgggtatctaccggcgcagcccctaaccctgaacaaatggccatgctccgtgctgatgctgcggcaaggcgtgaacaaagagctgcagcgcaagcggataagtttgatcagaccgctaaccggttgatggaagaatataaccggcttccggatgccacgggagatctggcatacctgaaaaacaaacaggtcattgccgctaacggccttaaagctgatgagcggggcaacgttgtgatccctctgtttaatgctgatggcgagttcaggacccttgagcgcatctggtccgacggtagtaaacaccttgaaaaagacggccaggcatgggggagttttttcgttgtgggtggccaactgaaggatggtgacaacctgctttatgccgagggatatgcgacagcagccagtatcagcgaagccatcaatcagcctgtgatcatgacggttaatgccggcaacatggtggaagttgctggccgcctgaaagacacctttccaaacagcactcattacttccttgctgacaatgacatttataaagacgagaacgttgggctagaaaaagcgacagaagctgcagagctgactgccggccatgttctggtccctgcctttagcaacccgaaagaagggcttactgactacaacgatttgcatgtctctgaagggctggagcaagtcagattgcaggtagaaggagccattaatcaaatgaacagggtagacactatgccgactgataacccgaacattactgacgttaatcacagctcgactgacagcgctgccgttgcagctcctgaaaaagccgcgccggtggccagcacccctgcagctgctgaaccggtggaagccgcgccggtggccagcacccctgcagctgctgaagcggtggaagccgcgccggtggccagcgcccctgcagctgctgaagcggtggaagccgcgccggtggccagcgcccctgcagctgctgaaccggtggaaaccgcgccggtggcaagcacccctgaagctgctgaaccggtggaagccgcgccggtggccagcgcccctgcagctgccgaaccggaatacgtcgcaccaacagatcgagacggcgatgatgaagagagtaatagcgccagtgatcttgaggcgtatgccgctatgatggcctttggcggtgaaacaactgacactgctcagcaaaagcctgaagggatagcgcccagcgaacctgtagctaataccgccccggctgaaaatgaaaccgaggagaagaaaaaaacaaacgaagaactggaaaacggcatacgctgggcaaccaacgataatgcagaaacgccaccaaaagaacgtatagacctggaagggttaattgcccggtttgggtacagagcagagaaagactatacggcgtatacgcttgatgggcaggatgcgttttacgattacggttcacatctgcgtatggctacccctgaagccagccagagtgacgagatgatcctggcggccgttcttaccgcggataagcaacatcatcgtggtattgaaatcaccggcagtgacgaatttaaagagcgagtgtttaaccttatcagtgaatacaacattgaggtgaaactgactaacccagaacaaagggttaaattgctcgaactcaaaaaagcaaatgaaacagcaaaagaaacggcaaaagaaaatgttgctgattcaaatgcgaataagcaggaaggggcaaacgaaaaggcaaattcatcagttcagcagaatggaatgcgctgggaagaatcggcctctgtacagccgaagaagcagaatgcatctgatactcaaaaggaagataaagctgcgggtgaatcctgggagaaaggcgttatcgtcgcgcatggacgtgctaagtatgagtggaaagatgatgaatccatgaactactttgtcaccctcaaaaatgaacatggagagaagacactctgggggaaagaccttgagagagcggttaaagatgctggtgtagacaccgaacgtcttgttacagtcagaaagctgggatttgtggatgttgaggtgaatgcacctgtccgtgatgaaagcaacaagatcgttcgttatgaaactatccagaccaaacgtaatgagtgggaagtaaaacctgtatacccacagccaacagcagggaatgaacaggcctataagccttctgaactggttccgtatgatgctgcaaccttccagaagctccgcacaacaattcagcagattaccggtgttgatctcagccgtgaggccgtgcctgaaaaagaactgtattggttcctgccgaatggtaagccagctcctgaaagcatgacaaaacccacaggctataaactccctcaagagtcaaaaaccgcgggtacacctttgctggtcgcaccgcataaaaaaggcgaacgcccggattattaccttgttgagtctcgtaaaggattcatgcaggggatcgcaaaggataaggaatctggcgagtaccatagcgtaattggtaaagtgaacaagcgtattgataaagatggaaactggaaaacgtatatgacactttcagcaatcaataaaaatgcagaaagtggaattgttttgcacggatacggccatgttgatcagggaggtaaaaacctgctttacaaaaacacaattgcaaatgaaaaacagcctcagaatttacaaccggcctcagatgaggtaaaaaacaaaacagtaatgaagcagctttttcatccttctaatgctgcaaaaagaaaagatgatgaaaggagagaacagactcatgcaccagtcgcaaagtcagcgcctcggccataaaacaaaaaccctactcgtagcagtagggttttcctgcatttctgtgagctcatttgcagccacacagacctgtgcagcatacgaactggcaaaagctggtgctgatgctgcctataaacaggaggttgaacggattaacaacgccgctaaaagtgaatccagtacatctgattctctcggacagtgcctggggtcgctatcggtatcactaaaaatccctcaatttccgtctatcagcgacatcattgataaggtgaaggatgaggtatgcaatacggttaaatcgaagataaatgagaagtgggggagcctgactaatacaacgctggacccgtggtcaacgatttcatcccgactgccagatacgtcaggtatcctgcccaacgcatcttctaagaatgtgcaattaaacaatccggcagctgcgagtggaagtggtgaggatgcaaacagtgatgcattcccgttccatttgtaactaccggaagagtcctttcgggattttatcgaatagcttgccggttcgtatttcaacgcggcggtgttccgccgcggttagtaaacgcataaattctgtggcaggtagaacatcacgttttgagtctacatacgtatcaagcgaatcttcattcatgaaacgtctgacagtatgcagccagtcttcagctgatttcatattttttgagctcatcgtcgctcatctcggcaaacgctgcaggtcggcgaccttttcctgtccaggttatggactttccatcaacttcgatccggtacttcacttttccaaaagtggatttacttttcgtggacggaccaaagagattttcgaagatctggagagcttcatcagccgtaatattgtgttcttccatgaaccgctttgtggctgcggctttctcttcagtggccttctgttcttccttaaatttctcgatcattccctgtgcaatgtcggaccacatattaatgagttgttcaaggttttctggcgggattttgcgcaaaacggcggtggcttttcgtcgttcgcacatgataactgcacaggattcgtaatcattatgttcgctcatgtatacctcattgttcattaacgatctgcgtgattttctaacacaaacttttgaaaagagaaactctatgaaaacacaaaatggtgaaaatattatggatcaaactgactcggatcaaaactcagtcgttgatgatgacagcaacgcccttgctccagctctggctgaagagagtaaccgaacggtacaagaaatcgtatcgaaacatggtttaaaagtaattttgattcaaacgctaatcatattagtcgcaatcattatcattggtcttcagggttactggcttcaccagaaaaaaatcaaatatattgcaacgaatgggggcatgatcacagaagttcatccaaccgatgaacccgcttacacggcggaagaggtgatggactttgccaaccgtacaatgatcaaatcgctttcactgaactttgtgaattacgaaaaccaattaaccagcgtccgcggcgattttagcgctgaagggtacgttagtttccgtaaagcgctgtccgatagtgggctgctgaaagatatcagtgaaaaacgtctgaacgtaaaactttccacaactcctggaaccctgatcacggaaggtcttatccgtggcaccaaaacctatgcctggcaataccgcattccggtgactgttcagcttgttgggcaggctcaggactacagaccgcaaccttacgatttgatggttcaggtacgccgcgtcaaagaagacgaagatccacggggtattcaagtagcacaggccattctcaaaccccggaactactgaggtcaatcatgaagattaaactgttaactgtacttatggccgcactgttttcggcaaattctgtatctgcagctgatgcgccgactggtaatggagcgccgcagcaaacggcgccagctgtgcctggtggtgatattgctcaatggcagcaggccagttccgggttgcccgtaaatcagcagtcacaggtgcaacagcctccacagcaaacagcacaggaacaaggtcagcagcaggcgcaaggccaacaaccacaggcacaggcccaacagcggccaacgactgcactgcctcctgcacccatgccgcagattcctccaccacagcagcaggttactcatgttaatccgcagacactggaagagttacctgcacctgttctgcagcagatccagcctattacccctggtcaggtgcggtcggcccgcagcgcaatggaagatatgagcgcggcggccaaaacctcaccattaacgccgatcccacgaatttcaagccagacggtatcactgtcagctggcgcatccattccgcaggtacgggtatttccaaaccaggcaacgacagtgacgttttcagatgcaaccggacacccctgggtattaggtgcccctccatataattcgagtccctgtgcttcaggtggtgaactgtgtgttggctatattccaggatcggcggtattcacaatccagccgaccaatgcctatgccagcgggaacatcacagtgcttctgaaggggctggccacgccggtcattattaacgtgaagggggcagaaccctccgttaaatcaaaaaccgttgatgttgattatcgtctggatctccgtatcccgaagcgcagtcctgacacaccggtttataccgctacgcctgagaaaaaaatctcactgtatgacaagcagctgcagagcgtgctcgatggcatcccgcctgaagatgctcagcggttgaaacttaaaaacgcgccagggagtaccaaagtctggcagatcggtgatgagctctacgtcagaagtaacatgcagctgcaggatgagtttgaaaaaacgctttccgctatcgatggcacacatgtttacgtgctgccggcaacaccagtgctgactttttctgttaatggtcaggcgcgtgacgttgaagttgagctgaattaaggggaacgacgatggctacagataaaggtaaagaaaccagaaaaatgatgctctacgtcggtggtgctgctgcagtggtggcgcttctgggagggtactggttgtttggctcatcagatgagccggcaacgggcgggtcgcaggtgaaagcccaggggctggaagggaaaactggcagggaaaaagctaatgatccccaatacaacaagctgctgaatgagtggaatgttgataattcagataaagcggctctcacaggggatagcttcatctccacgttgtcagccagtaatccggtggccgttgattacggcaccaaagcaccgccaccaaaatattcgtgggaatcgtcagcaaagccgtcgggcgataacagtaaggcgaatgatgctgagcagaaagagcgtgagcgtcaggctaaagcggtggctacgttactgcagaaaatagaccaggccagaacaccagttgtcacgggtgttgctcttgcaacccctctgggagcagaaggaaatgacaagaacggcggtaccttctcagggtggaccgattcggtctatccgcagaacaaaaagccggatgctgaaaaacaacagggggcgaagaataaggccaaagataaaggagcccggcttgtatcggcttacacgcttgtgccggcagtaatggatacggcgatggacagtgacgatcagaacagtattgctattgcccatgttccaactggagcgcaggccggcgccaaattctattctggccaaaaccgtcttgccggcgatggtattcgaattcacttcaccggcatggagctgaacggagtgcagtgtaaggtcgatgcgtatggcgtggcttccgactcgttgcgcgcggccgtttcctcaaatgtaaataaccgctggttcagccgtattattctgccggctgtcgctaacggattaggccgaactggtcagctctatgcggacagcaattcgcagatgattatcactgacggcgggaacgcatatcgttcaactgatacacctgatggcaaagcggtagccggtaccattattggcgggatgggtgagcaagcagggcgtgtcctggccgacgatgcagcccgcctgccgattaagcaggttacggttgaccgtaaccaactcataggcatccagtttgtcgcgccagtgtatgaaagcgactgtggcgagaatttggaaaatgcttctgcagagcaggcgcagcagcaagcaactccgacactatctagtgtgcaacctcagcagatgcctcagcctccggctcctaattaccctcagcaaaactttccaagctaccccggctacgggtatggcaccaataacccgtattaccgttaaggagattgataatgaatctgtttgaagatgacaataaaaaagaagcagctcctgcccaggaaccggtgtctgctaagactgctgaaaagccaaaaccgacagtgacgccagcagcaaaagcaaacaaaaagccgcttaagattagtgactatgcatggattggtggtatcgctgtcatagctatcctgcttttagtctggttgtttgggggctccgatagtggcactgcgcctgcggccgggaatggcacgatccagtcttcagggcaacgtcagaacgtacaggctgcccctgccgggcaggattctggtgacttccgcgagcagatttccgggattttgctgcagcaaaaggaacggctggacgcgatagaatcaacttctaaaaatggaatgatgatcctttccagtcagttacaaagcgctaatgagcaaatcaaaaatctgaacaatcaggttcaggaactgaaaatgaaagccgctggttatgggccttcatttagtaatcagagtggaacaatttcgggggcaacgtcgcaggcattacctttgttgagaggtttttcaattaatgatttatctggcgatcttgcttgggtaaaatacaaaaaccagacgtatgctgtgaaggtcggtagtcaacttggcggagtcaccattacgggtatcgatactgaaaatagaatcgttactacaagtaaaggccttatccgctaaacaggacctaaaaaggcgaaactatggatttagaatcggtattaattcaggttgctaacggtggtcaggaaccgctgacaaggctaatcataggtgtcgctgcagtcgttgggatcattggtgttattggatatctgatgagcctgcggaagcaggccaggcattcaacaaaaggtgtggaagttggcagaatttttgccggtatcatcttctgtgtatgcctcatcacgttgaaggcacagatgaactcaggcggtgccactttaggatttggcgatgttagcttcggacccgtcagttatgcgagcgaatctacgtttggcatgggagccgcagcagtcaacgcagtgctggggattgttagaattctcggcgcatactttttttacaggggtataaaggggttgaaagactcttaccttgagggccatacggagctgtcagcttcgggtacgagaggggcatccattgtgaaaattgtatgtggactacttctgatgttcgatacacaaacgctcgatgcgttgcagagcacattaaacattcactggtaatcaaaaaggatactttatgaaaattgttaagcttatcaaagacaaggcattatctgcatccatcgcagcgtatcagctgcgcggaacggtggcaaaatcgttagtggtattgccgctggcattactgagcaaagaggctgcggctgaaacgactgtctgggagaaaatcaataccttcacggaaggtcttgccagtacccagcctggtctgattaccggcggtaaagtcattggtgttgtgttctttattgtcgggcttgtttctcttttccttaaaaacaaacgcgggaaagatatcagtggcagcttcatcttctggtcaatggttgttggtgcttgcctggtcggcctgaccgcatggatctcttctgttggttcaactgttggcgttgacgcaaccctgtaatgtcccttcggccccgccttgcggggccaatatcggagttccctcatgaaagattttaagctcaccaggaatgaacaacctgccagatgccatgtgtgcgggataaagagcaaagatgttttctccctcgcagaagatggacagaaagagagaatgctttgctctgtatgcctgtcgctcgaacatccggagaaaatgaaactagagactcaaaccggacttctgctgaagagaaatacaaaccgaaaactctacagcctgctgtttcgttcaattgcaaaggcggaaaagtcaacggacgattcttaccgtgaaaaggcgcagaaacttaaacagatattgctcagcacaactgacgtagttctaaacgaaaccggcagtgatgatggatacgatttactgcacctgataaaaaaacaccatcagttacttcccatgaattttgacgatgagtttgtcgcttctgttgcagagtcagacgaattgccggatgtaagtgactggaatgaaatttacaagggtaagagaggttttaaatgatttctttctttgaggatgtgatcgaaggtatctcacgcaatctgacatcaaatgactcgttaaaatactgtgatttgacaacggttgtagggcttacgtcgcaggatcgcgttgagcatcctgaaatcagggctccgtacattctgaccactaagaacaacgattttcttaccgttattgaggttcagggcgctaaatcatcgtttgacgagaagtcgtttaatgacttcatcatgcacctatcaaactccttgtcgaattcctttattaacagcggccataagttgtctgtggtgttcgaacgcgactttaccaagaaccaggaagagctgaataatgtttataaaccgcaggtagcagcaatagagcgtctgcagatagacattaaagacctgatcgccgatgacgcaaaaaaaatcgccgctcattacagccgtgaacgcgcttacatcgtgctgtatacctcgaaggggatgattgcgaaggatgaacttaaaaacgaacaggcaaaagctgccggcgtactgaaggatatcccgcagacgcgcttcagtcagaacccaatcgaatttcagcttgaaggtttgaagatcacgcatgacgcgatgctgtggcgcctgattggtatgcttcagggggatgatgaatcaggaatggggcttctggttgatgtcctcgatgtcaaacacgctggcgcaatgatgcgtcagatgctgttccggaattcaacatcagaaaactggtaccctcgcacgccgtttgaccaaaatctgcgaatttatggcgccccacggaaagacaatatcgactccgtgctgccgccgcggctgaatatccagatcatggaaggggaactggaagaagatggtggcctgatttactgtgatggtaagtattacggctctgttgcccttgagttaccaccacttcaccctgtcaaatttaagcagttattcaacgcgattaaccggaaaatcccgtaccggataaaatatgactttataggcgggggcaaaaaagcactgttctggccatcagtaatcatatctttccttcgtttcattccctcactcggtaatatcgcgcgtgatatggattatgtccgggcccagagcgaaaccgatccaacggctatcatggccatcaactttgccacctggggcgacactaaggatgaagccaaacgcaacctggcggctttaactaaggcgattgaaggctggggtgtatcaggtgtgtccaagacatatggcaatccaggtagcgcactgattgcatccgttccggggctgacgacggccacacctggctcattgcattatccgccgttgagtgaaggcttgcggatgttaccttttgaacgtccggccagtccctggcatggtaaaggcaatatcaacttcatcacgctcgatggcaagctgttcccctatcaaatcgccagtcccctgcaggagaaatttaccgatgtcatcaccggtgttcctggttccggtaaaagcgtgctggccaaccgccttaacctgagttcgatttaccgggctgataaaaaactgccttatctgaccatcattgataagggctacagtgcaaaaggtatcgccgatctgattagcgctgagctgccgtcagagaaacgtcaccagattgccagtatcaccctgaacaatgacgaatcgcactgtattaacatctgcgatacgcagttaggttcaaggtatctgacgcagtttgaggaagtattcttaaagcagatgcttaacgcattctgtattgatccggcaattggccagccgccagatgcaatgagcgttggccaaatcgtcagtaaggttgtatcaaacgtatacaaagcaaaagcgcattcaagtgctaacttgtttaagtataacgacccggtgattaatgaagctctgaaagaatcaggtctggataaagagcttggtgaagactggtttgaaagagctacatggtgggaagtcgttgataagctgtttgccaaaaaatatcttcatgctgcgacggttgctcagcgttatgctgtgccaaccattcacgattttattgctgaactacaaactgaatcatttaaaaaccaatacgctgacgtaaaagttaatgggagtgagcctgtagtaggcttcgttgcacgttgcttccaggctgctgcagctgagtatgccattttctcagggataacggtttatgatttcagcccggagactcgcattgcgatcctggacatgcaaaacgtcctcggtgacagaacgacacctgcaggtaagctcaagtccggcattatgtatctttttgcgcgccagatggctgtgcgtaactactacctgcctcagtccgcagaaaccttcattcctgctttaccggagcagtacagggcatatcaccaggcgcgcatcagggagctttcagaagaggttaaacataccttttatgatgagtgtcataactttgccggcatcgactttattcaaaatgcgctgaacaccgctgaccttgaagatcgtaaattcaacgtccgtactgccttctcgtctcagtatctgagccatatgccttcgagtgtgcttaaaaccatgaacagcctgtttatgatgcgcctgacggaaggggatgaagagtatctgaagcaattagagatcaacatcccggccgatatcctgcgtcgtttcagacagcttccgcaaggggtttatccggacggaagtggtactgcattcctcggtatttttaaaacaaaacgtggtcttatttgccacattctgaaaaacacattaggaccaaaactgctttgggctttgaactcatctgcaaaagatagagcgctcagagatgtgctttatgaagagctgggaacgaaaaaagcaagggaagaattagcaaatagattccctatgggaagtgcatcaagtatcattgatgaaatggtggtcaatcagggtggtgagcgtgattctgaagaagaatcacaaactatggcgatgaagctcgctaaagaaatcatttctgatgtgagaaggggattcagatgataaaaatcggaaaagcaaaacggtcagtgatggccgtttcaattctggccttcagcgtacagtcagcatttgcctatacggtcaacgttaacagcagcattcccatctcaacgcaggttatgccggcgttgagcacagctaatggcctgttaggtaatatccaggccacgctgattaacattggtacggcaatcagccagcagggcgatcgccaggcagcattaatgcagcaggtggctgaaactaaccgacagttcgaagtcgagcaaaaacgtaatgaccggctgctgcaggcacaggataagtaccgtgttccggatgatatctgtgcgcagtctattggcggtggcgccgcgggggtatcctctgcggccgctggcggtcagcgaacgctgggaagcgccagttctgcgaaagatgttaaggctatttttgaagtgccggcacgcgcaaccgacgtggatgcatcttcaacggccgaagttcacagccagtactgcgacagttcggattatgctgcatttggtgggacttcattctgcccaagtgtatctgacatgccgcgggcagatacgtcgctgagctcgttgctttatggcgccggcaaagaaggcaaggcacctgatctgacgtttacagatgagcagacagatgcggcgctgcgctatatgaaaaataccgcgttgcgcagtgctggtcgtcagctgagtcgtggggaagttaaaggcgccagcgggcgaatttacctgggcatgattcagcaataccaggcgctcaccgacgcggccgcacaacctcagatggaaatgatcgccaactccaaaccgaacccggccactgatgccgcgcttgcagatgcgcgcaaagtgccggaagtcgaatcctatttccaggcgacggccagtaacagggcgaaacagcttaaccgtatgtctgccagagagtttgaggaattcgaagtaggtcgccgttacagcaaccctgcttaccaggcgacgctgaccaacaagaacaccgaagatttgatgcgtgagcaaatcaatatcgccaatctgaacaactggttagtgctgaaaaccaaacagcagctggagaaacaaaatgtgctgcttggccagatcctggcgaacgacgcatatcaaacatacaaaccgatgctggcagcacagctggagagtgtgaacgcgggagtatccagaaaatgaccgacgaaaataaaacaggggataaagacacagccaaatcagggaaattaaaaaaaggccttgatgttgtaacaggtgtcaatgacctcccggaagggaaggctaaacgaacgatatattatattactggcatcagcgatatatattttattatcgcgtcagtaaagcaaacattcagcctgctctttcagcgggcttcattcgttaagaagcaaataaaaaaccttgatggtccacctgttgattctgatgcaaatcagccgttcgccgaagttatgaaacggagcaatagacctgtatcagaattgttggataaggccagtttatacaagaaatactggctttgttgtttttttgcattagtcctcattctgctttttttaacgtctggttatgccagattgcttctaaatggctcaccgaacatgagcttgttaagagcaactctcacctgtggcgtcctgtttgctgctgggatctttactttcattaaggctctcacatgtgagttcatggggtggcagttacgaaaccaggctcactctgatgcagagcaaggtacgttacgttatttccttaatgatgggggagtgcgtaatacatttaatttttcgcaggctggtcaagagcgggggccacatgagtaaattaaaaaggttgcttctatctcttttcgttgtggcgtcgccttcatattcagcggatttggggattgatggcataactgaagcggctaaacgttcaggtgacctatcgcgtcaaatgttacataccgtttttggtgaggtagttaataatccgtttagcccttccggcgatggcttacttaataacgttttcttcactgtcaacgaagttatcgcgattcttgcccttgtctatatggggataatcagcattaagaaacttcaccaggcaggtcagttgggaagtttcattgagggtgatggcaataacgcattcaaaattgttaaaacgacttttgggtggttgttgcttgtacctaccgttgtcggttggtctgtggcgcagctgctttttctatggtgcggttcaatcattggcgtaggatctgccaatgttattgcagataaaacggcttcggaattagcgagtggtaaagctgtctatatagcccctgttatgccagaaatggcgtctgttgcaaaagggatgtttgaaacaaacctttgtgctttgggtgtaaatcaggggattgctcaaatggaggcttctggccaacattatgagaataacgccagaatgcaataccaaacaggtgacagcacatattctatatctgtaaacaacggctcagctgtatgtgggaccgtttcgctgccacaacgtccagcaggatggacttctatcttcgctagcggttatgcggattccgtctacagcgttcagcaacaagcaacggacacgttgtggggaaaaatgaagactgctgcagagcagtttaatgcagcatatatggcaaaaatgcgttccggggacggtgagctgccagatgttgaaagcgcgatccagactgctgcgcgtgattaccagcttcaggttcaacatgctgctaattcagcagccggtgacgacgaaattgttcaaaaaatggagtcggatatcaaagagaaagggtggctatatcttggtgtgtactatcacacattagctactgcgaataccgagatgaaagatgttgctaacctgaagccggtagtttcaggaatgagtaacgatggcgatatcggctctatcgattactttaaaggcttgttccaggcttaccattctcaactgaaaaatagtaattatacgccgcctcttggcaccgaatctaacgcacttgcaaggaacattgatcaaaaaagccttgaaaatgccgccacaactggagacggaacgtcccttattacaaaaattttcgacttcaacgttacaaactggcttgcaacgagtaactacggcactggtgatggttacagtgacgtgaccaaccctttactcaaaatgaaagctatcggcgattacacgttaggtgtagctgaggttggactcgccagttggaccgctatcaatgttgcagtgaaggtttcagaagggaagagtctacccggttttgctgcgggaatgatgaacaaacttacagggattcgagatgctattgctggcgtattagaatcggtttcgttcttggtttacccgctgttttatagcctctttggtatcggtcttgcactatctatctggctgccgtttataccgatgatttattggtttgttgctatggcagattggctggttacgcttatgacaggtgtattggcgtcttccctgtgggcagctacccatatcaacattggccagagtaatgatgagcggagtacatatggttatgtgttccttatcgatgtaatgattcggccgatgctcatggtaatggggtttatttttgcttctctggccatcgtagctttgggaacagcgttaaacatgatgtttaaattcgctatggaacaggtccagtctgactctttcactggcctgctgtcatcgatcggatttttgatgttatatgcaaggctttgcacaggtatggtggctcgcgtatttgcacttcctgcgaggatgccgaactatgttataaactggattggacagaagatgaacgattcagtgttgggtgatatgcagaaccatgttcatgacatctttgctgcgttcggtcgtggagccaaaaatatgaaccgtaacaaccctaagcaatttaatcctgacgcaggtgtagataagagcaaggacggcatcaaaggagcttaaatatgaatgcgttatcaaacactgactttaagaaaattagtaacaatgcgcgtccgctccaaatatcaataaagcgtacagggatatggaaatcatctgcagaacgcataaaaatagggatgaaaactatcagcatctgaagagtaaactgacagacgctggatggttcataaacagcaattattgagtttaacccgccgaatatggcgggttttttgtgtatactcaagtggttatagtcgtatgttgtgggaggaagtagtgaacagagttactgagcgccaggaacagaagagtaacccggagttccagagagagtaccggaaccgtatccgggaaacacacgaccagttaaacctttatctccccaaagatgcaaaaagaatgttgatagagatcacaaaagttaaaaaactcactcaggctgaagtcattgaatttcttgtcaaaagttattacgacggattgaaatttgacgattctgaagagtgatcttactatgtaaaagcgggttcgcaggtattgccccgcttagaaacgcatgatatgttaagcggcgtttacataaccttacggggttattacagaatttagcacgggaagagcggcccggagatgtaccgcttggggtgtggcaggagccacaaaaagtaaaaccccctgattcatcctctcactttggccggtcagagatgaaatcagggggcgcaaactgccttgctggcagaaaggattatagcaaatgcgtaccctattgcaatacagcccggccaataagtgcaggggctggtgaatcaaaagaatccatacctacagctttctgacattgagtcagtagaaggtctttcgccggagttcatctcctggcttgaaagccagtcccctaaagaatcgcccctgcagctgcttctcgatgaaggcaccaggcccgtgcgcaaaacccgccgtcgtcgcggtacgcactcaacggcgtgtctctgcccggagcccagctggtaccgaccggacaatttcaaaaagctccctggccagcttggccatgcctacaaccgccttgttcgccgcgatcgcaaaacgggcgcgttaagtctgcgtatgcggatcagccgccacccgtacttcgtgcagctgcgcgagcaggctggccggcagcgcgacttccgcccggagcgggagaaattactcgatgcgattgtgccgctcctggtcagcaccgttgaccgggctacccatattgacactatcaacctcagtaaaatggcgtggcagctcagcgaaaaggacagcgaagggaacgtgatccggaaagtgacggtaccgcgggtttgccggctgctgcagcacatgatggaattcgggttactggcgctgcctgaaggcgtcacatgggacccgtttaaccgcaagtggttccccaagcatgtggtgctgacggaacgcctctggaagatgatcggtgttgatctcgataagctgtatgcggagcaggccgaacaggtggccgctgaagctgcaggctggataacccgtgatgaacatggccaggcggaagaaatctctgttaaagccgcgcgccggcgctggtatgaaaaaatgatgcacgccacgctggttcgccggcgtgaagccgccctgaagggtaagcgcgagaagaagctgaaagcgctggcagaacagccctatgacgaccgggcttacgccatgtccgttcatctgatacggacgctgccgaaagacgaactgcacgcgctgtcgcctgagcagtttacccagcgagtacgcagccacctgtatcagctcgatctggggctggagagggaatccggtccccctgattatcactgattttctccccctgtaattctcttaccgccgcctggcggttttcgtgctggccagccggcgcgacctgctcaaatttcctccagttttaacgtgtggccaacgtgattttttgacgttctggcgccgccggtggataaccggcagcgaaatcaccactatccacagttatccgcaaaggaggaaataaagtcacctgcaatcgtggtcaaaacaagctgcaatacactcttaacttctttaaaaagatctttattcaataaagagatcactgttattcaggctcaattttctgtggattatgggaatatgcctggcctttcagcgccgctacgcggcacgacggccagaatggtcgctatgctcccaggttagcacccggcaaatgccgggcagggtggccagcagaaacctccagtccggattcttctttgccccaccgtgcttttccccgccgagccaacggctcggcagaaaaaagcatgatggatgttgtaactatgaacacccccgtaagacggcaggggtgcgacacgcccccctgacgtcaacgctactacccacgcgcccggtgtgccgctaacgtcacaccgcccgctcgcagtagccagggggtaatgtggccacagagccatctgcagggcagtctggcaggataatcgtctgcactgacgtgcaatgcctttcaggcaatcagcaccggtgccagctgcccgttttaaaattaacagggaaaagaggatcgatttagccgcggttatccacgtcagccgctctcaggcgcgttaaacagggtttgttcaggtgcggagccaggaacgagaagtattttccacactccaggcattgaattttttccgttaattcttctttatcagccatcgtttggcctcctgattaacaacgccggcaaacgccggcgttcagggttacatctcgatcatcggaaacagatcgtcttcctggtggtaaagcacgtcgtctgtgtccgtcccatcaagccagtcagcgaacagttcaaacaggcacaccgacatttcctccggtgaaattgcgccacactgcagctcgtagaagacctcaattttttccagcaggctgttggccgctttgtcgtagccgaacagcttgatcggcgggaagtgcattgcatccgggttcgtttccgccagttcatcatccttacgttccggatagatgctgtacagcgtcgccggcgatggcggtaatacctcgataaagcggtttatccgcatcggcagttcgctggaaaacttctcattgtccggaatgtaaaacgctgcgctgcgtaccagggcatcctggaaagaaattaccccctgaccttcggtctgcttacgcagctcacgaatgttcacgcggtccttttcacggatttggtacagatccccttcccggaccgttttggaaaacgtgccgtgttcggcttcctgcccggagagctctgaataataatcctggccgacggtttcgcgcagtatttcaaaggttttccggtcttcaatcgactcaaaatatttcacgcgggtgttggcgatcagcgaatccacctcgccgttcgcgccgcgggcgagcgacgggtgatcctgggaggaaaatacgcccattttttccagtgaacgcatctgcgcactcagcgtatccataccgctggtgaaatactggcccagctcgtcataaatcagcccgtatggcgcctgattattcagcgcgtgagtgagcaggacttcctcttttttcccttccagctggtagcccaggtccttactgatggtcattcgctgcagcgtcacatataaacggccaagcgtggccgcttcaccgcgtgacagctccagcgacgggatcatgacgatgagaatgcggtcattgtggaaaatatcgtccatccggatatcgccgctgtcttccggaaaaacatggccataagtttcattgaacagggagagcgtcttcaggaactgacggctcatgtagttatgctgttcgaaggcgcgggcttcccacgtttccgggtattccgcgttggccagcaggaagcctggtgtattttccagataactttccagcgcggcgtaaccctcgctgtgccagccgtttttctttgcctcaacgtacagcgcggccattgccggcagcgacatgtttttctgaatagtgcgctgtgacaaaagcagttcgccgcgggcgcgcttataacacagggcattgatcagcgccgacatcatggcttttgccgtatcctgccactgtgccgaatctccgcccacctgggggagcatggattccatcaactggatgatgaaggtcggcggggcaacgctgaagaggttgactgagttggattgcgcggggatgctttcctgcttcaccaggttcacgaacctgtcaatactgccggtcagcaggttgaggacgtaataatcatcctcacggccgaaacggcgcgccagcgaccaggtggcgaaggccagcttgttatctgctttaccgtctgagaggcagtagccacggcaccacagcaggaagttaacgataaacccatagaaggtttcggttttacccgacccggtggtaccaaaaaataccatgtggcgcagcaggtccgtcatgttcagccacagctcgcggccgaacaggcgtccccgctcatacccgacatataaaattcccttcgctttctttcgctcccggaccttatgcgggatgcgaatgggccccagacggtattccgcctgggtttccgtggtcagggtgtcgtcgaacatttcgcagtctttcggcattcgcagcggtggccggaatttacgatccgtgaatgccaccatcagcggcggaaggatgaggatgctcagcagcatggtaaaggggagaaacagacccagcacggtcagtatggccagcaggccaaactgtacgttcggagtgaggaaccactccatgaacgaaccatctgggcggatgacttttttactgtctacagattcctgttgcatagcggctccgggttattctcctattgcctgctgcaggcgggcatcgtctttgacgacggaaaccgggatcacatcacccgaatcggcaacgatggacggggtgccgataaacccgaatttcgcaaatgccatattgttcgcgtcaaccaggcgggcgccggtggtatccggggctgttttcggatcgtcaattttcgggttcatggtggtgtatcccatgttaatcgtgtcttcccagcttttcagtcgggtatctttcccggtatcgagtacaggagcggcgcggaccatcgagcgcaggtcgccataaacggacacggggaaaatctcgacgttatagcgtttgctcagttcctgcagccggggttccagcgcgcggcagtgcgggcacatcggatcggaaaacacatacagggtgtgtttatgcccggttgaaagcgagatcgtaaagcgtccgctggtgacggcatctttcagtgacagcaagtcgttatcagccagttttttaggggcttcaggtgctgcctgaacctgcagctgctccggcgcagtgacaggctgctgcagacccggcgcgggagcatacgagtttgcctgcagtggctgtgccggctgatgcatattcatcaacgcgccgttgatggagacgataaagcccagcgcggcgactggccagaagataaagttagacagccggcgcacggcattgagtccgcgctgctgtttcagctttttacggatattgttcagcgcctcgatggcctgatattcgttttcaaagcggaacagctgcaccgcttcaccagaaaacgtattgtgcgtcaggatgtggccgctgccggcatcatggccaagtccaaaatgacaggctgacagggggaaggagtgcagggcttgttcctggtagcttccgtcagcggaaagataaagtgtgttgcgatcggtttttacggtgagtttcatacttcctcgcttagagttccaggtgatcgggacgggttgccggctgaggtttagccggcgtcggttcatcgtctgtcagtcgcagactgttgttcggggtgtcgtgcggagctgcaggcggcgtagccggctcgtcttccgcttcagcgtcagattcaaatgcaaatggcggtataacgtcttcctgccagtgagcgaccggaatggcgggatcgcggcggtcaatatggcggtaatggagcagatccagctcaaggcccagcacggccgcccgcacggttggcgggaatttcgccgccttttcttcatcgagcgtcagccggtaactttcgcactgactccaggcaataacgccacctccttcaatgaagaacttcccgcggccggcgcttgaaagtgcataccataacgtgcggtccacgcctttcagccaccggaaatttgcggatggcagctgcaggtcatcatcgaacagcgcgtagagtagcgaacgggagctgcggcgcccctttgcaaattctctgaacgcttcgcactggctggcgacggtccagagcccggcggccagcgaataatcagggaagccgtctttagtccgcaggcaggaacggttgagatcgtcgagaatggatttagccgttggccggtcgcccataaaccgcagatgtacaaacgaggctgcgagtgctttttcccagggtgccagggaaagggccgccccttcgggagccaggtttacctgtgacaggaataccttatgtgcggcttttttacggaggcggcgtttagcggtatcaatcaggttatgttcctgggcaaactcaacgggtgactgcgcgctgcgtgcttcttccggatcttcattcagcagcagctgatcgaggtggccaaaccgggcgaggatatgggctatccccggatagttttttaccatcacccagggcaggttccggatggtcagtttacgggtcagacggtagtaaggattgcgcgagataacccagacgaacaacccgatgattaaaatgaacaggggaaagagcacgctggccgtgtcgttcatcacgctgataaagtcccacagcgaaacctggtgattataccgcgccgccgacagcagcatgtcccggcgctcggtcgcattacccggtgtaatggcgtccggcagtacaccgtagatccagtaaagcaggcgacaggctccgtaaatcacataatcacgaaagccaaaccacatcactgtgatcatgatgagaagggccccgataatcatcaggccatcatcccctccccctttccgttgtggctggttttgcatcagaacctcagagaattttggtaaaccttcccgatataccaggccttacgcgccggcgtattggagtgatagcggccgacaccctgccagaaatcccccccttcgagaatgatgttgtatttgaggatgtaggcactggccattgcagaggcgcaaaagtcgtgctgcagctgctccgctctcacaccaaaatcagccgccagcttattcgcccagatggtgtttatctggaatgcgccgtaatcgtaagtaccgtttttgtttttatttttcattcccggtttcccgccttcagtcagccacaaggcgaggaatccacggagaggaatgtgataaatctgtgctgcttcaaccatgcaggttggcagcgggattgagggcggggcatcaaccataacaatctcctaatgatttctatttcgagtaaaaaatataccatcaaaaggagatttttttaaatcattttggtgcgcataattgagagggtgtcagaggaattttatttgccttcgacagagacaatgttgccctcattcgcattttcccatgtcgggatttcgccactcagcgtgcagcgaagcgtctcttttccgacggtaagccgtgcgccactgctgtagtaatgactttcatacaggcaggcggttttcttttgttccggggtcagtgtaagactggcattctgatcctttgccagcagttgtacaagatgttggtttgtctgtacctgttcacccagaatgctgctgagcaacgaatcggttttcggcgcggatacactctgagcaaaggcactgcaactgattacggatgcggtcagaatgagagcaatcttcgtgttggatttcatttcactttctccattaaaaaaatcggtaatcattttctggatcatttaaattaggtctgaaaaccggcctgagtgtcaagccgattctgtttaaaaattaac

SEQ ID NO: 4 - DNA, 22664 bp, synthetic construct
  misc_feature 1..22664: note = tra-mob region
  source 1..22664: mol_type = other DNA
cccatgcccggcctgacaggccgggagctatatcataatgtgggatgctatatgcgaaaggtgaaggttaatactgcggacctgaagcatcaggattctgacctgtagagccattttgcgtgcgctgctcttcgtcaactccagtggttttttttcggctgttgctggttcgttttttaccaactcgtccggcagtaatgtccttgatatcacgtaatgtaacatctgaaaaacccgcatcgttgaggaatttcagtatctctttatcttcataccctttcgaatctttcagcttcttaatgtcagacttaagttcatcgagtacgtgctttttctcaagcctttctgacgtcacatcaggcaagtcgtcgagaatgcttttgaattttccgatgtcctcactgctatagaatgctttcttttttgcaggcatatgccctcccaataatgtcaaaatcgaccatgcctaaccggagaaaatcggacaaggctattcaggtgattttacatgatgcccggcatgattcaattgctctgcctgccaggcccttcgctgctggcattcaaacagcgccgatggtagaattcaatgaggtatttaagtacgggacaagatgtgtttttggagtaccgccgacacgcggcggccgtccaaaaacgtcttggtcagggcaagccccgacaccccctaacgaggttagctatctgatgagccgtagcgagagcagaaaaacagacgatcgaatccaggtgagatgttccacagaagtgaaagctaaactcaccgaaaaggcccatgaagcagggcttagtcttagtcaatatcttatcaaatctgggcttggaaagcgcattcaatcgaaaggtaattacaatgcacttgccgctcttgtaaagataacagcccttcaaaaacatctgtttaacgaaggtgccggagttcatagcaaggagtattcagagattctgatcgaggtcaaaaaggccgcacagaaactgcaacaggagatggatggtgatacctaaaatcattgagggccgaagggacaaaaagtccagctttggtcagctgattaagtacatggcagacaagccgtctcaggagttgactgatacagttcagcctactcctgagacggctttagctgttaaatcggatatgttcgaagggttaaacaattacctgacccgtaagcagaaagtgatcagcacaccggtggatgttgagccaggtgtacagcgtgttgtggttggtgatgttacctgccagtacaataccttctcgctcgatggtgcagcacaggaaatgaattcagtttctcagcaaagcacacgctgtaaagatccagttatgcactatgttctttcatggcctgactatgaaaagccgaatgatgatcaggttttcgattcggtgaagtttacactggcatcgatgggcatgtccgatcaccagtatgttgctgccatccaccgggatacagataacctgcatgtgcatgttgctgttaaccgcatcaatccccagacatataaagcagcctcatcgagctttaccaaagacactcttcatcaggcgtgccgcttactggaattgaagaatggttggtcccattcaaacggtgcatatgtggttaatgaccgtcaacagattgtccggaaccctcacagcaagaaagagcggggcaactggcgctcactcgatcgcatcaacaagatggagaataaagagggggttgaaacgctctatcgctatatcgttggtgatgaacaggtaggcggtagtcgtcaaaacttgatccatgtttcagctgggcttcgtgaagccaaaagctgggatgatgtgcataagacatttgcagacatcggtcttcgtgtcgaaaaagcacaggggaaaaagggctacgtcattacccacgaacatcaaaaccagaagacggctgttaaggcgagtctggtatttaacaaggcgcaatataccctgaagtcgatggaagagcgctttggtgaataccagccttcacatattgaaccggccaaagtatcggtatttaaaaccgcatatacccctggtgcttatcgtcgagatgccaataaacgtctgcagcgtaaaattgagcgcgcagaagaaagaatgctgctcaaggggcgctatcgcgcctaccggaacaatttaccggtatattctccggataaggatcgcatcgccgatgaataccggaaaattgcccaacatacccggttggtgaaaaataacgtccggcattctgtttcagatcctcataccagaaagctgatgtacaacctggctgagttcaaacgcctgcaggcggttgccaatctgcggcttagcttgcgtgaagaacgcaacggtttcagagcggctaacccacggctttcttaccgggagtgggtcgaacaggaagcactgaagggtgataaggcagcgctcagccaaatgaggggctttgcctattcctccaggaagaaggagaagtataaacagcagctggttgagcagatcgggttcaaccgtacctttaacgccatcaccagtcatgatcgtgatgatgtggccgtaatggcctcagcacgtcatggtgtcaagccacggcttttgaaagacggtactgttatctttgagcgcgacggtaaaccggtagcggctgatcgtggtcatattgtgctgacagaaagtaatggaatcgacaaagagaaaacggctgatcttgcaattgcattaacgattgctggtaaagccaaatctgtacgtgttgatggtgacggcgagtttaaggaactgtgctgtaatcgtatcgtcgatgcagctgtgaaccacaatcaccctgtcgcgcagggcattaccttcacagatgccgctcagcaggcgtatgctcaaaacgagaaacatcgtttgattcgtgagcaaaataatagtaaaaatgaaatgcaattcagaagtgaaagagacgacaaattcaatccgaagtaaaatggagtaagaaatgaaaatcaaaatgcaaacggtcctcgcggccgtttctgtgttagctcttgccgggtgtgcctctcaaagcgaaaaggaaaaacaaatctctgcacagcagctggtagatcaggaattgcgtgagcaagccattaagataaaactcgcacaggatgagctttaccaggctggagccattaacaggacccgttacaaattccctaccattatcaatgcaaacagtcagtatgttcgtgtgagctggcagggtgatgcttacgaattactggagcaactggctaaacagcgtggccttcaattcaccagtcaggggattaagttaccgcttccactcaatatcgatgttggtggttcgaaggttacgtttgaacagttgctgaccctgatccgacaacaaacgcagtaccgtgccaccatcaaccagaaacccggtgaactgcagctgctttacctcacaccatcgaagaaaggttcattcctcagagggacccgcccatgaaacttaaactgatcgtgcttctgacgttaccgctgcttgctgcgaacgttgcacgcgcggacgactccgatgttaaaggcctggattactacatggacccgccaagccaaagtgacgatgaggccgatgttactggcagtatgctaaacgacgcagcgcacaccattggatttcgcggcggtaaggcagaacgcgctaaagaaattcgcgccgcgctggaaaatcagcgcagcaaccttgattacatgtattcgttccagccgctgatctcttctgaaggctacctgcctccagtcattactgaagctaaagacgttgcacacattaccaatgagcaaatccgaacggccaaccgtgcctatgacattgtggtgccggcgcgctttgtcagtaacccaccgacatggaaaagctatcttctgaccgggctgatggcgcagcggatcgaattgcctgaaccggcggcaatgccgaaggacgggaaacaacgtgatatctggaaaaaagctgtcgcgctgggctggtctgacggtcgccaaaaagctgatgagatattcactgctaatttcaatcgcctcacacgggattacaccggcatgttgcgctacagcacgctactgcagcagggcatgattaaggctccagtcatcacacagcagcagcaaacagttacaggggataaaaaccgcctcatgctgggcgataaaacgaaacgtatgaagcagcaagctgagtttgacatcaacaaacgcagctggaaaccgaccatacgttaaggggcagtagatggaagcattcaattttggtccaacgctgacgccccagacgctggaacgttttctcgtacactgctcaaacaatgaagtgtccgatattctgttgcagggcggagataaaatctgggtagaacgtcatgggcgccagttgcccatgacatcttacccgctcgacaacctggagctggaacgtaccgttgataaggtgttcggtcaggagatcaaagggacgctgaaaagtgctgcggtcgttaataccgctatccagctaagaggtgatgaagcgggcacgattggactcggccgcggcgaaacccgccggttccgcgcgaactttactcaggccgatatttataaaacagaagcggcaatgtctctgacactccgtgttctgaatgaggttattcctcagcttaaacaaatgggtattgagccggacctgtttaattctctgttgccggctgccggcctcggactgatatgcggcgagacgggatcgggcaagtcaaccttgatggccagcatattccagtattgcggtgaaacgtaccaggaccggaaaatcatcacggttgaagatcccatcgagtatcgcctgggaagtcgagactggattgcgccggcgccggcacagtcacagattgggcgcgatattgattccttccacaattttatcactctctccgggctccgtcgttctccgaagattatgggggttggtgagctccttaaccgcctgtcatttgaagctgctgtactggcaggtaaatccggccacttttgtcttggcacactgcacgtcaaaacggtcggagaagccatttcccgatcattgcagaactatccgccggaaatgcgtgaagctgccgcctttgacctgctgagtatcctttcttacatcatcgtgcaacgcttgcttaaaacaaaagacggccggcgcaaggctgtccgtgaatatctcgtcattgataacagcctgcgccgcaaactctatgacgttgattacagcaagtggggccgctacatcgacgacctgttgatcgctgaaaaccgcagtctgattcagaatgcctgggcaatgcaccaggaaggtttgatcgacgaagcggaagtcatcgaggtcgcaggatatatggactatctggcgttaaaagagggcaagtatggacattaacaaatggcgttatgccggccggccactgacggtttttggtgtgcctgttatctcgtttcttgtgtacttcatctggtttccgttcccgtcagtaaaaacctttgttatctgcacctgtgtggttctcttttactttctgctggccatgatgggctacacgttaccggtgctgtaccaggtcattctccgggttatccgcgggaaaaaacttaccgggcggccgtggtggtaccggcgctctcagcgataacacataactacaggggtagacagtatggcgaaacctgaaattgcaactgaaagaacccgtctgagtattccctttgacgatcgccaacgtgcaatacgtgcggccggcaaactggctgatggcagtaatgctctggactacgtgaaggaagaaaaggtgtggtatgcacagccgggggccaacctgagccggctgaaagagtggatttttgaccccaataaggtgatcgaagaaccgcccctggatattcaagccattgaagatgaatttacggcgtggcttgaagagcgcggtgccattatcaaagaccccatagagttcgatggccagaagcattatgtcgatacagtggacggtaaagctggttcaagaaagggggtgtatgccgcctttctggatggccgacctgcagggtggtacagggactataagaacggtggtgaaattcagaaatgggtatctaccggcgcagcccctaaccctgaacaaatggccatgctccgtgctgatgctgcggcaaggcgtgaacaaagagctgcagcgcaagcggataagtttgatcagaccgctaaccggttgatggaagaatataaccggcttccggatgccacgggagatctggcatacctgaaaaacaaacaggtcattgccgctaacggccttaaagctgatgagcggggcaacgttgtgatccctctgtttaatgctgatggcgagttcaggacccttgagcgcatctggtccgacggtagtaaacaccttgaaaaagacggccaggcatgggggagttttttcgttgtgggtggccaactgaaggatggtgacaacctgctttatgccgagggatatgcgacagcagccagtatcagcgaagccatcaatcagcctgtgatcatgacggttaatgccggcaacatggtggaagttgctggccgcctgaaagacacctttccaaacagcactcattacttccttgctgacaatgacatttataaagacgagaacgttgggctagaaaaagcgacagaagctgcagagctgactgccggccatgttctggtccctgcctttagcaacccgaaagaagggcttactgactacaacgatttgcatgtctctgaagggctggagcaagtcagattgcaggtagaaggagccattaatcaaatgaacagggtagacactatgccgactgataacccgaacattactgacgttaatcacagctcgactgacagcgctgccgttgcagctcctgaaaaagccgcgccggtggccagcacccctgcagctgctgaaccggtggaagccgcgccggtggccagcacccctgcagctgctgaagcggtggaagccgcgccggtggccagcgcccctgcagctgctgaagcggtggaagccgcgccggtggccagcgcccctgcagctgctgaaccggtggaaaccgcgccggtggcaagcacccctgaagctgctgaaccggtggaagccgcgccggtggccagcgcccctgcagctgccgaaccggaatacgtcgcaccaacagatcgagacggcgatgatgaagagagtaatagcgccagtgatcttgaggcgtatgccgctatgatggcctttggcggtgaaacaactgacactgctcagcaaaagcctgaagggatagcgcccagcgaacctgtagctaataccgccccggctgaaaatgaaaccgaggagaagaaaaaaacaaacgaagaactggaaaacggcatacgctgggcaaccaacgataatgcagaaacgccaccaaaagaacgtatagacctggaagggttaattgcccggtttgggtacagagcagagaaagactatacggcgtatacgcttgatgggcaggatgcgttttacgattacggttcacatctgcgtatggctacccctgaagccagccagagtgacgagatgatcctggcggccgttcttaccgcggataagcaacatcatcgtggtattgaaatcaccggcagtgacgaatttaaagagcgagtgtttaaccttatcagtgaatacaacattgaggtgaaactgactaacccagaacaaagggttaaattgctcgaactcaaaaaagcaaatgaaacagcaaaagaaacggcaaaagaaaatgttgctgattcaaatgcgaataagcaggaaggggcaaacgaaaaggcaaattcatcagttcagcagaatggaatgcgctgggaagaatcggcctctgtacagccgaagaagcagaatgcatctgatactcaaaaggaagataaagctgcgggtgaatcctgggagaaaggcgttatcgtcgcgcatggacgtgctaagtatgagtggaaagatgatgaatccatgaactactttgtcaccctcaaaaatgaacatggagagaagacactctgggggaaagaccttgagagagcggttaaagatgctggtgtagacaccgaacgtcttgttacagtcagaaagctgggatttgtggatgttgaggtgaatgcacctgtccgtgatgaaagcaacaagatcgttcgttatgaaactatccagaccaaacgtaatgagtgggaagtaaaacctgtatacccacagccaacagcagggaatgaacaggcctataagccttctgaactggttccgtatgatgctgcaaccttccagaagctccgcacaacaattcagcagattaccggtgttgatctcagccgtgaggccgtgcctgaaaaagaactgtattggttcctgccgaatggtaagccagctcctgaaagcatgacaaaacccacaggctataaactccctcaagagtcaaaaaccgcgggtacacctttgctggtcgcaccgcataaaaaaggcgaacgcccggattattaccttgttgagtctcgtaaaggattcatgcaggggatcgcaaaggataaggaatctggcgagtaccatagcgtaattggtaaagtgaacaagcgtattgataaagatggaaactggaaaacgtatatgacactttcagcaatcaataaaaatgcagaaagtggaattgttttgcacggatacggccatgttgatcagggaggtaaaaacctgctttacaaaaacacaattgcaaatgaaaaacagcctcagaatttacaaccggcctcagatgaggtaaaaaacaaaacagtaatgaagcagctttttcatccttctaatgctgcaaaaagaaaagatgatgaaaggagagaacagactcatgcaccagtcgcaaagtcagcgcctcggccataaaacaaaaaccctactcgtagcagtagggttttcctgcatttctgtgagctcatttgcagccacacagacctgtgcagcatacgaactggcaaaagctggtgctgatgctgcctataaacaggaggttgaacggattaacaacgccgctaaaagtgaatccagtacatctgattctctcggacagtgcctggggtcgctatcggtatcactaaaaatccctcaatttccgtctatcagcgacatcattgataaggtgaaggatgaggtatgcaatacggttaaatcgaagataaatgagaagtgggggagcctgactaatacaacgctggacccgtggtcaacgatttcatcccgactgccagatacgtcaggtatcctgcccaacgcatcttctaagaatgtgcaattaaacaatccggcagctgcgagtggaagtggtgaggatgcaaacagtgatgcattcccgttccatttgtaactaccggaagagtcctttcgggattttatcgaatagcttgccggttcgtatttcaacgcggcggtgttccgccgcggttagtaaacgcataaattctgtggcaggtagaacatcacgttttgagtctacatacgtatcaagcgaatcttcattcatgaaacgtctgacagtatgcagccagtcttcagctgatttcatattttttgagctcatcgtcgctcatctcggcaaacgctgcaggtcggcgaccttttcctgtccaggttatggactttccatcaacttcgatccggtacttcacttttccaaaagtggatttacttttcgtggacggaccaaagagattttcgaagatctggagagcttcatcagccgtaatattgtgttcttccatgaaccgctttgtggctgcggctttctcttcagtggccttctgttcttccttaaatttctcgatcattccctgtgcaatgtcggaccacatattaatgagttgttcaaggttttctggcgggattttgcgcaaaacggcggtggcttttcgtcgttcgcacatgataactgcacaggattcgtaatcattatgttcgctcatgtatacctcattgttcattaacgatctgcgtgattttctaacacaaacttttgaaaagagaaactctatgaaaacacaaaatggtgaaaatattatggatcaaactgactcggatcaaaactcagtcgttgatgatgacagcaacgcccttgctccagctctggctgaagagagtaaccgaacggtacaagaaatcgtatcgaaacatggtttaaaagtaattttgattcaaacgctaatcatattagtcgcaatcattatcattggtcttcagggttactggcttcaccagaaaaaaatcaaatatattgcaacgaatgggggcatgatcacagaagttcatccaaccgatgaacccgcttacacggcggaagaggtgatggactttgccaaccgtacaatgatcaaatcgctttcactgaactttgtgaattacgaaaaccaattaaccagcgtccgcggcgattttagcgctgaagggtacgttagtttccgtaaagcgctgtccgatagtgggctgctgaaagatatcagtgaaaaacgtctgaacgtaaaactttccacaactcctggaaccctgatcacggaaggtcttatccgtggcaccaaaacctatgcctggcaataccgcattccggtgactgttcagcttgttgggcaggctcaggactacagaccgcaaccttacgatttgatggttcaggtacgccgcgtcaaagaagacgaagatccacggggtattcaagtagcacaggccattctcaaaccccggaactactgaggtcaatcatgaagattaaactgttaactgtacttatggccgcactgttttcggcaaattctgtatctgcagctgatgcgccgactggtaatggagcgccgcagcaaacggcgccagctgtgcctggtggtgatattgctcaatggcagcaggccagttccgggttgcccgtaaatcagcagtcacaggtgcaacagcctccacagcaaacagcacaggaacaaggtcagcagcaggcgcaaggccaacaaccacaggcacaggcccaacagcggccaacgactgcactgcctcctgcacccatgccgcagattcctccaccacagcagcaggttactcatgttaatccgcagacactggaagagttacctgcacctgttctgcagcagatccagcctattacccctggtcaggtgcggtcggcccgcagcgcaatggaagatatgagcgcggcggccaaaacctcaccattaacgccgatcccacgaatttcaagccagacggtatcactgtcagctggcgcatccattccgcaggtacgggtatttccaaaccaggcaacgacagtgacgttttcagatgcaaccggacacccctgggtattaggtgcccctccatataattcgagtccctgtgcttcaggtggtgaactgtgtgttggctatattccaggatcggcggtattcacaatccagccgaccaatgcctatgccagcgggaacatcacagtgcttctgaaggggctggccacgccggtcattattaacgtgaagggggcagaaccctccgttaaatcaaaaaccgttgatgttgattatcgtctggatctccgtatcccgaagcgcagtcctgacacaccggtttataccgctacgcctgagaaaaaaatctcactgtatgacaagcagctgcagagcgtgctcgatggcatcccgcctgaagatgctcagcggttgaaacttaaaaacgcgccagggagtaccaaagtctggcagatcggtgatgagctctacgtcagaagtaacatgcagctgcaggatgagtttgaaaaaacgctttccgctatcgatggcacacatgtttacgtgctgccggcaacaccagtgctgactttttctgttaatggtcaggcgcgtgacgttgaagttgagctgaattaaggggaacgacgatggctacagataaaggtaaagaaaccagaaaaatgatgctctacgtcggtggtgctgctgcagtggtggcgcttctgggagggtactggttgtttggctcatcagatgagccggcaacgggcgggtcgcaggtgaaagcccaggggctggaagggaaaactggcagggaaaaagctaatgatccccaatacaacaagctgctgaatgagtggaatgttgataattcagataaagcggctctcacaggggatagcttcatctccacgttgtcagccagtaatccggtggccgttgattacggcaccaaagcaccgccaccaaaatattcgtgggaatcgtcagcaaagccgtcgggcgataacagtaaggcgaatgatgctgagcagaaagagcgtgagcgtcaggctaaagcggtggctacgttactgcagaaaatagaccaggccagaacaccagttgtcacgggtgttgctcttgcaacccctctgggagcagaaggaaatgacaagaacggcggtaccttctcagggtggaccgattcggtctatccgcagaacaaaaagccggatgctgaaaaacaacagggggcgaagaataaggccaaagataaaggagcccggcttgtatcggcttacacgcttgtgccggcagtaatggatacggcgatggacagtgacgatcagaacagtattgctattgcccatgttccaactggagcgcaggccggcgccaaattctattctggccaaaaccgtcttgccggcgatggtattcgaattcacttcaccggcatggagctgaacggagtgcagtgtaaggtcgatgcgtatggcgtggcttccgactcgttgcgcgcggccgtttcctcaaatgtaaataaccgctggttcagccgtattattctgccggctgtcgctaacggattaggccgaactggtcagctctatgcggacagcaattcgcagatgattatcactgacggcgggaacgcatatcgttcaactgatacacctgatggcaaagcggtagccggtaccattattggcgggatgggtgagcaagcagggcgtgtcctggccgacgatgcagcccgcctgccgattaagcaggttacggttgaccgtaaccaactcataggcatccagtttgtcgcgccagtgtatgaaagcgactgtggcgagaatttggaaaatgcttctgcagagcaggcgcagcagcaagcaactccgacactatctagtgtgcaacctcagcagatgcctcagcctccggctcctaattaccctcagcaaaactttccaagctaccccggctacgggtatggcaccaataacccgtattaccgttaaggagattgataatgaatctgtttgaagatgacaataaaaaagaagcagctcctgcccaggaaccggtgtctgctaagactgctgaaaagccaaaaccgacagtgacgccagcagcaaaagcaaacaaaaagccgcttaagattagtgactatgcatggattggtggtatcgctgtcatagctatcctgcttttagtctggttgtttgggggctccgatagtggcactgcgcctgcggccgggaatggcacgatccagtcttcagggcaacgtcagaacgtacaggctgcccctgccgggcaggattctggtgacttccgcgagcagatttccgggattttgctgcagcaaaaggaacggctggacgcgatagaatcaacttctaaaaatggaatgatgatcctttccagtcagttacaaagcgctaatgagcaaatcaaaaatctgaacaatcaggttcaggaactgaaaatgaaagccgctggttatgggccttcatttagtaatcagagtggaacaatttcgggggcaacgtcgcaggcattacctttgttgagaggtttttcaattaatgatttatctggcgatcttgcttgggtaaaatacaaaaaccagacgtatgctgtgaaggtcggtagtcaacttggcggagtcaccattacgggtatcgatactgaaaatagaatcgttactacaagtaaaggccttatccgctaaacaggacctaaaaaggcgaaactatggatttagaatcggtattaattcaggttgctaacggtggtcaggaaccgctgacaaggctaatcataggtgtcgctgcagtcgttgggatcattggtgttattggatatctgatgagcctgcggaagcaggccaggcattcaacaaaaggtgtggaagttggcagaatttttgccggtatcatcttctgtgtatgcctcatcacgttgaaggcacagatgaactcaggcggtgccactttaggatttggcgatgttagcttcggacccgtcagttatgcgagcgaatctacgtttggcatgggagccgcagcagtcaacgcagtgctggggattgttagaattctcggcgcatactttttttacaggggtataaaggggttgaaagactcttaccttgagggccatacggagctgtcagcttcgggtacgagaggggcatccattgtgaaaattgtatgtggactacttctgatgttcgatacacaaacgctcgatgcgttgcagagcacattaaacattcactggtaatcaaaaaggatactttatgaaaattgttaagcttatcaaagacaaggcattatctgcatccatcgcagcgtatcagctgcgcggaacggtggcaaaatcgttagtggtattgccgctggcattactgagcaaagaggctgcggctgaaacgactgtctgggagaaaatcaataccttcacggaaggtcttgccagtacccagcctggtctgattaccggcggtaaagtcattggtgttgtgttctttattgtcgggcttgtttctcttttccttaaaaacaaacgcgggaaagatatcagtggcagcttcatcttctggtcaatggttgttggtgcttgcctggtcggcctgaccgcatggatctcttctgttggttcaactgttggcgttgacgcaaccctgtaatgtcccttcggccccgccttgcggggccaatatcggagttccctcatgaaagattttaagctcaccaggaatgaacaacctgccagatgccatgtgtgcgggataaagagcaaagatgttttctccctcgcagaagatggacagaaagagagaatgctttgctctgtatgcctgtcgctcgaacatccggagaaaatgaaactagagactcaaaccggacttctgctgaagagaaatacaaaccgaaaactctacagcctgctgtttcgttcaattgcaaaggcggaaaagtcaacggacgattcttaccgtgaaaaggcgcagaaacttaaacagatattgctcagcacaactgacgtagttctaaacgaaaccggcagtgatgatggatacgatttactgcacctgataaaaaaacaccatcagttacttcccatgaattttgacgatgagtttgtcgcttctgttgcagagtcagacgaattgccggatgtaagtgactggaatgaaatttacaagggtaagagaggttttaaatgatttctttctttgaggatgtgatcgaaggtatctcacgcaatctgacatcaaatgactcgttaaaatactgtgatttgacaacggttgtagggcttacgtcgcaggatcgcgttgagcatcctgaaatcagggctccgtacattctgaccactaagaacaacgattttcttaccgttattgaggttcagggcgctaaatcatcgtttgacgagaagtcgtttaatgacttcatcatgcacctatcaaactccttgtcgaattcctttattaacagcggccataagttgtctgtggtgttcgaacgcgactttaccaagaaccaggaagagctgaataatgtttataaaccgcaggtagcagcaatagagcgtctgcagatagacattaaagacctgatcgccgatgacgcaaaaaaaatcgccgctcattacagccgtgaacgcgcttacatcgtgctgtatacctcgaaggggatgattgcgaaggatgaacttaaaaacgaacaggcaaaagctgccggcgtactgaaggatatcccgcagacgcgcttcagtcagaacccaatcgaatttcagcttgaaggtttgaagatcacgcatgacgcgatgctgtggcgcctgattggtatgcttcagggggatgatgaatcaggaatggggcttctggttgatgtcctcgatgtcaaacacgctggcgcaatgatgcgtcagatgctgttccggaattcaacatcagaaaactggtaccctcgcacgccgtttgaccaaaatctgcgaatttatggcgccccacggaaagacaatatcgactccgtgctgccgccgcggctgaatatccagatcatggaaggggaactggaagaagatggtggcctgatttactgtgatggtaagtattacggctctgttgcccttgagttaccaccacttcaccctgtcaaatttaagcagttattcaacgcgattaaccggaaaatcccgtaccggataaaatatgactttataggcgggggcaaaaaagcactgttctggccatcagtaatcatatctttccttcgtttcattccctcactcggtaatatcgcgcgtgatatggattatgtccgggcccagagcgaaaccgatccaacggctatcatggccatcaactttgccacctggggcgacactaaggatgaagccaaacgcaacctggcggctttaactaaggcgattgaaggctggggtgtatcaggtgtgtccaagacatatggcaatccaggtagcgcactgattgcatccgttccggggctgacgacggccacacctggctcattgcattatccgccgttgagtgaaggcttgcggatgttaccttttgaacgtccggccagtccctggcatggtaaaggcaatatcaacttcatcacgctcgatggcaagctgttcccctatcaaatcgccagtcccctgcaggagaaatttaccgatgtcatcaccggtgttcctggttccggtaaaagcgtgctggccaaccgccttaacctgagttcgatttaccgggctgataaaaaactgccttatctgaccatcattgataagggctacagtgcaaaaggtatcgccgatctgattagcgctgagctgccgtcagagaaacgtcaccagattgccagtatcaccctgaacaatgacgaatcgcactgtattaacatctgcgatacgcagttaggttcaaggtatctgacgcagtttgaggaagtattcttaaagcagatgcttaacgcattctgtattgatccggcaattggccagccgccagatgcaatgagcgttggccaaatcgtcagtaaggttgtatcaaacgtatacaaagcaaaagcgcattcaagtgctaacttgtttaagtataacgacccggtgattaatgaagctctgaaagaatcaggtctggataaagagcttggtgaagactggtttgaaagagctacatggtgggaagtcgttgataagctgtttgccaaaaaatatcttcatgctgcgacggttgctcagcgttatgctgtgccaaccattcacgattttattgctgaactacaaactgaatcatttaaaaaccaatacgctgacgtaaaagttaatgggagtgagcctgtagtaggcttcgttgcacgttgcttccaggctgctgcagctgagtatgccattttctcagggataacggtttatgatttcagcccggagactcgcattgcgatcctggacatgcaaaacgtcctcggtgacagaacgacacctgcaggtaagctcaagtccggcattatgtatctttttgcgcgccagatggctgtgcgtaactactacctgcctcagtccgcagaaaccttcattcctgctttaccggagcagtacagggcatatcaccaggcgcgcatcagggagctttcagaagaggttaaacataccttttatgatgagtgtcataactttgccggcatcgactttattcaaaatgcgctgaacaccgctgaccttgaagatcgtaaattcaacgtccgtactgccttctcgtctcagtatctgagccatatgccttcgagtgtgcttaaaaccatgaacagcctgtttatgatgcgcctgacggaaggggatgaagagtatctgaagcaattagagatcaacatcccggccgatatcctgcgtcgtttcagacagcttccgcaaggggtttatccggacggaagtggtactgcattcctcggtatttttaaaacaaaacgtggtcttatttgccacattctgaaaaacacattaggaccaaaactgctttgggctttgaactcatctgcaaaagatagagcgctcagagatgtgctttatgaagagctgggaacgaaaaaagcaagggaagaattagcaaatagattccctatgggaagtgcatcaagtatcattgatgaaatggtggtcaatcagggtggtgagcgtgattctgaagaagaatcacaaactatggcgatgaagctcgctaaagaaatcatttctgatgtgagaaggggattcagatgataaaaatcggaaaagcaaaacggtcagtgatggccgtttcaattctggccttcagcgtacagtcagcatttgcctatacggtcaacgttaacagcagcattcccatctcaacgcaggttatgccggcgttgagcacagctaatggcctgttaggtaatatccaggccacgctgattaacattggtacggcaatcagccagcagggcgatcgccaggcagcattaatgcagcaggtggctgaaactaaccgacagttcgaagtcgagcaaaaacgtaatgaccggctgctgcaggcacaggataagtaccgtgttccggatgatatctgtgcgcagtctattggcggtggcgccgcgggggtatcctctgcggccgctggcggtcagcgaacgctgggaagcgccagttctgcgaaagatgttaaggctatttttgaagtgccggcacgcgcaaccgacgtggatgcatcttcaacggccgaagttcacagccagtactgcgacagttcggattatgctgcatttggtgggacttcattctgcccaagtgtatctgacatgccgcgggcagatacgtcgctgagctcgttgctttatggcgccggcaaagaaggcaaggcacctgatctgacgtttacagatgagcagacagatgcggcgctgcgctatatgaaaaataccgcgttgcgcagtgctggtcgtcagctgagtcgtggggaagttaaaggcgccagcgggcgaatttacctgggcatgattcagcaataccaggcgctcaccgacgcggccgcacaacctcagatggaaatgatcgccaactccaaaccgaacccggccactgatgccgcgcttgcagatgcgcgcaaagtgccggaagtcgaatcctatttccaggcgacggccagtaacagggcgaaacagcttaaccgtatgtctgccagagagtttgaggaattcgaagtaggtcgccgttacagcaaccctgcttaccaggcgacgctgaccaacaagaacaccgaagatttgatgcgtgagcaaatcaatatcgccaatctgaacaactggttagtgctgaaaaccaaacagcagctggagaaacaaaatgtgctgcttggccagatcctggcgaacgacgcatatcaaacatacaaaccgatgctggcagcacagctggagagtgtgaacgcgggagtatccagaaaatgaccgacgaaaataaaacaggggataaagacacagccaaatcagggaaattaaaaaaaggccttgatgttgtaacaggtgtcaatgacctcccggaagggaaggctaaacgaacgatatattatattactggcatcagcgatatatattttattatcgcgtcagtaaagcaaacattcagcctgctctttcagcgggcttcattcgttaagaagcaaataaaaaaccttgatggtccacctgttgattctgatgcaaatcagccgttcgccgaagttatgaaacggagcaatagacctgtatcagaattgttggataaggccagtttatacaagaaatactggctttgttgtttttttgcattagtcctcattctgctttttttaacgtctggttatgccagattgcttctaaatggctcaccgaacatgagcttgttaagagcaactctcacctgtggcgtcctgtttgctgctgggatctttactttcattaaggctctcacatgtgagttcatggggtggcagttacgaaaccaggctcactctgatgcagagcaaggtacgttacgttatttccttaatgatgggggagtgcgtaatacatttaatttttcgcaggctggtcaagagcgggggccacatgagtaaattaaaaaggttgcttctatctcttttcgttgtggcgtcgccttcatattcagcggatttggggattgatggcataactgaagcggctaaacgttcaggtgacctatcgcgtcaaatgttacataccgtttttggtgaggtagttaataatccgtttagcccttccggcgatggcttacttaataacgttttcttcactgtcaacgaagttatcgcgattcttgcccttgtctatatggggataatcagcattaagaaacttcaccaggcaggtcagttgggaagtttcattgagggtgatggcaataacgcattcaaaattgttaaaacgacttttgggtggttgttgcttgtacctaccgttgtcggttggtctgtggcgcagctgctttttctatggtgcggttcaatcattggcgtaggatctgccaatgttattgcagataaaacggcttcggaattagcgagtggtaaagctgtctatatagcccctgttatgccagaaatggcgtctgttgcaaaagggatgtttgaaacaaacctttgtgctttgggtgtaaatcaggggattgctcaaatggaggcttctggccaacattatgagaataacgccagaatgcaataccaaacaggtgacagcacatattctatatctgtaaacaacggctcagctgtatgtgggaccgtttcgctgccacaacgtccagcaggatggacttctatcttcgctagcggttatgcggattccgtctacagcgttcagcaacaagcaacggacacgttgtggggaaaaatgaagactgctgcagagcagtttaatgcagcatatatggcaaaaatgcgttccggggacggtgagctgccagatgttgaaagcgcgatccagactgctgcgcgtgattaccagcttcaggttcaacatgctgctaattcagcagccggtgacgacgaaattgttcaaaaaatggagtcggatatcaaagagaaagggtggctatatcttggtgtgtactatcacacattagctactgcgaataccgagatgaaagatgttgctaacctgaagccggtagtttcaggaatgagtaacgatggcgatatcggctctatcgattactttaaaggcttgttccaggcttaccattctcaactgaaaaatagtaattatacgccgcctcttggcaccgaatctaacgcacttgcaaggaacattgatcaaaaaagccttgaaaatgccgccacaactggagacggaacgtcccttattacaaaaattttcgacttcaacgttacaaactggcttgcaacgagtaactacggcactggtgatggttacagtgacgtgaccaaccctttactcaaaatgaaagctatcggcgattacacgttaggtgtagctgaggttggactcgccagttggaccgctatcaatgttgcagtgaaggtttcagaagggaagagtctacccggttttgctgcgggaatgatgaacaaacttacagggattcgagatgctattgctggcgtattagaatcggtttcgttcttggtttacccgctgttttatagcctctttggtatcggtcttgcactatctatctggctgccgtttataccgatgatttattggtttgttgctatggcagattggctggttacgcttatgacaggtgtattggcgtcttccctgtgggcagctacccatatcaacattggccagagtaatgatgagcggagtacatatggttatgtgttccttatcgatgtaatgattcggccgatgctcatggtaatggggtttatttttgcttctctggccatcgtagctttgggaacagcgttaaacatgatgtttaaattcgctatggaacaggtccagtctgactctttcactggcctgctgtcatcgatcggatttttgatgttatatgcaaggctttgcacaggtatggtggctcgcgtatttgcacttcctgcgaggatgccgaactatgttataaactggattggacagaagatgaacgattcagtgttgggtgatatgcagaaccatgttcatgacatctttgctgcgttcggtcgtggagccaaaaatatgaaccgtaacaaccctaagcaatttaatcctgacgcaggtgtagataagagcaaggacggcatcaaaggagcttaaatatgaatgcgtt

SEQ ID NO: 5 - DNA, 5288 bp, synthetic construct
  misc_feature 1..5288: note = trb region
  source 1..5288: mol_type = other DNA
ggcaaacgccggcgttcagggttacatctcgatcatcggaaacagatcgtcttcctggtggtaaagcacgtcgtctgtgtccgtcccatcaagccagtcagcgaacagttcaaacaggcacaccgacatttcctccggtgaaattgcgccacactgcagctcgtagaagacctcaattttttccagcaggctgttggccgctttgtcgtagccgaacagcttgatcggcgggaagtgcattgcatccgggttcgtttccgccagttcatcatccttacgttccggatagatgctgtacagcgtcgccggcgatggcggtaatacctcgataaagcggtttatccgcatcggcagttcgctggaaaacttctcattgtccggaatgtaaaacgctgcgctgcgtaccagggcatcctggaaagaaattaccccctgaccttcggtctgcttacgcagctcacgaatgttcacgcggtccttttcacggatttggtacagatccccttcccggaccgttttggaaaacgtgccgtgttcggcttcctgcccggagagctctgaataataatcctggccgacggtttcgcgcagtatttcaaaggttttccggtcttcaatcgactcaaaatatttcacgcgggtgttggcgatcagcgaatccacctcgccgttcgcgccgcgggcgagcgacgggtgatcctgggaggaaaatacgcccattttttccagtgaacgcatctgcgcactcagcgtatccataccgctggtgaaatactggcccagctcgtcataaatcagcccgtatggcgcctgattattcagcgcgtgagtgagcaggacttcctcttttttcccttccagctggtagcccaggtccttactgatggtcattcgctgcagcgtcacatataaacggccaagcgtggccgcttcaccgcgtgacagctccagcgacgggatcatgacgatgagaatgcggtcattgtggaaaatatcgtccatccggatatcgccgctgtcttccggaaaaacatggccataagtttcattgaacagggagagcgtcttcaggaactgacggctcatgtagttatgctgttcgaaggcgcgggcttcccacgtttccgggtattccgcgttggccagcaggaagcctggtgtattttccagataactttccagcgcggcgtaaccctcgctgtgccagccgtttttctttgcctcaacgtacagcgcggccattgccggcagcgacatgtttttctgaatagtgcgctgtgacaaaagcagttcgccgcgggcgcgcttataacacagggcattgatcagcgccgacatcatggcttttgccgtatcctgccactgtgccgaatctccgcccacctgggggagcatggattccatcaactggatgatgaaggtcggcggggcaacgctgaagaggttgactgagttggattgcgcggggatgctttcctgcttcaccaggttcacgaacctgtcaatactgccggtcagcaggttgaggacgtaataatcatcctcacggccgaaacggcgcgccagcgaccaggtggcgaaggccagcttgttatctgctttaccgtctgagaggcagtagccacggcaccacagcaggaagttaacgataaacccatagaaggtttcggttttacccgacccggtggtaccaaaaaataccatgtggcgcagcaggtccgtcatgttcagccacagctcgcggccgaacaggcgtccccgctcatacccgacatataaaattcccttcgctttctttcgctcccggaccttatgcgggatgcgaatgggccccagacggtattccgcctgggtttccgtggtcagggtgtcgtcgaacatttcgcagtctttcggcattcgcagcggtggccggaatttacgatccgtgaatgccaccatcagcggcggaaggatgaggatgctcagcagcatggtaaaggggagaaacagacccagcacggtcagtatggccagcaggccaaactgtacgttcggagtgaggaaccactccatgaacgaaccatctgggcggatgacttttttactgtctacagattcctgttgcatagcggctccgggttattctcctattgcctgctgcaggcgggcatcgtctttgacgacggaaaccgggatcacatcacccgaatcggcaacgatggacggggtgccgataaacccgaatttcgcaaatgccatattgttcgcgtcaaccaggcgggcgccggtggtatccggggctgttttcggatcgtcaattttcgggttcatggtggtgtatcccatgttaatcgtgtcttcccagcttttcagtcgggtatctttcccggtatcgagtacaggagcggcgcggaccatcgagcgcaggtcgccataaacggacacggggaaaatctcgacgttatagcgtttgctcagttcctgcagccggggttccagcgcgcggcagtgcgggcacatcggatcggaaaacacatacagggtgtgtttatgcccggttgaaagcgagatcgtaaagcgtccgctggtgacggcatctttcagtgacagcaagtcgttatcagccagttttttaggggcttcaggtgctgcctgaacctgcagctgctccggcgcagtgacaggctgctgcagacccggcgcgggagcatacgagtttgcctgcagtggctgtgccggctgatgcatattcatcaacgcgccgttgatggagacgataaagcccagcgcggcgactggccagaagataaagttagacagccggcgcacggcattgagtccgcgctgctgtttcagctttttacggatattgttcagcgcctcgatggcctgatattcgttttcaaagcggaacagctgcaccgcttcaccagaaaacgtattgtgcgtcaggatgtggccgctgccggcatcatggccaagtccaaaatgacaggctgacagggggaaggagtgcagggcttgttcctggtagcttccgtcagcggaaagataaagtgtgttgcgatcggtttttacggtgagtttcatacttcctcgcttagagttccaggtgatcgggacgggttgccggctgaggtttagccggcgtcggttcatcgtctgtcagtcgcagactgttgttcggggtgtcgtgcggagctgcaggcggcgtagccggctcgtcttccgcttcagcgtcagattcaaatgcaaatggcggtataacgtcttcctgccagtgagcgaccggaatggcgggatcgcggcggtcaatatggcggtaatggagcagatccagctcaaggcccagcacggccgcccgcacggttggcgggaatttcgccgccttttcttcatcgagcgtcagccggtaactttcgcactgactccaggcaataacgccacctccttcaatgaagaacttcccgcggccggcgcttgaaagtgcataccataacgtgcggtccacgcctttcagccaccggaaatttgcggatggcagctgcaggtcatcatcgaacagcgcgtagagtagcgaacgggagctgcggcgcccctttgcaaattctctgaacgcttcgcactggctggcgacggtccagagcccggcggccagcgaataatcagggaagccgtctttagtccgcaggcaggaacggttgagatcgtcgagaatggatttagccgttggccggtcgcccataaaccgcagatgtacaaacgaggctgcgagtgctttttcccagggtgccagggaaagggccgccccttcgggagccaggtttacctgtgacaggaataccttatgtgcggcttttttacggaggcggcgtttagcggtatcaatcaggttatgttcctgggcaaactcaacgggtgactgcgcgctgcgtgcttcttccggatcttcattcagcagcagctgatcgaggtggccaaaccgggcgaggatatgggctatccccggatagttttttaccatcacccagggcaggttccggatggtcagtttacgggtcagacggtagtaaggattgcgcgagataacccagacgaacaacccgatgattaaaatgaacaggggaaagagcacgctggccgtgtcgttcatcacgctgataaagtcccacagcgaaacctggtgattataccgcgccgccgacagcagcatgtcccggcgctcggtcgcattacccggtgtaatggcgtccggcagtacaccgtagatccagtaaagcaggcgacaggctccgtaaatcacataatcacgaaagccaaaccacatcactgtgatcatgatgagaagggccccgataatcatcaggccatcatcccctccccctttccgttgtggctggttttgcatcagaacctcagagaattttggtaaaccttcccgatataccaggccttacgcgccggcgtattggagtgatagcggccgacaccctgccagaaatcccccccttcgagaatgatgttgtatttgaggatgtaggcactggccattgcagaggcgcaaaagtcgtgctgcagctgctccgctctcacaccaaaatcagccgccagcttattcgcccagatggtgtttatctggaatgcgccgtaatcgtaagtaccgtttttgtttttatttttcattcccggtttcccgccttcagtcagccacaaggcgaggaatccacggagaggaatgtgataaatctgtgctgcttcaaccatgcaggttggcagcgggattgagggcggggcatcaaccataacaatctcctaatgatttctatttcgagtaaaaaatataccatcaaaaggagatttttttaaatcattttggtgcgcataattgagagggtgtcagaggaattttatttgccttcgacagagacaatgttgccctcattcgcattttcccatgtcgggatttcgccactcagcgtgcagcgaagcgtctcttttccgacggtaagccgtgcgccactgctgtagtaatgactttcatacaggcaggcggttttcttttgttccggggtcagtgtaagactggcattctgatcctttgccagcagttgtacaagatgttggtttgtctgtacctgttcacccagaatgctgctgagcaacgaatcggttttcggcgcggatacactctgagcaaaggcactgcaactgattacggatgcggtcagaatgagagcaatcttcgtgttggatttcatttcactttctccattaaaaaaatcggtaatcattttctggatcattta

SEQ ID NO: 6 - DNA, 707 bp, synthetic construct
  misc_feature 1..707: note = tir sequence
  source 1..707: mol_type = other DNA
cggcctgagtgtcaagccgattctgtttaaaaattaacaggggataaaatgattcagtcacgtaataaatatctgcaatttatgctggttctgctggcagcctgggccatagcctggggggccctgtttgtcatggggcagaccgttctgctctatgggctggggaaaaatacgctgttcttcattcatggtgctgttctggtgtacctgctgtgtgtgttcctggtttaccgccgctgggtcgcaccgctaccggttgttggccagctgcgtaacgttggcgcaccgtggctggtcggtgcgatggccgtggtgtatgtcggagaatttctgctgggtaaagtgctggacctgcccgctgagccgtttatgacgaaactttttgccgataaatccatacccgacgtgatcctgacgttgctggccatctttatccttgcaccgctgaatgaggaaacgctgttccgggggatcatgctgaacgtcttccgttcacggtaccgctggacgatgtggctgggggcgctgataacgtcattgctgttcgtcgccgcgcacagccagtatcagaacctgctgaccctggcagaactgttcctggtggggttgattacatcagctgcccggatcagaagcggcggcctgctgctgccggtattgctgcatatggaagcaaccgcgctgggtttactgtttggctgaaagtt

SEQ ID NO: 7 - DNA, 703 bp, synthetic construct
  misc_feature 1..703: note = pemIK sequence
  source 1..703: mol_type = other DNA
gttaaagcctggtatgtttttttagtggatgttatatttaaatataacttttatggaggtgaacaatgcataccacccgactgaagaaggttggcggctcagttatgctgaccgttccaccggcactgctgaatgcgctgtcgctgggtacagataatgaagttggcatggtcattgataatggccggctgattgtggagccgcacagacgcccgcagtattcactggctgagctactagcgcagtgcgatccgaatgctgaaatctcggctgaagaacgtgaatggctggatgcgccggtggctggtcaggaggaaatctgacatggaaagaggggaaatctggcttgtctcgcttgaccctaccgcaggtcatgagcagcagggaacgcggccggtgctgattgtcacgccggctgcttttaaccgcgtgacccgtctgcctgttgttgtgcccgtgaccagcggaggcaattttgcccgcactgctggctttgcggtgtcgctggacggcgccggcatacgtaccaccggcgttgtgcgttgcgatcaaccccggacgatcgatatgaaagcccgcggcggcaaacgactcgaacgggtgccagaggctatcatggatgacgttcttggtcgtctggccacgattctgacttgacccattgtttcgacagagaaccatcatgatgaaatcaggaaatttg

SEQ ID NO: 8 - DNA, 1996 bp, synthetic construct
  misc_feature 1..1996: note = mucAB sequence
  source 1..1996: mol_type = other DNA
tttgtggttttttatctgcgttgtcacatgcctggtgatggcgtatttatgcagcatggttaatccatatgatgacagaactttgtcctccgcctttaccagctttctgaggccaatcggggccgggtttggtacggcgattgtccttatcctgatcactagcattgcgcgagagctgcggtgtttctgcaataaataaaagcgtgccggcccgatatttatgccggcattgcctgtttcctgaagtctgcaatatactgtatataaaaacagttattggaggctagattatggcgctggatctcgtttatccccggccttcagtggaaggcgccgcacttcctttttttgccgaacccgttgcggccggctttcccagtccggccgcaggctatgaggcaaatgaacttaacctgcatgattactgcgtgcggcgtccggccgcaacgtacttcttgcgtgtgtccggagattctatgcgtgatgcccgcattcatgatggcgatgtgctggtcgtcgatcgggcagaagagccccggcacggcagcatcgtgattgccagcatcgataatgaatttacggtcaagcagctgcagctccgcccttacccctgcctgatgccgctgaatccggcttttccgccaatcaggtttgatccggaggcgctgcagatatggggtgtggttacgttcactgttatgagtcaccgcccgtgtacgccttaatcgacgtcaattcgatgtactgcagctgcgaagaggcgttccgccctgatttgcgggggcgccctgtggtcgtcctcagcaacaacgacggggcgcttgttgcggtcaatcgcgcggcgaaagcgctgggagtgcgccgcggggaaccctatttccgctgccgtcgcctgctggaacagcataacgtcgccgtattcagttccaactacaccctttacgcctcattctccgccagatttgcaaccgtagtggaaagtctggcgccgcggacgtttgtttactcaattgatgagctctttgcggatgcgtctcacatggagggcgtgatgtcgccgcaggcgtttggccagcttttgcgggctgaagtgctgcgccagacaacgctcacctgtggagtgggcgtcgcgccaacgaagacgctggccaagctgtgcaaccacgccgcaaagacgtggcctgccaccgggggggttgtggcgctcagtgatcctgtgagactgaaaaggctgatgagcctggtgtcggccggcgatgtgtggggggttggccatcgtaccgaaaaagggctggcgacaatggggatcaaaacggcgctggcgctcgcagagatggatatccgacttgcacgccggctgtacggtgtgacgctggaacggaccatccgtgagctgcgtggcgaggcctgctttgccctggaagaaggtaccggcgcgaaacagcagcttgttgtatcccgctcgtttggatcgcgggtcacgcaactggcgcagatgcagcaggcggtgactaaatatgccacacgggcaggggaaaagttgcgccaggaaaaccgtctggcgaaggtggtaacggtctttatccgcagcagtgcttatgacgccggcggtgtgccctacagtaaccaggctacggaagtgctttccgtgccaagccaggatacccgcgacatcatcgccgcggcgcagctggggctgtcgcgtatctggcgtgagggtgtgcggtatgcaaaagccggcgtgatgctcagcgatctgcagggacatgaaacccagctggatctgtttgcgccggctgcagtgcggccgggcagcgaaaaactgatggccaccatcgacaaaataaacagggaaggaaggtcaagggtcttcttcgccggggaaggtattgatccggaatatgccatgcgccgtgaaatgttgtctccggcctacaccacgcagtggaaggatattcccgcggcgtttatgcgataacagcgatgtgcaggccag

SEQ ID NO: 9 - DNA, 654 bp, synthetic construct
  misc_feature 1..654: note = Exc sequence
  source 1..654: mol_type = other DNA
atgaatgcgttatcaaacactgactttaagaaaattagtaacaatgcgcgtcctaaaaggcttggatactacgcatcatggctctggatgatgtttgtattcctctttctgttcgttagcgctttcgtgtttctgagcggcttatactggacggttagagacggttcaattcaggagcactggtttagtacagtcggtatctttgtgatcgatgcagtgctgatctggatgttgaaaaaggacttcaggagccgcaagctctaccaagtaactgcttcaatgaagaactctggtttcttcgagcctcataaggactgcgagcactatgacctggcccggagaacttacatcggctttgacttcagtactggaatcataggagtagcatccctgtatgccacatccagtattaaacgtgagcggctattttttgaagccgaaacagtcgagtcatgggagtcagtcggcagggagttaatcataaacctgcgaaatacggggcttacgacgataacgatcacagctccaaatatcaataaagcgtacagggatatggaaatcatctgcagaacgcataaaaatagggatgaaaactatcagcatctgaagagtaaactgacagacgctggatggttcataaacagcaattattga

SEQ ID NO: 10 - DNA, 28562 bp, synthetic construct
  misc_feature 1..28562: note = MRR-transposeable elements
  source 1..28562: mol_type = other DNA
tgaagacaggggaatgttgctctttttgcagtgaaggccacagagagcgttttaggcgcttattggggtctgacgctcagtggaacgaaaactcacgttaagggattttggtcatgagattatcaaaaaggatcttcacctagatccttttaaattaaaaatgaagttttaaatcaatctaaagtatatatgagtaaacggcactgttgcaaatagtcggtggtgataaacttatcatccccttttgctgatggagctgcacatgaacccattcaaaggccggcattttcagcgtgacatcattctgtgggccgtacgctggtactgcaaatacggcatcagttaccgtgagctgcaggagatgctggctgaacgcggagtgaatgtcgatcactccacgatttaccgctgggttcagcgttatgcgcctgaaatggaaaaacggctgcgctggtactggcgtaacccttccgatctttgcccgtggcacatggatgaaacctacgtgaaggtcaatggccgctgggcgtatctgtaccgggccgtcgacagccggggccgcactgtcgatttttatatctcctcccgtcgtaacagcaaagctgcataccggtttctgggtaaaatcctcaacaacgtgaagaagtggcagatcccgcgattcatcaacacggataaagcgcccgcctatggtcgcgcgcttgctctgctcaaacgcgaaggccggtgcccgtctgacgttgaacaccgacagattaagtaccggaacaacgtgattgaatgcgatcatggcaaactgaaacggataatcggcgccacgctgggatttaaatccatgaagacggcttacgccaccatcaaaggtattgaggtgatgcgtgcactacgcaaaggccaggcctcagcattttattatggtgatcccctgggcgaaatgcgcctggtaagcagagtttttgaaatgtaaggcctttgaataagacaaaaggctgcctcatcgctaactttgcaacagtgccccctcggctaccacctccattgtcgttttcagaagacggctgcactgaacgtcagaagccgactgcactatagcagcggaggggttggatccatcaggcaacgacgggctgctgccggccatcagcggacgcagggaggactttccgcaaccggccgttcgatgcggcaccgatggccttcgcgcaggggtagtgaatccgccaggattgacttgcgctgccctacctctcactagtgaggggcggcagcgcatcaagcggtgagcgcactccggcaccgccaactttcagcacatgcgtgtaaatcatcgtcgtagagacgtcggaatggccgagcagatcctgcacggttcgaatgtcgtaaccgctgcggagcaaggccgtcgcgaacgagtggcggagggtgtgcggtgtggcgggcttcgtgatgcctgcttgttctacggcacgtttgaaggcgcgctgaaaggtctggtcatacatgtgatggcgacgcacgacaccgctccgtggatcggtcgaatgcgtgtgctgcgcaaaaacccagaaccacggccaggaatgcccggcgcgcggatacttccgctcaagggcgtcgggaagcgcaacgccgctgcggccctcggcctggtccttcagccaccatgcccgtgcacgcgacagctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccgcatgcccgttccatacagaagctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttggtaagctgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagggcagtcgccctaaaacaaagttagaaaagggaaagtatgagcaagttatctgtattctttatatttttgttttgtagcattgctaccgcagcagagcctttgccagatttaaaaattgaaaaacttgatgaaggcgtttatgttcatacttcgtttgaagaagttaacgggtggggcgttgttcctaaacatggtttggttgttcttgtagatgctgaagcttatctaattgacactccatttacggctaaagatactgaaaagttagtcacttggtttgtggaacgtggctataaaataaaaggcagtatttcctctcattttcatagtgacagcacgggcggaatagagtggcttaattctcaatccatccccacgtatgcgtctgaattaactaatgagctgcttaaaaaagacggtaaggttcaagctaaaaattcatttggcggggttaactattggctagttaaaaataaaattgaagttttttatccaggcccaggacacactccagataacctagtagtttggctgcctgaaaggaaaatattattcggtggttgttttattaaaccgtacggtctaggtaatttgggtgacgcaaatttagaagcttggccaaagtccgctaaattattaatatccaaatatggtaaggcaaaactggttgttccaagtcacagtgaagctggagacgcatcactcttgaaacttacattagagcaggcggttaaagggttaaacgaaagtaaaaaaccatcaaaactaagcaactaaatttctaacaagtcgttgcagcatcgtgcgctgcgcgcactggacagtttttaagtcgcggttttatggttttgcttcgcaaaaatattccataaaaccacaacttaaaaactgccgctgaactcggcgttagatgctttgctgtgcgcacaaatttcggccagcaacaagactgttttttttcttaaatcgaacctaaaatttattcgcggaactccatggataaatattttgaaaaattggttatttctggctacgtccattatttttgaggtcattgcaacctctgcgctcaagtctagtgagggctttactaggttagtaccgtcttttatcgtcgtagcgggatacgctgctgctttttatttcctgtcgctgacactcaaatcgattcctgttggaatcgcctacgcagtttggtcgggcctcgggatcgtcttggtcactgcgattgcatgggttttgcatggtcaaaaactagatatgtggggatttgttggtgtcggcttcattatcagcggcgttgctgtgctcaacttgctatctaaggcaagtgttcactaaaacggtcgcatctaaccattccgtcgagagggaccgcccacaagctgcgcttgcgggttcccttcgcggcttcgccgctacggcggcccctcacgtcaaacgttaggcatcacaaagtacagcatcgtgaccaacagcaacgattccgtcacactgcgcctcatgactgagcatgaccttgcgatgctctatgagtggctaaatcgatctcatatcgtcgagtggtggggcggagaagaagcacgcccgacacttgctgacgtacaggaacagtacttgccaagcgttttagcgcaagagtccgtcactccatacattgcaatgctgaatggagagccgattgggtatgcccagtcgtacgttgctcttggaagcggggacggatggtgggaagaagaaaccgatccaggagtacgcggaatagaccagtcactggcgaatgcatcacaactgggcaaaggcttgggaaccaagctggttcgagctctggttgagttgctgttcaatgatcccgaggtcaccaagatccaaacggacccgtcgccgagcaacttgcgagcgatccgatgctacgagaaagcggggtttgagaggcaaggtaccgtaaccaccccagatggtccagccgtgtacatggttcaaacacgccaggcattcgagcgaacacgcagtgatgcctaacccttccatcgagggggacgtccaagggctggcgcccttggccgcccctcatgtcaaacgttagacggcaaagtcacagaccgcgggatctcttatgaccaactactttgatagccccttcaaaggcaagctgctttctgagcaagtgaagaaccccaatatcaaagttgggcggtacagctattactctggctactatcatgggcactcattcgatgactgcgcacggtatctgtttccggaccgtgatgacgttgataagttgatcatcggtagtttctgctctatcgggagtggggcttcctttatcatggctggcaatcaggggcatcggtacgactgggcatcatctttcccgttcttttatatgcaggaagaacctgcattctcaagcgcactcgatgccttccaaaaagcaggtaatactgtcattggcaatgacgtttggatcggctctgaggcaatggtcatgcccggaatcaagatcgggcacggtgcggtgataggcagccgctcgttggtgacaaaagatgtggagccttacgctatcgttggcggcaatcccgctaagaagattaagaaacgcttcaccgatgaggaaatttcattgcttctggagatggagtggtggaattggtcactggagaagatcaaagcggcaatgcccatgctgtgctcgtctaatattgttggcctgcacaagtattggctcgagtttgccgtctaacaattcaatcaagccgatgccgcttcgcggcacggcttatttcaggcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagcttgccccttccgccgttgtcataatcggttatggcatcgcattttattttctttctctggttctgaaatccatccctgtcggtgttgcttatgcagtctggtcgggactcggcgtcgtcataattacagccattgcctggttgcttcatgggcaaaagcttgatgcgtggggctttgtaggtatggggctcataattgctgcctttttgctcgcccgatccccatcgtggaagtcgctgcggaggccgacgccatggtgacggtgttcggcattctgaatctcaccgaggactccttcttcgatgagagccggcggctagaccccgccggcgctgtcaccgcggcgatcgaaatgctgcgagtcggatcagacgtcgtggatgtcggaccggccgccagccatccggacgcgaggcctgtatcgccggccgatgagatcagacgtattgcgccgctcttagacgccctgtccgatcagatgcaccgtgtttcaatcgacagcttccaaccggaaacccagcgctatgcgctcaagcgcggcgtgggctacctgaacgatatccaaggatttcctgaccctgcgctctatcccgatattgctgaggcggactgcaggctggtggttatgcactcagcgcagcgggatggcatcgccacccgcaccggtcaccttcgacccgaagacgcgctcgacgagattgtgcggttcttcgaggcgcgggtttccgccttgcgacggagcggggtcgctgccgaccggctcatcctcgatccggggatgggatttttcttgagccccgcaccggaaacatcgctgcacgtgctgtcgaaccttcaaaagctgaagtcggcgttggggcttccgctattggtctcggtgtcgcggaaatccttcttgggcgccaccgttggccttcctgtaaaggatctgggtccagcgagccttgcggcggaacttcacgcgatcggcaatggcgctgactacgtccgcacccacgcgcctggagatctgcgaagcgcaatcaccttctcggaaaccctcgcgaaatttcgcagtcgcgacgccagagaccgagggttagatcatgcctagcattcaccttccggccgcccgctaaatatctccttttgggttgttaataaaacatccaataagttgactgtgcgtgaaaaagaaagttttgtgtgatggcgttgaagatcgcaccgttaagctcttatgtgggatggtgcagagctcgacgactaccgataaaacgcaaccgccgcaaacagacaagaaaaagccccaactgataacagttggggcttcagtattgtgattggtggagcaatagcaccctgaacccaaaaccttctcgctcaaccggtagtggctgataacaactcgtgagggctattgcgggttaagcatttagcgatgtctagggccagactggacgtctgaacgcaagccgctgatactgtacataaccacagtatcagcggaggatacccatgtcgctggcaaggaacgccacggcgagtcaatcgcccactcaaacaaacggttacgaacgccaccaacccgaccagacgctgctctaccagctggttgagcagcactacccagccttcaaagcctcactcgaagcccaaggtcaacacctgcctcgctacatccaacaagaattcaacgacctcctccaatgtggccgtctggagtatggtttcatgcgggttcgctgcgaggattgtcatcacgagcgtctggtcgccttcagctgtaaacgacgcggcttttgccctagctgcggtgcccgccggatggccgagagtgcggcgctgctgatagacgaagtcttccccaaggagcccattcgccagtgggtgctcagctttcctttccagctacgctttttgctggctcgccatccccagctgatgggccaggtcttgagtatcgtctatcgtacactctcaactcatctgatcaaaaaagccggttacaccaaagcctctgcacaaactggctcagtgactcttatccaacgctttggctccgcgctaaatctcaatgtccactaccacatgctgtttctcgatggtgtctatgccgaagatgactatggcaagcaacgcttccatcgtgtcaaggcacccacttacgatgagctgaatacgctcgctcacaccctcagccatcgcatcgctcgctgcatggaaaagcgtgggattttggagcgtgatgccgagaatacgtggttgacactggaagagggcgaagacgatacgctgactcaattacatggtgcttcggttacgtatcgcattgccgtcggcccccagcaagggcgcaaagtcttcaccctgcaaaccttgccagggcgtgaggataaagccgactcaagcagtcgagtagccaaccatgctggtttctcgctacacgccggtgtgatggccgaagcgcatcagcgggataagcttgagcgcttgtgtcgctacattagtcggccagcggtttcagaaaaacgtctggcattaaccgccaatgggcaggtgcgttacgagctcaaaactccgtaccgcaatggcaccacccatgtgatcttcgagccgctggacttcatcgccaaactcgctgcgttggtacctaagccgcgagtcaacctcacacgcttccacggcgtctttgcaccgaacagcaaacaccgagttcaagtaacacccgccaagcggggcaagaagcccgacaaatcggaaggtctcgatactaactggcgtgacaagagtcctgcagagcgccaccgcgccatgacctggatgcaacgcctcaagcgagtcttcaatattgatattgaagtctgcgaacactgcggcggtcacgtcaaagtgattgccagcatcgaagatccgaaggtcattgagcagattctcaagcatctgaaacagaaaacagccaaggcgaatgccgccaagcagcgtgagctgccaccagaacgagcgccgccactgactcccagcctgttcgatccatcacagagtcgtctctttgactgacgaccccaaatccaacactgctcaacactgccaacttttaaacggggcggtggggcagtttgtatctctcgagctatcaggctagagattttaccgccaaatcgaaccttattagagcggtttaggctggaccggcagttaaaattggggcttgagcggtaaacgagtgagggaatttcaggtaagatacttcggatgaggagcaaaaaggtggtttatacttcctataccccataaaggaggcatccggttgcgttaagcggaattcaacggtgttgttgtccagtttgcgcacgctttggacgttatcggcaaactgcaggctgtcgaagtaggggaaactgctgccgttcacattgtgccacggatggtggcgatcaaagatgcgctcgaaggtaaacaccacatcgtctgcattcagtttgcgggtgggggtgaaccaggccgtcttctgaaactgaacgtcacgacgcagatggaagcggtaggttgcgccgttatccagcacctcccagctttcagcaagctcaggcaccagccgataggtgtagggatctacatcaagcagtcggtcatacaattgggccgctaacgtatcgacgatgagaccgctgctcgttttttgtgggttgaacgtattgacttgcccgctgacgcaatagacaaacccactgtcacggatatcagcgtgcggagcttgctcaggcgcagcagcagcctgaccactcagaaatccagccatcacgatcagagatgataaaaccaggcgcataattttaatggattatataaagaggctatcttactaatacttaatgacatttgccattaccgtttgtttttgggagtaacaggctcgataagcgcgagttcaacaccgcgacatggtcaaaattcatacctgctatccactttgaatctactctgttaactatcttagcattttcatggcccatctgacttgctataaacgtgggggcgtacctgtcgttgtttactgattgatatcagtgtgtgctcggaagatggagtatggcatcgataacgaagtgtggagcgagcatttagagtgtcactatcaatgctgaactataatcattcaggaagcagtacacgttatgtgagttgtaatagctaaagtatctataattatttcaatatgttaattccattttttcttcatggcatcttaatcttctcacccatttatggtgatgatttccattggttattaaatgcattttggctttgatcctggttatgaacggttttctgttgccatttaacttctaatggaatgaattgtatgaataaattactgagccttgttgtttcacttgccctattgcctggagccgtttttgcagcgtcgactccagactgtgtgaaggtgaataaagcgcaaatcgaagcgctatttgataagtggaacgagtcattgaaaacaggtaacgcgcagacggtatctgaaaattatcttagcgatgcggtattgttgcctactgtatccaataaagcgcgtttaacggacgctgaacgtgttgattatttcgagcattttcttgcgaaaaagccgaccgggaaaattgacatgcgtaccattcgcctggggtgtaataaggcgatagacaccggtacctacacatttaccttcgcggacaaatcaacggtttcagcccgctacacattcacttatgcatgggacggtaaagagtggaaaatttccacacaccactcttcagcgatgcctgaagggtaaaaagctgtgcagggggcggaatattcaccgctccctgaagactagccaataatcgcgatgccaagacgttccatgagcaacgatgcctggtagttgtccaacttaacgccttgtaaatcaacgcgccgaatatctaagtcacccaactccgaattggtcagatcgcaatgtgtgaagtttgctgctcgccagtcgaaagtcgaaaactcgccgccggagagatctgaaccactgaacgtcgcgcccagtacctgggcccccatccaacggttttcccacagctcacacttttccaacacgactttcgaaaaattggcgtagcttagatttgtgttagtgatatatgcactgcaaaaccaggtgcgagtagtgatcatattcataaaacttgcgccgcgaaaatctgcgccttgcgcacgacagtggcgaatttcaatgccaagcgcactggcattgcgaaaatccgccatggataaatcacagcttttaaaaatggcatctttcagcatcgcacgactaaaattgcaccctttctggctttcacgatcatagaactgacagccgataaattcagtgccgctcaggtcggcacctgaaaaatcacagttaaaaaatgtactattttcaattttctcaccggtgaagcggtttctgttaattttttcgccaacgagtgccagagccatattttgtgcctgtttttttatacagtaatggcgtcatggtaaaccctgatgaggttatgcgtcaaatccgccaatataacatctgcaaatgtgcgttaaatctggtgttttttcagcaaagcgcgaagctgatggtaagtcagacccagtaattcagcggcttttttctggttaaattttgcctgctgtaagctggtttgtagaaagtctttctcttgctgctgctggaattcacgcagatccagcggtaaccctacagacatctgtttagtttccggcgcctgcggctgcgtctggttctgaaatccatccctgtcggtgttgcttatgcagtctggtcgggactcggcgtcgtcataattacagccattgcctggttgcttcatgggcaaaagcttgatgcgtggggctttgtaggtatggggctcataattgctgcctttttgctcgcccgatccccatcgtggaagtcgctgcggaggccgacgccatggtgacggtgttcggcattctgaatctcaccgaggactccttcttcgatgagagccggcggctagaccccgccggcgctgtcaccgcggcgatcgaaatgctgcgagtcggatcagacgtcgtggatgtcggaccggccgccagccatccggacgcgaggcctgtatcgccggccgatgagatcagacgtattgcgccgctcttagacgccctgtccgatcagatgcaccgtgtttcaatcgacagcttccaaccggaaacccagcgctatgcgctcaagcgcggcgtgggctacctgaacgatatccaaggatttcctgaccctgcgctctatcccgatattgctgaggcggactgcaggctggtggttatgcactcagcgcagcgggatggcatcgccacccgcaccggtcaccttcgacccgaagacgcgctcgacgagattgtgcggttcttcgaggcgcgggtttccgccttgcgacggagcggggtcgctgccgaccggctcatcctcgatccggggatgggatttttcttgagccccgcaccggaaacatcgctgcacgtgctgtcgaaccttcaaaagctgaagtcggcgttggggcttccgctattggtctcggtgtcgcggaaatccttcttgggcgccaccgttggccttcctgtaaaggatctgggtccagcgagccttgcggcggaacttcacgcgatcggcaatggcgctgactacgtccgcacccacgcgcctggagatctgcgaagcgcaatcaccttctcggaaaccctcgcgaaatttcgcagtcgcgacgccagagaccgagggttagatcatgcctagcattcaccttccggccgcccgctagcggaccctggtcaggttccgcgaaggtgggcgcagacatgctgggctcgtcaggatcaaactgcactatgaggcggcggttcataccgcgccaggggagcgaatggacagcgaggagcctccgaacgttcgggtcgcctgctcgggtgatatcgacgaggttgtgcggctgatgcacgacgctgcggcgtggatgtccgccaagggaacgcccgcctgggacgtcgcgcggatcgaccggacattcgcggagaccttcgtcctgagatccgagctcctagggatcgcctcagaaaacggaatctatggtcactcccgtttttgcaacaccgattttgacgacaagttggcttgcttgaatctatccggcgtctgaatgggattttattcccgcgcctcgatgagttccgcgcctgatgaacctccagaaaatatacggcttcaatgagcctttccgttttacaggttcctcaacaggccggtgggccgttagtatcatcaatatcagtattcgcaaaaccagatgaatgattgtttaaactggtgtatttctgcctttatgcttcgtaagtttgctgtcgcgccgtcagtgcccaggctattctggccagcttgtttgccagagcacaggtgacgacaaagttgcttttccgacacaacaactccctgacccagtcggccaacttgccagactggtgttccagtttttgtatgaataccctggcacactgaaccaacaaagttcggatctttttgttgccccgcttgctaatccctaacaatgtcgtccgacctcccgtgctgtactgtcggggtaccagccctgttgccgccgcaaagtcacggctgctggcgtactgcttcccgtcgccaatctcagttgaaatagtactggcagtcagcgttccaacgcagggaatactcagcaagcgctgtccaacctcatcttcgtccaactttcgtttcaactgagattccagatctttaatctgctcaacaagatagtgataatgctgttgtaatttcagcagtaactggctgagataaagaggcaaactactgtcctcaagaagggtactcagtcgactaataacggcagcacctcgcggaacgctgataccaaattccagcagaaaagcatgcatctgattagttgttttcaccttatcctgaaccagggattcacggacacgatgcagagctcgcattgcctgctgagattcggttctgggctgcacgaaacgcatagatggacgtgatgctgcttcacagatagcttcagcatcaacgaagtcatttttgttgcttttaacgaatgggcggacaaattgcggtgatatcagctttggaaaatgccctaactctgccagcttgcgtgccataaagtgagaaccgccacaggcttccatcgcgatggttgttgccgggcatgtcgccagaaattcgattagctttggtcgggtgaattttttacggtaaacggccttcccacgatgatcctgacaatgaatatggaaagagttcttacccagatcgataccaataagcgcaatgttttccatgatggttctccgaatgaaagcctgtcctcagcatagtactgggaaggagggagtgaccatctcattaaataaagcacgctaagccgtaagtaagcgtgctcctgtgaaagccacagctaaaactgcgtagtacacatagaggtatttttttctagatttatatcgaatttcgttatcgaatttcgcttattattgatcaactcgttgcagatcatgaaggcgaaagtatgacgaataaccctaccgatgacagcagaccatggtcggtctttcttatttttctgcggcttggattgacatcttttggcggccccattgcgcacttgggctacttccgcgccgaatttgtcacacggcggcgctggctctccgaacggagctatgctgacttggtcgcgctttgtcagttcttgccagggcctgcaagcagccaggtcggcatagcggtaggactgtctcgggctggatacagcggggcgctggctgcttgggctggcttcacgctgccgtctgccatagccttgatcctttttgcgctcggcatctccagctatggcgattacgtctcgcagggcgcgttgcatggcttaaaagtggtggctgtggccgtggtcgctcaagcagtatggggcatggcgcgtaacctatgcacggatgggctgcgagtcaccatcatggcaattgctacctgcgtcgttttacttgtgccgtccgcgtggggacaggttggcgtgattgctatcgcaggcatcgcaggccggttattgttcaagccagcgaaagttgttgagcatgaccccctacctatcacggtcagtcaccgggccggcgtgctttggctctcgctgttctttgtcttgctgattggcctgccggtgttggccgaactgatgccaagtcaaaccatggcaatggtggattccttctatcgtgtcggatcactggtgttcggcggtggtcacgttgtgctgccattactgcaagccgaagtggtgccctccggctgggtcaacaatgaatcctttctcgcggggtacggggcagctcaagcggtgcccggccctttgttcacgttcgccgcgtttcttggtgcctcgatgaacaccgccccgtcgggctggatcggcggcattgtgtgtctgctggctatcttcgcgccctcgttcttgctggtcgtcggatcaatgccattttgggagcgtttgcgccgcaatacaggcatccaagctgcgctggccgggatcaatgccgctgtagtcggcttgctgctggccgcgctgtatcagcctgtatggactagcgccatctttcagccgcaagacttcggcttggcattagttgcccttgtcgcacttatgttctggaagctcccgccgtggctggttgtcctcggtagcggtgcagctgggtggctgttgagcgtcgctctgtgaggcacaaaaaatgactgacaaagacctctacggcggtttgatccgcctgcacatccttcaccatgcagccgaggaacctgtctttgggctggggatcatcgaagagctacgccgacacggctacgagatgagcgctggcaccgtgtacccgatgctgcacggcctggaaaaaaaaggctatctgacctcacgccacgaacgcaccgggcgacgtgaacggcgtgtttatgacatcactgaacaaggcagaactgcacttgctgatgcgaaaacaaaggtcaaggagctgttcggagagttggtagaaggtggttgagtttttgcttctatgtcaggttgagctataccctaactggattgtcattttcagaagacgactgcaccagttgattgggcgtaatggctgttgtgcagccagctcctgacagttcaatatcagaagtgatctgcaccaatctcgactatgctcaatactcgtgtgggctctgttgcaaaaatcgtgaagcttgagcatgcttggcggagattggacggacggaacgatgacggatttcaagtggcgccatttccagggtgatgtgatcctgtgggcggtgcgctggtattgtcgctatccgatcagctatcgcgaccttgaggaaatgctggcggaacgcggcatttcggtcgaccatacgacgatctatcgctgggtccagtgctacgccccggagatggagaagcggctgcgctggttctggcggcgtggctttgatccgagctggcgcctggatgaaacctacgtcaaggtgcggggcaagtggacctacctgtaccgggcagtcgacaagcggggcgacacgatcgatttctacctgtcgccgacccgcagcgccaaggcagcgaagcggttcctgggcaaggccctgcgaggcctgaagcactgggaaaagcctgccacgctcaataccgacaaagcgccgagctatggtgcagcgatcaccgaattgaagcgcgaaggaaagctggaccgggagacggcccaccggcaggtgaagtatctcaataacgtgatcgaggccgatcacggaaagctcaagatactgatcaagccggtgcgcggtttcaaatcgatccccacggcctatgccacgatcaagggattcgaagtcatgcgagccctgcgcaaaggacaggctcgcccctggtgcctgcagcccggcatcaggggcgaggtgcgccttgtggagagagcttttggcattgggccctcggcgctgacggaggccatgggcatgctcaaccaccatttcgcagcagccgcctgatcggcgcagagcgacagcctacctctgactgccgccaatctttgcaacagagcccgccgtgctagtctgctcggtgatggtggagtgaagccaacccgcaatcgggttatgaatctgcatcgcgattcgcaatcagctgtctcttgagcatgtcgaactcctgcgatgttatctcgccgtgctcgtgctgctacaccaacttgtccaactcatccgccacaccgctccccaccacctgcgatggccaggttgtttgctgggcgcattggcggagtatcgtctcgacccgggacacccacaccggcacaaccttacgggagtgattcactgtcaaagaatcggcccggtgctctgacgcaagtatcgggatggtcaccatttgtaagccgtagacctgagtggtgatcaagacttcgataccaccgaccgtaccggtactaatcgacgacggtcgtgttcgtcgcctgccgcagggactctgcacacctccgtttacgcatgtgcctggaggagttggaaatcgtcgtgttcgggaaacattaaacacaggatggcagcgatctgagccagcacatgatcagctagctcaccatccggatcgacggcccactgcatcgtcgcgccagcgatgaccgagtgcaggagcaactcagctgccgcaggagcacctgggggcagtcgcttgcggatcccctccaccaccgcgcggttccgctggatcgcaagcgtgcgtagctccggcacctggagctcgtaccaggagatgagatagttcaccgagaagtcgttgcgagtgttcatgctccgaacgagcacctgcaaaaattcccagagcccttgcggccctgcgcctatcggtatcgcattcaggtaatgccgcacctgctcgacgccgcgctccatcatcctcaccagcagcgtatcgcggttggtgaagcgctggattaacgctgcgcgggagagccccacctcctttgctactccgctgagcgtgaactctatgggaccgcaacgcttcagcactacggtggcggcctcgagtacctcgtcatcggacttgagcttggggcggggcatcagtgttcaccttctgtatgggttggggcggaggctgtggctgccgccgccattgtagcaaattgaagacggagcgagagtagagccacgagccccgcaaacacggccgatacaacgaggccagggagcggaccagcaaggtcgacaaacgcgccggccgcaagcataaccatgggcgaggctgacagcatcaccgccgagaccgtgccgagtacccggccgagaagttctggcggcgtgcggttgtagatggcagcgttgagaatgggagagactgagccggtcagcagtcccacgagcgcgcccaacaacatcagcaccggcacgcctggcaactgtgaaagcagaagcgagcccaccgcagagccacaaaatgccaccgccagccagttctgcgctgatatccgggcgccgaccgacgcatgaatggcaatgccaaggagaccaccagcccccatcattgaggagaacagcccgagctctgctacttggcgtcctgcatctacaaacagcgcaggcatgatgacgctgccgttggcgccaacgatgcccacgaagatcatcactataccaaagagagggcgcagcaggggttcgctccagagaaaagcgacgccggcgcgcatggagagagtcgccgtcgtggtcatcgtccgagcggcacgcgcgggaagcacccacgcgccgagcagacctgcaaggacggagcagaacgccgtcagcccgagcgttggcgcagcgccaagcaggccgattgcggcccccccaagggccgggccacctagaatcgcgacgttcccgatcaccgctttcagtgacgagacgcgctcaacggagagcccggcgacgtggccgagtttgggcagctcactgtcctgcgcggccataccgggtgcgtcgaacgcggcaccgagcaccacgcaagcgatcagcccagtgttcgagagggcgccaacggcatcgagcagtgggatgctcgccatggccacgccgcccaccacacccgagatcaatgcgacgggcgcgcgcccgaaccgatcgacgaggccaccaccaacccacgcgccgatgatggtcgcgatgacgctgctagcggccgtggcgcccgcccaggccgcgctctttgtatgagacaggacgaaccatggaagcgcgagggccgccaccgcgttgccgatccggaagagaaaggtcgccgcgaacagcgtcgcgagcgggctatatcgacgttcgctcattccgctgcggcgagctgcgccttcgccgcagcgaggtactcttcgttacccgagtcgagggcgaagagtgcgtaggtgaccgccccgaacgcaaggcgctccgcgatgtggtgggcgagccgcggccacacccggccaccggccgcttcatacgtgaggaggagcttcgcgagcccctcttcaccaaagaccataaggtgcgcggccatgtcgatggcagggtcatcaacgcgggcctcgctccagtcgatcatcccgctgacgcgctccgtgttgtcgatgagcacatggcccacgtagagatcgccatgcaccaccacggagaaatctggccacgacgaatcgtcgtcgagccagcgctgccaccggtggaggcgcttgtcgttcaccacgaactcgcgtcggacgcggtcaacgtcgtcggccaccttctgacgggcctgcgtcggtgtacggatgagcatccccgcatccacggcggcggaaatggggacggcatgcagggcggcgagcgcggtcgcgaagctctccgcgaagacctccgagtcctgcggcacgacccagtcgggcgtggacgaaccaggctggatgaccatcgcagtcgagtcttcgagcatgggataggcaacgagctcggcgttggccacgcgccagtccggcaccgcgaacggcaggcgattcttgagcattgccagcacccgcgcctctggttcgaccttcgcgcttacctcggctcggcgcgggatgcgcagcacccaccgacgtccatcgtcgacggtggcgatcacgatcctatagtcgagcccaagctcattgacagtcagcgggccatggagcttgagcccatgtcgggctgcaagtgcgtacagttgggaggtatcggcggtcgtgactacggtcatgattcactcctgagggcttgacgggtttagccacctaaatgtaacagtcacgtcggttatattcaatccggcactgttgcaaagttagcgatgaggcagccttttgtcttattcaaaggccttacatttcaaaaactctgcttaccaggcgcatttcgcccaggggatcaccataataaaatgctgaggcctggcctttgcgtagtgcacgcatcacctcaatacctttgatggtggcgtaagccgtcttcatggatttaaatcccagcgtggcgccgattatccgtttcagtttgccatgatcgcattcaatcacgttgttccggtacttaatctgtcggtgttcaacgtcagacgggcaccggccttcgcgtttgagcagagcaagcgcgcgaccataggcgggcgctttatccgtgttgatgaatcgcgggatctgccacttcttcacgttgttgaggattttacccagaaaccggtatgcagctttgctgttacgacgggaggagatataaaaatcgacagtgcggccccggctgtcgacggcccggtacagatacgcccagcggccattgaccttcacgtaggtttcatccatgtgccacgggcaaagatcggaagggttacgccagtaccagcgcagccgtttttccatttcaggcgcataacgctgaacccagcggtaaatcgtggagtgatcgacattcactccgcgttcagccagcatctcctgcagctcacggtaactgatgccgtatttgcagtaccagcgtacggcccacagaatgatgtcacgctgaaaatgccggcctttgaatgggttcatgtgcagctccatcagcaaaaggggatgataagtttatcaccaccgactatttgcaacagtgccgtttactcatatatactttagattgatttaaaacttcatttttaatttaaaaggatctaggtgaagatcctttttgataatctcatgaccaaaatcccttaacgtgagttttcgttccactgagcgtcagaccccgtatagtgttttgcagtttagaggagatatcgcgatgcatacgcggaaggcaataacggaggcgcttcaaaaactcggagtccaaaccggtgacctcttgatggtgcatgcctcacttaaagcgattggtccggtcgaaggaggagcggagacggtcgttgccgcgttacgctccgcggttgggccgactggcactgtgatgggatacgcgtcgtgggaccgatcaccctacgaggagactctgaatggcgctcggctggatgacgaagcccgccgtacctggctgccgttcgatcccgcaacagccgggacttaccgtgggttcggcctgctgaatcaatttctggttcaagcccccggcgcgcggcgcagcgcgcaccccgatgcatcgatggtcgcggttggtccgctggctgaaacgctgacggagcctcacgaactcggtcacgccttgggggaaggatcgcccgtcgagcggttcgttcgccttggcgggaaggccctgctgttgggtgcgccgctaaactccgttaccgcattgcactacgccgaggcggttgccgatatccccaacaaacggtgggtgacgtatgagatgccgatgcttggaagagacggtgaagtcgcctggaaaacggcatcggattacgattcaaacggcattctcgattgctttgctatcgaaggaaagccggatgcggttgaaactatagcaaatgcttacgtgaagctcggtcgccatcgagaaggtgtcgtgggctttgctcagtgctacctgttcgacgcgcaggacatcgtgacgttcggcgtcacctatcttgagaagcatttcggaaccactccgatcgtgcctccgcacgaggccgtcgagcgctcttgcgagccttcaggttagaggccgtcgacaatgataatctggatcaacggacctttcggcgccggaaagacgacgctcgctaagcggctgcgcgatcggcgttccaaatcgctgatctttgaccccgaggaaatcgggttcgtggtgaaagaaacggtccccatgccagcgagcggagactatcaggatctccccttgtggaggggacttacgatcgcggcggtcagggagattcgaaggaattactcgcaggacatcatcatcccaatgacgctcgtgcacccggactatctgactgagatactcgacggggtaaggcggatcgacgatcagctgctgcacatctttctgacgctcaacgaggacctattgcgtcaccggatcgcgaaccagaccatgcatcctgacccgaatcgaaatgcggagattcgagagtggcgattagcgaatgtcgcccgatgcttggccgcaagggaacggcttccatgcacaacccgtgttctcgatagtggtgcacacaccagcgatgaactcgcagcgatggtgctcgacggaatcgatgggcgcacctgatcgccttcgacgcctgcgcaaagcgtagcgcgagggtggcgggctcacgaccaaacgcccagaggtcgatcatcgcagggatgtttggctttgtggtgcggacgacgggactcgaacccgtactctcacagagaagcagattttcgtaccacctcgactttcgccgccgtctgatgacgttcgtggtctggactgtcccttcgccattgcccgaaggctttaggcgccgcccgtccagtctctacaccttcccccgaaggggcttggctcgggattggcttagggtattgcccgttagcgttccccgactttgagcggttctactccgcggatttccccgcgggcactccaattttaaagtctgctgcgtctaccgatttcgccacgtccgccttttttcgccgttcctagcgctcgtgcgatgcacctatgttgcacctagcgccgaatcgttcttcgtcatcctgaaaaaccacgtctcctaaagccttgcatagcttatcttttctccaccacgaacttttttgtgggatggtagaaaaaaagactttttaagtccgctggcttgccaggccttgttagcttgtacggtcatggttatcgggtaaagaatattgacggcatcgctggtgtcggtggctgaaaagccggctcccatcagggcaatagccatttcagatgcaggcgtacagggcaatggtcaacagctacagcctgtctgacgattccggcgtcatggctgcggcggctatcacgcattttttgttcggtcaggcggtgttttcgtacctcaatggttggagcgtgttgatcggacctggtacaggtttggacagcacgggctgcaaatacgcaagggatttaatgggcctggtggcgttcacggcttttatcgtgacgtttctgttcaggggctactcataatctcgtggctcggcggttcccggcacaccatgacagtaaggaaggaccctgtgtctcaactctcccagcttcgaagccccgccgccgtgcaggctgccatcgatgagttcgtgcaactgggccgcacgaaattcctggcgcgccacggctacggcaagtcccgcgacttcctggtacgtgatccgaagaccggcaccgattgcgattccaaggccatcgccggtgtggccttcggcaagcaatttcccgagcagggcccgctcactgctgacagcttctccggtggcgagacgaccgtcgttccggcgctgacgcggctcgggtttcgcatcattcgcatcggcgaagactggtccgaagaagaggtcctggccacggtcgaagactatttcgacatgctgcgtgccgaggcggctggggagccgtaccacaagtccgagcacaaccaggcactgcgccaactgctgaacggtcgcagcaagtcttcagtcgagctcaagcaccagaacattagcgccgtactcgatgccctgggcctgccctatatcaacggctacaagccacgcggcaacagccaactgctgctgcgtaaatccgtacacgcctacgttctggaacatcagcagacggtcggcgctcttgtcgatgccctggaggaggtaaaacttccgggtgacaaaacctaccgagcggctttggtagaaccacccgcccgtgaagtgcttgtgcgtaccccggcatctctacggcaacgcctaccgcgaaagttcgattatgccgctcgcgatgaagccaaccgcaagctgggccgggcaggggagcagtgggtgattggctacgaacagcaacgcctgaccgagctcggccacccagagctttttcagcggctggattgggtgtccgacacccagggagacggtgcggggttcgacatcctgtcgttcgaagaggacgcccatgagcgcttcatcgaggtgaaaaccaccaatggcggggtaggctcgtctttcttggtcagccacaacgaactcgaattctccaaggaggcgggcgatcaattccatctgtatcgcgtgttccagtttcgggacggtccgcgcctgttcacgctacccggcgacctcagccaacatgtgcatctcaagccgacggactaccgggcgagtttccggagtttggtggggtaaaggcagggttctgttgagccgaatggctgtgtgcggccgattctgttgaaaaagtagcggcctccccatgccgttggcaaaattgctttgtcagcgagcgtgggggcgaacagcatgatgggacagttaccgggaggacagcagcgcctgttctactcgttcaatctggaagatcacgtcccggcccaacatctcctgcgcagcatcgaccagtgcttggatctcagtgatctacgtgcctacctggcagatttctatagccccatcgggcgtccctcgattgacccggagttgatggtgcgcatgctggtcgtcggctactgctatggcattcgttccgagcggcgattgtgcgaagaggtgcacctgaacctggcctatcgctggttctgccggttgggtctggaagacgaagtccccaatcactcgaccttctcgaagaatcgccatgggcgttttcgtgacagcgatctattccgctggttattcaatgaggtgctgcggcgctgcatggcagccggcctagtcaagggtgaaggtttcgccgtcgacgccagcatcattaaggcggatgccagccggcaacgtggggtggcgggagatgaggtcgattggaacgatccaaagctcagcagccgcgcagtgcgcgagtacctcgaagcccttgatgaagaggcgctggctgaggctcttcccaagaaaatttcgctcactgatcctcagtcccgttggacagcagcgccaggtggcccggccttttttgcctactccacgaattacctgatcgacactgagcacggtgtgatcatggacgtggaagctaccccggcgcaccgtaccgccgaagtcgattcgactaggacgatggtcgagcgtgtcgaggcgcagttcgatctcacaccggaacgccttatcggcgataccgcttatggcaccgccccgatgctggcctggatggtcgaagaaaaggacatcgaaccgcatgtgccggtgtgggacaagaccgagcgcaaggacgacagcctctccagtaacgactttcactggagtcaggacgccaatgaatatcgctgcccagccggcaaaccgctacgcagtgaatggcgcgccttcacccagcaaaggtcgcgggtaactaaggccaaaaccgtcatttaccgctccagccaaaccgactgcgccacctgcccgttgaaagcgaaatgctgccccaacacgccgaatcggaagatcgtccgcagcatccatgaggctgcccgcgacgtggctcgacgcatcgccaagacaccggagtacctcgtctctcgctgcgaacgaaagaaggtggagatgcttttcgcccacctcaaacggatcatgaaactcgaccgtttacgactgcgtggcctaacgggtgccactgacgaattcaccttggctgcgatggtgcagaacctgcgccgcatggccaagcttttgcctcaagggccaccgctgacgggataggtatgcctgctacgagcagaaaccctcaaattaacccttaaacctgagcaaggacgctcagtgaaacgccggaaggcaacttgaagtggcttgcagccacttcgacagcaggcacacctgatcggcaggctgccgctaaagctactttttcaacagaatcggccgatttgtgccggtcgtcaaggaccgcttcggatcactctcaaccggatcgcgggccagctgatttgctaaaacccgcgccaaactcaacgaaggatttcgcatgatccgttttttttggcgatcacgctcggcctgatggttggctgctcgtcgagttcaagttcgatctgatcagtgagcggtagcgcaagtggatcgcctcagccaagcagcgttgttgccggcggccggaagaaagccttgaactagtagcggaaaggatgaaactttatttttacgattctcagcattttccaaggttgagcgaaaattaggatgatactttaatgattccggcctccatccccacaaaacgcttgcagatggcagaattgaaataagtcattgttttaaatggatttttgcaggaattgctacgttgcagggtcctgtagttaggatgacactttattttcctcccacaggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctggtaaatgcttcaataatattgaaaaaggaagagtatgagtattcaacattttcgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggtgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgctgccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttatttgagtaaatgcgatttctgccagaagggcggcacaaaaaaacccgactggtgagatcgggtttttatgttttgccccggatggaggcgggaacacctaaacaattgacggttaaatattacgccatgagtgcagtaaatgcaaaccacccggaatagggtatactgcgcgggtcaatctgttgacgttcttctccagcccccgcttacgcggcgtgaacggattaaagccctggttgaaaaacaggtggtgctgtttcaatgccctggctggagtgggaacatctaaacaattgacgtgaggtagccgctgtgacaggcaatgtaagggcgatcagcggtgttcctgctctgtatggagcaaacggatgattaacttgttaatcgtcgttctgcgggctgttgttgcagtgtctaacgctgtgatagcggtcctggagctgatccggcagtattttgactgaccggaaacgtaactaaaggcggggggaccactccccgccttttaacacaaaccttcagtttacagtctgtttagcggccgacacccacttatacagacacccaaccgacaggttcagatcagcagcaattgccttaactttttcccccgttacttttctggcctccatccttttcacggcttcagccctgagcgcttctggtaccactttcactactggcgagtgcttccagatctgatgtgtctgcgctacgtttcgcgcagccactttatgcagcctggtgctggttgcacgttccggggcgtgcttccttccttcagccatgagcgcgagctgttcatcgggattaatctcgcctcgccgtatgcggttcaatacattctctctgcactgacgggaatggctgacgctggcaaggggggtgtgtaacggaatagcccagcgttcgcggtattccctggcgttcatctgatggg